(19) United States
(12) Patent Application Publication
(10) Pub. No.: US 2017/0320837 A1
(43) Pub. Date: Nov. 9, 2017

(54) METHOD FOR TREATING INFLAMMATORY DISORDERS

(71) Applicant: Synta Pharmaceuticals Corp., Lexington, MA (US)

(72) Inventors: Dan Zhou, Lexington, MA (US); Shuzhen Qin, West Roxbury, MA (US); Weiwen Ying, Groton, MA (US)

(21) Appl. No.: 15/465,104

(22) Filed: Mar. 21, 2017

Related U.S. Application Data
(63) Continuation of application No. 14/055,398, filed on Oct. 16, 2013, now abandoned.
Application No. 14/055,398 is a continuation of application No. 12/312,106, filed on May 17, 2010, now abandoned.
(60) Provisional application No. 60/854,675, filed on Oct. 26, 2006.

(51) Int. Cl. C07D 249/12; C07D 403/10; A61K 31/4196

(52) U.S. Cl. CPC C07D 249/12; A61K 31/4196; C07D 403/10

(57) ABSTRACT

The present invention relates to a method for suppressing the immune system in a subject in need thereof, and a method for treating an inflammatory or immune disorder in a subject in need thereof.

### RELATED APPLICATIONS

This application claims the benefit of U.S. Provisional Application No. 60/854,675, filed Oct. 26, 2006, the entire teachings of which are incorporated herein by reference.

### FIELD OF THE INVENTION

The invention relates to a method of modulating glucocorticoid receptors in a subject and methods for immunosuppression and for treating inflammatory and autoimmune disorders. In addition, the invention relates to a method for a method for monitoring the treatment of a patient with an Hsp90 inhibitor, and a method for optimizing dosing for a subject undergoing cancer therapy with an Hsp90 inhibitor.

### BACKGROUND OF THE INVENTION

Heat shock proteins (HSPs) are a class of chaperone proteins that are up-regulated in response to elevated temperature and other environmental stresses, such as ultraviolet light, nutrient deprivation, and oxygen deprivation. HSPs act as chaperones to other cellular proteins (called client proteins) and facilitate their proper folding and repair, and aid in the refolding of misfolded client proteins. There are several known families of HSPs, each having its own set of client proteins. The Hsp90 family is one of the most abundant HSP families, accounting for about 1-2% of proteins in a cell that is not under stress and increasing to about 4-6% in a cell under stress. Inhibition of Hsp90 results in degradation of its client proteins via the ubiquitin proteasome pathway.

Steroidal glucocorticoids are commonly used to treat a variety of inflammatory disorders due to their powerful anti-inflammatory effects. Glucocorticoid receptors (GR) assemble into a multiprotein complex in the cytoplasm in which Hsp90 is required to maintain the receptor in a conformation capable of binding glucocorticoid with high affinity. In humans, the main glucocorticoid is cortisol which binds to the GR complex in the cytoplasm and produces a conformational change in the receptor that results in dissociation of Hsp90 and other proteins in the complex. This allows the receptor to translocate to the nucleus where it can bind to DNA and regulate gene expression, thereby reduce inflammation.

Although glucocorticoids are commonly used to treat inflammatory disorder and are highly efficacious, they have severe side effects. For example, long term use of glucocorticoids can result in osteoporosis, muscle wasting, hypertension, insulin resistance, truncal obesity and fat redistribution, and inhibition of wound repair. Therefore, a need exists for new inflammatory drugs which may reduce some of the side effects of glucocorticoids.

### SUMMARY OF THE INVENTION

The present invention relates to the discovery that treatment of cells, such as peripheral blood mononuclear cells (PMBCs) that have been stimulated with an inflammatory stimulus, such as INFγ/LPS or SAC, with an Hsp90 inhibitor reduce, the expression of GR in the PMBCs and reduce the production of inflammatory cytokines.

In one embodiment, the present invention provides a method of modulating the activity of glucocorticoid receptors in a cell, comprising administering to the cell an effective amount of an Hsp90 inhibitor represented by formula (I):

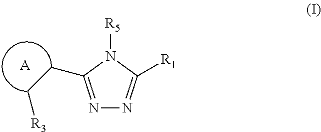

or a tautomer, a pharmaceutically acceptable salt, solvate, clathrate, or prodrugs thereof. In formula (I), ring A is an aryl or a heteroaryl, wherein the aryl or the heteroaryl are optionally further substituted with one or more substituents in addition to R₃;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR,₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;

R₅ is an optionally substituted heteroaryl or an optionally substituted 8 to 14 membered aryl;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₂₆ is a lower alkyl;

p, for each occurrence, is, independently, 0, 1 or 2; and

m, for each occurrence, is independently, 1, 2, 3, or 4.

In one embodiment, ring A of the the compounds of formula (I) is not a substituted [1,2,3]triazole, and/or compounds represented by formula (I) do not include 3-(2,4-dihydroxy-phenyl)-4-(7-naphthalen-1-yl)-5-mercapto-triazole.

In another embodiment, the present invention provides a method of modulating the activity of glucocorticoid receptors in a cell, comprising administering to the cell an effective amount of an Hsp90 inhibitor represented by formula (II):

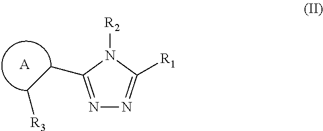

or a tautomer, a pharmaceutically acceptable salt, solvate, clathrate, or prodrug thereof. In formula (II), ring A, R₁, and R₃ are defined as for formula (I); and

R₂ is a substituted phenyl, wherein the phenyl group is substituted with:
i) one substituent selected from nitro, cyano, a haloalkoxy, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxylalkyl, alkoxyalkyl, guanadino, —NR₁₀R₁₁, —O—R₂₀, —C(O)R₇, —C(O)OR₂₀, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁, or
ii) two to five substituents selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, —F, —Br, —I, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; and

R₂₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.

In one embodiment, compounds represented by formula (II) do not include 3-(2,4-dihydroxy-phenyl)-4-(7-naphthalen-1-yl)-5-mercapto-triazole, 3-(2,4-dihydroxyphenyl)-4-(2,5-dimethoxyphenyl)-5-mercapto-triazole, 3-(1-phenyl-5-amino-pyrazol-4-yl)-4-(2,4-dichloropheny)-5-mercapto-triazole, or 3-(2-hydroxy-phenyl)4-(2,4-dimethylphenyl)-5-mercapto-triazole.

In another embodiment, the present invention provides a method of modulating the activity of glucocorticoid receptors in a cell, comprising administering to the cell an effective amount of an Hsp90 inhibitor represented by formula (III):

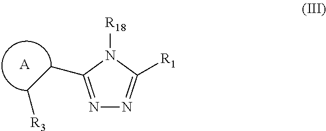

or a tautomer, a pharmaceutically acceptable salt, solvate, clathrate, and prodrug thereof. In formula (III), ring A, R₁, and R₃ are defined as for formula (I); and

R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

In one embodiment, compounds represented by formula (III) do not include compounds in which R₁₈ is not cyclohexyl.

In another embodiment, the present invention provides a method of modulating the activity of glucocorticoid receptors in a cell, comprising administering to the cell an effective amount of an Hsp90 inhibitor represented by formula (IV) or formula (V):

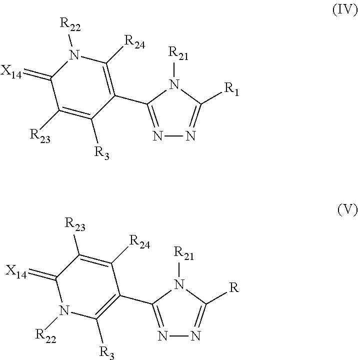

or a tautomer, a pharmaceutically acceptable salt, solvate, clathrate, or prodrug thereof. In formulas (IV) and (V), R₁ and R₃ are defined as for formula (I); and

X₁₄ is O, S, or NR₇;

R₂₁ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₂₂, for each occurrence, is independently a substituent selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, a haloalkyl, —C(O)R₇, —C(O)OR₇, —OC(O)R₇, —C(O)NR₁₀R₁₁, —NR₈C(O)R₇, —S(O)ₚR₇, —S(O)ₚOR₇, or —S(O)ₚNR₁₀R₁₁; and

R₂₃ and R₂₄, for each occurrence, are independently a substituent selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁.

In another embodiment, the present invention provides a method of modulating the activity of glucocorticoid receptors in a cell, comprising administering to the cell an effective amount of an Hsp90 inhibitor represented by formula (VI):

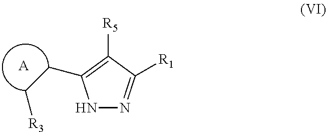

or a tautomer, a pharmaceutically acceptable salt, solvate, clathrate, or a prodrug thereof. In formula (VI):

ring A is an aryl or a heteroaryl, wherein the aryl or the heteroaryl are optionally further substituted with one or more substituents in addition to R₃;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;

R₅ is an optionally substituted heteroaryl or an optionally substituted 8 to 14-membered aryl;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₂₆ is a lower alkyl;

p, for each occurrence, is, independently, 0, 1 or 2; and

m, for each occurrence, is independently, 1, 2, 3, or 4.

In one embodiment, the present invention provides a method of modulating the activity of glucocorticoid receptors in a cell, comprising administering to the cell an effective amount of an Hsp90 inhibitor represented by formula (VII):

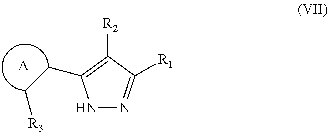

or a tautomer, a pharmaceutically acceptable salt, solvate, clathrate, or a prodrug thereof. In formula (VII), R₂′ is an optionally substituted phenyl group. Preferably, R₂′ is substituted with one or more group represented by R₃₀, wherein R₃₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. The remainder of the variables in structural formula (VII) have values defined above with reference to structural formula (VI).

In one embodiment, the present invention provides a method of modulating the activity of glucocorticoid receptors in a cell, comprising administering to the cell an effective amount of an Hsp90 inhibitor represented by formula (VIII):

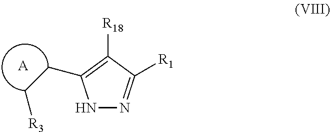

or a tautomer, a pharmaceutically acceptable salt, solvate, clathrate, or a prodrug thereof. In formula (VIII), R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁. The remainder of the variables in structural formula (VIII) have values defined above with reference to structural formula (VI).

In one embodiment, the present invention provides a method of modulating the activity of glucocorticoid receptors in a cell, comprising administering to the cell an effective amount of an Hsp90 inhibitor represented by formula (IX):

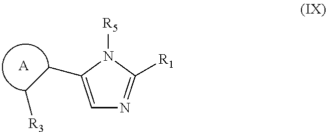

or a tautomer, pharmaceutically acceptable salt, solvate, clathrate, or a prodrug thereof. In formula (IX):

ring A is an aryl or a heteroaryl, wherein the aryl or the heteroaryl are optionally further substituted with one or more substituents in addition to R₃;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₂, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;

R₅ is an optionally substituted heteroaryl or an optionally substituted 8 to 14-membered aryl;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₂₆ is a lower alkyl;

p, for each occurrence, is, independently, 0, 1 or 2; and

m, for each occurrence, is independently, 1, 2, 3, or 4.

In one embodiment, the present invention provides a method of modulating the activity of glucocorticoid receptors in a cell, comprising administering to the cell an effective amount of an Hsp90 inhibitor represented by formula (X):

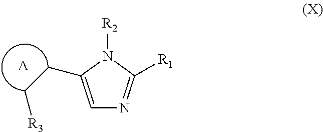

or a tautomer, pharmaceutically acceptable salt, solvate, clathrate, or a prodrug thereof. In formula (X), R₂′ is an optionally substituted phenyl group. Preferably, R₂′ is substituted with one or more group represented by R₃₀, wherein R₃₀, for each occurrence, are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. The remainder of the variables in structural formula (X) have values defined above with reference to structural formula (IX).

In one embodiment, the present invention provides a method of modulating the activity of glucocorticoid receptors in a cell, comprising administering to the cell an effective amount of an Hsp90 inhibitor represented by formula (XI):

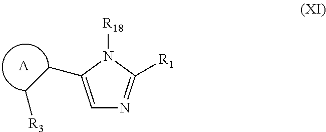

or a tautomer, pharmaceutically acceptable salt, solvate, clathrate, or a prodrug thereof. In formula (XI), R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁. The remainder of the variables in structural formula (XI) have values defined above with reference to structural formula (IX).

In another embodiment, the present invention is a method of treating an inflammatory disorder in a subject in need thereof, comprising administering a compound represented by any one of the formulas disclosed herein. In one embodiment, the inflammatory disorder is selected from the group consisting of transplant rejection, skin graft rejection, arthritis, rheumatoid arthritis, osteoarthritis and bone diseases associated with increased bone resorption; inflammatory bowel disease, ileitis, ulcerative colitis, Barrett's syndrome, Crohn's disease; asthma, adult respiratory distress syndrome, chronic obstructive airway disease; corneal dystrophy, trachoma, onchocerciasis, uveitis, sympathetic ophthalmitis, endophthalmitis; gingivitis, periodontitis; tuberculosis; leprosy; uremic complications, glomerulonephritis, nephrosis; sclerodermatitis, psoriasis, eczema; chronic demyelinating diseases of the nervous system, multiple sclerosis, AIDS-related neurodegeneration, Alzheimer's disease, infectious meningitis, encephalomyelitis, Parkinson's disease, Huntington's disease, amyotrophic lateral sclerosis viral or autoimmune encephalitis; autoimmune disorders, immune-complex vasculitis, systemic lupus and erythematodes; systemic lupus erythematosus (SLE); cardiomyopathy, ischemic heart disease hypercholesterolemia, atherosclerosis, preeclampsia; chronic liver failure, brain and spinal cord trauma.

In another embodiment, the present invention is a method of treating an immune disorder in a subject in need thereof, comprising administering a compound represented by any one of the formulas disclosed herein. In one embodiment, the immune disorder is selected from the group consisting of multiple sclerosis, myasthenia gravis, Guillain-Barré, autoimmune uveitis, autoimmune hemolytic anemia, pernicious anemia, autoimmune thrombocytopenia, temporal arteritis, anti-phospholipid syndrome, vasculitides such as Wegener's granulomatosis, Behcet's disease, psoriasis, dermatitis herpetiformis, pemphigus vulgaris, vitiligo, Crohn's disease, ulcerative colitis, primary biliary cirrhosis, autoimmune hepatitis, Type 1 or immune-mediated diabetes mellitus, Grave's disease. Hashimoto's thyroiditis, autoimmune oophoritis and orchitis, autoimmune disorder of the adrenal gland, rheumatoid arthritis, systemic lupus erythematosus, scleroderma, polymyositis, dermatomyositis, ankylosing spondylitis, and Sjogren's syndrome.

In another embodiment, the present invention is a method of suppressing an immune response in a subject in need thereof, comprising administering a compound represented by any one of the formulas disclosed herein, or a tautomer, pharmaceutically acceptable salt, solvate, clathrate, or a prodrug thereof. In one embodiment, the subject in need of immunosuppression is a subject that has received an organ or tissue transplant, such as a skin graft, heart, kidney, lung, liver, pancreas, cornea, bowel, stomach, and the like. In another embodiment, the subject in need of immunosuppression is a subject that has received stem cell transplantation. The transplant may be a syngeneic transplant (i.e., from a donor that has the same genetic make up), an allographic transplant (i.e., from a donor of the same species) or a xenographic transplant (i.e., from a donor that is a different species).

In another embodiment, the present invention is a method of inhibiting the production of inflammatory cytokines, such as G-CSF, GM-CSF, IL-12, IL-1β, IL-23, IL-6, IL-8, and TNF-α, in a subject in need of such treatment. The method comprises administering to the subject an effective amount of a compound represented by any one of the formulas disclosed herein.

In another embodiment of the present invention is a pharmaceutical composition comprising a compound represented by any one of the formulas disclosed herein and a pharmaceutically acceptable carrier. The pharmaceutical compositions can be used in therapy, e.g., to treat a mammal with an inflammatory or immune disorder. In one embodiment, the pharmaceutical composition includes one or more additional therapeutic agent, such as one or more additional anti-inflammatory agent or one or more immunosuppressant.

In another embodiment, the present invention is the use of a compound of anyone of the formulas disclosed herein for the manufacture of a medicament for treating a mammal with an inflammatory or autoimmune disorder or for treatment of a mammal in need of immunosuppression.

In another embodiment, the invention relates to a method for monitoring the treatment of a patient who will be treated with, who is currently being treated with, or who has been treated with an Hsp90 inhibitor, comprising the steps of:
a) obtaining a sample of peripheral blood mononuclear cells (PBMCs) from a patient before administering a dosage of an Hsp90 inhibitor;
b) obtaining a sample of PBMCs from the patient after administering a dosage of an Hsp90 inhibitor;
c) determining the amount of glucocorticoid receptor protein in the PBMCs of the patient before receiving the dosage;
d) determining the amount of glucocorticoid receptor protein in the PBMCs of the patient after receiving the dosage; and
e) comparing the amount of glucocorticoid receptor protein in the PBMCs before receiving the dosage with the amount of glucocorticoid receptor protein after receiving the dosage, wherein the greater a decreased in glucocorticoid receptor protein after receiving the dosage indicates greater activity of the Hsp90 inhibitor.

In another embodiment, the invention relates to a method for optimizing dosing for a subject undergoing cancer therapy, wherein the dosing includes administration of an Hsp90 inhibitor, comprising the steps of:
a) changing dosing of the Hsp90 inhibitor during therapy;
b) comparing the amount of glucocorticoid receptor protein in PBMCs in a control sample with the amount of glucocorticoid receptor protein in PBMCs in a test sample;
c) comparing side effects from the Hsp90 inhibitor between the test sample and the control sample;
d) optimizing dosing of the Hsp90 inhibitor based on the dosing in step a) in combination with the results of step b) or step c), wherein the test sample is taken from the subject after changing the dosing; and the control sample is taken from the subject before changing the dosing.

The compounds shown in Tables 5, 6, and 7, or compounds of any formula herein, or tautomers, pharmaceutically acceptable salts, solvates, clathrates, hydrates, polymorphs or prodrugs thereof, inhibit the activity of Hsp90 and, thereby cause the degradation of Hsp90 client proteins. The glucocorticoid receptor is a client protein of Hsp90 and binds to Hsp90 when it is in the conformation that is able to bind glucocorticoid ligands such as cortisol. Once a glucocorticoid binds to GR, the receptor disassociates with Hsp90 and translocates to the nucleus where it modulates gene expression to reduce inflammatory responses such as proinflammatory cytokine production. Thus, glucocorticoids are given to patients in need of immunosuppression and patients with inflammatory and autoimmune disorders. Unfortunately, although glucocorticoids are effective at relieving inflammation, they have a number of sever side effects including osteoporosis, muscle wasting, hypertension, insulin resistance, truncal obesity and fat redistribution, and inhibition of wound repair. Inhibition of Hsp90 causes changes in GR activity which results in reduction of inflammatory responses similar to those seen for glucocorticoids. However, since the mechanism for reducing inflammation is different than that of glucocorticoids, it is expected that some or all of the side effects of glucocorticoid treatment will be reduced or eliminated.

### BRIEF DESCRIPTION OF THE FIGURES

FIG. 1 is a graph showing the ATPase activity of Hsp90 when untreated, when treated with 40 mM of Geldanamycin (a known Hsp90 inhibitor as a positive control), and when treated with 40 μM or 4 μM of Compound 108 of the invention.

FIG. 2 is gel showing the amount of Her2, an Hsp90 client protein, in cells that are untreated, in cells that have been treated with 0.5 μM, 2 μM, or 5 μM of 17AAG, a known Hsp90 inhibitor, and in cells that have been treated with 0.5 μM, 2 μM, or 5 μM of Compound 108 or Compound 49.

FIG. 3 is a graph of the results of a competitive binding assay in which Hela S3 cells are incubated with tritium labeled dexamethason which binds to glucocorticoid receptors. The cells were then incubated with Compound 226, paclitaxel, radicicol, or cold dexamethason. Displacement of tritium labeled dexamethason indicates that a compound binds to the glucocorticoid receptor. Compound 226, paclitaxel, and radicicol did not displace the tritium labeled dexamethason and therefore do not bind directly to the glucocorticoid receptor.

FIGS. 4A-I are graphs of a first experiment showing the percent inhibition of CCK-8, G-CSF, GM-CSF, IL-10, IL-12(p70), IL-1β, IL-6, IL-8, and TNFα in human PBMCs that have been stimulated with lipopolysaccharide (LPS) followed by treatment with dexamethason.

FIGS. 5A-I are graphs of a second experiment showing the percent inhibition of CCK-8, G-CSF, GM-CSF, IL-10, IL-12(p70), IL-1β, IL-6, IL-8, and TNFα in human PBMCs that have been stimulated with LPS followed by treatment with dexamethason.

FIGS. 6A-I are graphs of a first experiment showing the percent inhibition of CCK-8, G-CSF, GM-CSF, IL-10, IL-12(p70), IL-1β, IL-6, IL-8, and TNFα in human PBMCs that have been stimulated with Staphylococcus Aureus Cowan (SAC) followed by treatment with dexamethason.

FIGS. 7A-I are graphs of a second experiment showing the percent inhibition of CCK-8, G-CSF, GM-CSF, IL-10, IL-12(p70), IL-1β, IL-6, IL-8, and TNFα in human PBMCs that have been stimulated with SAC followed by treatment with dexamethason.

FIGS. 8A-I are graphs showing the percent inhibition of CCK-8, G-CSF, GM-CSF, IL-10, IL-12(p70), IL-1β, IL-6, IL-8, and TNFα in human PBMCs that have been stimulated with LPS followed by treatment with 17-DMAG.

FIGS. 9A-I are graphs of a first experiment showing the percent inhibition of CCK-8, G-CSF, GM-CSF, IL-10, IL-12(p70), IL-1β, IL-6, IL-8, and TNFα in human PBMCs that have been stimulated with SAC followed by treatment with 17-DMAG.

FIGS. 10A-I are graphs of a second experiment showing the percent inhibition of CCK-8, G-CSF, GM-CSF, IL-10, IL-12(p70), IL-1β, IL-6, IL-8, and TNFα in human PBMCs that have been stimulated with SAC followed by treatment with 17-DMAG.

FIGS. 11A-I are graphs of a first experiment showing the percent inhibition of CCK-8, G-CSF, GM-CSF, IL-10, IL-12(p70), IL-1β, IL-6, IL-8, and TNFα in human PBMCs that have been stimulated with LPS followed by treatment with Compound 226.

FIGS. 12A-I are graphs of a second experiment showing the percent inhibition of CCK-8, G-CSF, GM-CSF, IL-10, IL-12(p70), IL-1β, IL-6, IL-8, and TNFα in human PBMCs that have been stimulated with LPS followed by treatment with Compound 226.

FIGS. 13A-I are graphs of a first experiment showing the percent inhibition of CCK-8, G-CSF, GM-CSF, IL-10, IL-12(p70), IL-1β, IL-6, IL-8, and TNFα in human PBMCs that have been stimulated with SAC followed by treatment with Compound 226.

FIGS. 14A-I are graphs of a second experiment showing the percent inhibition of CCK-8, G-CSF, GM-CSF, IL-10, IL-12(p70), IL-1β, IL-6, IL-8, and TNFα in human PBMCs that have been stimulated with SAC followed by treatment with Compound 226.

FIG. 15A is an immunoblot showing a dose dependent decrease in the expression of glucocorticoid receptors in rat PBMCs treated for 16 hrs with 17-DMAG, Compound 226 or Compound 287.

FIG. 15B is an immunoblot showing a dose dependent decrease in the expression of glucocorticoid receptors in human PBMCs treated for 16 hrs with 17-DMAG, Compound 226 or Compound 287.

FIG. 16A is an immunoblot showing a dose dependent decrease in the expression of glucocorticoid receptors in Kasumi-1 AML cancer cells treated for 16 hrs with 17-DMAG, Compound 226 or Compound 287.

FIG. 16B is an immunoblot showing a dose dependent decrease in the expression of glucocorticoid receptors in MV-4-11 AML cancer cells treated for 16 hrs with 17-DMAG, Compound 226 or Compound 287.

FIG. 16C is an immunoblot showing a dose dependent decrease in the expression of glucocorticoid receptors in rat HeLa cells treated for 16 hrs with 17-DMAG or Compound 226.

FIG. 16D is an immunoblot showing a dose dependent decrease in the expression of glucocorticoid receptors in human PBMCs treated for 16 hrs with 17-DMAG, Compound 226 or Compound 287.

FIG. 16E is an immunoblot showing a dose dependent decrease in the expression of glucocorticoid receptors in human renal cells treated for 16 hrs with 17-DMAG, Compound 226 or Compound 287.

FIG. 17 is an immunoblot showing the effects on rat PBMCs of 4 days of repeat dosing of vehicle, 17-DMAG, Paclitaxel, Compound 264 and Compound 226.

### DETAILED DESCRIPTION OF THE INVENTION

The present invention provides compounds that inhibit Hsp90 activity which can be used for immunosuppression and for the treatment of inflammatory and immune disorders. The present invention also provides a method for monitoring the treatment of a patient that has been, will be, or is currently being treated with an Hsp90 inhibitor and a method for optimizing dosing for a subject undergoing cancer therapy, wherein the dosing includes administration of an Hsp90 inhibitor.

A. Terminology

Unless otherwise specified, the below terms used herein are defined as follows:

As used herein, the term “alkyl” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls,include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. The term “(C₁-C₆)alkyl” means a saturated straight chain or branched non-cyclic hydrocarbon having from 1 to 6 carbon atoms. Representative (C₁-C₆)alkyl groups are those shown above having from 1 to 6 carbon atoms. Alkyl groups included in compounds of this invention may be optionally substituted with one or more substituents.

As used herein, the term “alkenyl” means a saturated straight chain or branched non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Alkenyl groups may be optionally substituted with one or more substituents.

As used herein, the term “alkynyl” means a saturated straight chain or branched non-cyclic hydrocarbon having from 2 to 10 carbon atoms and having at lease one carbon-carbon triple bond. Representative straight chain and branched alkynyls include acetylenyl, propynyl, 1-butynyl, 2-butynyl, 1-pentynyl, 2-pentynyl, 3-methyl-l-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups may be optionally substituted with one or more substituents.

As used herein, the term “cycloalkyl” means a saturated, mono- or polycyclic alkyl radical having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, -cyclodecyl, octahydro-pentalenyl, and the like. Cycloalkyl groups may be optionally substituted with one or more substituents.

As used herein, the term “cycloalkenyl” means a mono- or poly-cyclic non-aromatic alkyl radical having at least one carbon-carbon double bond in the cyclic system and from 3 to 20 carbon atoms. Representative cycloalkenyls include cyclopentenyl, cyclopentadienyl, cyclohexenyl, cyclohexadienyl,cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl and the like. Cycloalkenyl groups may be optionally substituted with one or more substituents.

As used herein, the term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker.

As used herein, an “haloalkoxy” is an haloalkyl group which is attached to another moiety via an oxygen linker.

As used herein, the term an “aromatic ring” or “aryl” means a hydrocarbon monocyclic or polycyclic radical in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be optionally substituted with one or more substituents.

As used herein, the term “heterocyclyl” means a monocyclic (typically having 3- to 10-members) or a polycyclic (typically having 7- to 20-members) heterocyclic ring system which is either a saturated ring or a unsaturated non-aromatic ring. A 3- to 10-membered heterocycle can contain up to 5 heteroatoms; and a 7- to 20-membered heterocycle can contain up to 7 heteroatoms. Typically, a heterocycle has at least on carbon atom ring member. Each heteroatom is independently selected from nitrogen, which can be oxidized (e.g., N(O)) or quaternized; oxygen; and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents. Only stable isomers of such substituted heterocyclic groups are contemplated in this definition.

As used herein, the term “heteroaromatic”, “heteroaryl” or like terms means a monocyclic or polycyclic heteroaromatic ring comprising carbon atom ring members and one or more heteroatom ring members. Each heteroatom is independently selected from nitrogen, which can be oxidized (e.g., N(O)) or quaternized; oxygen; and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings. Heteroaryl groups may be optionally substituted with one or more substituents.

As used herein, the term “(C₅)heteroaryl” means an aromatic heterocyclic ring of 5 members, wherein at least one carbon atom of the ring is replaced with a heteroatom such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like.

As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl and the like. Heteroaralkyl groups may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include any substituent which will form a stable compound of the invention. Examples of substituents for an alkyl, alkylene, alkenyl, alkynyl, cycloalkyl, cycloalkenyl, heterocyclyl, aryl, aralkyl, heteroaryl, and heteroarylalkyl include an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, a haloalkyl, —C(O)NR₂₈R₂₉, —C(S)NR₂₈R₂₉, —C(NR₃₂)NR₂₈R₂₉, —NR₃₀C(O)R₃₁, —NR₃₀C(S)R₃₁, —NR₃₀C(NR₃₂)R₃₁, halo, —OR₃₀, cyano, nitro, haloalkoxy, —C(O)R₃₀, —C(S)R₃₀, —C(NR₃₂)R₃₀, —NR₂₈R₂₉, —C(O)OR₃₀, —C(S)OR₃₀, —C(NR₃₂)OR₃₀, —OC(O)R₃₀, —OC(S)R₃₀, —OC(NR₃₂)R₃₀, —NR₃₀C(O)NR₂₈R₂₉, —NR₃₀C(S)NR₂₈R₂₉, —NR₃₀C(NR₃₂)NR₂₈R₂₉, —OC(O)NR₂₈R₂₉, —OC(S)NR₂₈R₂₉, —OC(NR₃₂)NR₂₈R₂₉, —NR₃₀C(O)OR₃₁, —NR₃₀C(S)OR₃₁, —NR₃₀C(NR₃₂)OR₃₁, —S(O)ₕR₃₀, —OS(O)ₚR₃₀, —NR₃₀S(O)ₚR₃₀, —S(O)ₚNR₂₈R₂₉, —OS(O)ₚNR₂₈R₂₉, or —NR₃₀S(O)ₚNR₂₈R₂₉, wherein R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocyclyl or optionally substituted heteroaryl.

R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; and

R₃₂, for each occurrence is, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, —C(O)R₃₀, —C(O)NR₂₈R₂₉, —S(O)ₚR₃₀, or —S(O)ₚNR₂₈R₂₉; and

h is 0, 1 or 2.

In addition, alkyl, cycloalkyl, alkylene, a heterocyclyl, and any saturated portion of a alkenyl, cycloalkenyl, alkynyl, aralkyl, and heteroaralkyl groups, may also be substituted with ═O, ═S, ═N—R₃₂.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms, respectively.

Unless indicated otherwise, the compounds of the invention containing reactive functional groups (such as (without limitation) carboxy, hydroxy, thiol, and amino moieties) also include protected derivatives thereof. “Protected derivatives” are those compounds in which a reactive site or sites are blocked with one ore more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. Greene, Protecting Groups in Organic Synthesis, John Wiley & Sons, Inc. 1981.

As used herein, the term “compound(s) of this invention” and similar terms refers to a compound of formula (I) through (LXXII) and Tables 5, 6, and 7, or a tautomer, a pharmaceutically acceptable salt, solvate, clathrate, hydrate, polymorph or prodrug thereof, and also include protected derivatives thereof.

The compounds of the invention may contain one or more chiral centers and/or double bonds and, therefore, exist as stereoisomers, such as double-bond isomers (i.e., geometric isomers), enantiomers, or diastereomers. According to this invention, the chemical structures depicted herein, including the compounds of this invention, encompass all of the corresponding compounds' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds of this invention are preferred.

As used herein, the term “pharmaceutically acceptable salt,” is a salt formed from, for example, an acid and a basic group of one of the compounds of formula (I) through (LXXII) and Tables 5, 6, and 7. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate; besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of formula (I) through (LXXII) and Tables 5, 6, and 7 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formula (I) through (LXXII) and Tables 5, 6, and 7 having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

As used herein, the term “pharmaceutically acceptable solvate,” is a solvate formed from the association of one or more pharmaceutically acceptable solvent molecules to one of the compounds of formula (I) through (LXXII) and Tables 5, 6, and 7. The term solvate includes hydrates (e.g., hemihydrate, monohydrate, dihydrate, trihydrate, tetrahydrate, and the like).

A pharmaceutically acceptable carrier may contain inert ingredients which do not unduly inhibit the biological activity of the compounds. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, that further includes a stoichiometric or non-stoichiometric amount of water bound by non-covalent intermolecular forces.

As used herein, he term “clathrate” means a compound of the present invention or a salt thereof in the form of a crystal lattice that contains spaces (e.g., channels) that have a guest molecule (e.g., a solvent or water) trapped within.

As used herein and unless otherwise indicated, the term “prodrug” means a derivative of a compound that can hydrolyze, oxidize, or otherwise react under biological conditions (in vitro or in vivo) to provide a compound of this invention. Prodrugs may become active upon such reaction under biological conditions, or they may have activity in their unreacted forms. Examples of prodrugs contemplated in this invention include, but are not limited to, analogs or derivatives of compounds of formula (I) through (LXXII) and Tables 5, 6, and 7 that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of formula (I) through (LXXII), and Tables 5, 6, and 7, that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as improved water solubility, improved circulating half-life in the blood (e.g., because of reduced metabolism of the prodrug), improved uptake, improved duration of action, or improved onset of action; or 2) is itself biologically inactive but is converted in vivo to a biologically active compound. Examples of biohydrolyzable amides include, but are not limited to, lower alkyl amides, a-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, “Hsp90” includes each member of the family of heat shock proteins having a mass of about 90-kiloDaltons. For example, in humans the highly conserved Hsp90 family includes cytosolic Hsp90α and Hsp90β isoforms, as well as GRP94, which is found in the endoplasmic reticulum, and HSP75/TRAP1, which is found in the mitochondrial matrix.

The glucocorticoid receptor is a member of the steroid hormone nuclear receptor family which includes glucocorticoid receptors (GR), androgen receptors (AR), mineralocorticoid receptors (MR), estrogen receptors (ER), and progesterone receptors (PR). Glucocorticoid receptors bind glucocorticoids such as cortisol, corticosterone, and cortisone.

“Immunosuppression” refers to impairment of any component of the immune system resulting in decreased immune function. This impairment may be measured by any conventional means including whole blood assays of lymphocyte function, detection of lymphocyte proliferation and assessment of the expression of T cell surface antigens. The antisheep red blood cell (SRBC) primary (IgM) antibody response assay (usually referred to as the plaque assay) is one specific method. This and other methods are described in Luster, M. I., Portier, C., Pait, D. G., White, K. L., Jr., Gennings, C., Munson, A. E., and Rosenthal, G. J. (1992). “Risk Assessment in Immunotoxicology I: Sensitivity and Predictability of Immune Tests.” Fundam. Appl. Toxicol., 18, 200-210. Measuring the immune response to a T-cell dependent immunogen is another particularly useful assay (Dean, J. H., House, R. V., and Luster, M. I. (2001). “Immunotoxicology: Effects of, and Responses to, Drugs and Chemicals.” In Principles and Methods of Toxicology: Fourth Edition (A. W. Hayes, Ed.), pp. 1415-1450, Taylor & Francis, Philadelphia, Penn.). In one embodiment, a decrease in the expression of glucocorticoid receptors in PBMCs indicates impairment of immune function. A patient in need of immunosuppression is within the judgement of a physician, and can include patients with immune or inflammatory disorders. In one embodiment, patients that have undergone or will be undergoing an organ, tissue, bone marrow, or stem cell transplantation are in need of immunosuppression to prevent inflammation and/or rejection of the transplanted organ or tissue.

The compounds of this invention can be used to treat subjects with immune disorders. As used herein, the term “immune disorder” and like terms means a disease, disorder or condition caused by the immune system of an animal, including autoimmune disorders. Immune disorders include those diseases, disorders or conditions that have an immune component and those that are substantially or entirely immune system-mediated. Autoimmune disorders are those wherein the animal's own immune system mistakenly attacks itself, thereby targeting the cells, tissues, and/or organs of the animal's own body. For example, the autoimmune reaction is directed against the nervous system in multiple sclerosis and the gut in Crohn's disease. In other autoimmune disorders such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus. Specific autoimmune disorders that may be ameliorated using the compounds and methods of this invention include without limitation, autoimmune disorders of the nervous system (e.g., multiple sclerosis, myasthenia gravis, autoimmune neuropathies such as Guillain-Barré, and autoimmune uveitis), autoimmune disorders of the blood (e.g., autoimmune hemolytic anemia, pernicious anemia, and autoimmune thrombocytopenia), autoimmune disorders of the blood vessels (e.g., temporal arteritis, anti-phospholipid syndrome, vasculitides such as Wegener's granulomatosis, and Behcet's disease), autoimmune disorders of the skin (e.g., psoriasis, dermatitis herpetiformis, pemphigus vulgaris, and vitiligo), autoimmune disorders of the gastrointestinal system (e.g., Crohn's disease, ulcerative colitis, primary biliary cirrhosis, and autoimmune hepatitis), autoimmune disorders of the endocrine glands (e.g., Type 1 or immune-mediated diabetes mellitus, Grave's disease. Hashimoto's thyroiditis, autoimmune oophoritis and orchitis, and autoimmune disorder of the adrenal gland); and autoimmune disorders of multiple organs (including connective tissue and musculoskeletal system diseases) (e.g., rheumatoid arthritis, systemic lupus erythematosus, scleroderma, polymyositis, dermatomyositis, spondyloarthropathies such as ankylosing spondylitis, and Sjogren's syndrome). In addition, other immune system mediated diseases, such as graft-versus-host disease and allergic disorders, are also included in the definition of immune disorders herein. Because a number of immune disorders are caused by inflammation, there is some overlap between disorders that are considered immune disorders and inflammatory disorders. For the purpose of this invention, in the case of such an overlapping disorder, it may be considered either an immune disorder or an inflammatory disorder. “Treatment of an immune disorder” herein refers to administering a compound represented by any of the formulas disclosed herein to a subject, who has an immune disorder, a symptom of such a disease or a predisposition towards such a disease, with the purpose to cure, relieve, alter, affect, or prevent the autoimmune disorder, the symptom of it, or the predisposition towards it.

As used herein, the term “allergic disorder” means a disease, condition or disorder associated with an allergic response against normally innocuous substances. These substances may be found in the environment (such as indoor air pollutants and aeroallergens) or they may be non-environmental (such as those causing dermatological or food allergies). Allergens can enter the body through a number of routes, including by inhalation, ingestion, contact with the skin or injection (including by insect sting). Many allergic disorders are linked to atopy, a predisposition to generate the allergic antibody IgE. Because IgE is able to sensitize mast cells anywhere in the body, atopic individuals often express disease in more than one organ. For the purpose of this invention, allergic disorders include any hypersensitivity that occurs upon re-exposure to the sensitizing allergen, which in turn causes the release of inflammatory mediators. Allergic disorders include without limitation, allergic rhinitis (e.g., hay fever), sinusitis, rhinosinusitis, chronic or recurrent otitis media, drug reactions, insect sting reactions, latex reactions, conjunctivitis, urticaria, anaphylaxis and anaphylactoid reactions, atopic dermatitis, asthma and food allergies.

As used herein, the term “asthma” means a pulmonary disease, disorder or condition characterized by reversible airway obstruction, airway inflammation, and increased airway responsiveness to a variety of stimuli.

Compounds represented by any of the formulas disclosed herein can be used to prevent or to treat subjects with inflammatory disorders. As used herein, an “inflammatory disorder” means a disease, disorder or condition characterized by inflammation of body tissue or having an inflammatory component. These include local inflammatory responses and systemic inflammation. Examples of such inflammatory disorders include: transplant rejection, including skin graft rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune disorders, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis; as well as various other diseases with significant inflammatory components, including preeclampsia; chronic liver failure, brain and spinal cord trauma. There may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy. “Treatment of an inflammatory disorder” herein refers to administering a compound or a composition of the invention to a subject, who has an inflammatory disorder, a symptom of such a disorder or a predisposition towards such a disorder, with the purpose to cure, relieve, alter, affect, or prevent the inflammatory disorder, the symptom of it, or the predisposition towards it.

An “effective amount” is the quantity of compound in which a beneficial outcome is achieved when the compound is administered to a subject or alternatively, the quantity of compound that possess a desired activity in-vivo or in-vitro. In the case of inflammatory disorders and autoimmune disorders, a beneficial clinical outcome includes reduction in the extent or severity of the symptoms associated with the disease or disorder and/or an increase in the longevity and/or quality of life of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of inflammatory disorder, autoimmune disorder, allergic disorder, or the degree of immunosuppression sought. The skilled artisan will be able to determine appropriate dosages depending on these and other factors. Effective amounts of the disclosed compounds typically range between about 1 mg/mm² per day and about 10 grams/mm² per day, and preferably between 10 mg/mm² per day and about 1 gram/mm².

The dosages of an immunosuppressant or anti-inflammatory agent other than compounds of the invention, which have been or are currently being used as an immunosuppressant or to prevent, treat, manage, or ameliorate an immune or inflammatory disorder, or one or more symptoms thereof, can be used in the combination therapies of the invention. Preferably, dosages lower than those which have been or are currently being used suppress the immune system or to prevent, treat, manage, or ameliorate an immune or inflammatory disorder, or one or more symptoms thereof, are used in the combination therapies of the invention. The recommended dosages of agents currently used for immunosuppression or for prevention, treatment, management, or amelioration of an immune or inflammatory disorder, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

As used herein, the terms “treat”, “treatment” and “treating” refer to the reduction or amelioration of the progression, severity and/or duration of an immune or inflammatory disorder, or the amelioration of one or more symptoms (preferably, one or more discernible symptoms) of an immune or inflammatory disorder resulting from the administration of one or more therapies (e.g., one or more therapeutic agents such as a compound represented by any of the formulas disclosed herein). In specific embodiments, the terms “treat”, “treatment” and “treating” refer to the amelioration of at least one measurable physical parameter of an immune or inflammatory disorder, such production of an inflammatory cytokine, not necessarily discernible by the patient. In other embodiments the terms “treat”, “treatment” and “treating” refer to the inhibition of the progression of an immune or inflammatory disorder, either physically by, e.g., stabilization of a discernible symptom, physiologically by, e.g., stabilization of a physical parameter, or both. In other embodiments the terms “treat”, “treatment” and “treating” refer to the reduction or stabilization of inflammation.

As used herein, the terms “prevent”, “prevention” and “preventing” refer to the reduction in the risk of acquiring or developing a given immune or inflammatory disorder, or the reduction or inhibition of the recurrence or an immune or inflammatory disorder. In one embodiment, a compound of the invention is administered as a preventative measure to a patient, preferably a human, having a genetic predisposition to any of the disorders described herein.

The term “inhibiting the production of inflammatory cytokines” means inhibiting the synthesis of a cytokine involved in inflammation (e.g. by inhibiting transcription (mRNA expression), or translation (protein expression)) and/or inhibiting secretion of the cytokine in a cell that has the ability to produce and/or secrete the cytokine (e.g., T lymphocyte). Likewise, the term “inhibiting production of G-CSF, IL-12, IL-1β, IL-23, IL-6, IL-8, and TNF-α, means inhibiting the synthesis (e.g. by inhibiting transcription, or translation) and/or inhibiting the secretion in a cell that has the ability to produce and/or secrete these cytokines.

As used herein, the terms “therapeutic agent” and “therapeutic agents” refer to any agent(s) which can be used for immunosuppression or in the treatment, management, or amelioration of an immune or inflammatory disorder or one or more symptoms thereof. In certain embodiments, the term “therapeutic agent” refers to a compound represented by any formula disclosed herein. In certain other embodiments, the term “therapeutic agent” does not refer to a compound disclosed herein. Preferably, a therapeutic agent is an agent which is known to be useful for, or has been or is currently being used for immunosuppression or in the treatment, management, prevention, or amelioration an immune or inflammatory disorder or one or more symptoms thereof.

As used herein, the term “synergistic” refers to a combination of a compound represented by any of the formulas disclosed herein and another therapy (e.g., a prophylactic or therapeutic agent), which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a proliferative disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy for immunosuppression or for prevention, management or treatment of an immune or inflammatory disorder. In addition, a synergistic effect can result in improved efficacy of agents for immunosuppression or for prevention, management or treatment of an immune or inflammatory disorder. Finally, a synergistic effect of a combination of therapies (e.g., a combination of prophylactic or therapeutic agents) may avoid or reduce adverse or unwanted side effects associated with the use of either therapy alone.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction. Unwanted side effects of treatment with glucocorticoids include osteoporosis, muscle wasting, hypertension, insulin resistance, truncal obesity and fat redistribution, and inhibition of wound repair.

As used herein, the term “in combination” refers to the use of more than one therapies (e.g., one or more prophylactic and/or therapeutic agents). The use of the term “in combination” does not restrict the order in which therapies (e.g., prophylactic and/or therapeutic agents) are administered to a subject in need of immunosuppression or a subject who has an immune or inflammatory disorder. A first therapy (e.g., a prophylactic or therapeutic agent such as a compound represented by anyone of the formulas disclosed herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-inflammatory agent) to a subject in need of immunosuppression or a subject that has an immune or inflammatory disorder.

As used herein, the terms “therapies” and “therapy” can refer to any protocol(s), method(s), and/or agent(s) that can be used for immunosuppression or in the prevention, treatment, management, or amelioration of an immune or inflammatory disorder or one or more symptoms thereof.

As used herein, a “protocol” includes dosing schedules and dosing regimens. The protocols herein are methods of use and include prophylactic and therapeutic protocols.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a reaction that is “substantially complete” means that the reaction contains more than about 80% by weight of the desired product, more preferably more than about 90% by weight of the desired product, even more preferably more than about 95% by weight of the desired product, and most preferably more than about 97% by weight of the desired product.

As used herein, a racemic mixture means about 50% of one enantiomer and about 50% of is corresponding enantiomer relative to a chiral center in the molecule. The invention encompasses all enantiomerically-pure, enantiomerically-enriched, diastereomerically pure, diastereomerically enriched, and racemic mixtures of the compounds of the invention.

Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds of the invention are defined herein by their chemical structures and/or chemical names. Where a compound is referred to by both a chemical structure and a chemical name, and the chemical structure and chemical name conflict, the chemical structure is determinative of the compound's identity.

When administered to a patient, e.g., to a non-human animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, the compounds of the invention are purified via conventional techniques. As used herein, “purified” means that when isolated, the isolate contains at least 95%, preferably at least 98%, of a compound of the invention by weight of the isolate either as a mixture of stereoisomers or as a diastereomeric or enantiomeric pure isolate. An “isolated agent” can be a synthetic or naturally occurring molecule having a molecular weight of about 1000 daltons or less, or a natural product having a molecular weight of greater than 1000 daltons. For example, an isolated agent can be an antibody, or fragment thereof, or an antibiotic.

As used herein, a composition that is “substantially free” of a compound means that the composition contains less than about 20% by weight, more preferably less than about 10% by weight, even more preferably less than about 5% by weight, and most preferably less than about 3% by weight of the compound.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

B. The Compounds of the Invention

The present invention encompasses the use of compounds having formula (I) through (LXXII), or any embodiment thereof, or a compound shown in Table 5, 6, or 7, and tautomers, pharmaceutically acceptable salts, solvates, clathrates, hydrates, polymorphs and prodrugs thereof, to modulate the activity of glucocorticoid receptors, to suppress the immune system of a subject or to treat or prevent an immune or inflammatory disorder. In one aspect, the invention provides a method of modulating the activity of glucocorticoid receptors in a cell, comprising administering to the cell an effective amount of a compound represented by formula (I) as set forth below:

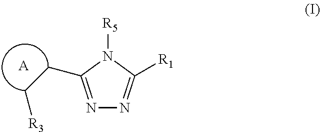

and tautomers, pharmaceutically acceptable salts, solvates, clathrates, and prodrugs thereof, wherein ring A, R₁, R₃ and R₅ are defined as above.

In one embodiment, in the compounds of formula (I), R₅ is an optionally substituted naphthyl.

In another embodiment, in the compounds of formula (I), R₅ is represented by the following formula:

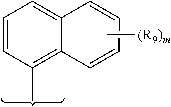

wherein:

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring; and

m is zero or an integer from 1 to 7, wherein R₇, R₈, R₁₀, R₁₁, and p are defined as above.

In another embodiment, in the compounds represented by formula (I), R₅ is represented by one of the following formulas:

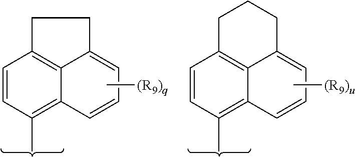

wherein R₉ is defined as above;

q is zero or an integer from 1 to 7; and

u is zero or an integer from 1 to 8.

In another embodiment, in the compounds represented by formula (I), R₅ is selected from the group consisting of:

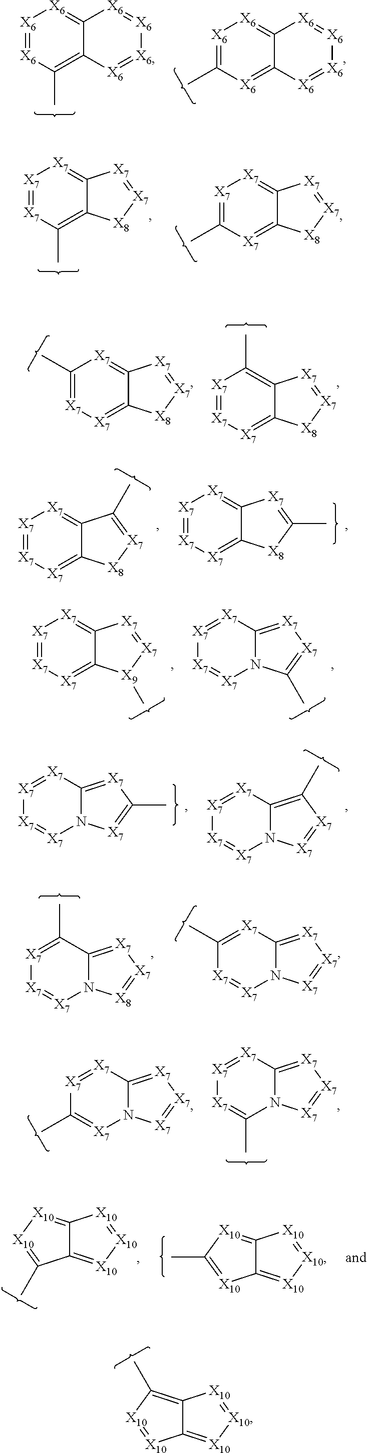

wherein:

X₆, for each occurrence, is independently CH, CR₉, N, N(O), N⁺(R₁₇), provided that at least three X₆ groups are independently selected from CH and CR₉;

X₇, for each occurrence, is independently CH, CR₉, N, N(O), N⁺(R₁₇), provided that at least three X₇ groups are independently selected from CH and CR₉;

X₈, for each occurrence, is independently CH₂, CHR₉, CR₉R₉, O, S, S(O)p, NR₇, or NR₁₇;

X₉, for each occurrence, is independently N or CH;

X₁₀, for each occurrence, is independently CH, CR₉, N, N(O), N⁺(R₁₇), provided that at least one X₁₀ is selected from CH and CR₉;

R₁₇, for each occurrence, is independently —H, an alkyl, an aralkyl, —C(O)R₇, —C(O)OR₇, or —C(O)NR₁₀R₁₁; wherein R₇, R₉, R₁₀, R₁₁ and p are defined as above.

In another embodiment, in the compounds represented by formula (I), R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl,an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo(b)thienyl.

In another embodiment, in the compounds represented by formula (I), R₅ is an optionally substituted indolyl. Preferably, R₅ is an indolyl represented by the following structural formula:

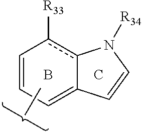

wherein:

R₃₃ is —H, a halo, lower alkyl, a lower alkoxy, a lower haloalkyl, a lower haloalkoxy, and lower alkyl sulfanyl;

R₃₄ is H, a lower alkyl, or a lower alkylcarbonyl; and

Ring B and Ring C are optionally substituted with one or more substituents.

In another embodiment, in the compounds represented by formula (I), R₅ is selected from the group consisting of:

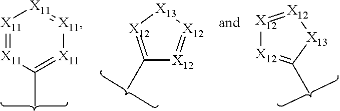

wherein:

X₁₁, for each occurrence, is independently CH, CR₉, N, N(O), or N⁺(R₁₇), provided that at least one X₁₁ is N, N(O), or N⁺(R₁₇) and at least two X₁₁ groups are independently selected from CH and CR₉;

X₁₂, for each occurrence, is independently CH, CR₉, N, N(O), N⁺(R₁₇), provided that at least one X₁₂ group is independently selected from CH and CR₉;

X₁₃, for each occurrence, is independently O, S, S(O)p, NR₇, or NR₁₇; wherein R₇, R₉ and R₁₇ are defined as above.

In another embodiment, in compounds represented by formula (I), or any of the embodiments of formula (I) in which particular groups are disclosed, the compound is represented by formula (XII):

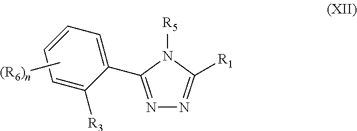

wherein R₁, R₃, and R₅ are defined as above; and

R₆, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; and

n is zero of an integer from 1 to 4, wherein R₇, R₈, R₁₀, R,₁₁, and p are defined as above.

In another embodiment, in compounds represented by formula (I), or any of the embodiments of formula (I) in which particular groups are disclosed, the compound is represented by structural formula (XIII):

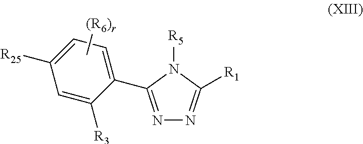

wherein R₁, R₃, R₅, and R₆ are defined as above; and

R₂₅ is a halo, a haloalkyl, a haloalkoxy, a heteroalkyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇;

k is 1, 2, 3, or 4; and

r is zero or an integer from 1 to 3, wherein R₇, R₈, R₁₀, R₁₁, and p are defined as above.

In another embodiment, in compounds represented by formula (I), or any of the embodiments of formula (I) in which particular groups are disclosed, R₁ and R₃ are each, independently, —OH, —SH, or —NHR₇.

In another embodiment, in compounds represented by formula (I), or any of the embodiments of formula (I) in which particular groups are disclosed, the compound is represented by structural formula (XIV):

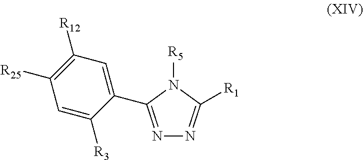

wherein R₁, R₃, R₅, and R₂₅ are defined as above; and

R₁₂ is an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇, wherein R₇, R₈, R₁₀, R₁₁, and p are defined as above. In a prefered embodiment, R₁ is —SH or —OH; R₃ and R₂₅ are —OH; R₁₂ is a lower alkyl, lower alkoxy, a lower alkyl sulfanyl, or —NR₁₀R₁₁; and R₉, for each occurrence, is independently selected from the group consisting of —OH, —SH, halo, a lower haloalkyl, cyano, a lower alkyl, a lower alkoxy, and a lower alkyl sulfanyl.

In another embodiment, in compounds represented by formula (I), or any of the embodiments of formula (I) in which particular groups are disclosed, the compound is represented by one of the following structural formulas:

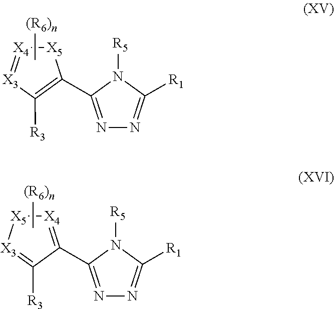

wherein R₁, R₃, R₅, R₆ and n are as defined above; and

X₃ and X₄ are each, independently, N, N(O), N⁺(R₁₇), CH or CR₆; and

X₅ is O, S, NR₁₇, CH═CH, CH═CR₆, CR₆═CH, CR₆═CR₆, CH═N, CR₆═N, CH═N(O), CR₆═N(O), N═CH, N═CR₆, N(O)═CH, N(O)═CR₆, N⁺(R₁₇)═CH, N⁺(R₁₇)═CR₆, CH═N⁺(R₁₇), CR₆═N⁺(R₁₇), or N═N; wherein R₁₇ is defined as above.

In another embodiment, in compounds represented by formula (I), or any of the embodiments of formula (I) in which particular groups are disclosed, the compound is selected from the group consisting of:

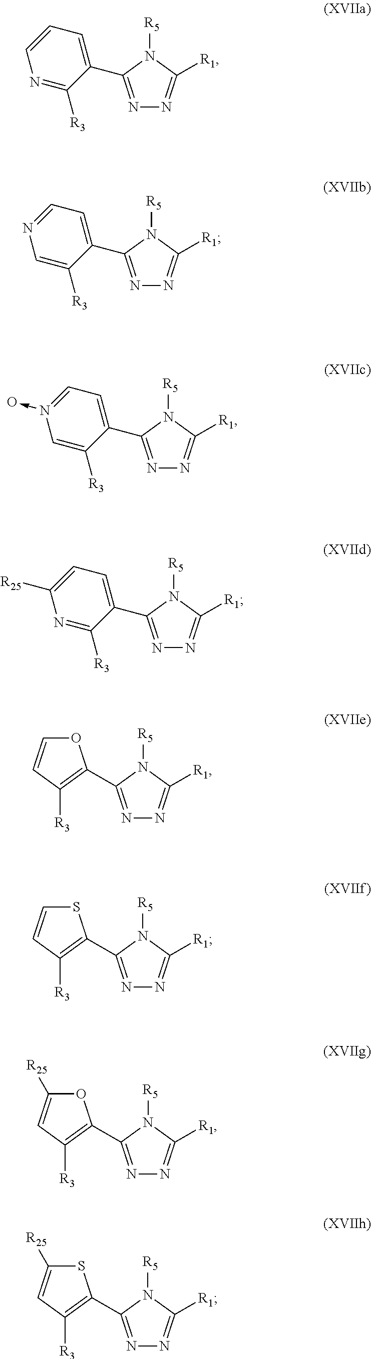

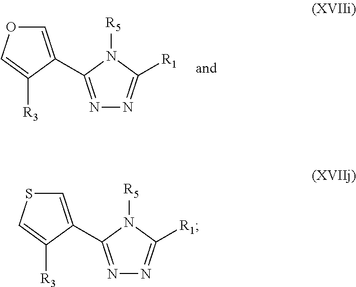

wherein R₁, R₃, R₅, and R₂₅ are defined as above.

In one embodiment, the cell is in a subject in need of treatment for an inflammatory disorder and administration of a compound represented by formula (I), or any of the embodiments of formula (I) disclosed herein, to the subject modulates the activity of glucocorticoid receptors in a cell that is in the subject.

In one embodiment, the cell is in a subject in need of treatment for an immune disorder and administration of a compound represented by formula (I), or any of the embodiments of formula (I) disclosed herein, to the subject modulates the activity of glucocorticoid receptors in a cell that is in the subject.

In one embodiment, the cell is in a subject in need of immunosuppression and administration of a compound represented by formula (I), or any of the embodiments of formula (I) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound of formula (I) or any of the embodiments of formula (I) disclosed herein.

In another aspect, the invention provides a method of treating or preventing an immune disorder in a subject, comprising administering to the subject an effective amount of a compound of formula (I) or any of the embodiments of formula (I) disclosed herein.

In another aspect, the invention provides a method of suppressing the immune system in a subject in need thereof, comprising administering to the subject an effective amount of a compound represented by formula (I), or any of the embodiments of formula (I) disclosed herein.

In another aspect, the invention provides a method of modulating the activity of glucocorticoid receptors in a cell, comprising administering to the cell an effective amount of a compound represented by formula (II) as set forth below:

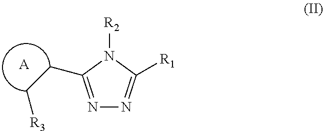

and tautomers, pharmaceutically acceptable salts, solvates, clathrates, and prodrugs thereof, wherein ring A, R₁ and R₃ are defined as above; and

R₂ is a substituted phenyl, wherein the phenyl group is substituted with:
i) one substituent selected from nitro, cyano, a haloalkoxy, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxylalkyl, alkoxyalkyl, guanadino, —NR₁₀R₁₁, —O—R₂₀, —C(O)R₇, —C(O)OR₂₀, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁, or
ii) two to five substituents selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, —F, —Br, —I, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚR₇, —NR₈S(O)ₚR₇, or —S(O)ₚNR₁₀R₁₁;

R₂₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

p, for each occurrence, is, independently, 0, 1 or 2.

In one embodiment, the compounds represented by formula (II) do not include 3-(2,4-dihydroxy-phenyl)-4-(7-naphthalen-1-yl)-5-mercapto-triazole, 3-(2,4-dihydroxyphenyl)-4-(2,5-dimethoxyphenyl)-5-mercapto-triazole, 3-(1-phenyl-5-amino-pyrazol-4-yl)-4-(2,4-dichloropheny)-5-mercapto-triazole, and 3-(2-hydroxy-phenyl)4-(2,4-dimethylphenyl)-5-mercapto-triazole.

In another embodiment, in compounds represented by formula (II), or any of the embodiments of formula (II) in which particular groups are disclosed, the compound is represented by structural formula (XVIII):

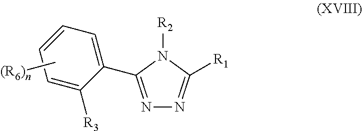

wherein R₁, R₂, R₃, R₆, and n are defined as above.

In another embodiment, in compounds represented by formula (II), or any of the embodiments of formula (II) in which particular groups are disclosed, the compound is represented by structural formula (XIX):

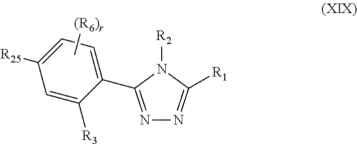

wherein R₁, R₂, R₃, R₆, R₂₅ and r are defined as above.

In another embodiment, in compounds represented by formula (II), or any of the embodiments of formula (II) in which particular groups are disclosed, R₁ and R₃ are each, independently, —OH, —SH, or —NHR₇.

In another embodiment, in compounds represented by formula (II), or any of the embodiments of formula (II) in which particular groups are disclosed, the compound is represented by structural formula (XX):

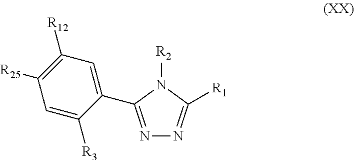

wherein R₁, R₂, R₃, R₁₂ and R₂₅ are defined as above. In a preferred embodiment, R₁ is —SH or —OH; R₃ and R₂₅ are —OH; R₁₂ is a lower alkyl, lower alkoxy, a lower alkyl sulfanyl, or —NR₁₀R₁₁; and R₉, for each occurrence, is independently selected from the group consisting of —OH, —SH, halo, a lower haloalkyl, cyano, a lower alkyl, a lower alkoxy, and a lower alkyl sulfanyl.

In another embodiment, in compounds represented by formula (II), or any of the embodiments of formula (II) in which particular groups are disclosed, the compound is represented by one of the following structural formulas:

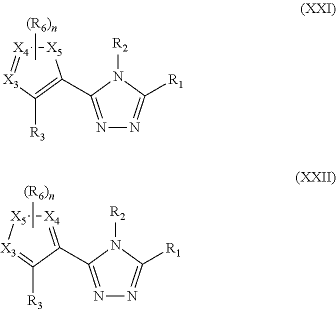

wherein R₁, R₂, R₃, R₆, X₃, X₄, X₅ and n are defined as above.

In another embodiment, in compounds represented by formula (II), or any of the embodiments of formula (II) in which particular groups are disclosed, the compound is selected from the group consisting of:

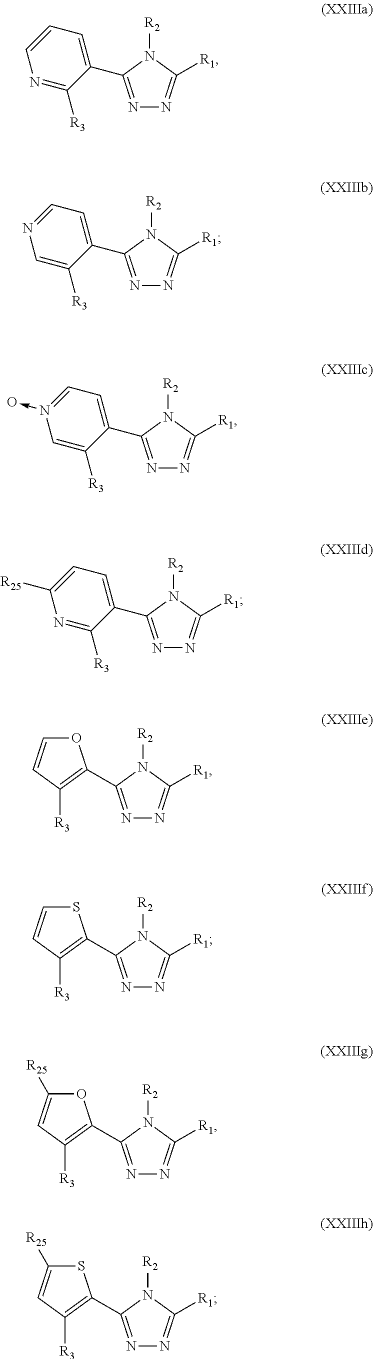

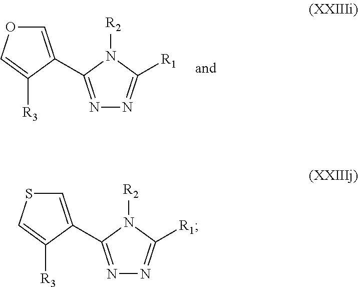

wherein R₁, R₂, R₃, and R₂₅ are defined as above.

In one embodiment, the cell is in a subject in need of treatment for an inflammatory disorder and administration of a compound represented by formula (II), or any of the embodiments of formula (II) disclosed herein, to the subject modulates the activity of glucocorticoid receptors in a cell that is in the subject.

In one embodiment, the cell is in a subject in need of treatment for an immune disorder and administration of a compound represented by formula (II), or any of the embodiments of formula (II) disclosed herein, to the subject modulates the activity of glucocorticoid receptors in a cell that is in the subject.

In one embodiment, the cell is in a subject in need of immunosuppression and administration of a compound represented by formula (II), or any of the embodiments of formula (II) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound of formula (II), or any of the embodiments of formula (II) disclosed herein.

In another aspect, the invention provides a method of treating or preventing an immune disorder in a subject, comprising administering to the subject an effective amount of a compound of formula (II), or any of the embodiments of formula (II) disclosed herein.

In another aspect, the invention provides a method of suppressing the immune system in a subject in need thereof, comprising administering to the subject an effective amount of a compound represented by formula (II), or any of the embodiments of formula (II) disclosed herein.

In another aspect, the invention provides a method of modulating the activity of glucocorticoid receptors in a cell, comprising administering to the cell an effective amount of a compound represented by formula (III) as set forth below:

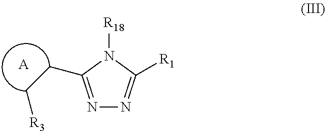

and tautomers, pharmaceutically acceptable salts, solvates, clathrates, and prodrugs. In formula (III), ring A, R₁, and R₃ are defined as above; and

R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above.

In one embodiment, in formula (III) R₁₈ is not cyclohexyl.

In another embodiment, in formula (III) R₁₈ is an optionally substituted cycloalkyl or an optionally substituted cycloalkenyl.

In another embodiment, in formula (III) R₁₈ is a substituted alkyl.

In another embodiment, in compounds represented by formula (III), or any of the embodiments of formula (III) in which particular groups are disclosed, the compound is represented by structural formula (XXIV):

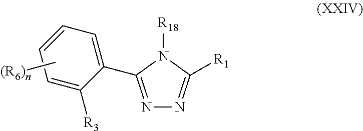

wherein R₁, R₃, R₆, R₁₈, and n are defined as above.

In another embodiment, in compounds represented by formula (III), or any of the embodiments of formula (III) in which particular groups are disclosed, the compound is represented by structural formula (XXV):

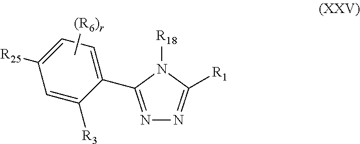

wherein R₁, R₃, R₆, R₁₈, R₂₅ and r are defined as above.

In another embodiment, in compounds represented by formula (III), or any of the embodiments of formula (III) in which particular groups are disclosed, R₁ and R₃ are each, independently, —OH, —SH, or —NHR₇.

In another embodiment, in compounds represented by formula (III), or any of the embodiments of formula (III) in which particular groups are disclosed, the compound is represented by structural formula (XXVI):

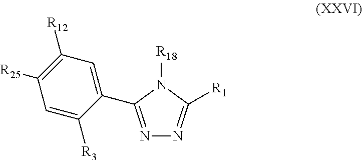

wherein R₁, R₃, R₁₂, R₁₈, and R₂₅ are defined as above. In a preferred embodiment, R₁ is —SH or —OH; R₃ and R₂₅ are —OH; and R₁₂ is a lower alkyl, lower alkoxy, a lower alkyl sulfanyl, or —NR₁₀R₁₁.

In another embodiment, in compounds represented by formula (III), or any of the embodiments of formula (III) in which particular groups are disclosed, the compound is represented by one of the following structural formulas:

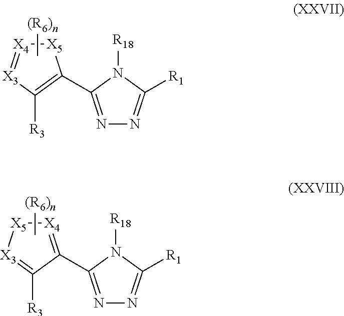

wherein R₁, R₃, R₆, R₁₈, X₃, X₄, X₅, and n are defined as above.

In another embodiment, in compounds represented by formula (III), or any of the embodiments of formula (III) in which particular groups are disclosed, the compound is selected from the group consisting of:

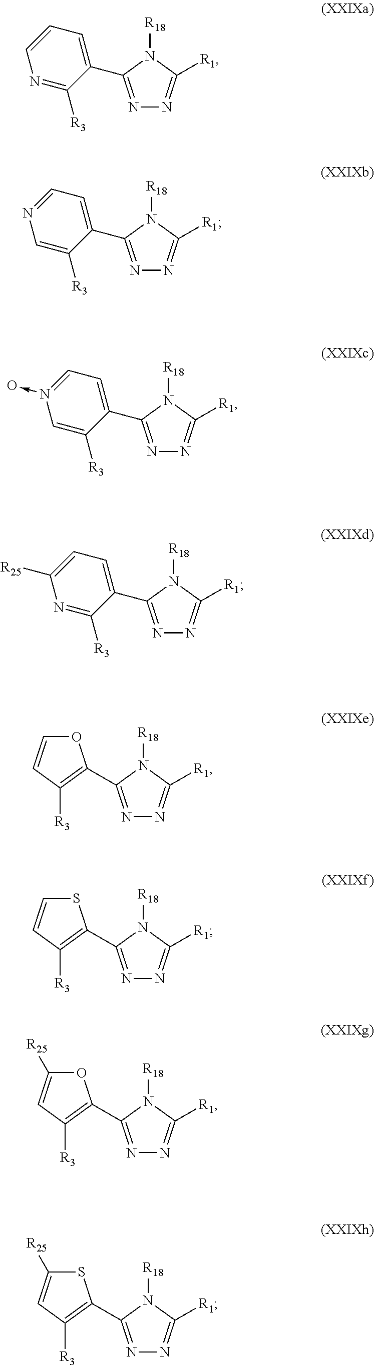

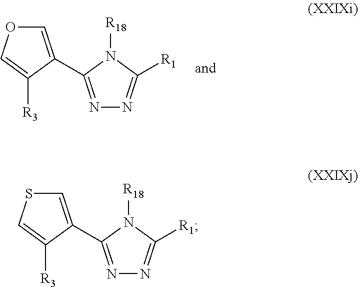

wherein R₁, R₃, R₁₈, and R₂₅ are defined as above.

In one embodiment, the cell is in a subject in need of treatment for an inflammatory disorder and administration of a compound represented by formula (III), or any of the embodiments of formula (III) disclosed herein, to the subject modulates the activity of glucocorticoid receptors in a cell that is in the subject.

In one embodiment, the cell is in a subject in need of treatment for an immune disorder and administration of a compound represented by formula (III), or any of the embodiments of formula (III) disclosed herein, to the subject modulates the activity of glucocorticoid receptors in-a cell that is in the subject.

In one embodiment, the cell is in a subject in need of immunosuppression and administration of a compound represented by formula (III), or any of the embodiments of formula (III) disclosed herein, to the subject modulates the activity of glucocorticoid receptors in a cell that is in the subject.

In another aspect, the invention provides a method of treating or preventing an inflammatory disorder in a subject, comprising administering to the cell an effective amount of a compound of formula (III), or any of the embodiments of formula (III) disclosed herein.

In another aspect, the invention provides a method of treating or preventing an immune disorder in a subject, comprising administering to the subject an effective amount of compounds of formula (III), or any of the embodiments of formula (III) disclosed herein.

In another aspect, the invention provides a method of suppressing the immune system in a subject in need thereof, comprising administering to the subject an effective amount of a compound represented by formula (III), or any of the embodiments of formula (III) disclosed herein.

In another aspect, the invention provides a method of modulating the activity of glucocorticoid receptors in a cell, comprising administering to the cell an effective amount of a compound represented by formula (IV) or (V) as set forth below:

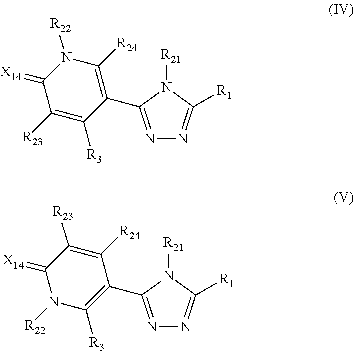

and tautomers, pharmaceutically acceptable salts, solvates, clathrates, and prodrugs thereof. In formulas (IV) and (V), R₁ and R₃ are as defined above; and

X₁₄ is O, S, or NR₇;

R₂₁ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₂₂, for each occurrence, is independently a substituent selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally-substituted heteroaryl, an optionally substituted aralkyl, or an optionally substituted heteraralkyl, a haloalkyl, —C(O)R₇, —C(O)OR₇, —OC(O)R₇, —C(O)NR₁₀R₁₁, —NR₈C(O)R₇, —S(O)ₚR₇, —S(O)ₚOR₇, or —S(O)ₚNR₁₀R₁₁; and

R₂₃ and R₂₄, for each occurrence, are independently a substituent selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

wherein R₇, R₈, R₁₀, R₁₁ and p are defined as above.

In one embodiment, in formulas (IV) and (V), R₂₁ is an optionally substituted alkyl, an optionally substituted cycloalkyl, an optionally substituted aryl or an optionally substituted heteroaryl.

In another embodiment, in the formulas (IV) and (V), R₁ is —OH, —SH, or —NHR₇.

In another embodiment, in the formulas (IV) and (V), R₂₂ is an alkyl, an aralkyl, —C(O)R₇, —C(O)OR₇, or —C(O)NR₁₀R₁₁.

In another embodiment, in the formulas (IV) and (V), X₁₄ is O.

In one embodiment, the cell is in a subject in need of treatment for an inflammatory disorder and administration of a compound represented by formula (IV) or (V), or any of the embodiments of formula (IV) or (V) disclosed herein, to the subject modulates the activity of glucocorticoid receptors in a cell that is in the subject.

In one embodiment, the cell is in a subject in need of treatment for an immune disorder and administration of a compound represented by formula (IV) or (V), or any of the embodiments of formula (IV) or (V) disclosed herein, to the subject modulates the activity of glucocorticoid receptors in a cell that is in the subject.

In one embodiment, the cell is in a subject in need of immunosuppression and administration of a compound represented by formula (IV) or (V), or any of the embodiments of formula (IV) or (V) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (IV) or (V), or any of the embodiments of formula (IV) or (V) disclosed herein.

In another aspect, the invention provides a method of treating or preventing an immune disorder in a subject, comprising administering to the subject an effective amount of a compound represented by formula (IV) or (V), or any of the embodiments of formula (IV) or (V) disclosed herein.

In another aspect, the invention provides a method of suppressing the immune system in a subject in need thereof, comprising administering to the subject an effective amount of a compound represented by formula (IV) or (V), or any of the embodiments of formula (IV) or (V) disclosed herein.

In another aspect, the invention provides a method of modulating the activity of glucocorticoid receptors in a cell, comprising administering to the cell an effective amount of a compound represented by formula (XXX), as set forth below:

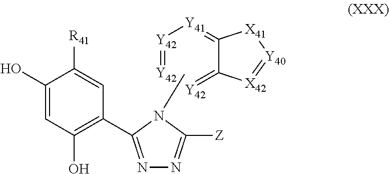

and tautomers, pharmaceutically acceptable salts, solvates, clathrates, and prodrugs thereof, wherein:

X₄₁ is O, S, or NR₄₂;

X₄₂ is CR₄₄ or N;

Y₄₀ is N or CR₄₃;

Y₄₁ is N or CR₄₅;

Y_{42,} for each occurrence, is independently N, C or CR₄₆;

Z is OH, SH, or NHR₇;

R₄₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

R₄₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;

R₄₃ and R₄₄ are, independently, —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;

R₄₅ is —H, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;

R₄₆, for each occurrence, is independently selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

R₇, R₈, R₁₀, R₁₁, R₂₆, p, and m are defined as above.

In one embodiment, in formula (XXX), X₄₁ is NR₄₂ and X₄₂ is CR₄₄.

In another embodiment, in formula (XXX), X₄₁ is NR₄₂ and X₄₂ is N.

In another embodiment, in formula (XXX), R₄₁ is selected from the, group consisting of —H, lower alkyl, lower alkoxy, lower cycloalkyl, and lower cycloalkoxy.

In another embodiment, in formula (XXX), R₄₁ is selected from the group consisting of —H, methyl, ethyl, propyl, isopropyl, cyclopropyl, methoxy, ethoxy, propoxy, and cyclopropoxy.

In another embodiment, in formula (XXX), X₄₁ is NR₄₂, and R₄₂ is selected from the group consisting of —H, a lower alkyl, a lower cycloalkyl, —C(O)N(R₂₇)₂, and —C(O)OH, wherein R₂₇, for each occurrence, is independently is —H or a lower alkyl.

In another embodiment, in formula (XXX), X₄₁ is NR₄₂, and R₄₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In one embodiment, Y₄₀ is CR₄₃. Preferably, Y₄₀ is CR₄₃ and R₄₃ is H or a lower alkyl.

In another embodiment, in formula (XXX), R₄₃ and R₄₄ are, independently, selected from the group consisting of —H, methyl, ethyl, propyl, isopropyl, cyclopropyl, methoxy, ethoxy, propoxy, and cyclopropoxy.

In another embodiment, in formula (XXX), X₄₂ is CR₄₄; Y is CR₄₃; and R₄₃ and R₄₄ together with the carbon atoms to which they are attached form a cycloalkenyl, an aryl, heterocyclyl, or heteroaryl ring. In one aspect of this embodiment, R₄₃ and R₄₄ together with the carbon atoms to which they are attached form a C₅-C₈ cycloalkenyl or a C₅-C₈ aryl.

In another embodiment, in formula (XXX), R₄₅ is selected from the group consisting of —H, —OH, —SH, —NH₂, a lower alkoxy, a lower alkyl amino, and a lower dialkyl amino.

In another embodiment, in formula (XXX), R₄₅ is selected from the group consisting of —H, —OH, methoxy and ethoxy.

In another embodiment, in formula (XXX), X₄₁ is O.

In another embodiment, the compound is selected from the group consisting of:

### 3-(2,4-dihydroxy-5-ethyl-phenyl)-4-(2-methyl-7-methoxy-benzofuran-4-yl)-5-mercapto-[1,2,4]triazole,

### 3-(2,4-dihydroxy-5-ethyl-phenyl)-4-(benzofuran-5-yl)-5-mercapto-[1,2,4]triazole,

### 3-(2,4-dihydroxy-5-ethyl-phenyl)-4-(2-methyl-1,3-benzoxaz-5-yl)-5-mercapto-[1,2,4]triazole, and

tautomers, pharmaceutically acceptable salts, solvates, clathrates, and prodrugs thereof.

In another embodiment, in formula (XXX), Z is —OH.

In another embodiment, the compound is selected from the group consisting of:

### 3-(2,4-dihydroxy-5-ethyl-phenyl)-4-(1,3-dimethyl-indol-5-yl)-5-hydroxy-[1,2,4]triazole,

### 3-(2,4-dihydroxy-5-isopropyl-phenyl)-4-(1,3-dimethyl-indol-5-yl)-5-hydroxy-[1,2,4]triazole,

### 3-(2,4-dihydroxy-5-isopropyl-phenyl)-4-(1-methyl-indol-5-yl)-5-hydroxy-[1,2,4]triazole,

### 3-(2,4-dihydroxy-5-isopropyl-phenyl)-4-(1-isopropyl-indol-4-yl)-5-hydroxy-[1,2,4]triazole, and

tautomers, pharmaceutically acceptable salts, solvates, clathrates, and prodrugs thereof.

In another embodiment, Z is —SH.

In another embodiment, the compound is selected from the group consisting of:

### 3-(2,4-dihydroxy-5-isopropyl-phenyl)-4-(1-methyl-indazol-5-yl)-5-mercapto-[1,2,4]triazole,

### 3-(2,4-dihydroxy-5-isopropyl-phenyl)-4-(1-methyl-indazol-6-yl)-5-mercapto-[1,2,4]triazole, and

tautomers, pharmaceutically acceptable salts, solvates, clathrates, and prodrugs thereof.

In one embodiment, the cell is in a subject in need of treatment for an inflammatory disorder and administration of a compound represented by formula (XXX), or any of the embodiments of formula (XXX) disclosed herein, to the subject modulates the activity of glucocorticoid receptors in a cell that is in the subject.

In one embodiment, the cell is in a subject in need of treatment for an immune disorder and administration of a compound represented by formula (XXX), or any of the embodiments of formula (XXX) disclosed herein, to the subject modulates the activity of glucocorticoid receptors in a cell that is in the subject.

In one embodiment, the cell is in a subject in need of immunosuppression and administration of a compound represented by formula (XXX), or any of the embodiments of formula (XXX) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXX), or any of the embodiments of formula (XXX) disclosed herein.

In another aspect, the invention provides a method of treating or preventing an immune disorder in a subject, comprising administering to the subject an effective amount of a compound represented by formula (XXX), or any of the embodiments of formula (XXX) disclosed herein.

In another aspect, the invention provides a method of suppressing the immune system in a subject in need thereof, comprising administering to the subject an effective amount of a compound represented by formula (XXX), or any of the embodiments of formula (XXX) disclosed herein.

In another aspect, the invention provides a method of modulating the activity of glucocorticoid receptors in a cell, comprising administering to the cell an effective amount of a compound represented by formula (XXXI):

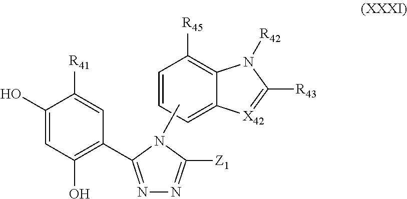

and tautomers, pharmaceutically acceptable salts, solvates, clathrates, and prodrugs thereof, wherein:

Z₁ is —OH or —SH;

X₄₂, R₄₁, R₄₂, R₄₃, and R₄₅ are defined as above.

In one embodiment, in formula (XXXI), Z₁ is —OH.

In another embodiment, in formula (XXXI), Z₁ is —SH.

In another embodiment, in formula (XXXI), R₄₁ is selected from the group consisting of —H, lower alkyl, lower alkoxy, lower cycloalkyl, and lower cycloalkoxy.

In another embodiment, in formula (XXXI), R₄₁ is selected from the group consisting of —H, methyl, ethyl, propyl, isopropyl, cyclopropyl, methoxy, ethoxy, propoxy, and cyclopropoxy.

In another embodiment, in formula (XXXI), R₄₂ is selected from the group consisting of lower alkyl, lower cycloalkyl, —C(O)N(R₂₇)₂, or —C(O)OH, wherein R₂₇, for each occurrence, is independently is —H or a lower alkyl.

In another embodiment, in formula (XXXI), R₄₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In another embodiment, R₄₃ is H or a lower alkyl.

In another embodiment, in formula (XXXI), X₄₂ is CR₄₄, and R₄₃ and R₄₄ are, independently, selected from the group consisting of —H, methyl, ethyl, propyl, isopropyl, cyclopropyl, methoxy, ethoxy, propoxy, and cyclopropoxy.

In another embodiment, in formula (XXXI), X₄₂ is CR₄₄, and R₄₃ and R₄₄, taken together with the carbon atoms to which they are attached, form a cycloalkenyl, aryl, heterocyclyl, or heteroaryl ring. Preferably, in this embodiment, R₄₃ and R₄₄, taken together with the carbon atoms to which they are attached, form a C₅-C₈ cycloalkenyl or a C₅-C₈ aryl.

In another embodiment, in formula (XXXI), R₄₅ is selected from the group consisting of —H, —OH, —SH, —NH₂, a lower alkoxy, a lower alkyl amino, and a lower dialkyl amino.

In another embodiment, in formula (XXXI), R₄₅ is selected from the group consisting of —H, —OH, methoxy, and ethoxy.

In another embodiment, in formula (XXXI), X₄₃ is CR₄₄.

In another embodiment, the compound is selected from the group consisting of:

### 3-(2,4-dihydroxyphenyl)-4-(1-ethyl-indol-4-yl)-5-mercapto-[1,2,4]triazole,

### 3-(2,4-dihydroxyphenyl)-4-(1-isopropyl-indol-4-yl)-5-mercapto-[1,2,4]triazole,

### 3-(2,4-dihydroxyphenyl)-4-(indol-4-yl)-5-mercapto-[1,2,4]triazole,

### 3-(2,4-dihydroxyphenyl)-4-(1-methoxyethyl-indol-4-yl)-5-mercapto-[1,2,4]triazole,

### 3-(2,4-dihydroxy-5-ethyl-phenyl)-4-(1-isopropyl-indol-4-yl)-5-mercapto-[1,2,4]triazole,

### 3-(2,4-dihydroxyphenyl)-4-(1-dimethylcarbamoyl-indol-4-yl)-5-mercapto-[1,2,4]triazole,

### 3-(2,4-dihydroxy-5-ethyl-phenyl)-4-(1-propyl-indol-4-yl)-5-mercapto-[1,2,4]triazole,

### 3-(2,4-dihydroxy-5-ethyl-phenyl)-4-(1,2,3-trimethyl-indol-5-yl)-5-mercapto-[1,2,4]triazole,

### 3-(2,4-dihydroxy-5-ethyl-phenyl)-4-(2,3-dimethyl-indol-5-yl)-5-mercapto-[1,2,4]triazole,

### 3-(2,4-dihydroxy-5-ethyl-phenyl)-4-(1-acetyl-2,3-dimethyl-indol-5-yl)-5-mercapto-[1,2,4]triazole,

### 3-(2,4-dihydroxy-5-ethyl-phenyl)-4-(1-isopropyl-7-methoxy-indol-4-yl)-5-mercapto-[1,2,4]triazole,

### 3-(2,4-dihydroxy-5-ethyl-phenyl)-4-(1-propyl-2,3-dimethyl-indol-5-yl)-5-mercapto-[1,2,4]triazole,

### 3-(2,4-dihydroxy-5-ethyl-phenyl)-4-(N-methyl-tetrahydrocarbozol-7-yl)-5-mercapto-[1,2,4]triazole,

### 3-(2,4-dihydroxy-5-ethyl-phenyl)-4-(N-methyl-cyclononan[a]indol-5-yl)-5-mercapto-[1,2,4]triazole,

### 3-(2,4-dihydroxy-5-ethyl-phenyl)-4-(1-n-butyl-indol-4-yl)-5-mercapto-[1,2,4]triazole,

### 3-(2,4-dihydroxy-5-ethyl-phenyl)-4-(1-n-pentyl-indol-4-yl)-5-mercapto-[1,2,4]triazole,

### 3-(2,4-dihydroxy-5-ethyl-phenyl)-4-(1-n-hexyl-indol-4-yl)-5-mercapto-[1,2,4]triazole,

### 3-(2,4-dihydroxy-5-cyclopropyl-phenyl)-4-(1-(1-methylcyclopropyl)-indol-4-yl)-5-mercapto-[1,2,4]triazole,

### 3-(2,4-dihydroxy-5-cyclopropyl-phenyl)-4-(1-isopropyl-7-methoxy-indol-4-yl)-5-mercapto-[1,2,4]triazole,

### 3-(2,4-dihydroxy-5-cyclopropyl-phenyl)-4-(1,2,3-trimethyl-indol-5-yl)-5-mercapto-[1,2,4]triazole,

### 3-(2,4-dihydroxy-5-ethyl-phenyl)-4-(1-isopropyl-7-methoxy-indol-4-yl)-5-mercapto-[1,2,4]triazole disodium salt,

### 3-(2,4-dihydroxy-5-tert-butyl-phenyl)-4-(1-isopropyl-7-methoxy-indol-4-yl)-5-mercapto-[1,2,4]triazole,

### 3-(2,4-dihydroxy-5-cyclopropyl-phenyl)-4-(1-propyl-7-methoxy-indol-4-yl)-5-mercapto-[1,2,4]triazole,

### 3-(2,4-dihydroxy-5-ethyl-phenyl)-4-(1-methyl-3-ethyl-indol-5-yl)-5-mercapto-[1,2,4]triazole,

### 3-(2,4-dihydroxy-5-ethyl-phenyl)-4-(1,3-dimethyl-indol-5-yl)-5-mercapto-[1,2,4]triazole,

### 3-(2,4-dihydroxy-5-isopropyl-phenyl)-4-(1-isopropyl-7-methoxy-indol-4-yl)-5-mercapto-[1,2,4]triazole,

### 3-(2,4-dihydroxy-5-ethyl-phenyl)-4-(1-methyl-3-isopropyl-indol-5-yl)-5-mercapto-[1,2,4]triazole,

### 3-(2,4-dihydroxy-5-ethyl-phenyl)-4-(N-ethyl-carbozol-7-yl)-5-mercapto-[1,2,4]triazole,

### 3-(2,4-dihydroxy-5-ethyl-phenyl)-4-(1-isopropyl-7-hydroxy-indol-4-yl)-5-mercapto-[1,2,4]triazole,

### 3-(2,4-dihydroxy-5-ethyl-phenyl)-4-(1-isopropyl-7-ethoxy-indol-4-yl)-5-mercapto-[1,2,4]triazole,

### 3-(2,4-dihydroxy-5-ethyl-phenyl)-4-(1,2-dimethyl-indol-5-yl)-5-mercapto-[1,2,4]triazole,

### 3-(2,4-dihydroxy-5-ethyl-phenyl)-4-(N-methyl-indol-5-yl)-5-mercapto-[1,2,4]triazole,

### 3-(2,4-dihydroxy-5-isopropyl-phenyl)-4-(1,3-dimethyl-indol-5-yl)-5-mercapto-[1,2,4]triazole,

### 3-(2,4-dihydroxy-5-cyclopropyl-phenyl)-4-(1,3-dimethyl-indol-5-yl)-5-mercapto-[1,2,4]triazole,

### 3-(2,4-dihydroxy-5-cyclopropyl-phenyl)-4-(1-methyl-indol-5-yl)-5-mercapto-[1,2,4]triazole,

### 3-(2,4-dihydroxy-5-isopropyl-phenyl)-4-(1H-indol-5-yl)-5-mercapto-[1,2,4]triazole,

### 3-(2,4-dihydroxy-5-ethyl-phenyl)-4-(1,2-dimethyl-indol-5-yl)-5-mercapto-[1,2,4]triazole,

### 3-(2,4-dihydroxy-5-isopropyl-phenyl)-4-(1-ethyl-indol-5-yl)-5-mercapto-[1,2,4]triazole,

### 3-(2,4-dihydroxy-5-isopropyl-phenyl)-4-(1-propyl-indol-5-yl)-5-mercapto-[1,2,4]triazole, and

tautomers, pharmaceutically acceptable salts, solvates, clathrates, and prodrugs thereof.

In another embodiment, in formula (XXXI), X₄₂ is N.

In another embodiment, the compound is selected from the group consisting of

### 3-(2,4-dihydroxy-5-ethyl-phenyl)-4-(1-ethyl-benzimidazol-4-yl)-5-mercapto-[1,2,4]triazole,

### 3-(2,4-dihydroxy-5-ethyl-phenyl)-4-(1-ethyl-benzimidazol-4-yl)-5-mercapto-[1,2,4]triazole HCL salt,

### 3-(2,4-dihydroxy-5-ethyl-phenyl)-4-(2-methyl-3-ethyl-benzimidazol-5-yl)-5-mercapto-[1,2,4]triazole,

### 3-(2,4-dihydroxy-5-ethyl-phenyl)-4-(1-ethyl-2-methyl-benzimidazol-5-yl)-5-mercapto-[1,2,4]triazole,

### 3-(2,4-dihydroxy-5-isopropyl-phenyl)-4-(1-methyl-2-trifluoromethyl-benzimidazol-5-yl)-5-mercapto-[1,2,4]triazole, and

tautomers, pharmaceutically acceptable salts, solvates, clathrates, and prodrugs thereof.

In one embodiment, the cell is in a subject in need of treatment for an inflammatory disorder and administration of a compound represented by formula (XXXI), or any of the embodiments of formula (XXXI) disclosed herein, to the subject modulates the activity of glucocorticoid receptors in a cell that is in the subject.

In one embodiment, the cell is in a subject in need of treatment for an immune disorder and administration of a compound represented by formula (XXXI), or any of the embodiments of formula (XXXI) disclosed herein, to the subject modulates the activity of glucocorticoid receptors in a cell that is in the subject.

In one embodiment, the cell is in a subject in need of immunosuppression and administration of a compound represented by formula (XXXI), or any of the embodiments of formula (XXXI) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXI), or any of the embodiments of formula (XXXI) disclosed herein.

In another aspect, the invention provides a method of treating or preventing an immune disorder in a subject, comprising administering to the subject an effective amount of a compound represented by formula (XXXI), or any of the embodiments of formula (XXXI) disclosed herein.

In another aspect, the invention provides a method of suppressing the immune system in a subject in need thereof, comprising administering to the subject an effective amount of a compound represented by formula (XXXI), or any of the embodiments of formula (XXXI) disclosed herein.

In another aspect, the invention provides a method of modulating the activity of glucocorticoid receptors in a cell, comprising administering to the cell an effective amount of a compound represented by formula (XXXII):

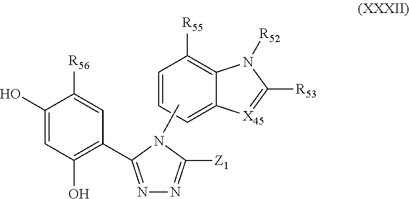

and tautomers, pharmaceutically acceptable salts, solvates, clathrates, and prodrugs thereof, wherein:

X₄₅ is CR₅₄ or N;

Z₁ is —OH or —SH;

R₅₂ is selected from the group consisting of —H, methyl, ethyl, n-propyl, isopropyl, n-butyl, n-pentyl, n-hexyl, —(CH₂)₂OCH₃, —CH₂C(O)OH, and —C(O)N(CH₃)₂;

R₅₃ and R₅₄ are each, independently, —H, methyl, ethyl, or isopropyl; or R₅₃ and R₅₄ taken together with the carbon atoms to which they are attached form a phenyl, cyclohexenyl, or cyclooctenyl ring;

R₅₅ is selected from the group consisting of —H, —OH, —OCH₃, and —OCH₂CH₃; and

R₅₆ is selected from the group consisting of —H, methyl, ethyl, isopropyl, and cyclopropyl.

In one embodiment, in formula (XXXII), Z₁ is —OH.

In another embodiment, in formula (XXXII), Z₁ is —SH.

In another embodiment, in formula (XXXII), R₅₃ is H or a lower alkyl.

In another embodiment, in formula (XXXII), X₄₅ is CR₅₄. Preferably, R₅₄ is H or a lower alkyl.

In another embodiment, X₄₅ is N.

In another embodiment, the compound is 3-(2,4-dihydroxy-5-isopropyl-phenyl)-4-(N-methyl-indol-5-yl)-5-mercapto-[1,2,4]triazole.

In one embodiment, the cell is in a subject in need of treatment for an inflammatory disorder and administration of a compound represented by formula (XXXII), or any of the embodiments of formula (XXXII) disclosed herein, to the subject modulates the activity of glucocorticoid receptors in a cell that is in the subject.

In one embodiment, the cell is in a subject in need of treatment for an immune disorder and administration of a compound represented by formula (XXXII), or any of the embodiments of formula (XXXII) disclosed herein, to the subject modulates the activity of glucocorticoid receptors in a cell that is in the subject.

In one embodiment, the cell is in a subject in need of immunosuppression and administration of a compound represented by formula (XXXII), or any of the embodiments of formula (XXXII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXII), or any of the embodiments of formula (XXXII) disclosed herein.

In another aspect, the invention provides a method of treating or preventing an immune disorder in a subject, comprising administering to the subject an effective amount of a compound represented by formula (XXXII), or any of the embodiments of formula (XXXII) disclosed herein.

In another aspect, the invention provides a method of suppressing the immune system in a subject in need thereof, comprising administering to the subject an effective amount of a compound represented by formula (XXXII), or any of the embodiments of formula (XXXII) disclosed herein.

In another aspect, the invention provides a method of modulating the activity of glucocorticoid receptors in a cell, comprising administering to the cell an effective amount of a compound represented by formula (XXXIII):

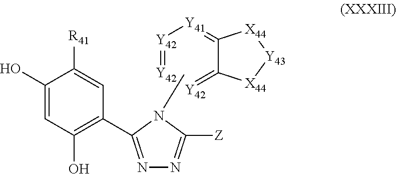

and tautomers, pharmaceutically acceptable salts, solvates, clathrates, and prodrugs thereof, wherein,

X₄₄, for each occurrence, is independently, O, NR₄₂ or C(R₄₆)₂;

Y₄₃ is NR₄₂ or C(R₄₆)₂;

Y₄₁, Y₄₂, Z, R₄₁, R₄₂, and R₄₆ are defined as above.

In one embodiment, in formula (XXXIII), R₄₁ is selected from the group consisting of —H, lower alkyl, lower alkoxy, lower cycloalkyl, and lower cycloalkoxy.

In another embodiment, in formula (XXXIII), R₄₁ is selected from the group consisting of —H, methyl, ethyl, propyl, isopropyl, cyclopropyl, methoxy, ethoxy, propoxy, and cyclopropoxy.

In another embodiment, in formula (XXXIII), R₄₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In another embodiment, in formula (XXXIII), Y₄₁ is CR₄₅. Preferably, R₄₅ is H, a lower alkoxy, or —OH.

In another embodiment, in formula (XXXIII), Y₄₂ is CH.

In another embodiment, in formula (XXXIII), Y₄₃ is CH₂.

In another embodiment, in formula (XXXIII), Y₄₃ is NR₄₂, wherein R₄₂ is H or a lower alkyl.

In another embodiment, in formula (XXXIII), one of X₄₄ is NR₄₂ and the other is CH₂ or C(R₆)₂. Preferably, one of X₄₄ is NR₄₂ and the other is CH₂.

In another embodiment, in formula (XXXIII), Z is —OH.

In another embodiment, Z is —SH.

In one embodiment, the cell is in a subject in need of treatment for an inflammatory disorder and administration of a compound represented by formula (XXXIII), or any of the embodiments of formula (XXXIII) disclosed herein, to the subject modulates the activity of glucocorticoid receptors in a cell that is in the subject.

In one embodiment, the cell is in a subject in need of treatment for an immune disorder and administration of a compound represented by formula (XXXIII), or any of the embodiments of formula (XXXIII) disclosed herein, to the subject modulates the activity of glucocorticoid receptors in a cell that is in the subject.

In one embodiment, the cell is in a subject in need of immunosuppression and administration of a compound represented by formula (XXXIII), or any of the embodiments of formula (XXXIII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXIII), or any of the embodiments of formula (XXXIII) disclosed herein.

In another aspect, the invention provides a method of treating or preventing an immune disorder in a subject, comprising administering to the subject an effective amount of a compound represented by formula (XXXIII), or any of the embodiments of formula (XXXIII) disclosed herein.

In another aspect, the invention provides a method of suppressing the immune system in a subject in need thereof, comprising administering to the subject an effective amount of a compound represented by formula (XXXIII), or any of the embodiments of formula (XXXIII) disclosed herein.

In another aspect, the invention provides a method of modulating the activity of glucocorticoid receptors in a cell, comprising administering to the cell an effective amount of a compound represented by formula (XXXIV):

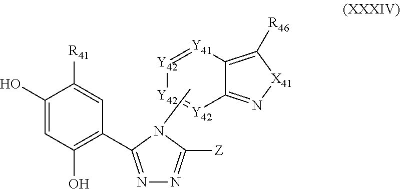

and tautomers, pharmaceutically acceptable salts, solvates, clathrates, and prodrugs thereof, wherein:

X₄₁, Y₄₁, Y₄₂, Z, R₇, R₈, R₁₀, R₁₁, R₄₁, R₄₆, and p are defined as above.

In one embodiment, in formula (XXXIV), R₄₁ is selected from the group consisting of —H, lower alkyl, lower alkoxy, lower cycloalkyl, and lower cycloalkoxy.

In another embodiment, in formula (XXXIV), R₄₁ is selected from the group consisting of —H, methyl, ethyl, propyl, isopropyl, cyclopropyl, methoxy, ethoxy, propoxy, and cyclopropoxy.

In another embodiment, in formula (XXXIV), X₄₁ is NR₄₂. Preferably, R₄₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂. More preferably, R₄₂ is H or a lower alkyl.

In another embodiment, in formula (XXXIV), X₄₁ is O.

In another embodiment, in formula (XXXIV), X₄₁ is S.

In another embodiment, in formula (XXXIV), Y₄₁ is CR₄₅. Preferably, R₄₅ is H, a lower alkoxy, or —OH.

In another embodiment, in formula (XXXIV), Y₄₂ is CH.

In another embodiment, in formula (XXXIV), R₄₆ is H or a lower alkyl.

In one embodiment, the compound is 3-(2,4-dihydroxy-5-isopropyl-phenyl)-4-(2-methyl-indazol-6-yl)-5-mercapto-[1,2,4]triazole.

In one embodiment, the cell is in a subject in need of treatment for an inflammatory disorder and administration of a compound represented by formula (XXXIV), or any of the embodiments of formula (XXXIV) disclosed herein, to the subject modulates the activity of glucocorticoid receptors in a cell that is in the subject.

In one embodiment, the cell is in a subject in need of treatment for an immune disorder and administration of a compound represented by formula (XXXIV), or any of the embodiments of formula (XXXIV) disclosed herein, to the subject modulates the activity of glucocorticoid receptors in a cell that is in the subject.

In one embodiment, the cell is in a subject in need of immunosuppression and administration of a compound represented by formula (XXXIV), or any of the embodiments of formula (XXXIV) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXIV), or any of the embodiments of formula (XXXIV) disclosed herein.

In another aspect, the invention provides a method of treating or preventing an immune disorder in a subject, comprising administering to the subject an effective amount of a compound represented by formula (XXXIV), or any of the embodiments of formula (XXXIV) disclosed herein.

In another aspect, the invention provides a method of suppressing the immune system in a subject in need thereof, comprising administering to the subject an effective amount of a compound represented by formula (XXXIV), or any of the embodiments of formula (XXXIV) disclosed herein.

In another aspect, the invention provides a method of modulating the activity of glucocorticoid receptors in a cell, comprising administering to the cell an effective amount of a compound represented by formula (XXXV):

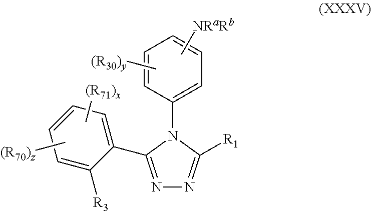

or a tautomer, pharmaceutically acceptable salt, solvate, clathrate or a prodrug thereof.

In formula (XXXV), R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)CR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂. Preferably, R₁ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. More preferably, R₁ is —OH, —SH, or —NHR₇. Even more preferably, R₁, is —SH or —OH;

R₃ is —OH, —SH, —NR₇H, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂. In another embodiment, —OR₂₆ and —SR₂₆, are additional values for R₃. Preferably, R₃ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. More preferably, R₃ is —OH, —SH, or —NHR₇. Even more preferably, R₃ is —SH or —OH;

R₇₀ for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ for each occurrence, is independently an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇. More preferably, R₇₀ for each occurrence, is independently a C1-C6 alkyl, a C1-C6 haloalkyl, a C1-C6 alkoxy, a C1-C6 haloalkoxy, a C1-C6 alkyl sulfanyl or a C3-C6 cycloalkyl. Even more preferably, R₇₀ for each occurrence, is independently cyclopropyl or isopropyl;

R₇ and R₈, for each occurrence, is independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. Preferably, R₇ and R₈, for each occurrence, is independently —H, C1-C3 alkyl, C1-C6 cycloalkyl, an optionally substituted aryl or an optionally substituted heteroaryl. More preferably, R₇ and R₈, for each occurrence, is independently —H or C1-C3 alkyl.

R₁₀ and R₁₁, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. Preferably, R₁₀ and R₁₁, for each occurrence, is independently —H, C1-C3 alkyl, C1-C6 cycloalkyl, an optionally substituted aryl or an optionally substituted heteroaryl. More preferably, R₁₀ and R₁₁, for each occurrence, is independently —H or C1-C3 alkyl.

Alternatively, R₁₀ and R₁₁, taken together with the nitrogen to which they are attached, form an optionally substituted heterocyclyl or an optionally substituted heteroaryl. Preferably R₁₀ and R₁₁, taken together with the nitrogen to which they are attached, form an optionally substituted imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl, thiadiazolyl, pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl. More preferably R₁₀ and R₁₁, taken together with the nitrogen to which they are attached, form an optionally substituted pyrrolidinyl, piperidinyl, piperazinyl, tetrahydroisoquinolinyl, morpholinyl or pyrazolyl.

R₇₁ for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably R₇₁ for each occurrence, is independently —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇. More preferably, R₇₁ for each occurrence, is independently —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. Even more preferably, R₇₁ for each occurrence, is independently —SH or —OH;

R₂₆ is a C1-C6 alkyl;

R₃₀ for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably R₃₀ for each occurrence, is independently an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇. More preferably, R₃₀ for each occurrence, is independently a hydrogen, —OH, —SH, halogen, cyano, a C1-C6 alkyl, C1-C6 haloalkyl, C1-C6 alkoxy, C1-C6 haloalkoxy or C1-C6 alkyl sulfanyl. Even more preferably, R₃₀ for each occurrence, is independently a hydrogen, methyl, ethyl, propyl, isopropyl, methoxy or ethoxy;

R₃₅ is —H, a C1-C4 alkyl or a C1-C4 acyl;

R^{a} and R^{b}, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl or heteroaryl, an optionally substituted aralkyl. Preferably, R^{a} and R^{b} for each occurrence, is independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl. More preferably, R^{a} and R^{b} for each occurrence, is independently a hydrogen, methyl, ethyl, propyl, isopropyl;

Alternatively, R^{a} and R^{b}, taken together with the nitrogen to which they are attached, form an optionally substituted heteroaryl or heterocyclyl. Preferably, R^{a} and R^{b} taken together with the nitrogen to which they are attached form a substituted or unsubstituted nonaromatic, nitrogen-containing heterocyclyl. More preferably, R^{a} and R^{b} taken together with the nitrogen to which they are attached, are:

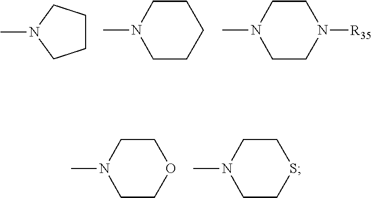

k is 1, 2, 3 or 4;

p, for each occurrence, is independently, 0, 1 or 2;

m, for each occurrence, is independently, 1, 2, 3 or 4;

z and y for each occurrence, is independently an integer from 0 to 4. Preferably z and y for each occurrence, is independently 0, 1, or 2. More preferably z and y for each occurrence, is independently 0 or 1; and

x is 0 or 1, provided that z+x is less than or equal to 4.

In a first preferred embodiment, the values for the variables in formula (IV) are as described in the following paragraphs;

R₇₀, R₇₁ and R₃₀, for each occurrence; is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ and R₃₀ are as just described and R₇₁ is —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇;

k is 1, 2, 3, or 4;

z and y for each occurrence, is independently an integer from 0 to 4;

x is 0 or 1, provided that n+x less than or equal to 4; and

the values and preferred values for the remainder of the variables in formula (XXXV) are as described immediately above.

In one embodiment, the cell is in a subject in need of treatment for an inflammatory disorder and administration of a compound represented by formula (XXXV), or any of the embodiments of formula (XXXV) disclosed herein, to the subject modulates the activity of glucocorticoid receptors in a cell that is in the subject.

In one embodiment, the cell is in a subject in need of treatment for an immune disorder and administration of a compound represented by formula (XXXV), or any of the embodiments of formula (XXXV) disclosed herein, to the subject modulates the activity of glucocorticoid receptors in a cell that is in the subject.

In one embodiment, the cell is in a subject in need of immunosuppression and administration of a compound represented by formula (XXXV), or any of the embodiments of formula (XXXV) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXV), or any of the embodiments of formula (XXXV) disclosed herein.

In another aspect, the invention provides a method of treating or preventing an immune disorder in a subject, comprising administering to the subject an effective amount of a compound represented by formula (XXXV), or any of the embodiments of formula (XXXV) disclosed herein.

In another aspect, the invention provides a method of suppressing the immune system in a subject in need thereof, comprising administering to the subject an effective amount of a compound represented by formula (XXXV), or any of the embodiments of formula (XXXV) disclosed herein.

In another aspect, the invention provides a method of modulating the activity of glucocorticoid receptors in a cell, comprising administering to the cell an effective amount of a compound represented by formula (XXXVI):

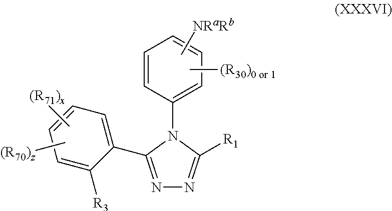

The values and preferred values for the variables in formula (XXXVI) are as described above for formula (XXXV). Alternatively, the values and preferred values for the variables in formula (XXXVI) are as described in the first preferred embodiment for formula (XXXV) immediately above.

In one embodiment, the cell is in a subject in need of treatment for an inflammatory disorder and administration of a compound represented by formula (XXXVI), or any of the embodiments of formula (XXXVI) disclosed herein, to the subject modulates the activity of glucocorticoid receptors in a cell that is in the subject.

In one embodiment, the cell is in a subject in need of treatment for an immune disorder and administration of a compound represented by formula (XXXVI), or any of the embodiments of formula (XXXVI) disclosed herein, to the subject modulates the activity of glucocorticoid receptors in a cell that is in the subject.

In one embodiment, the cell is in a subject in need of immunosuppression and administration of a compound represented by formula (XXXVI), or any of the embodiments of formula (XXXVI) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXVI), or any of the embodiments of formula (XXXVI) disclosed herein.

In another aspect, the invention provides a method of treating or preventing an immune disorder in a subject, comprising administering to the subject an effective amount of a compound represented by formula (XXXVI), or any of the embodiments of formula (XXXVI) disclosed herein.

In another aspect, the invention provides a method of suppressing the immune system in a subject in need thereof, comprising administering to the subject an effective amount of a compound represented by formula (XXXVI), or any of the embodiments of formula (XXXVI) disclosed herein.

In another aspect, the invention provides a method of modulating the activity of glucocorticoid receptors in a cell, comprising administering to the cell an effective amount of a compound represented by formula (XXXVII):

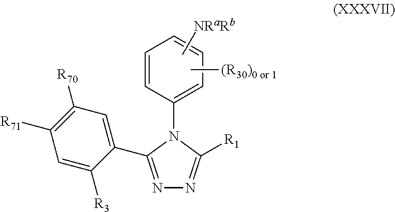

or a tautomer, pharmaceutically acceptable salt, solvate, clathrate or a prodrug thereof.

The values and preferred values for the variables in formula (XXXVII) are as described above for formula (XXXV). Preferably, the values and preferred values for the variables in formula (XXXVII) are as described for formula (XXXVI). More preferably, the values for the variables in formula (XXXVII) are described in the following paragraphs:

R₃₀ is an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; and the values and preferred values for the remainder of the variables are as described above for formula (XXXV). Preferably, the values and preferred values for the remainder of the variables in formula (XXXVII) are as described for formula (XXXVI).

More preferably for formula (XXXVII), R₇₀ is an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; the values for R₃₀ are as described in the preceding paragraph; and the values and preferred values for the remainder of the variables are as described above for formula (XXXV). Preferably, the values and preferred values for the variables in formula (XXXVII) are as described for formula (XXXVI).

In one embodiment, the cell is in a subject in need of treatment for an inflammatory disorder and administration of a compound represented by formula (XXXVII), or any of the embodiments of formula (XXXVII) disclosed herein, to the subject modulates the activity of glucocorticoid receptors in a cell that is in the subject.

In one embodiment, the cell is in a subject in need of treatment for an immune disorder and administration of a compound represented by formula (XXXVII), or any of the embodiments of formula (XXXVII) disclosed herein, to the subject modulates the activity of glucocorticoid receptors in a cell that is in the subject.

In one embodiment, the cell is in a subject in need of immunosuppression and administration of a compound represented by formula (XXXVII), or any of the embodiments of formula (XXXVII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXVII), or any of the embodiments of formula (XXXVII) disclosed herein.

In another aspect, the invention provides a method of treating or preventing an immune disorder in a subject, comprising administering to the subject an effective amount of a compound represented by formula (XXXVII), or any of the embodiments of formula (XXXVII) disclosed herein.

In another aspect, the invention provides a method of suppressing the immune system in a subject in need thereof, comprising administering to the subject an effective amount of a compound represented by formula (XXXVII), or any of the embodiments of formula (XXXVII) disclosed herein.

In another aspect, the invention provides a method of modulating the activity of glucocorticoid receptors in a cell, comprising administering to the cell an effective amount of a compound represented by formula (XXXVIII) or (XXXIX)

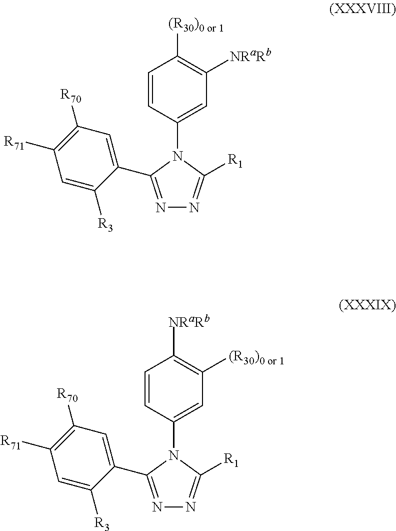

or a tautomer, pharmaceutically acceptable salt, solvate, clathrate or a prodrug thereof.

The values and preferred values for formulas (XXXVIII) and (XXXIX) are as described above for formula (XXXV). Preferably, the values and preferred values for formulas (XXXVIII) and (XXXIX) are as described above for formula (XXXVII). More preferably, the values for the variables in formulas (XXXVIII) and (XXXIX) are described in the following paragraphs:

R₁, R₃ or R₇₁ are each independently —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. Preferably, R₁ and R₃ are each, independently, —OH, —SH, or —NHR₇ and R₇₁ is as just described; and

the values and preferred values for the remainder of the variables are as described above for formula (XXXV) or formula (XXXVII).

In a first more preferred embodiment for formulas (XXXVIII) and (XXXIX), R₁, R₃ and R₇₁ are as described in the immediately preceding two paragraphs: and

R^{a} and R^{b} are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R^{a} and R^{b} taken together with the nitrogen to which they are attached form a substituted or unsubstituted nonaromatic, nitrogen-containing heterocyclyl; and

the values and preferred values for the remainder of the variables are as described above for formula (XXXV) formula (XXXVII).

In a second more preferred embodiment for formulas (XXXVIII) and (XXXIX), R₇₀ is a C1-C6 alkyl, a C1-C6 haloalkyl, a C1-C6 alkoxy, a C1-C6 haloalkoxy, a C1-C6 alkyl sulfanyl or a C3-C6 cycloalkyl; and the values and preferred values for the remainder of the variables are as described above for first more preferred embodiment for formulas (XXXVIII) and (XXXIX).

In a third more preferred embodiment for formulas (XXXVIII) and (XXXIX):

R₁ and R₃ are each, independently, —OH, —SH, or —NHR₇;

R₇₀ is a C1-C6 alkyl, a C1-C6 haloalkyl, a C1-C6 alkoxy, a C1-C6 haloalkoxy, a C1-C6 alkyl sulfanyl or a C3-C6 cycloalkyl;

R₇₁ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂;

R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl. Preferably, R₃₀ is methyl, ethyl, propyl, isopropyl, methoxy or ethoxy;

R^{a} and R^{b} are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R^{a} and R^{b} taken together with the nitrogen to which they are attached form a substituted or unsubstituted nonaromatic, nitrogen-containing heterocyclyl; and

the values and preferred values for the remainder of the variables are as described above for formula (XXXVII).

In a fourth more preferred embodiment for formulas (XXXVIII) and (XXXIX):

R₁, R₃ and R₇₁ for each occurrence, is independently —SH or —OH;

R₇₀ is cyclopropyl or isopropyl; and

the remainder of the variables are as described for the third more preferred embodiment for formulas (XXXVIII) and (XXXIX). More preferably R₃₀ is methyl,ethyl, propyl, isopropyl, methoxy or ethoxy. Even more preferably, R₃₀ is methyl, ethyl, propyl, isopropyl, methoxy or ethoxy and R^{a} and R^{b} are each independently a hydrogen, methyl, ethyl, propyl, isopropyl, or taken together with the nitrogen to which they are attached, are:

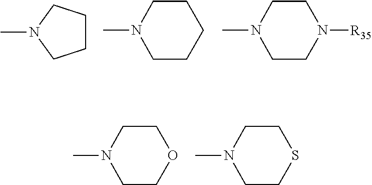

wherein R₃₅ is —H, a C1-C4 alkyl or a C1-C4 acyl; and

the values and preferred values for the remainder of the variables are as described above for formula (XXXVII).

In another preferred embodiment, the present invention is a compound represented by formula (XXXV), (XXXVI), (XXXVII), (XXXVIII) or (XXXIX), wherein R₁, R₃ and R₇₁ are —SH or —OH and R₆ is cyclopropyl or isopropyl and the remainder of the variables are as described for Formula (XXXV), (XXXVI), (XXXVII), (XXXVIII) or (XXXIX), respectively.

In one embodiment, the cell is in a subject in need of treatment for an inflammatory disorder and administration of a compound represented by formula (XXXVIII) or (XXXIX), or any of the embodiments of formula (XXXVIII) or (XXXIX) disclosed herein, to the subject modulates the activity of glucocorticoid receptors in a cell that is in the subject.

In one embodiment, the cell is in a subject in need of treatment for an immune disorder and administration of a compound represented by formula (XXXVIII) or (XXXIX), or any of the embodiments of formula (XXXVIII) or (XXXIX) disclosed herein, to the subject modulates the activity of glucocorticoid receptors in a cell that is in the subject.

In one embodiment, the cell is in a subject in need of immunosuppression and administration of a compound represented by formula (XXXVIII) or (XXXIX), or any of the embodiments of formula (XXXVIII) or (XXXIX) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XXXVIII) or (XXXIX), or any of the embodiments of formula (XXXVIII) or (XXXIX) disclosed herein.

In another aspect, the invention provides a method of treating or preventing an immune disorder in a subject, comprising administering to the subject an effective amount of a compound represented by formula (XXXVIII) or (XXXIX), or any of the embodiments of formula (XXXVIII) or (XXXIX) disclosed herein.

In another aspect, the invention provides a method of suppressing the immune system in a subject in need thereof, comprising administering to the subject an effective amount of a compound represented by formula (XXXVIII) or (XXXIX), or any of the embodiments of formula (XXXVIII) or (XXXIX) disclosed herein.

In another aspect, the invention provides a method of modulating the activity of glucocorticoid receptors in a cell, comprising administering to the cell an effective amount of a compound represented by formula (XL) or (XLI):

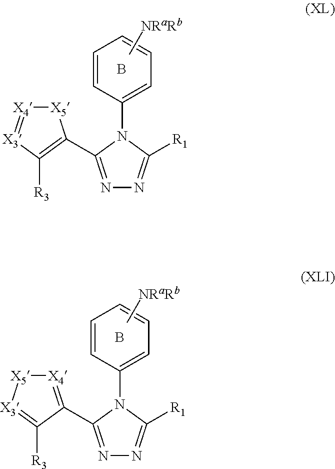

or a tautomer, pharmaceutically acceptable salt, solvate, clathrate or a prodrug thereof.

In formulas (XL) and (XLI), ring B is further optionally substituted with one or more substituents in addition to —NR^{a}R^{b}. Preferably ring B is substituted with (R₃₀)_{y} where y is 0, 1, 2, 3 or 4, preferably y is 0 or 1;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁, —OP(O)(OR₇)₂ or —SP(O)(OR₇)₂. Preferably, R₁ is —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂. More preferably, R₁ is —OH, —SH, or —NHR₇. Even more preferably, R₁ is —SH or —OH;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂. Preferably, R₃ is —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂. More preferably, R₃ is —OH, —SH, or —NHR₇. Even more preferably, R₃ is —SH or —OH;

R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇ or —SS(O)ₚNR₁₀R₁₁. Preferably, R₇₀ is for each occurrence, is independently an optionally substituted C1-C6 alkyl, an optionally substituted C3-C6 cycloalkyl, an optionally substituted C3-C6 cycloalkenyl, an optionally substituted heterocyclyl, a halo, a haloalkyl, a haloalkoxy, a heteroalkyl, an alkoxy, an alkylsulfanyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)OR₇, —S(O)ₚNR₁₀R₁₁, —S(O)ₚR₇, —OP(O)(OR₇)₂ or —SP(O)(OR₇)₂. More preferably, R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR_{7,} —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂. Even more preferably, R₇₀ is for each occurrence, is independently a C1-C6 alkyl, a C1-C6 haloalkyl, a C1-C6 alkoxy, a C1-C6 haloalkoxy, a C1-C6 alkyl sulfanyl or a C3-C6 cycloalkyl. Still more preferably, R₇₀ for each occurrence, is independently a cyclopropyl or isopropyl;

R₇ and R₈, for each occurrence, is independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. Preferably, R₇ and R₈, for each occurrence, is independently —H, C1-C3 alkyl, C1-C6 cycloalkyl, an optionally substituted aryl or an optionally substituted heteroaryl. More preferably, R₇ and R₈, for each occurrence, is independently —H or C1-C3 alkyl;

R₁₀ and R₁₁, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. Preferably, R₁₀ and R₁₁, for each occurrence, is independently —H, C1-C3 alkyl, C1-C6 cycloalkyl, an optionally substituted aryl or an optionally substituted heteroaryl. More preferably, R₁₀ and R₁₁, for each occurrence, is independently —H or C1-C3 alkyl;

alternatively, R₁₀ and R₁₁, taken together with the nitrogen to which they are attached, form an optionally substituted heterocyclyl or an optionally substituted heteroaryl. Preferably R₁₀ and R₁₁, taken together with the nitrogen to which they are attached, form an optionally substituted imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl, thiadiazolyl, pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl. More preferably R₁₀ and R₁₁, taken together with the nitrogen to which they are attached, form an optionally substituted pyrrolidinyl, piperidinyl, piperazinyl, tetrahydroisoquinolinyl, morpholinyl or pyrazolyl;

R₁₇, for each occurrence, is independently an alkyl or an aralkyl. Preferably R₁₇ for each occurrence is independently a C1-C6 alkyl;

R₂₆ is a C1-C6 alkyl;

R₃₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —H, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, or —SS(O)ₚNR₁₀R₁₁. Preferably R₃₀ for each occurrence, is independently an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)OR₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁ or —S(O)ₚR₇. More preferably, R₃₀ for each occurrence, is independently a hydrogen, —OH, —SH, halogen, cyano, a C1-C6 alkyl, C1-C6 haloalkyl, C1-C6 alkoxy, C1-C6 haloalkoxy or C1-C6 alkyl sulfanyl. Even more preferably, R₃₀ for each occurrence, is independently a hydrogen, methyl, ethyl, propyl, isopropyl, methoxy or ethoxy;

R^{a} and R^{b}, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl or heteroaryl, an optionally substituted aralkyl. Preferably, R^{a} and R^{b} for each occurrence, is independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl. More preferably, R^{a} and R^{b} for each occurrence, is independently a hydrogen, methyl, ethyl, propyl, isopropyl;

Alternatively, R^{a} and R^{b}, taken together with the nitrogen to which they are attached, form an optionally substituted heteroaryl or heterocyclyl. Preferably, R^{a} and R^{b} taken together with the nitrogen to which they are attached form a substituted or unsubstituted nonaromatic, nitrogen-containing heterocyclyl. More preferably, R^{a} and R^{b} taken together with the nitrogen to which they are attached, are:

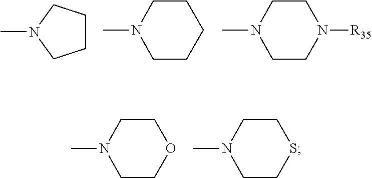

X₃′ and X₄′ are each, independently, N, N(O), N⁺(R₁₇), CH or CR₇₀;

X₅′ is O, S, NR₁₇, CH₂, CH(R₇₀), C(R₇₀)₂, CH═CH, CH═CR₇₀, CR₇₀═CH, CR₇₀═CR₇₀, CH═N, CR₇₀═N, CH═N(O), CR₇₀═N(O), N═CH, N═CR₇₀, N(O)═CH, N(O)═CR₇₀, N⁺(R₁₇)═CH, N⁺(R₁₇)═CR₇₀, CH═N⁺(R₁₇), CR₇₀═N⁺(R₁₇), or N═N, provided that at least one X₃′, X₄′ or X₅′ is a heteroatom;

k is 1, 2, 3, or 4;

p, for each occurrence, is independently, 0, 1 or 2; and

m, for each occurrence, is independently, 1, 2, 3, or 4.

In one embodiment, the cell is in a subject in need of treatment for an inflammatory disorder and administration of a compound represented by formula (XL) or (XLI), or any of the embodiments of formula (XL) or (XLI) disclosed herein, to the subject modulates the activity of glucocorticoid receptors in a cell that is in the subject.

In one embodiment, the cell is in a subject in need of treatment for an immune disorder and administration of a compound represented by formula (XL) and (XLI), or any of the embodiments of formula (XL) and (XLI) disclosed herein, to the subject modulates the activity of glucocorticoid receptors in a cell that is in the subject.

In one embodiment, the cell is in a subject in need of immunosuppression and administration of a compound represented by formula (XL) or (XLI), or any of the embodiments of formula (XL) or (XLI) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XL) or (XLI), or any of the embodiments of formula (XL) or (XLI) disclosed herein.

In another aspect, the invention provides a method of treating or preventing an immune disorder in a subject, comprising administering to the subject an effective amount of a compound represented by formula (XL) or (XLI), or any of the embodiments of formula (XL) or (XLI) disclosed herein.

In another aspect, the invention provides a method of suppressing the immune system in a subject in need thereof, comprising administering to the subject an effective amount of a compound represented by formula (XL) or (XLI), or any of the embodiments of formula (XL) or (XLI) disclosed herein.

In another aspect, the invention provides a method of modulating the activity of glucocorticoid receptors in a cell, comprising administering to the cell an effective amount of a compound represented by formula (XLII) or (XLIII):

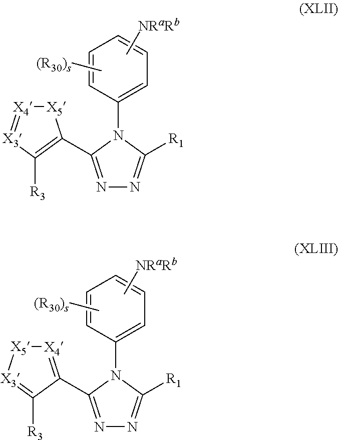

or a tautomer, pharmaceutically acceptable salt, solvate, clathrate or a prodrug thereof.

Preferably the values and preferred values for formulas (XLII) and (XLIII) are as described above for formulas (XL) and (XLI), and more preferably:

R₇₀ is for each occurrence, is independently an optionally substituted C1-C6 alkyl, an optionally substituted C3-C6 cycloalkyl, an optionally substituted C3-C6 cycloalkenyl, an optionally substituted heterocyclyl, a halo, a haloalkyl, a haloalkoxy, a heteroalkyl, an alkoxy, an alkylsulfanyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, —S(O)ₚR₇, —OP(O)(OR₇)₂ or —SP(O)(OR₇)₂;

R₃₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇ or —SS(O)ₚNR₁₀R₁₁;

s is 0, 1, 2, 3 or 4;

k is 1, 2, 3, or 4; and

the values and preferred values for the remainder of the variables are as described above for formulas (XL) and (XLI).

In one embodiment, the cell is in a subject in need of treatment for an inflammatory disorder and administration of a compound represented by formula (XLII) or (XLIII), or any of the embodiments of formula (XLII) or (XLIII) disclosed herein, to the subject modulates the activity of glucocorticoid receptors in a cell that is in the subject.

In one embodiment, the cell is in a subject in need of treatment for an immune disorder and administration of a compound represented by formula (XLII) or (XLIII), or any of the embodiments of formula (XLII) or (XLIII) disclosed herein, to the subject modulates the activity of glucocorticoid receptors in a cell that is in the subject.

In one embodiment, the cell is in a subject in need of immunosuppression and administration of a compound represented by formula (XLII) or (XLIII), or any of the embodiments of formula (XLII) or (XLIII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XLII) or (XLIII), or any of the embodiments of formula (XLII) or (XLIII) disclosed herein.

In another aspect, the invention provides a method of treating or preventing an immune disorder in a subject, comprising administering to the subject an effective amount of a compound represented by formula (XLII) or (XLIII), or any of the embodiments of formula (XLII) or (XLIII) disclosed herein.

In another aspect, the invention provides a method of suppressing the immune system in a subject in need thereof, comprising administering to the subject an effective amount of a compound represented by formula (XLII) or (XLIII), or any of the embodiments of formula (XLII) or (XLIII) disclosed herein.

In another aspect, the invention provides a method of modulating the activity of glucocorticoid receptors in a cell, comprising administering to the cell an effective amount of a compound represented by formula (XLIV) or (XLV):

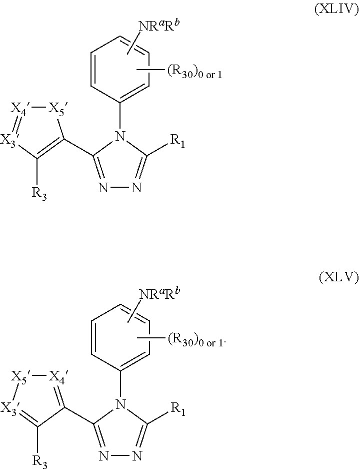

or a tautomer, pharmaceutically acceptable salt, solvate, clathrate or a prodrug thereof.

The values and preferred values for formulas (XLIV) and (XLV) are as described above for formulas (XL) and (XLI). Preferably the values and preferred values for formulas (XLIV) and (XLV) are as described for formulas (XLII) and (XLIII). More preferably, the values for formulas (XLIV) and (XLV) are described in the following paragraphs:

R₃₀ is an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; and

the values and preferred values for the remainder of the variables are as described above for formulas (XLIV) and (XLV) are as described above for formulas (XL) and (XLI). Preferably the values and preferred values for the remainder of the variables in formulas (XLIV) and (XLV) are as described for formulas (XLII) and (XLIII).

In one embodiment, the cell is in a subject in need of treatment for an inflammatory disorder and administration of a compound represented by formula (XLIV) and (XLV), or any of the embodiments of formula (XLIV) and (XLV) disclosed herein, to the subject modulates the activity of glucocorticoid receptors in a cell that is in the subject.

In one embodiment, the cell is in a subject in need of treatment for an immune disorder and administration of a compound represented by formula (XLIV) or (XLV), or any of the embodiments of formula (XLIV) or (XLV) disclosed herein, to the subject modulates the activity of glucocorticoid receptors in a cell that is in the subject.

In one embodiment, the cell is in a subject in need of immunosuppression and administration of a compound represented by formula (XLIV) or (XLV), or any of the embodiments of formula (XLIV) or (XLV) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (XLIV) or (XLV), or any of the embodiments of formula (XLIV) or (XLV) disclosed herein.

In another aspect, the invention provides a method of treating or preventing an immune disorder in a subject, comprising administering to the subject an effective amount of a compound represented by formula (XLIV) or (XLV), or any of the embodiments of formula (XLIV) or (XLV) disclosed herein.

In another aspect, the invention provides a method of suppressing the immune system in a subject in need thereof, comprising administering to the subject an effective amount of a compound represented by formula (XLIV) or (XLV), or any of the embodiments of formula (XLIV) or (XLV) disclosed herein.

In another aspect, the invention provides a method of modulating the activity of glucocorticoid receptors in a cell, comprising administering to the cell an effective amount of a compound represented by any one of formulas (XLVI)-(XLIX):

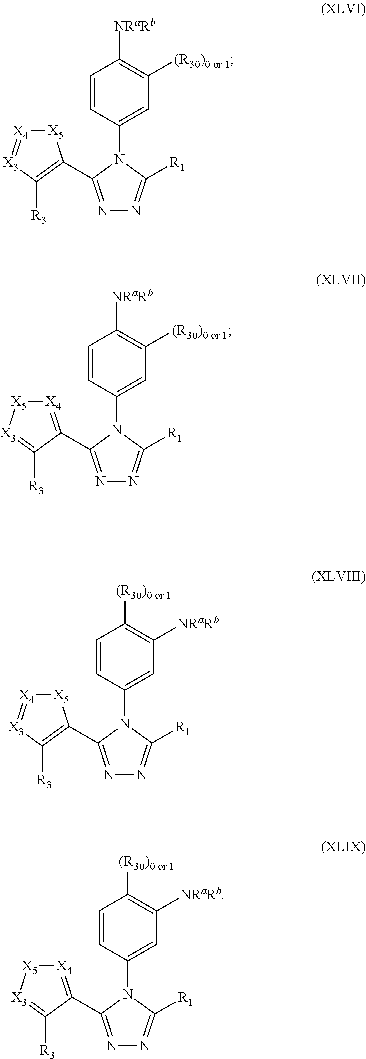

or a tautomer, pharmaceutically acceptable salt, solvate, clathrate or a prodrug thereof.

The values and preferred values for formulas (XLVI)-(XLIX) are as described above for formulas (XL) and (XLI). Preferably the values and preferred values for formulas (XLVI)-(XLIX) are as described above for formulas (XLIV) and (XLV). More preferably, the values for formulas (XLVI)-(XLIX) are provided below in the following paragraphs:

R₁ and R₃ are each independently —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂; and

R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂; and

the values and preferred values for the remainder of the variables are as described for formulas (XLIV) and (XLV).

Still more preferably for formulas (XLVI)-(XLIX), R₁, R₃ and R₇₀ are as described in the immediately preceding paragraphs; and

R^{a} and R^{b} are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R^{a} and R^{b} taken together with the nitrogen to which they are attached form a substituted or unsubstituted nonaromatic, nitrogen-containing heterocyclyl; and

the values and preferred values for the remainder of the variables are as described for formulas (XLIV) and (XLV).

Still more preferably for formulas (XLVI)-(XLIX), R₁, R₃, R₆, R^{a} and R^{b} are as described in the immediately preceding paragraphs; and

R₇₀ is a C1-C6 alkyl, a C1-C6 haloalkyl, a C1-C6 alkoxy, a C1-C6 haloalkoxy, a C1-C6 alkyl sulfanyl or a C3-C6 cycloalkyl; and

the values and preferred values for the remainder of the variables are as described above for formulas (XL) and (XLI). More preferably, the values and preferred values for the remainder of the variables are as described above for formulas (XLIV) and (XLV).

In one embodiment, the cell is in a subject in need of treatment for an inflammatory disorder and administration of a compound represented by any one of formulas (XLVI)-(XLIX), or any of the embodiments of formulas (XLVI)-(XLIX) disclosed herein, to the subject modulates the activity of glucocorticoid receptors in a cell that is in the subject.

In one embodiment, the cell is in a subject in need of treatment for an immune disorder and administration of a compound represented by any one of formulas (XLVI)-(XLIX), or any of the embodiments of formulas (XLVI)-(XLIX) disclosed herein, to the subject modulates the activity of glucocorticoid receptors in a cell that is in the subject.

In one embodiment, the cell is in a subject in need of immunosuppression and administration of a compound represented by any one of formulas (XLVI)-(XLIX), or any of the embodiments of formulas (XLVI)-(XLIX) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (XLVI)-(XLIX), or any of the embodiments of formulas (XLVI)-(XLIX) disclosed herein.

In another aspect, the invention provides a method of treating or preventing an immune disorder in a subject, comprising administering to the subject an effective amount of a compound represented by any one of formulas (XLVI)-(XLIX), or any of the embodiments of formulas (XLVI)-(XLIX) disclosed herein.

In another aspect, the invention provides a method of suppressing the immune system in a subject in need thereof, comprising administering to the subject an effective amount of a compound represented by any one of formulas (XLVI)-(XLIX), or any of the embodiments of formulas (XLVI)-(XLIX) disclosed herein.

In another aspect, the invention provides a method of modulating the activity of glucocorticoid receptors in a cell, comprising administering to the cell an effective amount of a compound represented by any one of formulas (La)-(Lp):

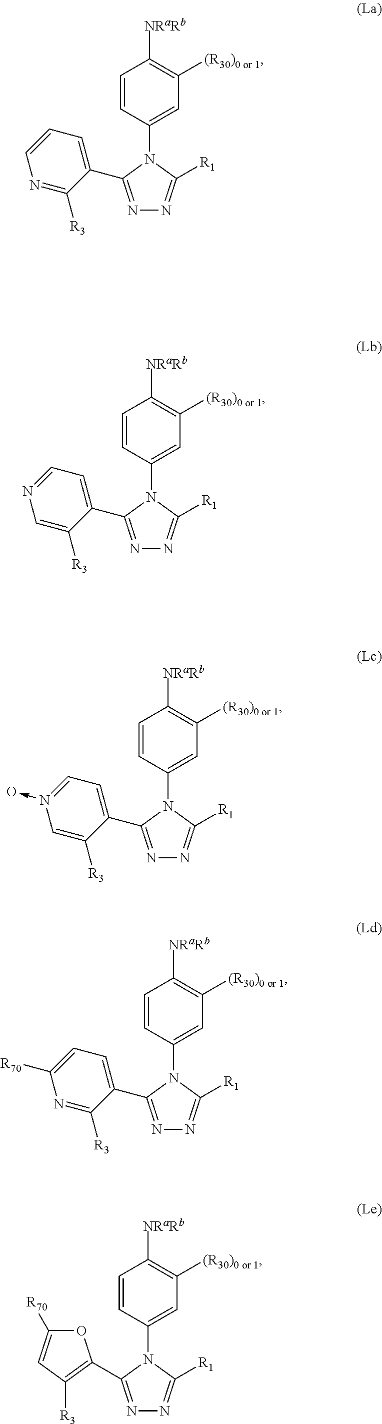

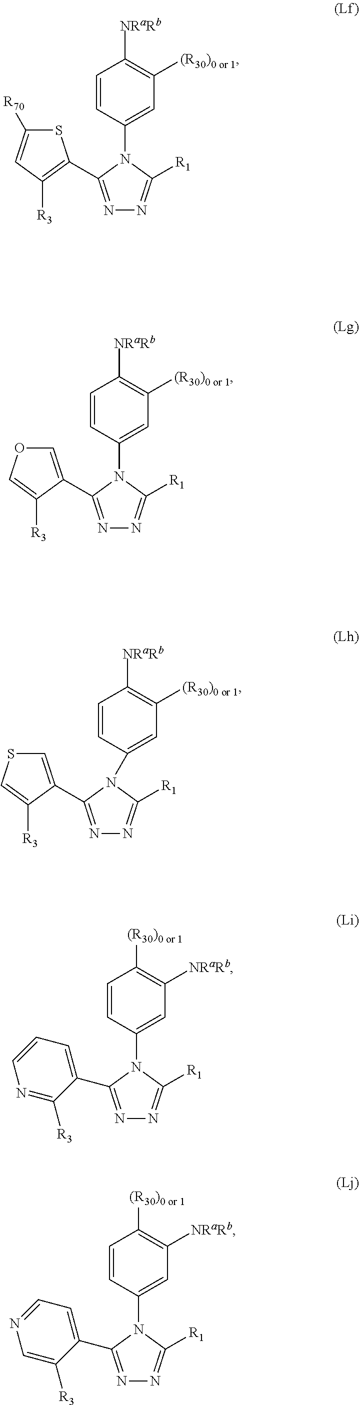

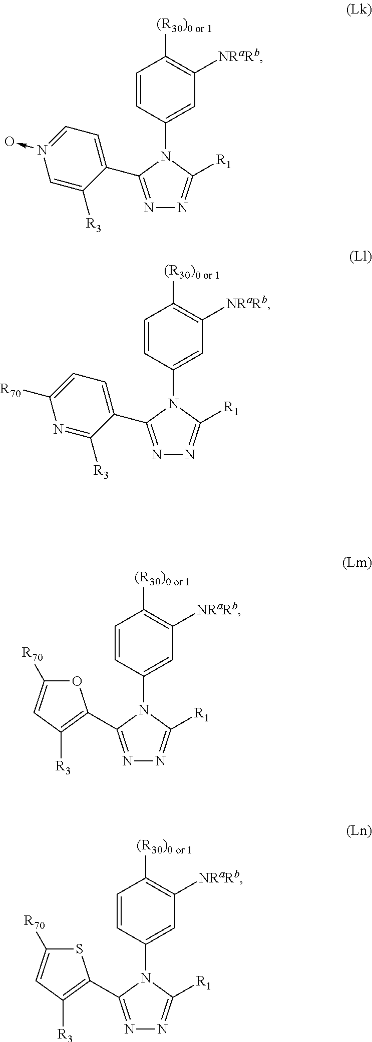

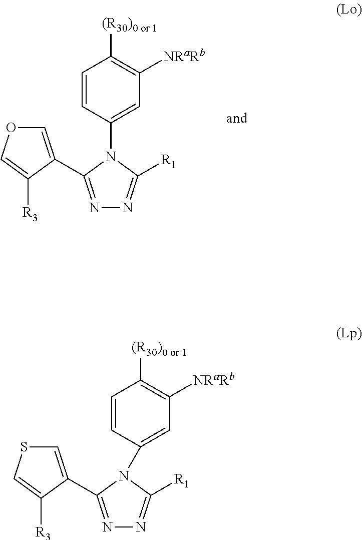

or a tautomer, pharmaceutically acceptable salt, solvate, clathrate or a prodrug thereof.

The values and preferred values for formulas (La) through (Lp) are as described above for formulas (XL) and (XLI). Preferably the values and preferred values for formulas (La)-(Lp) are as described for formulas (XLVI)-(XLIX). More preferably, R₁ and R₃ are each, independently, —OH, —SH, or —NHR₇. Even more preferable, R₁ and R₃ are each, independently, —OH, —SH, or —NHR₇; and R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl (preferably methyl, ethyl, propyl, isopropyl, methoxy or ethoxy). Even more preferably, R₁ and R₃ for each occurrence, is independently —SH or —OH; R₇₀ is cyclopropyl or isopropyl; and R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl (preferably methyl, ethyl, propyl, isopropyl, methoxy or ethoxy). Even more preferably yet, R₁, R₃, R₇₀ and R₃₀ are as just described and R^{a} and R^{b} are each independently a hydrogen, methyl, ethyl, propyl, isopropyl, or taken together with the nitrogen to which they are attached, are:

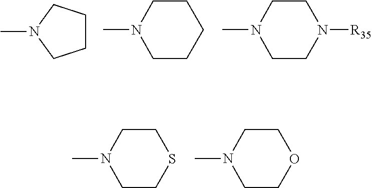

R₃₅ is —H, a C1-C4 alkyl or a C1-C4 acyl; and

the values and preferred values for the remainder of the variables are as defined for formulas (XLVI)-(XLIX).

In one embodiment, the cell is in a subject in need of treatment for an inflammatory disorder and administration of a compound represented by any one of formulas (La)-(Lp), or any of the embodiments of formulas (La)-(Lp) disclosed herein, to the subject modulates the activity of glucocorticoid receptors in a cell that is in the subject.

In one embodiment, the cell is in a subject in need of treatment for an immune disorder and administration of a compound represented by any one of formulas (La)-(Lp), or any of the embodiments of formulas (La)-(Lp) disclosed herein, to the subject modulates the activity of glucocorticoid receptors in a cell that is in the subject.

In one embodiment, the cell is in a subject in need of immunosuppression and administration of a compound represented by any one of formulas (La)-(Lp), or any of the embodiments of formulas (La)-(Lp) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (La)-(Lp), or any of the embodiments of formulas (La)-(Lp) disclosed herein.

In another aspect, the invention provides a method of treating or preventing an immune disorder in a subject, comprising administering to the subject an effective amount of a compound represented by any one of formulas (La)-(Lp), or any of the embodiments of formulas (La)-(Lp) disclosed herein.

In another aspect, the invention provides a method of suppressing the immune system in a subject in need thereof, comprising administering to the subject an effective amount of a compound represented by any one of formulas (La)-(Lp), or any of the embodiments of formulas (La)-(Lp) disclosed herein.

In another aspect, the invention provides a method of modulating the activity of glucocorticoid receptors in a cell, comprising administering to the cell an effective amount of a compound represented by formula (LIa) or (LIb):

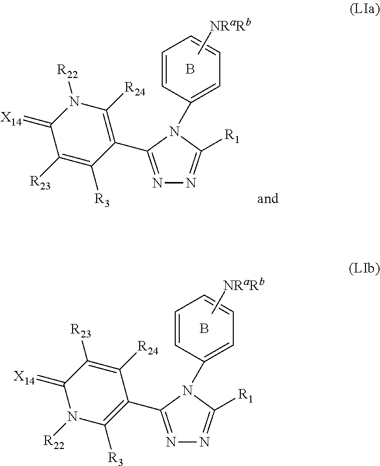

or a tautomer, pharmaceutically acceptable salt, solvate, clathrate or a prodrug thereof.

In formulas (LIa) and (LIb), ring B is further optionally substituted with one or more substituents in addition to —NR^{a}R^{b}. Preferably ring B is further substituted with (R₃₀)ₛ where s is 0, 1, 2, 3 or 4, preferably s is 0 or 1;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂ or —SP(O)(OR₇)₂. Preferably, R₁ is —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂. More preferably, R₁ is —OH, —SH, or —NHR₇. Even more preferably, R₁ is —SH or —OH;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂. Preferably, R₃ is —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂. More preferably, R₃ is —OH, —SH, or —NHR₇. Even more preferably, R₃ is —SH or —OH;

R₇ and R₈, for each occurrence, is independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. Preferably, R₇ and R₈, for each occurrence, is independently —H, C1-C3 alkyl, C1-C6 cycloalkyl, an optionally substituted aryl or an optionally substituted heteroaryl. More preferably, R₇ and R₈, for each occurrence, is independently —H or C1-C3 alkyl;

R₁₀ and R₁₁, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. Preferably, R₁₀ and R₁₁, for each occurrence, is independently —H, C1-C3 alkyl, C1-C6 cycloalkyl, an optionally substituted aryl or an optionally substituted heteroaryl. More preferably, R₁₀ and R₁₁, for each occurrence, is independently —H or C1-C3 alkyl;

Alternatively, R₁₀ and R₁₁, taken together with the nitrogen to which they are attached, form an optionally substituted heterocyclyl or an optionally substituted heteroaryl. Preferably R₁₀ and R₁₁, taken together with the nitrogen to which they are attached, form an optionally substituted imidazolyl, pyrrolyl, pyrazolyl, triazolyl, tetrazolyl, pyridinyl, pyrazinyl, pyrimidinyl, pyridazinyl, oxazolyl, iosoxazolyl, oxadiazolyl, thiazolyl, isothiazolyl, thiadiazolyl, pyrrolidinyl, piperidinyl, morpholinyl, pyrazinyl, thiomorpholinyl, pyrrolidinyl, piperidinyl, pyranzinyl, thiomorpholinyl, tetrahydroquinolinyl or tetrahydroisoquinolinyl. More preferably R₁₀ and R₁₁, taken together with the nitrogen to which they are attached, form an optionally substituted pyrrolidinyl, piperidinyl, piperazinyl, tetrahydroisoquinolinyl, morpholinyl or pyrazolyl;

R₂₂, for each occurrence, is independently —H, an optionally substituted alky, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl, a haloalkyl, —C(O)R₇, —C(O)OR₇, —OC(O)R₇, —C(O)NR₁₀R₁₁, —NR₈C(O)R₇, —S(O)ₚR₇, —S(O)ₚOR₇, or —S(O)ₚNR₁₀R₁₁. Preferably, R₂₂ is —H, an alkyl, an aralkyl, —C(O)R₇, —C(O)OR₇, or —C(O)NR₁₀R₁₁;

R₂₃ and R₂₄, for each occurrence, is independently —H, an optionally substituted alky, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁. Preferably, R₂₃ and R₂₄ for each occurrence is independently —H;

R₂₆ is a C1-C6 alkyl;

R^{a} and R^{b}, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl or heteroaryl, an optionally substituted aralkyl. Preferably, R^{a} and R^{b} for each occurrence, is independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl. More preferably, R^{a} and R^{b} for each occurrence, is independently a hydrogen, methyl, ethyl, propyl or isopropyl;

Alternatively, R^{a} and R^{b}, taken together with the nitrogen to which they are attached, form an optionally substituted heteroaryl or heterocyclyl. Preferably, R^{a} and R^{b} taken together with the nitrogen to which they are attached form a substituted or unsubstituted nonaromatic, nitrogen-containing heterocyclyl. More preferably, R^{a} and R^{b} taken together with the nitrogen to which they are attached, are:

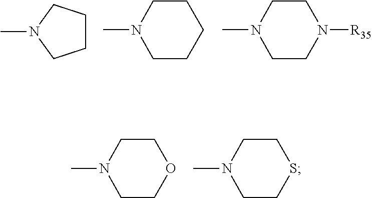

X₁₄ is O, S, or NR₇. Preferably, X₁₄ is O;

p, for each occurrence, is independently, 0, 1 or 2; and

m, for each occurrence, is independently, 1, 2, 3, or 4.

Preferably for the compound represented by formulas (LIa) and (LIb), R₁ is —OH, —SH, or —NHR₇; and R₂₂ is —H, an alkyl, an aralkyl, —C(O)R₇, —C(O)OR₇, or —C(O)NR₁₀R₁₁. More preferably, R₁ is —OH, —SH, or —NHR₇; R₂₂ is —H, an alkyl, an aralkyl, —C(O)R₇, —C(O)OR₇, or —C(O)NR₁₀R₁₁; and X₁₄ is O. The values and preferred values for the remainder of the variables are as described above.

In one embodiment, the cell is in a subject in need of treatment for an inflammatory disorder and administration of a compound represented by formula (LIa) or (LIb), or any of the embodiments of formula (LIa) or (LIb) disclosed herein, to the subject modulates the activity of glucocorticoid receptors in a cell that is in the subject.

In one embodiment, the cell is in a subject in need of treatment for an immune disorder and administration of a compound represented by formula (LIa) or (LIb), or any of the embodiments of formula (LIa) or (LIb) disclosed herein, to the subject modulates the activity of glucocorticoid receptors in a cell that is in the subject.

In one embodiment, the cell is in a subject in need of immunosuppression and administration of a compound represented by formula (LIa) or (LIb), or any of the embodiments of formula (LIa) or (LIb) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LIa) or (LIb), or any of the embodiments of formula (LIa) or (LIb) disclosed herein.

In another aspect, the invention provides a method of treating or preventing an immune disorder in a subject, comprising administering to the subject an effective amount of a compound represented by formula (LIa) or (LIb), or any of the embodiments of formula (LIa) or (LIb) disclosed herein.

In another aspect, the invention provides a method of suppressing the immune system in a subject in need thereof, comprising administering to the subject an effective amount of a compound represented by formula (LIa) or (LIb), or any of the embodiments of formula (LIa) or (LIb) disclosed herein.

In another aspect, the invention provides a method of modulating the activity of glucocorticoid receptors in a cell, comprising administering to the cell an effective amount of a compound represented by any one of formulas (VI)-(VIII):

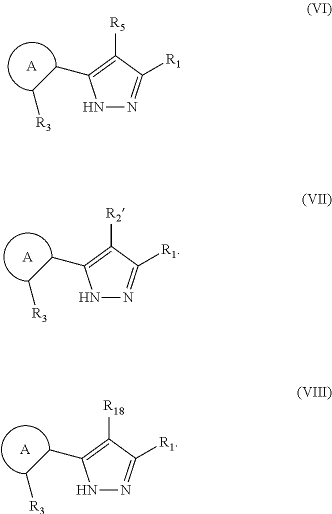

In formulas (VI-VIII):

ring A is an aryl or a heteroaryl, optionally further substituted with one or more substituents in addition to R₃. Preferably, Ring A is represented one of the following structural formulas:

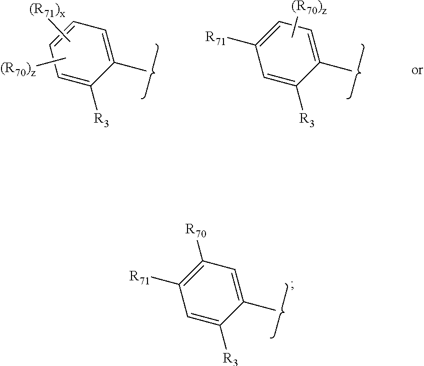

where z is 0, 1, 2, 3 or 4; x is 0 or 1; and z+x is less than or equal to 4.

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂. Preferably, R₁ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. More preferably, R₁ is —OH, —SH, or —NHR₇. Even more preferably, R₁, is —SH or —OH;

R₂′ is an optionally substituted phenyl group. Preferably, R₂′ is substituted with one or more group represented by R₃₀, wherein R₃₀, for each occurrence, are independently an -optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. More preferably, R₂′ is an optionally substituted indolyl group or a phenyl group substituted with NR₁₀R₁₁ and optionally with at least one other substituent represented by R₃₀;

R₃ is —OH, —SH, —NR₇H, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂. In another embodiment, —OR₂₆ and —SR₂₆, are additional values for R₃. Preferably, R₃ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. More preferably, R₃ is —OH, —SH, or —NHR₇. Even more preferably, R₃ is —SH or —OH;

R₅ is an optionally substituted heteroaryl; an optionally substituted 6 to 14-membered aryl.

R₇₀, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ is selected from the group consisting of —H, C1-C6 alkyl, C1-C6 alkoxy, C1-C6 cycloalkyl, and C1-C6 cycloalkoxy, more preferably from the group consisting of —H, methyl, ethyl, propyl, isopropyl, cyclopropyl, methoxy, ethoxy, propoxy, and cyclopropoxy.

R₇₁, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂.

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

R₂₆ is a lower alkyl;

p, for each occurrence, is, independently, 0, 1 or 2; and

m, for each occurrence, is independently, 1, 2, 3, or 4.

R₅ in structural formula (VI) is preferably represented by the following structural formula:

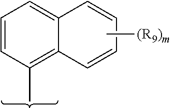

wherein:

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring; and

m is zero or an integer from 1 to 7.

More preferably, substituent R₅ in structural formula (VI) is represented by one of the following structural formulas:

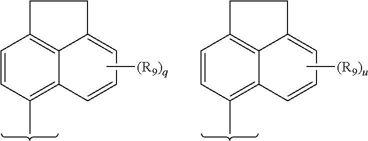

wherein:

R₉ is as defined as above, q is zero or an integer from 1 to 7 and u is zero or an integer from 1 to 8.

In another alternative, R₅ in structural formula (VI) is represented by the following structural formula:

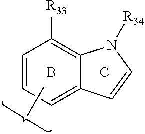

wherein:

R₃₃ is —H, a halo, lower alkyl, a lower alkoxy, a lower haloalkyl, a lower haloalkoxy, and lower alkyl sulfanyl; R₃₄ is H, a lower alkyl, or a lower alkylcarbonyl; and ring B and ring C are optionally substituted with one or more substituents.

In another alternative, R₅ in structural formula (VI) is selected from a group listed in Table 1.

TABLE 1

| # | R₅ |
|---|---|
| 1 | 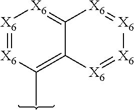 |
| 2 | 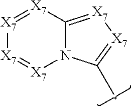 |
| 3 | 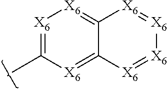 |
| 4 | 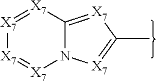 |
| 5 | 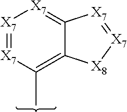 |
| 6 | 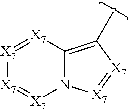 |
| 7 | 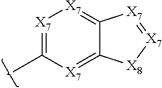 |
| 8 | 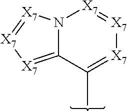 |
| 9 | 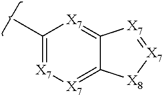 |
| 10 | 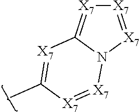 |
| 11 | 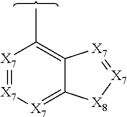 |
| 12 | 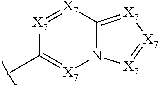 |
| 13 | 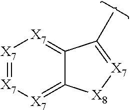 |
| 14 | 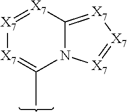 |
| 15 | 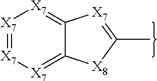 |
| 16 | 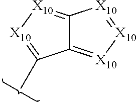 |
| 17 | 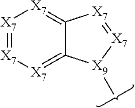 |
| 18 | 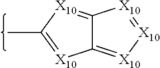 |
| 19 | 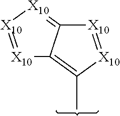 |

In the structural formulas of Table 1:

X₆, for each occurrence, is independently CH, CR₉, N, N(O), N⁺(R₁₇), provided that at least three X₆ groups are independently selected from CH and CR₉;

X₇, for each occurrence, is independently CH, CR₉, N, N(O), N⁺(R₁₇), provided that at least three X₇ groups are independently selected from CH and CR₉;

X₈, for each occurrence, is independently CH₂, CHR₉, CR₉R₉, O, S, S(O)ₚ, NR₇, or NR₁₇;

X₉, for each occurrence, is independently N or CH;

X₁₀, for each occurrence, is independently CH, CR₉, N, N(O), N⁺(R₁₇), provided that at least one X₁₀ is selected from CH and CR₉;

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring; and

R₁₇, for each occurrence, is independently —H, an alkyl, an aralkyl, —C(O)R₇, —C(O)OR₇, or —C(O)NR₁₀R₁₁.

Preferred R₅ groups from Table 1 are selected from the group consisting of an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl,an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo(b)thienyl.

In another alternative, R₅ in structural formula (VI) is selected from the group consisting of:

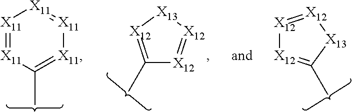

wherein:

X₁₁, for each occurrence, is independently CH, CR₉, N, N(O), or N⁺(R₁₇), provided that at least one X₁₁ is N, N(O), or N⁺(R₁₇) and at least two X₁₁ groups are independently selected from CH and CR₉;

X₁₂, for each occurrence, is independently CH, CR₉, N, N(O), N⁺(R₁₇), provided that at least one X₁₂ group is independently selected from CH and CR₉;

X₁₃, for each occurrence, is independently O, S, S(O)ₚ, NR₇, or NR₁₇;

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a hydroxyalkyl, alkoxyalkyl, haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring; and R₁₇, for each occurrence, is independently an alkyl or an aralkyl. The remainder of the variables have values defined above with reference to structural formula (I).

In one embodiment, the cell is in a subject in need of treatment for an inflammatory disorder and administration of a compound represented by any one of formulas (VI)-(VIII), or any of the embodiments of formulas (VI)-(VIII) disclosed herein, to the subject modulates the activity of glucocorticoid receptors in a cell that is in the subject.

In one embodiment, the cell is in a subject in need of treatment for an immune disorder and administration of a compound represented by any one of formulas (VI)-(VIII), or any of the embodiments of formulas (VI)-(VIII) disclosed herein, to the subject modulates the activity of glucocorticoid receptors in a cell that is in the subject.

In one embodiment, the cell is in a subject in need of immunosuppression and administration of a compound represented by any one of formulas (VI)-(VIII), or any of the embodiments of formulas (VI)-(VIII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (VI)-(VIII), or any of the embodiments of formulas (VI)-(VIII) disclosed herein.

In another aspect, the invention provides a method of treating or preventing an immune disorder in a subject, comprising administering to the subject an effective amount of a compound represented by any one of formulas (VI)-(VIII), or any of the embodiments of formulas (VI)-(VIII) disclosed herein.

In another aspect, the invention provides a method of suppressing the immune system in a subject in need thereof, comprising administering to the subject an effective amount of a compound represented by any one of formulas (VI)-(VIII), or any of the embodiments of formulas (VI)-(VIII) disclosed herein.

In another aspect, the invention provides a method of modulating the activity of glucocorticoid receptors in a cell, comprising administering to the cell an effective amount of a compound represented by formula (LII):

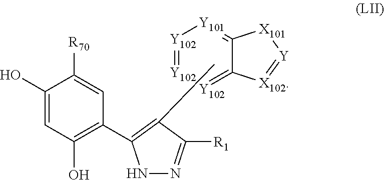

In structural formula (LII):

X₁₀₁ is O, S, or NR₁₀₂ and X₁₀₂ is CR₁₀₄ or N. Preferably, X₁₀₁ is NR₁₀₂ and X₁₀₂ is CR₁₀₄. Alternatively, X₁₀₁ is NR₁₀₂ and X₁₀₂ is N;

Y, for each occurrence, is independently N or CR₁₀₃;

Y₁₀₁ is N or CR₁₀₅;

Y₁₀₂ is N, C or CR₁₀₆;

R₁ is —OH, —SH, or NHR₇. Preferably, R₁ is —OH or —SH;

R₇₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy, cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ is selected from the group consisting of —H, C1-C6 alkyl, C1-C6 alkoxy, C1-C6 cycloalkyl, and C1-C6 cycloalkoxy, more preferably from the group consisting of —H, methyl, ethyl, propyl, isopropyl, cyclopropyl, methoxy, ethoxy, propoxy, and cyclopropoxy;

R₁₀₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁; preferably, R₁₀₂ is selected from the group consisting of —H, a C1-C6 alkyl, a C1-C6 cycloalkyl, —C(O)N(R₂₇)₂, and —C(O)OH, wherein R₂₇, for each occurrence, is independently is —H or a lower alkyl;

R₁₀₃ and R₁₀₄ are, independently, —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₁₀₃ and R₁₀₄ taken together with the carbon atoms to which they are attached form an optionally substituted cycloalkenyl, an optionally substituted aryl, an optionally substituted heterocyclyl, or an optionally substituted heteroaryl; preferably, R₁₀₃ and R₁₀₄ are independently, selected from the group consisting of —H, methyl, ethyl, propyl, isopropyl, cyclopropyl, methoxy, ethoxy, propoxy, and cyclopropoxy;

R₁₀₅ is —H, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁; preferably, R₁₀₅ is selected from the group consisting of —H, —OH, —SH, —NH₂, a C1-C6 alkoxy, a C1-C6 alkyl amino, and a C1-C6 dialkyl amino, more preferably from the group consisting of —H, —OH, methoxy and ethoxy; and

R₁₀₆, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁.

The remainder of the variables of the compounds of structural formula (LII) has values defined above with reference to structural formula (VI).

In one preferred set of values for the variables of the Hsp90 inhibitor represented by formula (LII), X₁₀₁ is NR₁₀₂, R₁₀₂ is selected from the group consisting of —H, a C1-C6 alkyl, a C1-C6 cycloalkyl, —C(O)N(R₂₇)₂, and —C(O)OH, each R₂₇, for each occurrence, is independently is —H or a lower alkyl, and the values for the remainder of the variables are as described above for formula (LII).

In a second preferred set of values for the variables of the Hsp90 inhibitor represented by formula (LII), X₁₀₁ is NR₁₀₂, R₁₀₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂ and the values for the remainder of the variables are as described above for formula (LII).

In third preferred set of values for the variables of the Hsp90 inhibitor represented by formula (LII), X₁₀₂ is CR₁₀₄; Y is CR₁₀₃; and R₁₀₃ and R₁₀₄ together with the carbon atoms to which they are attached form a cycloalkenyl, an aryl, heterocyclyl, or heteroaryl ring. Preferably, R₁₀₃ and R₁₀₄ together with the carbon atoms to which they are attached form a C₅-C₈ cycloalkenyl or a C₅-C₈ aryl and the values for the remainder of the variables are as described above for formula (LII).

In fourth preferred set of values for the variables of the Hsp90 inhibitor represented by formula (LII), R₁ is —OH or —SH and the values for the remainder of the variables are as described above for formula (LII).

In one embodiment, the cell is in a subject in need of treatment for an inflammatory disorder and administration of a compound represented by formula (LII), or any of the embodiments of formula (LII) disclosed herein, to the subject modulates the activity of glucocorticoid receptors in a cell that is in the subject.

In one embodiment, the cell is in a subject in need of treatment for an immune disorder and administration of a compound represented by formula (LII), or any of the embodiments of formula (LII) disclosed herein, to the subject modulates the activity of glucocorticoid receptors in a cell that is in the subject.

In one embodiment, the cell is in a subject in need of immunosuppression and administration of a compound represented by formula (LII), or any of the embodiments of formula (LII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LII), or any of the embodiments of formula (LII) disclosed herein.

In another aspect, the invention provides a method of treating or preventing an immune disorder in a subject, comprising administering to the subject an effective amount of a compound represented by formula (LII), or any of the embodiments of formula (LII) disclosed herein.

In another aspect, the invention provides a method of suppressing the immune system in a subject in need thereof, comprising administering to the subject an effective amount of a compound represented by formula (LII), or any of the embodiments of formula (LII) disclosed herein.

In another aspect, the invention provides a method of modulating the activity of glucocorticoid receptors in a cell, comprising administering to the cell an effective amount of a compound represented by formula (LIII):

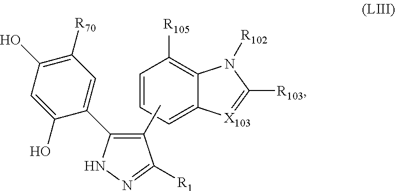

where X₁₀₃ is CR₁₀₄ or N and the remainder of the variables is defined above with reference with structural formulas (LII).

In one embodiment, the cell is in a subject in need of treatment for an inflammatory disorder and administration of a compound represented by formula (LIII), or any of the embodiments of formula (LIII) disclosed herein, to the subject modulates the activity of glucocorticoid receptors in a cell that is in the subject.

In one embodiment, the cell is in a subject in need of treatment for an immune disorder and administration of a compound represented by formula (LIII), or any of the embodiments of formula (LIII) disclosed herein, to the subject modulates the activity of glucocorticoid receptors in a cell that is in the subject.

In one embodiment, the cell is in a subject in need of immunosuppression and administration of a compound represented by formula (LIII), or any of the embodiments of formula (LIII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LIII), or any of the embodiments of formula (LIII) disclosed herein.

In another aspect, the invention provides a method of treating or preventing an immune disorder in a subject, comprising administering to the subject an effective amount of a compound represented by formula (LIII), or any of the embodiments of formula (LIII) disclosed herein.

In another aspect, the invention provides a method of suppressing the immune system in a subject in need thereof, comprising administering to the subject an effective amount of a compound represented by formula (LIII), or any of the embodiments of formula (LIII) disclosed herein.

In another aspect, the invention provides a method of modulating the activity of glucocorticoid receptors in a cell, comprising administering to the cell an effective amount of a compound represented by any one of formulas (LIVa)-(LIVi):

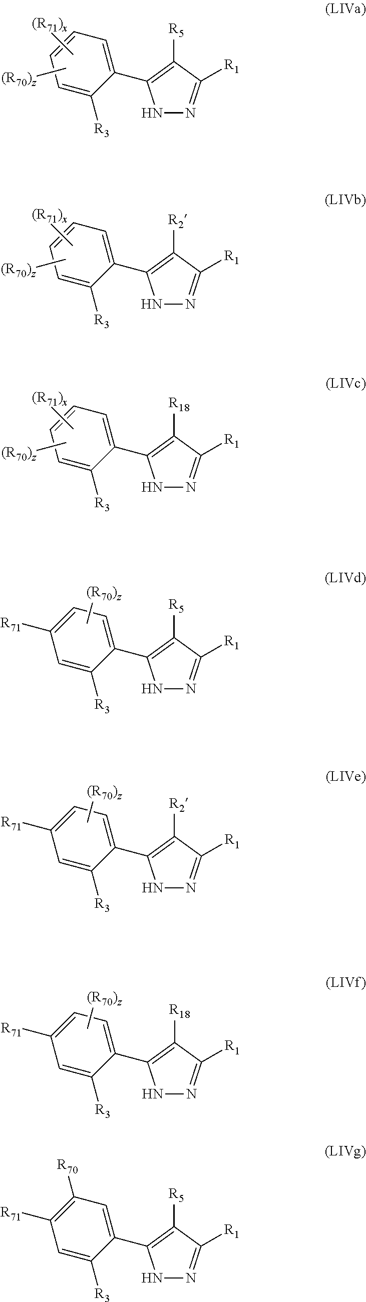

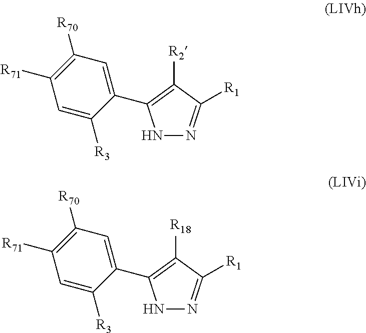

The values for the variables in structural formulas (LIVa)-(LIVi) are as described in structural formulas (VI), (VII), and (VIII).

In one preferred set of values for the variables of the Hsp90 inhibitor represented by structural formulas (LIVa)-(LIVi):

R₅ is as described for structural formula (VI), (VII), and (VIII) or a structural formula from Table 1;

R₇₀ and R₇₁, for each occurrence, are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

z in structural formula (LIVa)-(LIVc) is zero or an integer from 1 to 4; z in structural formula (LIVd)-(LIVf) is zero or an integer from 1 to 3;

x is 0 or 1;

z+x in structural formula (LIVa)-(LIVc) is less than or equal to 4; and

the remainder of the variables in formulas (LIVa)-(LIVi) have values defined above with reference to structural formula (VI), (VII) and (VIII).

A second preferred set of values for the variables of the Hsp90 inhibitor represented by structural formula (LIVa)-(LIVc) is provided in the following paragraphs:

R₇₁ is a halo, a haloalkyl, a haloalkoxy, a heteroalkyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; and k is 1, 2, 3, or 4; and R₁, R₃, R₇₀ and the remainder of the variables are as described in the first preferred set of values for the variables in structural formulas (LIVa)-(LIVc). Preferably, R₁ and R₃ are each, independently, —OH, —SH, or —NHR₇.

A third preferred set of values for the variables of the Hsp90 inhibitor represented by formula (LIVa)-(LIVc) is provided in the following paragraphs:

R₁ and R₃ are each, independently, —OH, —SH, or —NHR₇;

R₇₀ is an optionally substituted alkyl or cycloalkyl, an optionally substituted alkenyl, an optionally substituted alkynyl, cyano, halo, nitro, an optionally substituted cycloalkyl, haloalkyl, alkoxy, haloalkoxy, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇ and R₁ and R₃ and the remainder of the variables are as described in the second preferred set of values for the variables in structural formulas (LIVa)-(LIVc).

In a fourth preferred set of values for the variables of Structural Formulas (LIVa)-(LIVc):

R₁ is —SH or —OH;

R₃ and R₇₁ are —OH;

R₇₀ is a C1-C6 alkyl, a C3-C6 cycloalkyl, a C1-C6 alkoxy, a C1-C6 haloalkoxy, a C1-C6 alkyl sulfanyl, or —NR₁₀R₁₁; and

The remainder of the variables are as defined in Structural Formula (VI)-(VIII).

In one embodiment, the cell is in a subject in need of treatment for an inflammatory disorder and administration of a compound represented by any one of formulas (LIVa)-(LIVi), or any of the embodiments of formulas (LIVa)-(LIVi) disclosed herein, to the subject modulates the activity of glucocorticoid receptors in a cell that is in the subject.

In one embodiment, the cell is in a subject in need of treatment for an immune disorder and administration of a compound represented by any one of formulas (LIVa)-(LIVi), or any of the embodiments of formulas (LIVa)-(LIVi) disclosed herein, to the subject modulates the activity of glucocorticoid receptors in a cell that is in the subject.

In one embodiment, the cell is in a subject in need of immunosuppression and administration of a compound represented by any one of formulas (LIVa)-(LIVi), or any of the embodiments of formulas (LIVa)-(LIVi) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LIVa)-(LIVi), or any of the embodiments of formulas (LIVa)-(LIVi) disclosed herein.

In another aspect, the invention provides a method of treating or preventing an immune disorder in a subject, comprising administering to the subject an effective amount of a compound represented by any one of formulas (LIVa)-(LIVi), or any of the embodiments of formulas (LIVa)-(LIVi) disclosed herein.

In another aspect, the invention provides a method of suppressing the immune system in a subject in need thereof, comprising administering to the subject an effective amount of a compound represented by any one of formulas (LIVa)-(LIVi), or any of the embodiments of formulas (LIVa)-(LIVi) disclosed herein.

In another aspect, the invention provides a method of modulating the activity of glucocorticoid receptors in a cell, comprising administering to the cell an effective amount of a compound represented by any one of formulas (LVa)-(LVf):

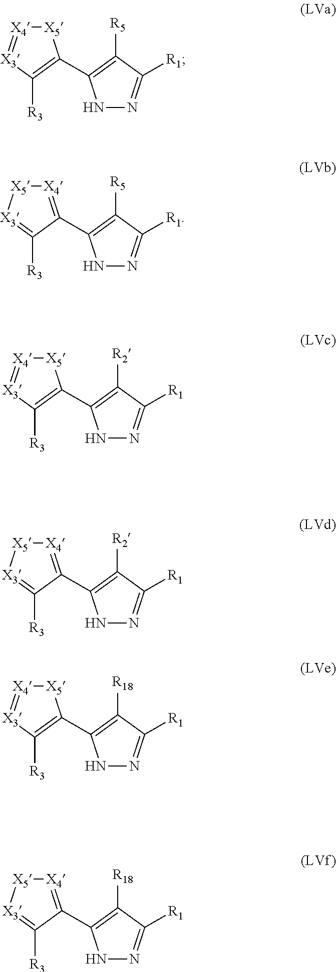

In formulas (LVa) and (LVb):

R₅ is as described for structural formula (VI) or a structural formula from Table 1;

X₃′ and X₄′ are each, independently, N, N(O), N⁺(R₁₇), CH or CR₇₀;

X₅′ is O, S, NR₁₇, CH₂, CH(R₇₀), C(R₇₀)₂, CH═CH, CH═CR₇₀, CR₇₀═CH, CR₇₀═CR₇₀, CH═N, CR₇₀═N, CH═N(O), CR₇₀═N(O), N═CH, N═CR₇₀, N(O)═CH, N(O)═CR₇₀, N⁺(R₁₇)═CH, N⁺(R₁₇)═CR₇₀, CH═N⁺(R₁₇), CR₇₀═N⁺(R₁₇), or N═N, provided that at least one X₃′, X₄′ or X₅′ is a heteroatom;

R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

R₁₇, for each occurrence, is independently an alkyl or an aralkyl; and n is zero or an integer from 1 to 4; and

the remainder of the variables has values defined above with reference to structural formulas (VI), (VII), and (VIII).

Preferably, Hsp90 inhibitor of structural formulas (LVa)-(LVf) are selected from Table 2a-c.

TABLE 2a

| Number | Compound |
|---|---|
| 1. | 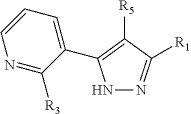 |
| 2. | 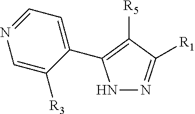 |
| 3. | 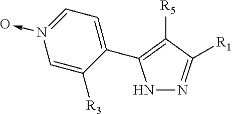 |
| 4. | 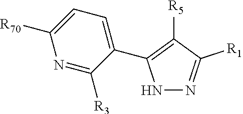 |
| 5. | 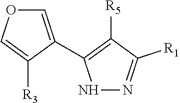 |
| 6. | 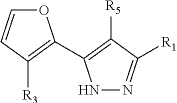 |
| 7. | 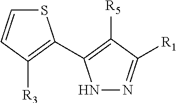 |
| 8. | 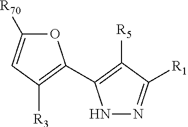 |
| 9. | 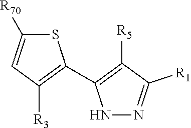 |
| 10. | 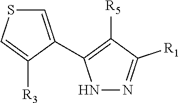 |

TABLE 2b

| Number | Compound |
|---|---|
| 1. | 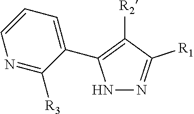 |
| 2. | 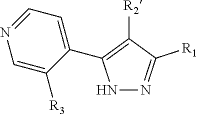 |
| 3. | 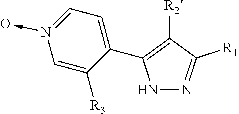 |
| 4. | 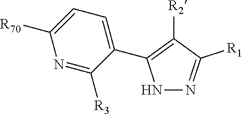 |
| 5. | 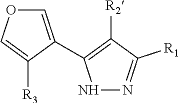 |
| 6. | 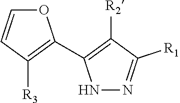 |
| 7. | 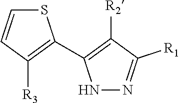 |
| 8. | 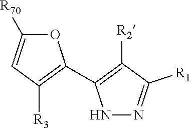 |
| 9. | 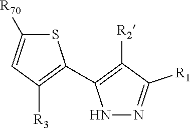 |
| 10. | 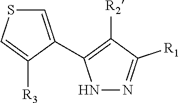 |

TABLE 2c

| Number | Compound |
|---|---|
| 1. | 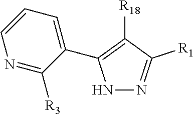 |
| | 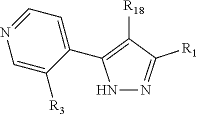 |
| 2. | 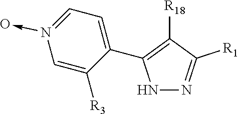 |
| | 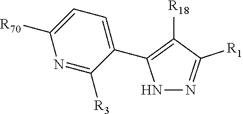 |
| 3. | 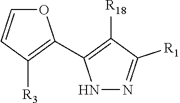 |
| 4. | 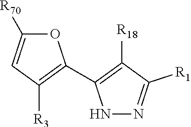 |
| | 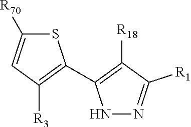 |
| 5. | 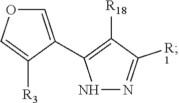 |
| | 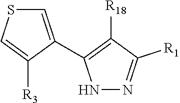 |
| 6. | |
| 7. | |
| 8. | 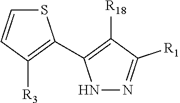 |
| 9. | |
| 10. | |

The values for the variables for the formulas in Tables 2a-c are as defined for structural formulas (LVa)-(LVf). Preferably, R₇₀ is a halo, a haloalkyl, a haloalkoxy, a heteroalkyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; and

k is 1, 2, 3, or 4.

In one embodiment, the cell is in a subject in need of treatment for an inflammatory disorder and administration of a compound represented by any one of formulas (LVa)-(LVf), or any of the embodiments of formulas (LVa)-(LVf) disclosed herein, to the subject modulates the activity of glucocorticoid receptors in a cell that is in the subject.

In one embodiment, the cell is in a subject in need of treatment for an immune disorder and administration of a compound represented by any one of formulas (LVa)-(LVf), or any of the embodiments of formulas (LVa)-(LVf) disclosed herein, to the subject modulates the activity of glucocorticoid receptors in a cell that is in the subject.

In one embodiment, the cell is in a subject in need of immunosuppression and administration of a compound represented by any one of formulas (LVa)-(LVf), or any of the embodiments of formulas (LVa)-(LVf) disclosed herein, to the subject modulates the activity of glucocorticoid receptors in a cell th&t is in the subject.

In another aspect, the invention provides a method of treating or preventing an inflammatory disorder in a subject, comprising administering to the subject an effective amount of a compound represented by any one of formulas (LVa)-(LVf), or any of the embodiments of formulas (LVa)-(LVf) disclosed herein.

In another aspect, the invention provides a method of treating or preventing an immune disorder in a subject, comprising administering to the subject an effective amount of a compound represented by any one of formulas (LVa)-(LVf), or any of the embodiments of formulas (LVa)-(LVf) disclosed herein.

In another aspect, the invention provides a method of suppressing the immune system in a subject in need thereof, comprising administering to the subject an effective amount of a compound represented by any one of formulas (LVa)-(LVf), or any of the embodiments of formulas (LVa)-(LVf) disclosed herein.

In another aspect, the invention provides a method of modulating the activity of glucocorticoid receptors in a cell, comprising administering to the cell an effective amount of a compound represented by formula (LVI):

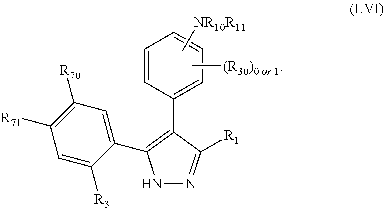

R₇₀ and R₇₁, for each occurrence, are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ is selected from an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇ and R₇₁ is as just described. The values for the remainder of the variables are as described for structural formulas (VI), (VII), and (VIII).

In one embodiment, the cell is in a subject in need of treatment for an inflammatory disorder and administration of a compound represented by formula (LVI), or any of the embodiments of formula (LVI) disclosed herein, to the subject modulates the activity of glucocorticoid receptors in a cell that is in the subject.

In one embodiment, the cell is in a subject in need of treatment for an immune disorder and administration of a compound represented by formula (LVI), or any of the embodiments of formula (LVI) disclosed herein, to the subject modulates the activity of glucocorticoid receptors in a cell that is in the subject.

In one embodiment, the cell is in a subject in need of immunosuppression and administration of a compound represented by formula (LVI), or any of the embodiments of formula (LVI) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LVI), or any of the embodiments of formula (LVI) disclosed herein.

In another aspect, the invention provides a method of treating or preventing an immune disorder in a subject, comprising administering to the subject an effective amount of a compound represented by formula (LVI), or any of the embodiments of formula (LVI) disclosed herein.

In another aspect, the invention provides a method of suppressing the immune system in a subject in need thereof, comprising administering to the subject an effective amount of a compound represented by formula (LVI), or any of the embodiments of formula (LVI) disclosed herein.

In another aspect, the invention provides a method of modulating the activity of glucocorticoid receptors in a cell, comprising administering to the cell an effective amount of a compound represented by formula (LVIIa) or (LVIIb):

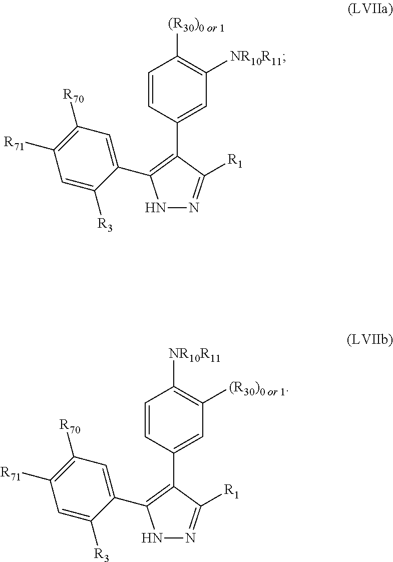

The variables in formulas (LVIIa) and (LVIIb) are defined above with reference to formula (LVI).

A first preferred set of values for the variables of structural formula (LVIIa) and (LVIIb) is provided in the following paragraph:

R₁, R₃ or R₇₁ are each independently selected from —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂, and p, R₇₀, R₇, R₈, R₁₀, R₁₁ and R₃₀ are as described for structural formula (LVI). Preferably, when R₁, R₃ and R₇₁ have these values, R₁₀ and R₁₁ are preferably each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; and p, R₇₀, R₇, and R₃₀ are as described for structural formula (LVI). More preferably, when R₁, R₃, R₁₀, R₁₁, and R₇₁ have these values, R₇₀ is preferably a C1-C6 alkyl, a C1-C6 haloalkyl, a C1-C6 alkoxy, a C1-C6 haloalkoxy, a C1-C6 alkyl sulfanyl or a C3-C6 cycloalkyl; and p, R₇, R₈ and R₃₀ are as described for structural formula (LVI).

A second preferred set of values for the variables of structural formula (LVIIa) and (LVIIb) is provided in the following paragraph:

R₁ and R₃ are each independently —OH or —SH; R₇₀ is preferably a C1-C6 alkyl, a C1-C6 haloalkyl, a C1-C6 alkoxy, a C1-C6 haloalkoxy, a C1-C6 alkyl sulfanyl or a C3-C6 cycloalkyl; R₁₀ and R₁₁ are preferably each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; R₇₁ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂; and p, R₇ R₈ and R₃₀ are as described for structural formula (LVI). Preferably, R₃₀ is a —OH, —SH, halogen, cyano, a C1-C6 alkyl, C1-C6 haloalkyl, C1-C6 alkoxy, C1-C6 haloalkoxy or C1-C6 alkyl sulfanyl and the remainder of the variables are as just described.

A third preferred set of values for the variables of structural formula (LVIIa) and (LVIIb) is provided in the following paragraph:

R₁, R₃ and R₇₁ are independently —SH or —OH; R₇₀ is cyclopropyl or isopropyl; R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; and R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl. Preferably, R₃₀ is a methyl, ethyl, propyl, isopropyl, methoxy or ethoxy. More preferably, R₁, R₃, R₇₀, R₇₁ and R₃₀ are as just described and and R₁₀ and R₁₁ are each independently a hydrogen, methyl, ethyl, propyl, isopropyl, or taken together with the nitrogen to which they are attached, are:

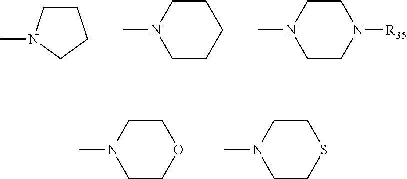

wherein R₃₅ is —H, a C1-C4 alkyl or a C1-C4 acyl.

In one embodiment, the cell is in a subject in need of treatment for an inflammatory disorder and administration of a compound represented by formula (LVIIa) or (LVIIb), or any of the embodiments of formula (LVIIa) or (LVIIb) disclosed herein, to the subject modulates the activity of glucocorticoid receptors in a cell that is in the subject.

In one embodiment, the cell is in a subject in need of treatment for an immune disorder and administration of a compound represented by formula (LVIIa) or (LVIIb), or any of the embodiments of formula (LVIIa) or (LVIIb) disclosed herein, to the subject modulates the activity of glucocorticoid receptors in a cell that is in the subject.

In one embodiment, the cell is in a subject in need of immunosuppression and administration of a compound represented by formula (LVIIa) or (LVIIb), or any of the embodiments of formula (LVIIa) or (LVIIb) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LVIIa) or (LVIIb), or any of the embodiments of formula (LVIIa) or (LVIIb) disclosed herein.

In another aspect, the invention provides a method of treating or preventing an immune disorder in a subject, comprising administering to the subject an effective amount of a compound represented by formula (LVIIa) or (LVIIb), or any of the embodiments of formula (LVIIa) or (LVIIb) disclosed herein.

In another aspect, the invention provides a method of suppressing the immune system in a subject in need thereof, comprising administering to the subject an effective amount of a compound represented by formula (LVIIa) or (LVIIb), or any of the embodiments of formula (LVIIa) or (LVIIb) disclosed herein.

In another aspect, the invention provides a method of modulating the activity of glucocorticoid receptors in a cell, comprising administering to the cell an effective amount of a compound represented by formula (LVIIIa) or (LVIIIb):

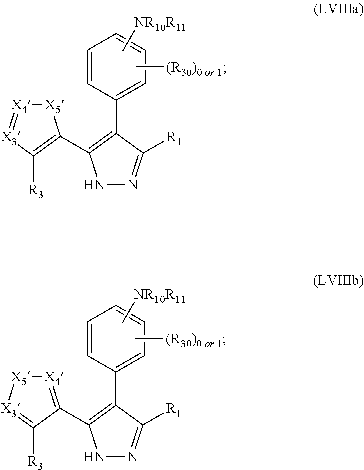

The values for the variables in structural formulas (LVIIIa) and (LVIIIb) are as described for structural formulas (LVc) and (LVd). Preferably, R₃₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. More preferably, R₃₀ is an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇.

In one embodiment, the cell is in a subject in need of treatment for an inflammatory disorder and administration of a compound represented by formula (LVIIIa) or (LVIIIb), or any of the embodiments of formula (LVIIIa) or (LVIIIb) disclosed herein, to the subject modulates the activity of glucocorticoid receptors in a cell that is in the subject.

In one embodiment, the cell is in a subject in need of treatment for an immune disorder and administration of a compound represented by formula (LVIIIa) or (LVIIIb), or any of the embodiments of formula (LVIIIa) or (LVIIIb) disclosed herein, to the subject modulates the activity of glucocorticoid receptors in a cell that is in the subject.

In one embodiment, the cell is in a subject in need of immunosuppression and administration of a compound represented by formula (LVIIIa) or (LVIIIb), or any of the embodiments of formula (LVIIIa) or (LVIIIb) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LVIIIa) or (LVIIIb), or any of the embodiments of formula (LVIIIa) or (LVIIIb) disclosed herein.

In another aspect, the invention provides a method of treating or preventing an immune disorder in a subject, comprising administering to the subject an effective amount of a compound represented by formula (LVIIIa) or (LVIIIb), or any of the embodiments of formula (LVIIIa) or (LVIIIb) disclosed herein.

In another aspect, the invention provides a method of suppressing the immune system in a subject in need thereof, comprising administering to the subject an effective amount of a compound represented by formula (LVIIIa) or (LVIIIb), or any of the embodiments of formula (LVIIIa) or (LVIIIb) disclosed herein.

In another aspect, the invention provides a method of modulating the activity of glucocorticoid receptors in a cell, comprising administering to the cell an effective amount of a compound represented by any one of formulas (LIXa)-(LIXd):

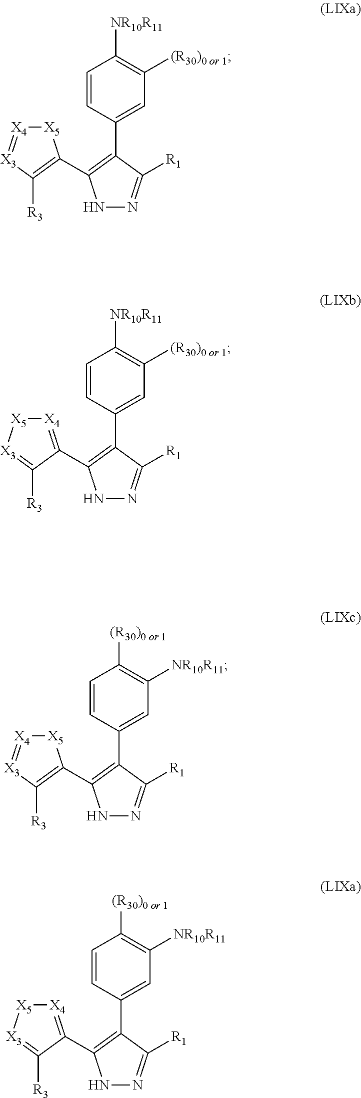

The values of the variables in structural formulas (LIXa)-(LIXd) are defined above with reference to structural formulas (LVIIIa) and (LVIIIb).

A first preferred set of values for the variables in structural formulas (LIXa)-(LIXd) are as described in the following paragraphs:

R₁ and R₃ are each independently —OH or —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂;

R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. Preferably, R₇₀ is a C1-C6 alkyl, a C1-C6 haloalkyl, a C1-C6 alkoxy, a C1-C6 haloalkoxy, a C1-C6 alkyl sulfanyl or a C3-C6 cycloalkyl; and

R₁₀ and R₁₁ and the remainder of the variables in structural formulas (LIXa)-(LIXd) are as described for structural formulas (LVIIIa) and (LVIIIb). Preferably, R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl.

In one embodiment, the cell is in a subject in need of treatment for an inflammatory disorder and administration of a compound represented by any one of formulas (LIXa)-(LIXd), or any of the embodiments of formulas (LIXa)-(LIXd) disclosed herein, to the subject modulates the activity of glucocorticoid receptors in a cell that is in the subject.

In one embodiment, the cell is in a subject in need of treatment for an immune disorder and administration of a compound represented by any one of formulas (LIXa)-(LIXd), or any of the embodiments of formulas (LIXa)-(LIXd) disclosed herein, to the subject modulates the activity of glucocorticoid receptors in a cell that is in the subject.

In one embodiment, the cell is in a subject in need of immunosuppression and administration of a compound represented by any one of formulas (LIXa)-(LIXd), or any of the embodiments of formulas (LIXa)-(LIXd) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LIXa)-(LIXd), or any of the embodiments of formulas (LIXa)-(LIXd) disclosed herein.

In another aspect, the invention provides a method of treating or preventing an immune disorder in a subject, comprising administering to the subject an effective amount of a compound represented by any one of formulas (LIXa)-(LIXd), or any of the embodiments of formulas (LIXa)-(LIXd) disclosed herein.

In another aspect, the invention provides a method of suppressing the immune system in a subject in need thereof, comprising administering to the subject an effective amount of a compound represented by any one of formulas (LIXa)-(LIXd), or any of the embodiments of formulas (LIXa)-(LIXd) disclosed herein.

In another aspect, the invention provides a method of modulating the activity of glucocorticoid receptors in a cell, comprising administering to the cell an effective amount of a compound represented by any one of formulas (LXa)-(LXp):

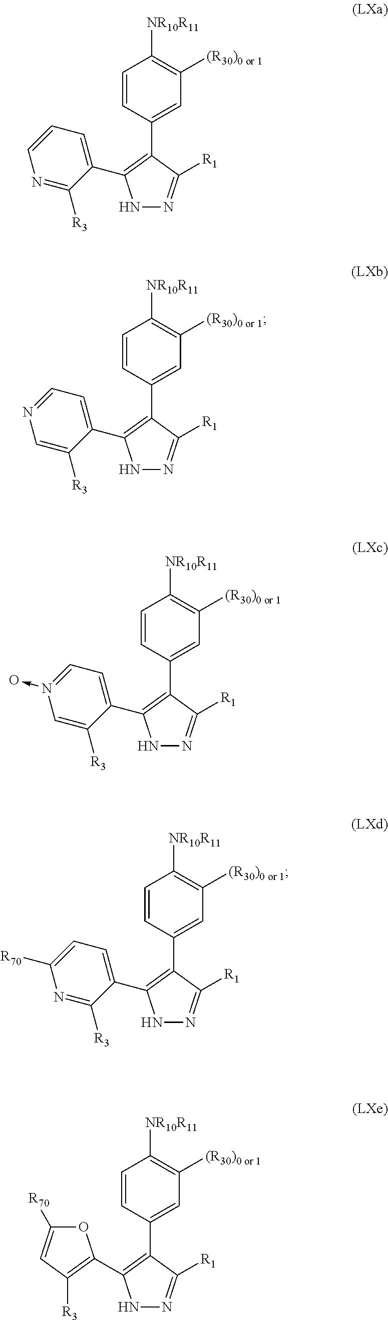

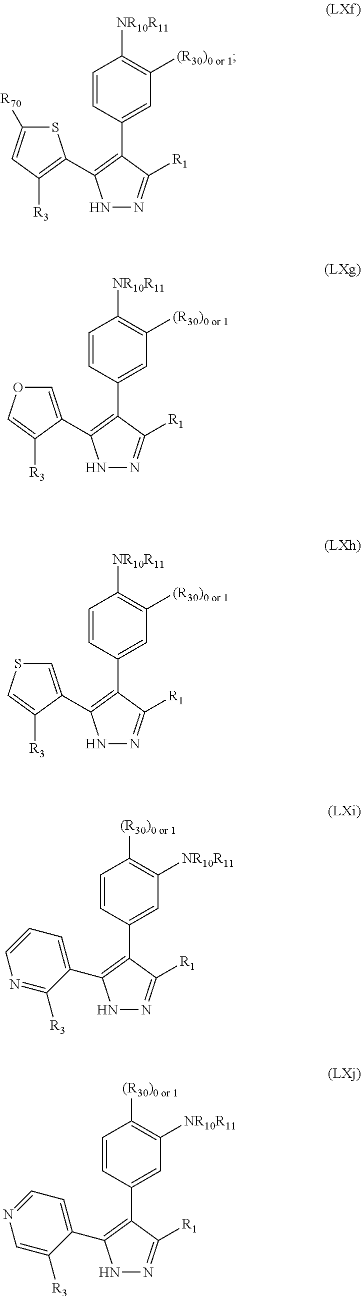

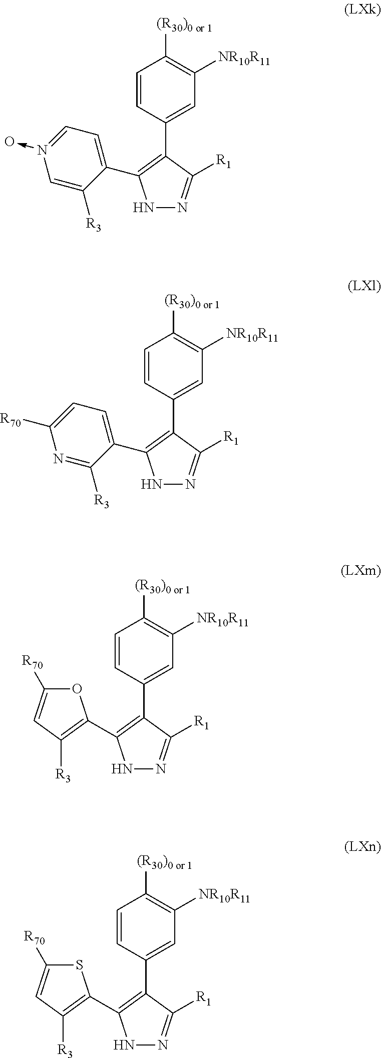

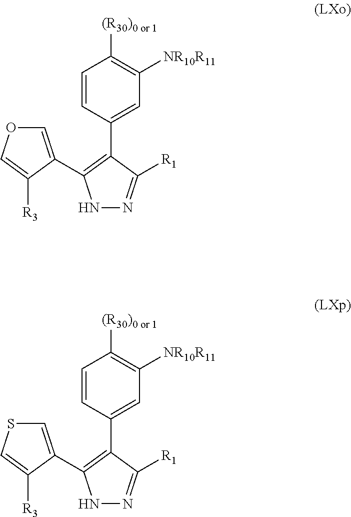

The values of the variables in structural formulas (LXa)-(LXp) are defined above with reference to structural formulas (XIXa)-(XIXd).

A first preferred set of values for the variables in structural formulas (LX) are as described in the following paragraphs:

R₁ and R₃ are each independently —OH or —SH, or —HNR₇;

R₇₀, is a C1-C6 alkyl, a C1-C6 haloalkyl, a C1-C6 alkoxy, a C1-C6 haloalkoxy, a C1-C6 alkyl sulfanyl or a C3-C6 cycloalkyl;

R₁₀ and R₁₁ and the remainder of the variables in structural formulas (LXa)-(LXp) are as described for structural formulas (LVIIIa) and (LVIIIb). Preferably, R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; and

R₃₀ and the remainder of the variables in structural formulas (LXa)-(LXp) are as described for structural formulas (LIXa)-(LIXd). Preferably, R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl.

A second preferred set of values for the variables in structural formulas (LXa)-(LXp) are as described in the following paragraphs:

R₁ and R₃ are independently —SH or —OH;

R₇₀ is cyclopropyl or isopropyl;

R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl;

R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl. Preferably, R₃₀ is a methyl, ethyl, propyl, isopropyl, methoxy or ethoxy; and the remainder of the variables are as described for formulas (LVIIIa) and (LVIIIb). More preferably, R₁₀ and R₁₁ are each independently a hydrogen, methyl, ethyl, propyl, isopropyl, or taken together with the nitrogen to which they are attached, are:

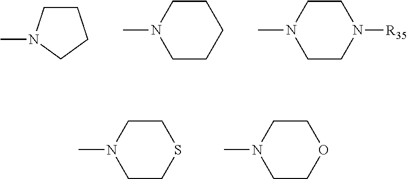

wherein R₃₅ is —H, a C1-C4 alkyl or a C1-C4 acyl.

In one embodiment, the cell is in a subject in need of treatment for an inflammatory disorder and administration of a compound represented by any one of formulas (LXa)-(LXp), or any of the embodiments of formulas (LXa)-(LXp) disclosed herein, to the subject modulates the activity of glucocorticoid receptors in a cell that is in the subject.

In one embodiment, the cell is in a subject in need of treatment for an immune disorder and administration of a compound represented by any one of formulas (LXa)-(LXp), or any of the embodiments of formulas (LXa)-(LXp) disclosed herein, to the subject modulates the activity of glucocorticoid receptors in a cell that is in the subject.

In one embodiment, the cell is in a subject in need of immunosuppression and administration of a compound represented by any one of formulas (LXa)-(LXp), or any of the embodiments of formulas (LXa)-(LXp) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LXa)-(LXp), or any of the embodiments of formulas (LXa)-(LXp) disclosed herein.

In another aspect, the invention provides a method of treating or preventing an immune disorder in a subject, comprising administering to the subject an effective amount of a compound represented by any one of formulas (LXa)-(LXp), or any of the embodiments of formulas (LXa)-(LXp) disclosed herein.

In another aspect, the invention provides a method of suppressing the immune system in a subject in need thereof, comprising administering to the subject an effective amount of a compound represented by any one of formulas (LXa)-(LXp), or any of the embodiments of formulas (LXa)-(LXp) disclosed herein.

In another aspect, the invention provides a method of modulating the activity of glucocorticoid receptors in a cell, comprising administering to the cell an effective amount of a compound represented by formula (LXIa) or (LXIb):

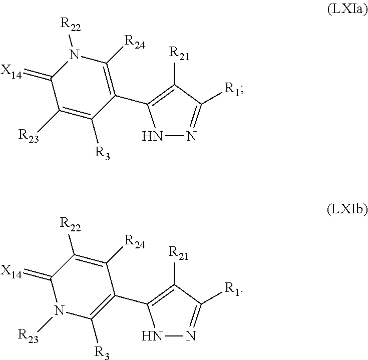

In formulas (LXIa) and (LXIb):

X₁₄ is O, S, or NR₇. Preferably, X₁₄ is O;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂. Preferably, R₁ is —OH, —SH, or —NHR₇;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₂₁ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl. Preferably, R₂₁ is an optionally substituted alkyl, an optionally substituted cycloalkyl, an optionally substituted aryl or an optionally substituted heteroaryl. Alternatively, R₂₁ is

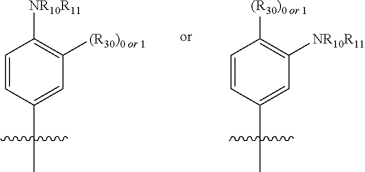

wherein

R₁₀ and R₁₁ is defined as above; and

R₃₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)OR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

z and q are independently an integer from 0 to 4; and

x is 0 or 1, provided that z+x less than or equal to 4.

R₂₂, for each occurrence, is independently a substituent selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, a haloalkyl, —C(O)R₇, —C(O)OR₇, —OC(O)R₇, —C(O)NR₁₀R₁₁, —NR₈C(O)R₇, —S(O)ₚR₇, —S(O)ₚOR₇, or —S(O)ₚNR₁₀R₁₁. Preferably, R₂₂ is an alkyl, an aralkyl, —C(O)R₇, —C(O)OR₇, or —C(O)NR₁₀R₁₁; and

R₂₃ and R₂₄, for each occurrence, are independently a substituent selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

R₂₆ is a lower alkyl;

p, for each occurrence, is, independently, 0, 1 or 2; and

m, for each occurrence, is independently, 1, 2, 3, or 4.

In one embodiment, the cell is in a subject in need of treatment for an inflammatory disorder and administration of a compound represented by formula (LXIa) or (LXIb), or any of the embodiments of formula (LXIa) or (LXIb) disclosed herein, to the subject modulates the activity of glucocorticoid receptors in a cell that is in the subject.

In one embodiment, the cell is in a subject in need of treatment for an immune disorder and administration of a compound represented by formula (LXIa) or (LXIb), or any of the embodiments of formula (LXIa) or (LXIb) disclosed herein, to the subject modulates the activity of glucocorticoid receptors in a cell that is in the subject.

In one embodiment, the cell is in a subject in need of immunosuppression and administration of a compound represented by formula (LXIa) or (LXIb), or any of the embodiments of formula (LXIa) or (LXIb) disclosed herein, to the subject modulates the activity of glucocorticoid receptors in a cell that'is in the subject.

In another aspect, the invention provides a method of treating or preventing an inflammatory disorder in a subject, comprising administering to the subject an effective amount of a compound represented by formula (LXIa) or (LXIb), or any of the embodiments of formula (LXIa) or (LXIb) disclosed herein.

In another aspect, the invention provides a method of treating or preventing an immune disorder in a subject, comprising administering to the subject an effective amount of a compound represented by formula (LXIa) or (LXIb), or any of the embodiments of formula (LXIa) or (LXIb) disclosed herein.

In another aspect, the invention provides a method of suppressing the immune system in a subject in need thereof, comprising administering to the subject an effective amount of a compound represented by formula (LXIa) or (LXIb), or any of the embodiments of formula (LXIa) or (LXIb) disclosed herein.

In another aspect, the invention provides a method of modulating the activity of glucocorticoid receptors in a cell, comprising administering to the cell an effective amount of a compound represented by formula (IX), (X) or (XI):

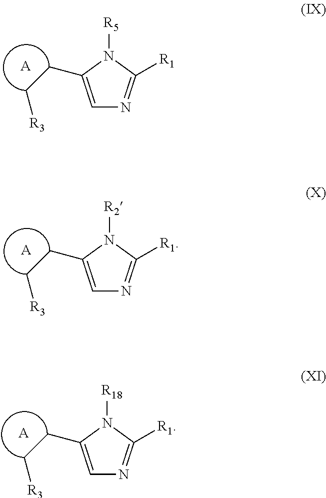

In formulas (LX)-(XI):

ring A is an aryl or a heteroaryl, optionally further substituted with one or more substituents in addition to R₃. Preferably, Ring A is represented one of the following structural formulas:

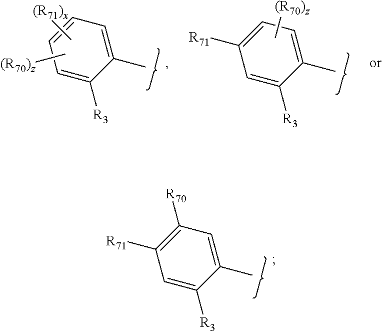

wherein z is 0, 1, 2, 3 or 4; x is 0 or 1; and z+x is less than or equal to 4.

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂. Preferably, R₁ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. More preferably, R₁ is —OH, —SH, or —NHR₇. Even more preferably, R₁, is —SH or —OH;

R₂′ is an optionally substituted phenyl group. Preferably, R₂′ is substituted with one or more group represented by R₃₀, wherein R₃₀, for each occurrence, are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. More preferably, R₂′ is an optionally substituted indolyl group or a phenyl group substituted with NR₁₀R₁₁ and optionally with at least one other substituent represented by R₃₀;

R₃ is —OH, —SH, —NR₇H, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂. In another embodiment, —OR₂₆ and —SR₂₆, are additional values for R₃. Preferably, R₃ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂. More preferably, R₃ is —OH, —SH, or —NHR₇. Even more preferably, R₃ is —SH or —OH.

R₅ is an optionally substituted heteroaryl; an optionally substituted 6 to 14-membered aryl.

R₇₀, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ is selected from the group consisting of —H, C1-C6 alkyl, C1-C6 alkoxy, C1-C6 cycloalkyl, and C1-C6 cycloalkoxy, more preferably from the group consisting of —H, methyl, ethyl, propyl, isopropyl, cyclopropyl, methoxy, ethoxy, propoxy, and cyclopropoxy.

R₇₁, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂.

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

R₂₆ is a lower alkyl;

p, for each occurrence, is, independently, 0, 1 or 2; and

m, for each occurrence, is independently, 1, 2, 3, or 4.

R₅ in structural formula (IX) is preferably represented by the following structural formula:

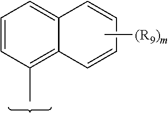

wherein:

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring, and m is zero or an integer from 1 to 7. More preferably, substituent R₅ is represented by one of the following structural formulas:

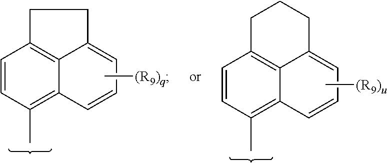

wherein:

R₉ is as defined as above; q is zero or an integer from 1 to 7; and u is zero or an integer from 1 to 8. The remainder of the variables have values defined above with reference to structural formula (IX).

In another alternative, R₅ in structural formula (IX) is represented by the following structural formula:

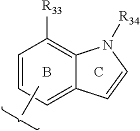

wherein:

R₃₃ is —H, a halo, lower alkyl, a lower alkoxy, a lower haloalkyl, a lower haloalkoxy, and lower alkyl sulfanyl; R₃₄ is H, a lower alkyl, or a lower alkylcarbonyl; and ring B and ring C are optionally substituted with one or more substituents. The remainder of the variables have values defined above with reference to structural formula (IX).

In another alternative, R₅ in structural formula (IX) is selected from a group listed in Table 3.

TABLE 3

| Number | Substituent R₅ |
|---|---|
| 1. | 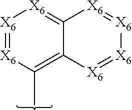 |
| 2. | 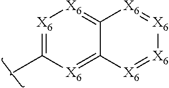 |
| 3. | 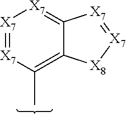 |
| 4. | 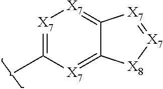 |
| 5. | 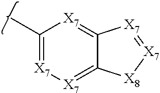 |
| 6. | 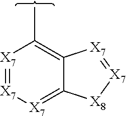 |
| 7. | 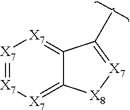 |
| 8. | 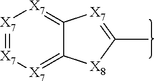 |
| 9. | 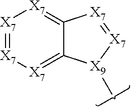 |
| 10. | 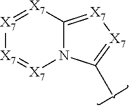 |
| 11. | 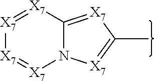 |
| 12. | 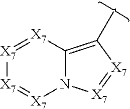 |
| 13. | 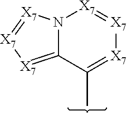 |
| 14. | 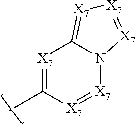 |
| 15. | 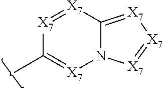 |
| 16. | 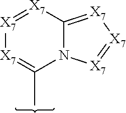 |
| 17. | 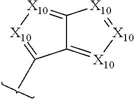 |
| 18. | 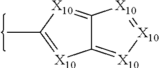 |
| 19. | 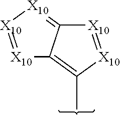 |

In the structural formulas of Table 3:

X₆, for each occurrence, is independently CH, CR₉, N, N(O), N⁺(R₁₇), provided that at least three X₆ groups are independently selected from CH and CR₉;

X₇, for each occurrence, is independently CH, CR₉, N, N(O), N⁺(R₁₇), provided that at least three X₇ groups are independently selected from CH and CR₉;

X₈, for each occurrence, is independently CH₂, CHR₉, CR₉R₉, O, S, S(O)p, NR₇, or NR₁₇;

X₉, for each occurrence, is independently N or CH;

X₁₀, for each occurrence, is independently CH, CR₉, N, N(O), N⁺(R₁₇), provided that at least one X₁₀ is selected from CH and CR₉;

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring; and

R₁₇, for each occurrence, is independently —H, an alkyl, an aralkyl, —C(O)R₇, —C(O)OR₇, or —C(O)NR₁₀R₁₁.

Preferred R₅ groups from Table 3 are selected from the group consisting of an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl,an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl; an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo(b)thienyl.

In another alternative, R₅ in structural formula (IX) is selected from the group consisting of:

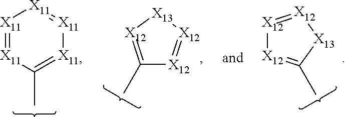

wherein:

X₁₁, for each occurrence, is independently CH, CR₉, N, N(O), or N⁺(R₁₇), provided that at least one X₁₁ is N, N(O), or N⁺(R₁₇) and at least two X₁₁ groups are independently selected from CH and CR₉;

X₁₂, for each occurrence, is independently CH, CR₉, N, N(O), N⁺(R₁₇), provided that at least one X₁₂ group is independently selected from CH and CR₉;

X₁₃, for each occurrence, is independently O, S, S(O)p, NR₇, or NR₁₇;

R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a hydroxyalkyl, alkoxyalkyl, haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁; or two R₉ groups taken together with the carbon atoms to which they are attached form a fused ring; and R₁₇, for each occurrence, is independently an alkyl or an aralkyl. The remainder of the variables have values defined above with reference to structural formula (IX).

In one embodiment, the cell is in a subject in need of treatment for an inflammatory disorder and administration of a compound represented by formula (IX), (X) or (XI), or any of the embodiments of formula (IX), (X) or (XI) disclosed herein, to the subject modulates the activity of glucocorticoid receptors in a cell that is in the subject.

In one embodiment, the cell is in a subject in need of treatment for an immune disorder and administration of a compound represented by formula (IX), (X) or (XI), or any of the embodiments of formula (IX), (X) or (XI) disclosed herein, to the subject modulates the activity of glucocorticoid receptors in a cell that is in the subject.

In one embodiment, the cell is in a subject in need of immunosuppression and administration of a compound represented by formula (IX), (X) or (XI), or any of the embodiments of formula (IX), (X) or (XI) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (IX), (X) or (XI), or any of the embodiments of formula (IX), (X) or (XI) disclosed herein.

In another aspect, the invention provides a method of treating or preventing an immune disorder in a subject, comprising administering to the subject an effective amount of a compound represented by formula (IX), (X) or (XI), or any of the embodiments of formula (IX), (X) or (XI) disclosed herein.

In another aspect, the invention provides a method of suppressing the immune system in a subject in need thereof, comprising administering to the subject an effective amount of a compound represented by formula (IX), (X) or (XI), or any of the embodiments of formula (IX), (X) or (XI) disclosed herein.

In another aspect, the invention provides a method of modulating the activity of glucocorticoid receptors in a cell, comprising administering to the cell an effective amount of a compound represented by formula (LXII):

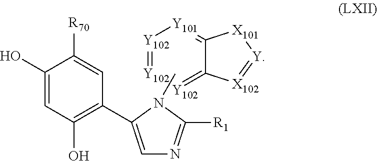

In structural formula (LXII):

X₁₀₁ is O, S, or NR₁₀₂ and X₁₀₂ is CR₁₀₄ or N. Preferably, X₁₀₁ is NR₁₀₂ and X₁₀₂ is CR₁₀₄. Alternatively, X₁₀₁ is NR₁₀₂ and X₁₀₂ is N;

Y, for each occurrence, is independently N or CR₁₀₃;

Y₁₀₁ is N or CR₁₀₅;

Y₁₀₂ is N, C or CR₁₀₆;

R₁ is OH, SH, or NHR₇. Preferably, R₁ is —OH or —SH;

R₇₀ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ is selected from the group consisting of —H, C1-C6 alkyl, C1-C6 alkoxy, C1-C6 cycloalkyl, and C1-C6 cycloalkoxy, more preferably from the group consisting of —H, methyl, ethyl, propyl, isopropyl, cyclopropyl, methoxy, ethoxy, propoxy, and cyclopropoxy;

R₁₀₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁; preferably, R₁₀₂ is selected from the group consisting of —H, a C1-C6 alkyl, a C1-C6 cycloalkyl, —C(O)N(R₂₇)₂, and —C(O)OH, wherein R₂₇, for each occurrence, is independently is —H or a lower alkyl;

R₁₀₃ and R₁₀₄ are, independently, —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₁₀₃ and R₁₀₄ taken together with the carbon atoms to which they are attached form an optionally substituted cycloalkenyl, an optionally substituted aryl, an optionally'substituted heterocyclyl, or an optionally substituted heteroaryl; preferably, R₁₀₃ and R₁₀₄ are independently, selected from the group consisting of —H, methyl, ethyl, propyl, isopropyl, cyclopropyl, methoxy, ethoxy, propoxy, and cyclopropoxy;

R₁₀₅ is —H, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁; preferably, R₁₀₅ is selected from the group consisting of —H, —OH, —SH, —NH₂, a C1-C6 alkoxy, a C1-C6 alkyl amino, and a C1-C6 dialkyl amino, more preferably from the group consisting of —H, —OH, methoxy and ethoxy; and

R₁₀₆, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁.

The remainder of the variables of the compounds of structural formula (LXII) has values defined above with reference to structural formula (IX).

In one preferred set of values for the variables of the Hsp90 inhibitor represented by formula (LXII), X₁₀₁ is NR₁₀₂, R₁₀₂ is selected from the group consisting of —H, a C1-C6 alkyl, a C1-C6 cycloalkyl, —C(O)N(R₂₇)₂, and —C(O)OH, wherein R₂₇, for each occurrence, is independently is —H or a lower alkyl and the values for the remainder of the variables are as described above for formula (LXII).

In a second preferred set of values for the variables of the Hsp90 inhibitor represented by formula (LXII), X₁₀₁ is NR₁₀₂, R₁₀₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂ and the values for the remainder of the variables are as described above for formula (LXII).

In third preferred set of values for the variables of the Hsp90 inhibitor represented by formula (LXII), X₁₀₂ is CR₁₀₄; Y is CR₁₀₃; and R₁₀₃ and R₁₀₄ together with the carbon atoms to which they are attached form a cycloalkenyl, an aryl, heterocyclyl, or heteroaryl ring. Preferably, R₁₀₃ and R₁₀₄ together with the carbon atoms to which they are attached form a C₅-C₈ cycloalkenyl or a C₅-C₈ aryl and the values for the remainder of the variables are as described above for formula (LXII).

In fourth preferred set of values for the variables of the Hsp90 inhibitor represented by formula (LXII), R₁ is —OH or —SH and the values for the remainder of the variables are as described above for formula (LXII).

In one embodiment, the cell is in a subject in need of treatment for an inflammatory disorder and administration of a compound represented by formula (LXII), or any of the embodiments of formula (LXII) disclosed herein, to the subject modulates the activity of glucocorticoid receptors in a cell that is in the subject.

In one embodiment, the cell is in a subject in need of treatment for an immune disorder and administration of a compound represented by formula (LXII), or any of the embodiments of formula (LXII) disclosed herein, to the subject modulates the activity of glucocorticoid receptors in a cell that is in the subject.

In one embodiment, the cell is in a subject in need of immunosuppression and administration of a compound represented by formula (LXII), or any of the embodiments of formula (LXII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LXII), or any of the embodiments of formula (LXII) disclosed herein.

In another aspect, the invention provides a method of treating or preventing an immune disorder in a subject, comprising administering to the subject an effective amount of a compound represented by formula (LXII), or any of the embodiments of formula (LXII) disclosed herein.

In another aspect, the invention provides a method of suppressing the immune system in a subject in need thereof, comprising administering to the subject an effective amount of a compound represented by formula (LXII), or any of the embodiments of formula (LXII) disclosed herein.

In another aspect, the invention provides a method of modulating the activity of glucocorticoid receptors in a cell, comprising administering to the cell an effective amount of a compound represented by formula (LXIII):

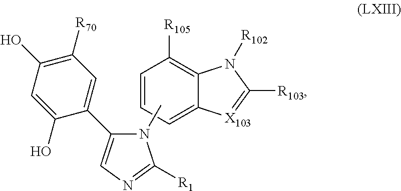

where X₁₀₃ is CR₁₀₄ or N and the remainder of the variables is defined above with reference with structural formulas (LXII).

In one embodiment, the cell is in a subject in need of treatment for an inflammatory disorder and administration of a compound represented by formula (LXIII), or any of the embodiments of formula (LXIII) disclosed herein, to the subject modulates the activity of glucocorticoid receptors in a cell that is in the subject.

In one embodiment, the cell is in a subject in need of treatment for an immune disorder and administration of a compound represented by formula (LXIII), or any of the embodiments of formula (LXIII) disclosed herein, to the subject modulates the activity of glucocorticoid receptors in a cell that is in the subject.

In one embodiment, the cell is in a subject in need of immunosuppression and administration of a compound represented by formula (LXIII), or any of the embodiments of formula (LXIII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LXIII), or any of the embodiments of formula (LXIII) disclosed herein.

In another aspect, the invention provides a method of treating or preventing an immune disorder in a subject, comprising administering to the subject an effective amount of a compound represented by formula (LXIII), or any of the embodiments of formula (LXIII) disclosed herein.

In another aspect, the invention provides a method of suppressing the immune system in a subject in need thereof, comprising administering to the subject an effective amount of a compound represented by formula (LXIII), or any of the embodiments of formula (LXIII) disclosed herein.

In another aspect, the invention provides a method of modulating the activity of glucocorticoid receptors in a cell, comprising administering to the cell an effective amount of a compound represented by any one of formulas (LXIVa)-(LXIVi):

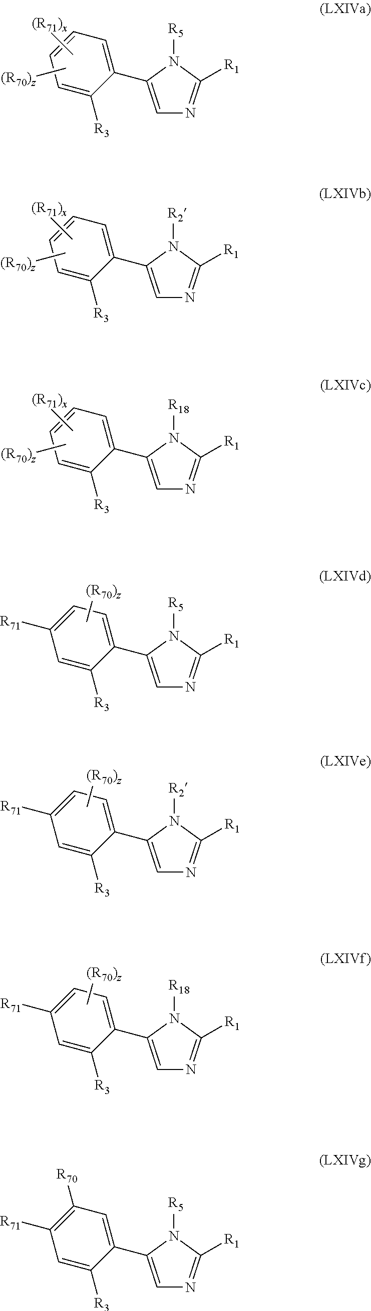

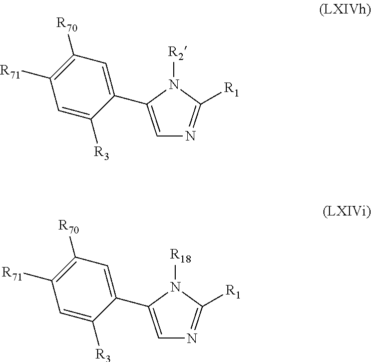

The values for the variables in structural formulas (LXIVa)-(LXIVi) are as described in structural formula (IX), (X), and (XI).

In one preferred set of values for the variables of the Hsp90 inhibitor represented by structural formulas (VIa-c)-(VIIIa-c):

R₅ is as described for structural formula (IX), (LXII), (LXIII) or a structural formula from Table 1;

R₇₀ and R₇₁, for each occurrence, are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

z in structural formula (VIa-c) is zero or an integer from 1 to 4; z in structural formula (VIIa-c) is zero or an integer from 1 to 3;

x is 0 or 1;

z+x in structural formula (LXIVa)-(LXIVc) is less than or equal to 4; and

the remainder of the variables in formulas (LXIVa)-(LXIVi) have values defined above with reference to structural formula (IX), (X), and (XI).

A second preferred set of values for the variables of the Hsp90 inhibitor represented by structural formula (LXIVa)-(LXIVi) is provided in the following paragraphs:

R₇₁ is a halo, a haloalkyl, a haloalkoxy, a heteroalkyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; and k is 1, 2, 3, or 4; and R₁, R₃, R₇₀ and the remainder of the variables are as described in the first preferred set of values for the variables in structural formulas (LXIVa)-(LXIVi). Preferably, R₁ and R₃ are each, independently, —OH, —SH, or —NHR₇.

A third preferred set of values for the variables of the Hsp90 inhibitor represented by formula (LXIVa)-(LXIVi) is provided in the following paragraphs:

R₁ and R₃ are each, independently, —OH, —SH, or —NHR₇;

R₇₀ is an optionally substituted alkyl or cycloalkyl, an optionally substituted alkenyl, an optionally substituted alkynyl, cyano, halo, nitro, an optionally substituted cycloalkyl, haloalkyl, alkoxy, haloalkoxy, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇ and R₁ and R₃ and the remainder of the variables are as described in the second preferred set of values for the variables in structural formulas (LXIVa)-(LXIVi).

In a fourth preferred set of values for the variables of Structural Formulas (LXIVa)-(LXIVi):

R₁ is —SH or —OH;

R₃ and R₂₅ are —OH;

R₇₀ is a C1-C6 alkyl, a C3-C6 cycloalkyl, a C1-C6 alkoxy, a C1-C6 haloalkoxy, a C1-C6 alkyl sulfanyl, or —NR₁₀R₁₁; and

The remainder of the variables are as defined in Structural Formula (IX), (X), and (XI).

In one embodiment, the cell is in a subject in need of treatment for an inflammatory disorder and administration of a compound represented by any one of formulas (LXIVa)-(LXIVi), or any of the embodiments of formulas (LXIVa)-(LXIVi) disclosed herein, to the subject modulates the activity of glucocorticoid receptors in a cell that is in the subject.

In one embodiment, the cell is in a subject in need of treatment for an immune disorder and administration of a compound represented by any one of formulas (LXIVa)-(LXIVi), or any of the embodiments of formulas (LXIVa)-(LXIVi) disclosed herein, to the subject modulates the activity of glucocorticoid receptors in a cell that is in the subject.

In one embodiment, the cell is in a subject in need of immunosuppression and administration of a compound represented by any one of formulas (LXIVa)-(LXIVi), or any of the embodiments of formulas (LXIVa)-(LXIVi) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LXIVa)-(LXIVi), or any of the embodiments of formulas (LXIVa)-(LXIVi) disclosed herein.

In another aspect, the invention provides a method of treating or preventing an immune disorder in a subject, comprising administering to the subject an effective amount of a compound represented by any one of formulas (LXIVa)-(LXIVi), or any of the embodiments of formulas (LXIVa)-(LXIVi) disclosed herein.

In another aspect, the invention provides a method of suppressing the immune system in a subject in need thereof, comprising administering to the subject an effective amount of a compound represented by any one of formulas (LXIVa)-(LXIVi), or any of the embodiments of formulas (LXIVa)-(LXIVi) disclosed herein.

In another aspect, the invention provides a method of modulating the activity of glucocorticoid receptors in a cell, comprising administering to the cell an effective amount of a compound represented by any one of formulas (LXVa)-LXVf):

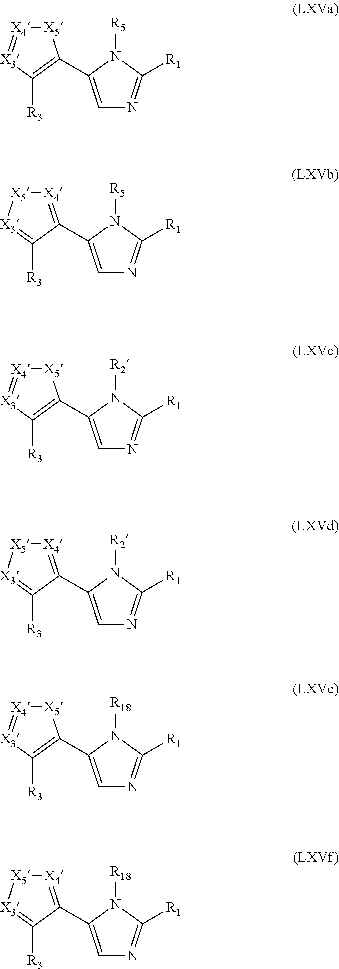

In formulas (LXVa) and (LXVb):

R₅ is as described for structural formula (IX), (LXII), or (LXIII), or a structural formula from Table 1;

X₃′ and X₄′ are each, independently, N, N(O), N⁺(R₁₇), CH or CR₇₀;

X₅′ is O, S, NR₁₇, CH₂, CH(R₇₀), C(R₇₀)₂, CH═CH, CH═CR₇₀, CR₇₀═CH, CR₇₀═CR₇₀, CH═N, CR₇₀═N, CH═N(O), CR₇₀═N(O), N═CH, N═CR₇₀, N(O)═CH, N(O)═CR₇₀, N⁺(R₁₇)═CH, N⁺(R₁₇)═CR₇₀, CH═N⁺(R₁₇), CR₆₀═N⁺(R₁₇), or N═N, provided that at least one X₃′, X₄′ or X₅′ is a heteroatom;

R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

R₁₇, for each occurrence, is independently an alkyl or an aralkyl; and n is zero or an integer from 1 to 4; and

the remainder of the variables has values defined above with reference to structural formulas (IX), (X), and (XI).

Preferably, Hsp90 inhibitor of structural formulas (LXVa)-LXVf) are selected from Table 4a-c.

TABLE 4a

| Number | Compound |
|---|---|
| 1. | 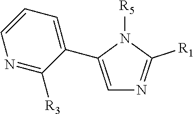 |
| 2. | 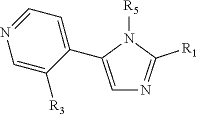 |
| 3. | 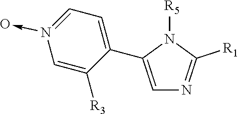 |
| 4. | 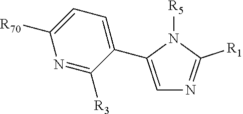 |
| 5. | 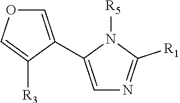 |
| 6. | 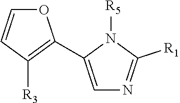 |
| 7. | 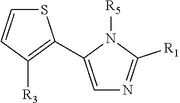 |
| 8. | 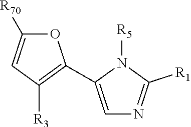 |
| 9. | 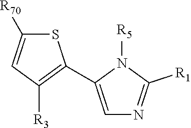 |
| 10. | 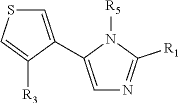 |

TABLE 4b

| Number | Compound |
|---|---|
| 1. | 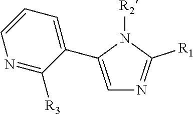 |
| 2. | 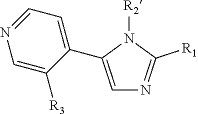 |
| 3. | 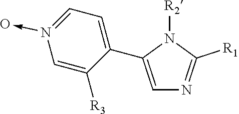 |
| 4. | 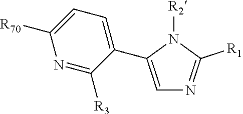 |
| 5. | 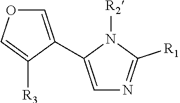 |
| 6. | 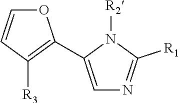 |
| 7. | 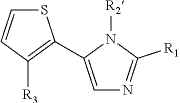 |
| 8. | 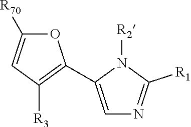 |
| 9. | 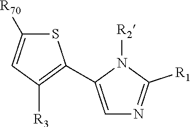 |
| 10. | 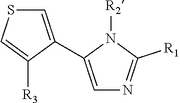 |

TABLE 4c

| Number | Compound |
|---|---|
| 1. | 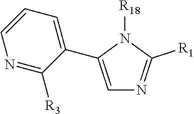 |
| | 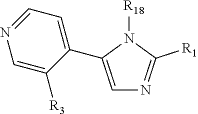 |
| 2. | 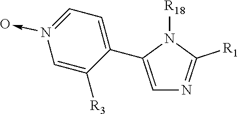 |
| | 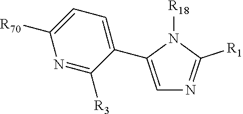 |
| 3. | 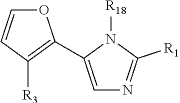 |
| 4. | 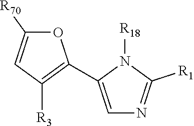 |
| | 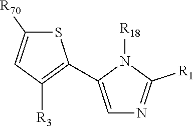 |
| 5. | 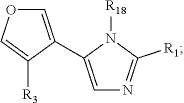 |
| | 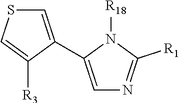 |
| 6. | |
| 7. | |
| 8. | 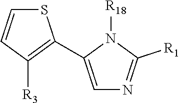 |
| 9. | |
| 10. | |

The values for the variables for the formulas in Tables 4a-c are as defined for structural formulas (LXVa)-(LXVf). Preferably, R₇₀ is a halo, a haloalkyl, a haloalkoxy, a heteroalkyl, —OH, —SH, —NHR₇, —(CH₂)ₖOH, —(CH₂)ₖSH, —(CH₂)ₖNR₇H, —OCH₃, —SCH₃, —NHCH₃, —OCH₂CH₂OH, —OCH₂CH₂SH, —OCH₂CH₂NR₇H, —SCH₂CH₂OH, —SCH₂CH₂SH, —SCH₂CH₂NR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇; and

k is 1, 2, 3, or 4.

In one embodiment, the cell is in a subject in need of treatment for an inflammatory disorder and administration of a compound represented by any one of formulas (LXVa)-LXVf), or any of the embodiments of formulas (LXVa)-LXVf) disclosed herein, to the subject modulates the activity of glucocorticoid receptors in a cell that is in the subject.

In one embodiment, the cell is in a subject in need of treatment for an immune disorder and administration of a compound represented by any one of formulas (LXVa)-LXVf), or any of the embodiments of formulas (LXVa)-LXVf) disclosed herein, to the subject modulates the activity of glucocorticoid receptors in a cell that is in the subject.

In one embodiment, the cell is in a subject in need of immunosuppression and administration of a compound represented by any one of formulas (LXVa)-LXVf), or any of the embodiments of formulas (LXVa)-LXVf) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LXVa)-LXVf), or any of the embodiments of formulas (LXVa)-LXVf) disclosed herein.

In another aspect, the invention provides a method of treating or preventing an immune disorder in a subject, comprising administering to the subject an effective amount of a compound represented by any one of formulas (LXVa)-LXVf), or any of the embodiments of formulas (LXVa)-LXVf) disclosed herein.

In another aspect, the invention provides a method of suppressing the immune system in a subject in need thereof, comprising administering to the subject an effective amount of a compound represented by any one of formulas (LXVa)-LXVf), or any of the embodiments of formulas (LXVa)-LXVf) disclosed herein.

In another aspect, the invention provides a method of modulating the activity of glucocorticoid receptors in a cell, comprising administering to the cell an effective amount of a compound represented by formula (LXVI):

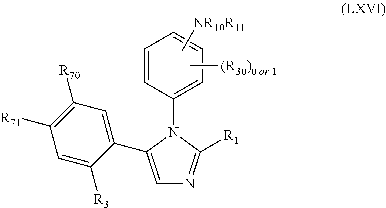

R₇₀ and R₇₁, for each occurrence, are independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. Preferably, R₇₀ is selected from an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇ and R₇₁ is as just described. The values for the remainder of the variables are as described for structural formulas (IX), (X), and (XI).

In one embodiment, the cell is in a subject in need of treatment for an inflammatory disorder and administration of a compound represented by formula (LXVI), or any of the embodiments of formula (LXVI) disclosed herein, to the subject modulates the activity of glucocorticoid receptors in a cell that is in the subject.

In one embodiment, the cell is in a subject in need of treatment for an immune disorder and administration of a compound represented by formula (LXVI), or any of the embodiments of formula (LXVI) disclosed herein, to the subject modulates the activity of glucocorticoid receptors in a cell that is in the subject.

In one embodiment, the cell is in a subject in need of immunosuppression and administration of a compound represented by formula (LXVI), or any of the embodiments of formula (LXVI) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LXVI), or any of the embodiments of formula (LXVI) disclosed herein.

In another aspect, the invention provides a method of treating or preventing an immune disorder in a subject, comprising administering to the subject an effective amount of a compound represented by formula (LXVI), or any of the embodiments of formula (LXVI) disclosed herein.

In another aspect, the invention provides a method of suppressing the immune system in a subject in need thereof, comprising administering to the subject an effective amount of a compound represented by formula (LXVI), or any of the embodiments of formula (LXVI) disclosed herein.

In another aspect, the invention provides a method of modulating the activity of glucocorticoid receptors in a cell, comprising administering to the cell an effective amount of a compound represented by formula (LXVIIa) or (LXVIIb):

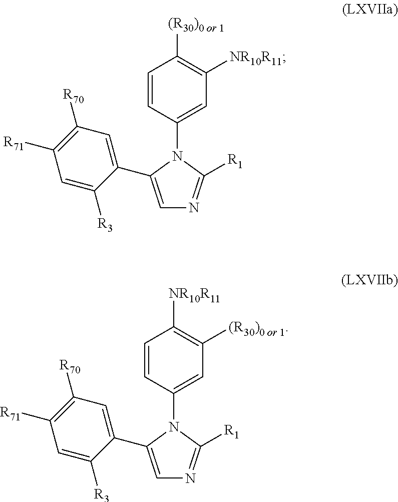

The variables in formulas (LXVIIa) and (LXVIIb) are defined above with reference to formula (LXVI).

A first preferred set of values for the variables of structural formula (LXVIIa) and (LXVIIb) is provided in the following paragraph:

R₁, R₃ or R₇₁ are each independently selected from —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂, and p, R₇₀, R₇, R₈, R₁₀, R₁₁ and R₃₀ are as described for structural formula (LXVI). Preferably, when R₁, R₃ and R₇₁ have these values, R₁₀ and R₁₁ are preferably each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; and p, R₇₀, R₇, and R₃₀ are as described for structural formula (LXVI). More preferably, when R₁, R₃, R₁₀, R₁₁, and R₇₁ have these values, R₇₀ is preferably a C1-C6 alkyl, a C1-C6 haloalkyl, a C1-C6 alkoxy, a C1-C6 haloalkoxy, a C1-C6 alkyl sulfanyl or a C3-C6 cycloalkyl; and p, R₇, R₈ and R₃₀ are as described for structural formula (LXVI).

A second preferred set of values for the variables of structural formula (LXVIIa) and (LXVIIb) is provided in the following paragraph:

R₁ and R₃ are each independently —OH, —SH; R₇₀ is preferably a C1-C6 alkyl, a C1-C6 haloalkyl, a C1-C6 alkoxy, a C1-C6 haloalkoxy, a C1-C6 alkyl sulfanyl or a C3-C6 cycloalkyl; R₁₀ and R₁₁ are preferably each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; R₇₁ is —OH, —SH, —NHR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR_{7,} —OP(O)(OR₇)₂ or —SP(O)(OR₇)₂; and p, R₇ R₈ and R₃₀ are as described for structural formula (LXVI). Preferably, R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl and the remainder of the variables are as just described.

A third preferred set of values for the variables of structural formula (LXVIIa) and (LXVIIb) is provided in the following paragraph:

R₁, R₃ and R₇₁ are independently —SH or —OH; R₇₀ is cyclopropyl or isopropyl; R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; and R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl. Preferably, R₃₀ is a methyl, ethyl, propyl, isopropyl, methoxy or ethoxy. More preferably, R₁, R₃, R₇₀, R₇₁ and R₃₀ are as just described and and R₁₀ and R₁₁ are each independently a hydrogen, methyl, ethyl, propyl, isopropyl, or taken together with the nitrogen to which they are attached, are:

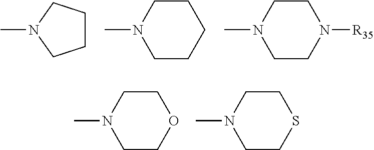

wherein R₃₅ is —H, a C1-C4 alkyl or a C1-C4 acyl.

In one embodiment, the cell is in a subject in need of treatment for an inflammatory disorder and administration of a compound represented by formula (LXVIIa) or (LXVIIb), or any of the embodiments of formula (LXVIIa) or (LXVIIb) disclosed herein, to the subject modulates the activity of glucocorticoid receptors in a cell that is in the subject.

In one embodiment, the cell is in a subject in need of treatment for an immune disorder and administration of a compound represented by formula (LXVIIa) or (LXVIIb), or any of the embodiments of formula (LXVIIa) or (LXVIIb) disclosed herein, to the subject modulates the activity of glucocorticoid receptors in a cell that is in the subject.

In one embodiment, the cell is in a subject in need of immunosuppression and administration of a compound represented by formula (LXVIIa) or (LXVIIb), or any of the embodiments of formula (LXVIIa) or (LXVIIb) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LXVIIa) or (LXVIIb), or any of the embodiments of formula (LXVIIa) or (LXVIIb) disclosed herein.

In another aspect, the invention provides a method of treating or preventing an immune disorder in a subject, comprising administering to the subject an effective amount of a compound represented by formula (LXVIIa) or (LXVIIb), or any-of the embodiments of formula (LXVIIa) or (LXVIIb) disclosed herein.

In another aspect, the invention provides a method of suppressing the immune system in a subject in need thereof, comprising administering to the subject an effective amount of a compound represented by formula (LXVIIa) or (LXVIIb), or any of the embodiments of formula (LXVIIa) or (LXVIIb) disclosed herein.

In another aspect, the invention provides a method of modulating the activity of glucocorticoid receptors in a cell, comprising administering to the cell an effective amount of a compound represented by formula (LXVIIIa) or (LXVIIIb):

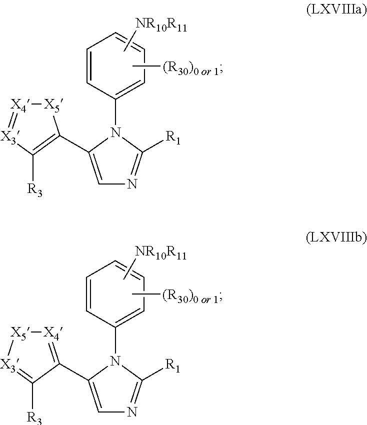

The values for the variables in structural formulas (LXVIIIa) and (LXVIIIb) are as described for structural formulas (LXVc) and (LXVd). Preferably, R₃₀ is an optionally substituted alkyl, an optionally substituted alkenyl, an optionally substituted alkynyl, an optionally substituted cycloalkyl, an optionally substituted cycloalkenyl, an optionally substituted heterocyclyl, an substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂. More preferably, R₃₀ is an optionally substituted alkyl, an optionally substituted alkenyl, an optionally substituted alkynyl, cyano, halo, nitro, an optionally substituted cycloalkyl, haloalkyl, an optionally substituted heterocyclyl, an optionally substituted aryl, an optionally substituted heteroaryl, an optionally substituted aralkyl, an optionally substituted heteroaralkyl, —OR₇, —SR₇, —NR₁₀R₁₁, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)R₇, —C(O)OR₇, —C(O)NR₁₀R₁₁, —C(O)SR₇, —C(S)R₇, —C(S)OR₇, —C(S)NR₁₀R₁₁, —C(S)SR₇, —C(NR₈)OR₇, —C(NR₈)R₇, —C(NR₈)NR₁₀R₁₁, —C(NR₈)SR₇, —S(O)ₚOR₇, —S(O)ₚNR₁₀R₁₁, or —S(O)ₚR₇.

In one embodiment, the cell is in a subject in need of treatment for an inflammatory disorder and administration of a compound represented by formula (LXVIIIa) or (LXVIIIb), or any of the embodiments of formula (LXVIIIa) or (LXVIIIb) disclosed herein, to the subject modulates the activity of glucocorticoid receptors in a cell that is in the subject.

In one embodiment, the cell is in a subject in need of treatment for an immune disorder and administration of a compound represented by formula (LXVIIIa) or (LXVIIIb), or any of the embodiments of formula (LXVIIIa) or (LXVIIIb) disclosed herein, to the subject modulates the activity of glucocorticoid receptors in a cell that is in the subject.

In one embodiment, the cell is in a subject in need of immunosuppression and administration of a compound represented by formula (LXVIIIa) or (LXVIIIb), or any of the embodiments of formula (LXVIIIa) or (LXVIIIb) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LXVIIIa) or (LXVIIIb), or any of the embodiments of formula (LXVIIIa) or (LXVIIIb) disclosed herein.

In another aspect, the invention provides a method of treating or preventing an immune disorder in a subject, comprising administering to the subject an effective amount of a compound represented by formula (LXVIIIa) or (LXVIIIb), or any of the embodiments of formula (LXVIIIa) or (LXVIIIb) disclosed herein.

In another aspect, the invention provides a method of suppressing the immune system in a subject in need thereof, comprising administering to the subject an effective amount of a compound represented by formula (LXVIIIa) or (LXVIIIb), or any of the embodiments of formula (LXVIIIa) or (LXVIIIb) disclosed herein.

In another aspect, the invention provides a method of modulating the activity of glucocorticoid receptors in a cell, comprising administering to the cell an effective amount of a compound represented by any one of formulas (LXIXa)-(LXIXd):

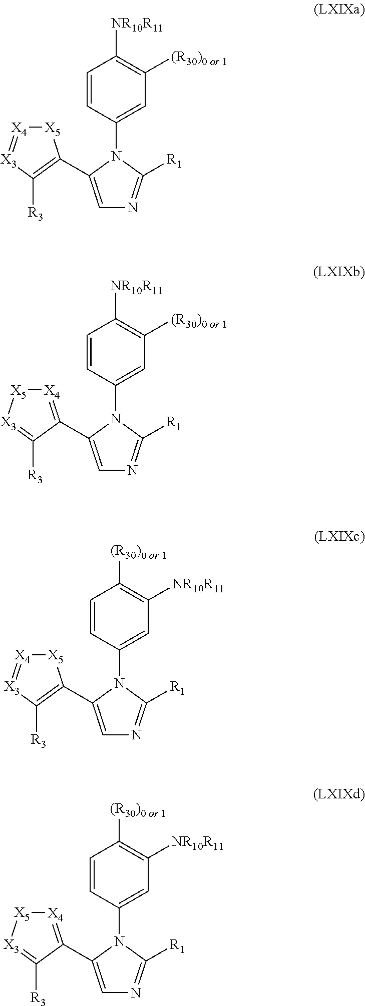

The values of the variables in structural formulas (LXIXa)-(LXIXd) are defined above with reference to structural formulas (LXVIIIa) and (LXVIIIb).

A first preferred set of values for the variables in structural formulas (LXIXa)-(LXIXd) are as described in the following paragraphs:

R₁ and R₃ are each independently —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂;

R₇₀, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, —OH, —SH, —HNR₇, —OC(O)NR₁₀R_{11,} —SC(O)NR₁₀R_{11,} —OC(O)R_{7,} —SC(O)R_{7,} —OC(O)OR_{7,} —SC(O)OR_{7,} —OS(O)ₚR_{7,} —S(O)ₚOR₇, —SS(O)ₚR_{7,} —OS(O)ₚOR_{7,} —SS(O)ₚOR_{7,} —OC(S)R_{7,} —SC(S)R_{7,} —OC(S)OR_{7,} —SC(S)OR_{7,} —OC(S)NR₁₀R_{11,} —SC(S)NR₁₀R_{11,} —OC(NR₈)R_{7,} —SC(NR₈)R_{7,} —OC(NR₈)OR_{7,} —SC(NR₈)OR₇, —OP(O)(OR₇)₂ or —SP(O)(OR₇)₂. Preferably, R₇₀ is a C1-C6 alkyl, a C1-C6 haloalkyl, a C1-C6 alkoxy, a C1-C6 haloalkoxy, a C1-C6 alkyl sulfanyl or a C3-C6 cycloalkyl; and

R₁₀ and R₁₁ and the remainder of the variables in structural formulas (LXIXa)-(LXIXd) are as described for structural formulas (LXVIIIa) and (LXVIIIb). Preferably, R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl.

In one embodiment, the cell is in a subject in need of treatment for an inflammatory disorder and administration of a compound represented by any one of formulas (LXIXa)-(LXIXd), or any of the embodiments of formulas (LXIXa)-(LXIXd) disclosed herein, to the subject modulates the activity of glucocorticoid receptors in a cell that is in the subject.

In one embodiment, the cell is in a subject in need of treatment for an immune disorder and administration of a compound represented by any one of formulas (LXIXa)-(LXIXd), or any of the embodiments of formulas (LXIXa)-(LXIXd) disclosed herein, to the subject modulates the activity of glucocorticoid receptors in a cell that is in the subject.

In one embodiment, the cell is in a subject in need of immunosuppression and administration of a compound represented by any one of formulas (LXIXa)-(LXIXd), or any of the embodiments of formulas (LXIXa)-(LXIXd) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LXIXa)-(LXIXd), or any of the embodiments of formulas (LXIXa)-(LXIXd) disclosed herein.

In another aspect, the invention provides a method of treating or preventing an immune disorder in a subject, comprising administering to the subject an effective amount of a compound represented by any one of formulas (LXIXa)-(LXIXd), or any of the embodiments of formulas (LXIXa)-(LXIXd) disclosed herein.

In another aspect, the invention provides a method of suppressing the immune system in a subject in need thereof, comprising administering to the subject an effective amount of a compound represented by any one of formulas (LXIXa)-(LXIXd), or any of the embodiments of formulas (LXIXa)-(LXIXd) disclosed herein.

In another aspect, the invention provides a method of modulating the activity of glucocorticoid receptors in a cell, comprising administering to the cell an effective amount of a compound represented by any one of formulas (LXXa)-(LXXp):

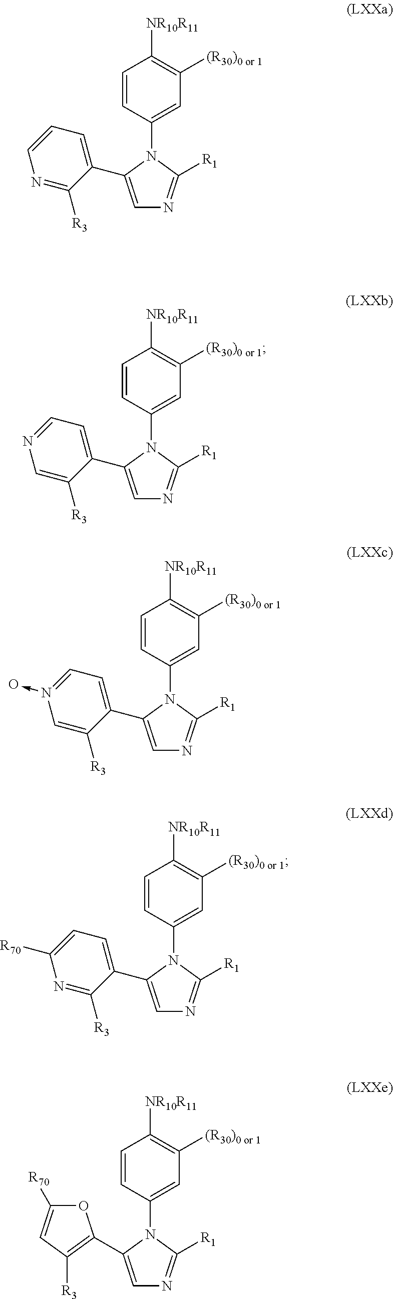

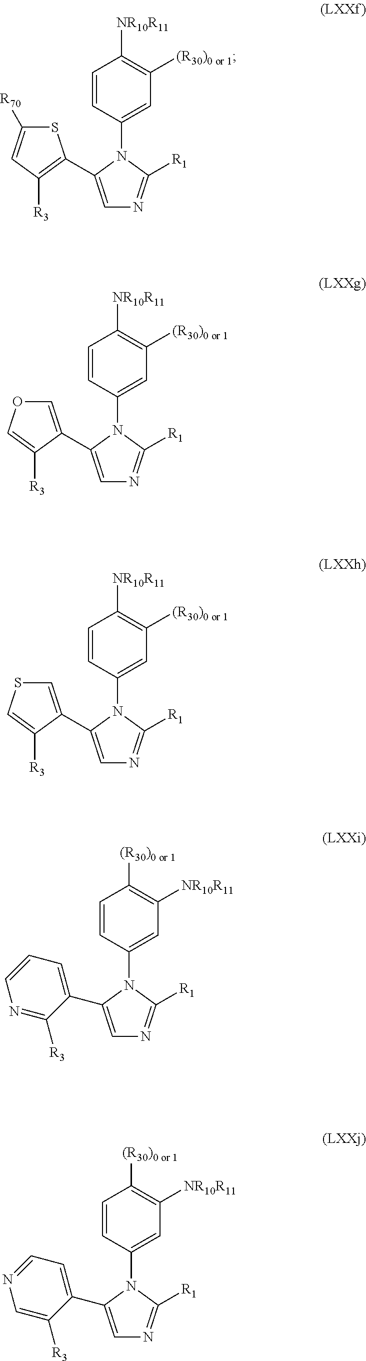

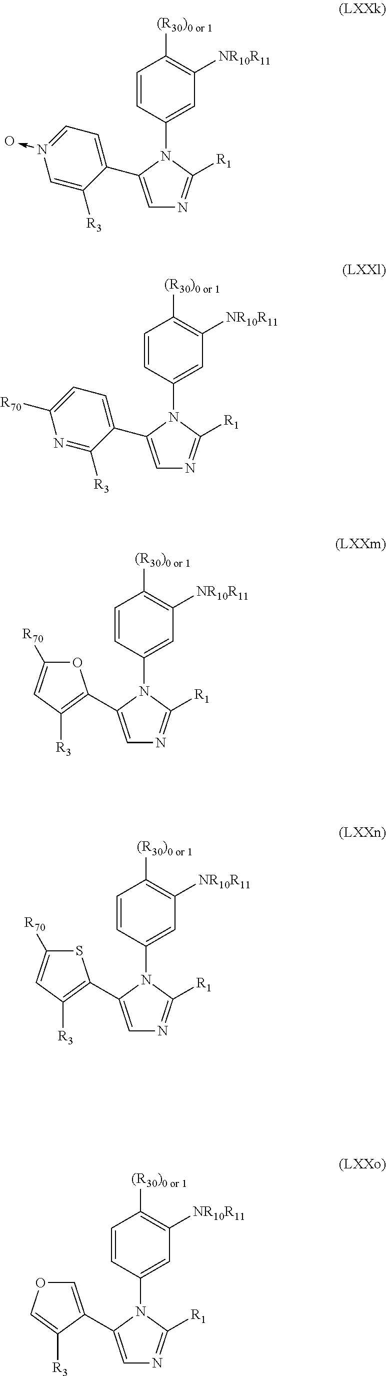

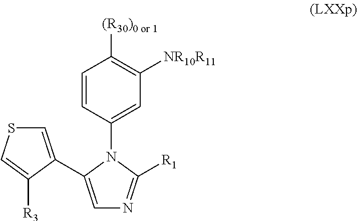

The values of the variables in structural formulas (LXXa)-(LXXp) are defined above with reference to structural formulas (LXIXa)-(LXIXd).

A first preferred set of values for the variables in structural formulas (XIVa-p) are as described in the following paragraphs:

R₁ and R₃ are each independently —OH, —SH, —HNR₇;

R₇₀, is a C1-C6 alkyl, a C1-C6 haloalkyl, a C1-C6 alkoxy, a C1-C6 haloalkoxy, a C1-C6 alkyl sulfanyl or a C3-C6 cycloalkyl;

R₁₀ and R₁₁ and the remainder of the variables in structural formulas (LXXa)-(LXXp) are as described for structural formulas (LXVIIIa) and (LXVIIIb). Preferably, R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁ taken together with the nitrogen to which they are attached form a substituted or unsubstituted nonaromatic, nitrogen-containing heterocyclyl; and

R₃₀ and the remainder of the variables in structural formulas (LXXa)-(LXXp) are as described for structural formulas (LXIXa)-(LXIXd). Preferably, R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl.

A second preferred set of values for the variables in structural formulas (LXXa)-(LXXp) are as described in the following paragraphs:

R₁ and R₃ are independently —SH or —OH;

R₇₀ is cyclopropyl or isopropyl;

R₁₀ and R₁₁ are each independently a hydrogen, a C1-C6 straight or branched alkyl, optionally substituted by —OH, —CN, —SH, amino, a C1-C6 alkoxy, alkylsulfanyl, alkylamino, dialkylamino or a cycloalkyl; or R₁₀ and R₁₁taken together with the nitrogen to which they are attached form a substituted or unsubstituted nonaromatic, nitrogen-containing heterocyclyl;

R₃₀ is —OH, —SH, halogen, cyano, a C1-C6 alkyl, C1-C6 haloalkyl, C1-C6 alkoxy, C1-C6 haloalkoxy or C1-C6 alkyl sulfanyl. Preferably, R₃₀ is a methyl, ethyl, propyl, isopropyl, methoxy or ethoxy; and the remainder of the variables are as described for formulas (LXVIIIa) and (LXVIIIb). More preferably, R₁₀ and R₁₁ are each independently a hydrogen, methyl, ethyl, propyl, isopropyl, or taken together with the nitrogen to which they are attached, are:

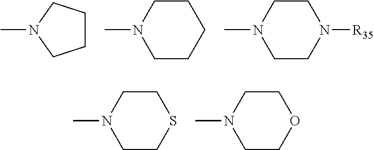

wherein R₃₅ is —H, a C1-C4 alkyl or a C1-C4 acyl.

In one embodiment, the cell is in a subject in need of treatment for an inflammatory disorder and administration of a compound represented by any one of formulas (LXXa)-(LXXp), or any of the embodiments of formulas (LXXa)-(LXXp) disclosed herein, to the subject modulates the activity of glucocorticoid receptors in a cell that is in the subject.

In one embodiment, the cell is in a subject in need of treatment for an immune disorder and administration of a compound represented by any one of formulas (LXXa)-(LXXp), or any of the embodiments of formulas (LXXa)-(LXXp) disclosed herein, to the subject modulates the activity of glucocorticoid receptors in a cell that is in the subject.

In one embodiment, the cell is in a subject in need of immunosuppression and administration of a compound represented by any one of formulas (LXXa)-(LXXp), or any of the embodiments of formulas (LXXa)-(LXXp) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by any one of formulas (LXXa)-(LXXp), or any of the embodiments of formulas (LXXa)-(LXXp) disclosed herein.

In another aspect, the invention provides a method of treating or preventing an immune disorder in a subject, comprising administering to the subject an effective amount of a compound represented by any one of formulas (LXXa)-(LXXp), or any of the embodiments of formulas (LXXa)-(LXXp) disclosed herein.

In another aspect, the invention provides a method of suppressing the immune system in a subject in need thereof, comprising administering to the subject an effective amount of a compound represented by any one of formulas (LXXa)-(LXXp), or any of the embodiments of formulas (LXXa)-(LXXp) disclosed herein.

In another aspect, the invention provides a method of modulating the activity of glucocorticoid receptors in a cell, comprising administering to the cell an effective amount of a compound represented by formula (LXXI) or (LXXII):

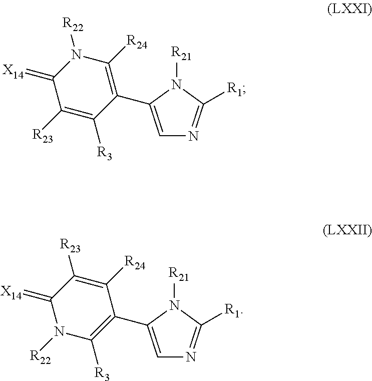

In formulas (LXXI) and (LXXII):

X₁₄ is O, S, or NR₇. Preferably, X₁₄ is O;

R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂. Preferably, R₁ is —OH, —SH, or —NHR₇;

R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;

R₇ and R₈, for each,occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₂₁ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl. Preferably, R₂₁ is an optionally substituted alkyl, an optionally substituted cycloalkyl, an optionally substituted aryl or an optionally substituted heteroaryl. Alternatively, R₂₁ is

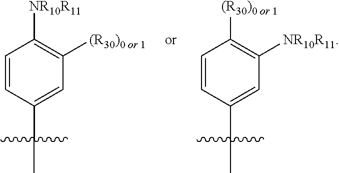

wherein

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl or heteroaryl, an optionally substituted aralkyl; or R₁₀ and R₁₁, taken together with the nitrogen to which they are attached, form an optionally substituted heteroaryl or heterocyclyl; and

R₃₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

z and q are independently an integer from 0 to 4; and

x is 0 or 1, provided that z+x less than or equal to 4.

R₂₂, for each occurrence, is independently —H or an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, a haloalkyl, —C(O)R₇, —C(O)OR₇, —OC(O)R₇, —C(O)NR₁₀R₁₁, —NR₈C(O)R₇, —S(O)ₚR₇, —S(O)ₚOR₇, or —S(O)ₚNR₁₀R₁₁. Preferably, R₂₂ is —H, an alkyl, an aralkyl, —C(O)R₇, —C(O)OR₇, or —C(O)NR₁₀R₁₁; and

R₂₃ and R₂₄, for each occurrence, are independently —H, a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

R₂₆ is a lower alkyl;

p, for each occurrence, is, independently, 0, 1 or 2; and

m, for each occurrence, is independently, 1, 2, 3, or 4.

In one embodiment, the cell is in a subject in need of treatment for an inflammatory disorder and administration of a compound represented by any one of formula (LXXI) or (LXXII), or any of the embodiments of formula (LXXI) or (LXXII) disclosed herein, to the subject modulates the activity of glucocorticoid receptors in a cell that is in the subject.

In one embodiment, the cell is in a subject in need of treatment for an immune disorder and administration of a compound represented by formula (LXXI) or (LXXII), or any of the embodiments of formula (LXXI) or (LXXII) disclosed herein, to the subject modulates the activity of glucocorticoid receptors in a cell that is in the subject.

In one embodiment, the cell is in a subject in need of immunosuppression and administration of a compound represented by formula (LXXI) or (LXXII), or any of the embodiments of formula (LXXI) or (LXXII) disclosed herein, to the subject modulates the activity of glucocorticoid receptors in a cell that is in the subject.

In another aspect, the invention provides a method of treating or preventing an inflammatory disorder in a subject, comprising administering to the subject an effective amount of a compound represented by formula (LXXI) or (LXXII), or any of the embodiments of formula (LXXI) or (LXXII) disclosed herein.

In another aspect, the invention provides a method of treating or preventing an immune disorder in a subject, comprising administering to the subject an effective amount of a compound represented by formula (LXXI) or (LXXII), or any of the embodiments of formula (LXXI) or (LXXII) disclosed herein.

In another aspect, the invention provides a method of suppressing the immune system in a subject in need thereof, comprising administering to the subject an effective amount of a compound represented by formula (LXXI) or (LXXII), or any of the embodiments of formula (LXXI) or (LXXII) disclosed herein.
i) Exemplary Compounds Useful in the Method of the Invention

Exemplary triazole compounds useful in the method of the invention are depicted in Table 5 below. Tautomers, pharmaceutically acceptable salts, solvates, clathrates, hydrates, polymorphs or prodrugs of the compounds in Table 5 are also useful in the method of the invention.

TABLE 5

| No. | Name |
|---|---|
| 1 | 3-(2-Hydroxyphenyl)-4-(naphthalen-1-yl)-5-mercapto-triazole |
| 2 | 3-(2,4-Dihydroxyphenyl)-4-[4-(2-methoxyethoxy)-naphthalen-1- |
| | yl]-5-mercapto-triazole |
| 3 | 3-(2,4-Dihydroxyphenyl)-4-(2-methyl-4-bromophenyl)-5-mercapto- |
| | triazole |
| 4 | 3-(2,4-Dihydroxyphenyl)-4-(4-bromophenyl)-5-mercapto-triazole |
| 5 | 3-(3,4-Dihydroxyphenyl)-4-(6-methoxy-naphthalen-1-yl)-5-mercapto- |
| | triazole |
| 6 | 3-(3,4-Dihydroxyphenyl)-4-(6-ethoxy-naphthalen-1-yl)-5-mercapto- |
| | triazole |
| 7 | 3-(3,4-Dihydroxyphenyl)-4-(6-propoxy-naphthalen-1-yl)-5-mercapto- |
| | triazole |
| 8 | 3-(2,4-Dihydroxy-5-ethyl-phenyl)-4-(5-methoxy-naphthalen-1-yl)-5- |
| | mercapto-triazole |
| 9 | 3-(3,4-Dihydroxyphenyl)-4-(6-isopropoxy-naphthalen-1-yl)-5- |
| | mercapto-triazole |
| 10 | 3-(2,4-Dihydroxyphenyl)-4-(2,6-diethylphenyl)-5-mercapto-triazole |
| 11 | 3-(2,4-Dihydroxyphenyl)-4-(2-methy-6-ethylphenyl)-5-mercapto- |
| | triazole |
| 12 | 3-(2,4-Dihydroxyphenyl)-4-(2,6-diisopropylphenyl)-5-mercapto- |
| | triazole |
| 13 | 3-(2,4-Dihydroxyphenyl)-4-(1-ethyl-indol-4-yl)-5-mercapto-triazole |
| 14 | 3-(2,4-Dihydroxyphenyl)-4-(2,3-dihydro-benzo[1,4]dioxin-5-yl)-5- |
| | mercapto-triazole |
| 15 | 3-(2,4-Dihydroxyphenyl)-4-(3-methylphenyl)-5-mercapto-triazole |
| 16 | 3-(2,4-Dihydroxyphenyl)-4-(4-methylphenyl)-5-mercapto-triazole |
| 17 | 3-(2,4-Dihydroxyphenyl)-4-(2-chlorophenyl)-5-mercapto-triazole |
| 18 | 3-(2,4-Dihydroxyphenyl)-4-(3-chlorophenyl)-5-mercapto-triazole |
| 19 | 3-(2,4-Dihydroxyphenyl)-4-(4-chlorophenyl)-5-mercapto-triazole |
| 20 | 3-(2,4-Dihydroxyphenyl)-4-(2-methoxyphenyl)-5-mercapto-triazole |
| 21 | 3-(2,4-Dihydroxyphenyl)-4-(3-methoxyphenyl)-5-mercapto-triazole |
| 22 | 3-(2,4-Dihydroxyphenyl)-4-(4-methoxyphenyl)-5-mercapto-triazole |
| 23 | 3-(2,4-Dihydroxyphenyl)-4-(3-fluorophenyl)-5-mercapto-triazole |
| 24 | 3-(2,4-Dihydroxyphenyl)-4-(2-ethylphenyl)-5-mercapto-triazole |
| 25 | 3-(2-Hydroxy-4-fluorophenyl)-4-(naphthalen-1-yl)-5-mercapto- |
| | triazole |
| 26 | 3-(2-Hydroxy-4-aminophenyl)-4-(naphthalen-1-yl)-5-mercapto-triazole |
| 27 | 3-(2,4-Dihydroxyphenyl)-4-(2-methyl-4-butyl-phenyl)-5-mercapto- |
| | triazole |
| 28 | 3-(2,4-Dihydroxyphenyl)-4-(2,4-dimethyl-phenyl)-5-mercapto-triazole |
| 29 | 3-(2,4-Dihydroxyphenyl)-4-(2,6-dimethyl-phenyl)-5-mercapto-triazole |
| 30 | 3-(2,4-Dihydroxyphenyl)-4-(2,6-dimethyl-phenyl)-5-mercapto-triazole |
| 31 | 3-(2,4-Dihydroxyphenyl)-4-(4-fluorophenyl)-5-mercapto-triazole |
| 32 | 3-(2,4-Dihydroxyphenyl)-4-(2-methylsulfanylphenyl)-5-mercapto- |
| | triazole |
| 33 | 3-(2,4-Dihydroxyphenyl)-4-(naphthalene-2-yl)-5-mercapto-triazole |
| 34 | 3-(2,4-Dihydroxyphenyl)-4-(2,3-dimethylphenyl)-5-mercapto-triazole |
| 35 | 3-(2,4-Dihydroxyphenyl)-4-(2-methyl-4-fluorophenyl)-5-mercapto- |
| | triazole |
| 36 | 3-(2,4-Dihydroxyphenyl)-4-(acenaphthalen-5-yl)-5-mercapto-triazole |
| 37 | 3-(2-Hydroxy-4-methoxy-phenyl)-4-(naphthalen-1-yl)-5-mercapto- |
| | triazole |
| 38 | 3-(2,4-Dihydroxyphenyl)-4-(2,3-dichlorophenyl)-5-mercapto-triazole |
| 39 | 3-(2,4-Dihydroxyphenyl)-4-(5-methoxynaphthalen-1-yl)-5-mercapto- |
| | triazole |
| 40 | 3-(2,4-Dihydroxyphenyl)-4-(pyren-1-yl)-5-mercapto-triazole |
| 41 | 3-(2,4-Dihydroxyphenyl)-4-(quinolin-5-yl)-5-mercapto-triazole |
| 42 | 3-(2,4-Dihydroxyphenyl)-4-(1,2,3,4-tetrahydronaphthalen-5-yl)-5- |
| | mercapto-triazole |
| 43 | 3-(2,4-Dihydroxyphenyl)-4-(anthracen-1-yl)-5-mercapto-triazole |
| 44 | 3-(2,4-Dihydroxyphenyl)-4-(biphenyl-2-yl)-5-mercapto-triazole |
| 45 | 3-(2,4-Dihydroxy-6-methyl-phenyl)-4-(naphthalene-1-yl)-5-mercapto- |
| | triazole |
| 46 | 3-(2,4-Dihydroxyphenyl)-4-(4-pentyloxyphenyl)-5-mercapto-triazole |
| 47 | 3-(2,4-Dihydroxyphenyl)-4-(4-octyloxyphenyl)-5-mercapto-triazole |
| 48 | 3-(2,4-Dihydroxyphenyl)-4-(4-chloronaphthalen-1-yl)-5-mercapto- |
| | triazole |
| 49 | 3-(2,4-Dihydroxy-5-ethyl-phenyl)-4-(naphthalen-1-yl)-5-mercapto- |
| | triazole |
| 50 | 3-(2,4-Dihydroxy-5-ethyl-phenyl)-4-(7-carboxymethoxy-naphthalen- |
| | 1-yl)-5-mercapto-triazole |
| 51 | 3-(2,4-Dihydroxyphenyl)-4-(2-methyl-quinolin-4-yl)-5-mercapto- |
| | triazole |
| 52 | 3-(3-Hydroxypyridin-4-yl)-4-(naphthalen-1-yl)-5-mercapto-triazole |
| 53 | 3-(2-Hydroxy-4-acetylamino-phenyl)-4-(naphthalen-1-yl)-5-mercapto- |
| | triazole |
| 54 | 3-(2,4-Dihydroxy-phenyl)-4-(1,2,3,4-tetrahydronaphthalen-1-yl)-5- |
| | mercapto-triazole |
| 55 | 3-(2,4-Dihydroxy-phenyl)-4-(2,3-dihydro-benzo[1,4]dioxin-5-yl)-5- |
| | mercapto-triazole |
| 56 | 3-(2,4-Dihydroxy-phenyl)-4-(3,5-dimethoxyphenyl)-5-mercapto- |
| | triazole |
| 57 | 3-(2,4-Dihydroxy-phenyl)-4-(2,3-dimethyl-1H-indol-4-yl)-5- |
| | mercapto-triazole |
| 58 | 3-(2,4-Dihydroxy-3-propyl-phenyl)-4-(naphthalen-1-yl)-5-mercapto- |
| | triazole |
| 59 | 3-(1-ethyl-4-hydroxy-6-oxo-1,6-dihydro-pyridin-3-yl)-4-(naphthalen- |
| | 1-yl)-5-mercapto-triazole |
| 60 | 3-(4-hydroxy-6-oxo-pyridin-3-yl)-4-(naphthalen-1-yl)-5-mercapto- |
| | triazole |
| 61 | 3-(2,4-Dihydroxy-phenyl)-4-(3,5-di-tert-butylphenyl)-5-mercapto- |
| | triazole |
| 62 | 3-(2,6-Dihydroxy5-fluoro-pyridin-3-yl) 4-(naphthalen-1-yl)-5- |
| | mercapto-triazole |
| 63 | 3-(2,4-Dihydroxy-5-methyl-phenyl)-4-(naphthalene-1-yl)-5-mercapto- |
| | triazole |
| 64 | 3-[2,4-Dihydroxy-phenyl]-4-(3-benzoylphenyl)-5-mercapto-triazole |
| 65 | 3-(2,4-Dihydroxy-phenyl)-4-(4-carboxy-naphthalen-1-yl)-5-mercapto- |
| | triazole |
| 66 | 3-(2,4-Dihydroxy-phenyl)-4-[4-(N,N-dimethylcarbamoyl)-naphthalen- |
| | 1-yl]-5-mercapto-triazole |
| 67 | 3-(2,4-Dihydroxy-phenyl)-4-(4-propoxy-naphthalen-1-yl)-5-mercapto- |
| | triazole |
| 68 | 3-(2,4-Dihydroxy-phenyl)-4-(4-isopropoxy-naphthalen-1-yl)-5- |
| | mercapto-triazole |
| 69 | 3-(2,4-Dihydroxy-phenyl)-4-(5-isopropoxy-naphthalen-1-yl)-5- |
| | mercapto-triazole |
| 70 | 3-(2,4-Dihydroxy-phenyl)-4-(isoquinolin-5-yl)-5-mercapto-triazole |
| 71 | 3-(2,4-Dihydroxy-phenyl)-4-(5-propoxy-naphthalen-1-yl)-5- |
| | mercapto-triazole |
| 72 | 3-(2-Hydroxy-4-methanesulfonamino-phenyl)-4-(naphthalen-1-yl)-5- |
| | mercapto-triazole |
| 73 | 3-(2,4-Dihydroxy-3,6-dimethyl-phenyl)-4-(naphthalen-1-yl)-5- |
| | mercapto-triazole |
| 74 | 3-(2,4-Dihydroxy-phenyl)-4-[7-(2-methoxyethoxy)-naphthalen-1-yl]- |
| | 5-mercapto-triazole |
| 75 | 3-(2,4-Dihydroxy-5-hexyl-phenyl)-4-(naphthalen-1-yl)-5-mercapto- |
| | triazole |
| 76 | 3-(2,4-Dihydroxy-5-ethyl-phenyl)-4-(4-methoxy-naphthalen-1-yl)-5- |
| | mercapto-triazole |
| 77 | 3-(2,4-Dihydroxy-5-ethyl-phenyl)-4-(6-methoxy-naphthalin-1-yl)-5- |
| | mercapto-triazole |
| 78 | 3-(2,4-Dihydroxy-3-chloro-5-ethyl-phenyl)-4-(naphthalen-1-yl)-5- |
| | mercapto-triazole |
| 79 | 3-(2,4-Dihydroxy-5-ethyl-phenyl)-4-(2,3-dimethy-4-methoxy- |
| | phenyl)-5-mercapto-triazole |
| 80 | 3-(2,4-Dihydroxy-phenyl)-4-(7-isopropoxy-naphthalen-1-yl)-5- |
| | mercapto-triazole |
| 81 | 3-(2,4-Dihydroxy-phenyl)-4-(7-ethoxy-naphthalen-1-yl)-5-mercapto- |
| | triazole |
| 82 | 3-(2,4-Dihydroxy-phenyl)-4-(7-propoxy-naphthalen-1-yl)-5-mercapto- |
| | triazole |
| 83 | 3-(2-Hydroxy-4-methoxymethyoxy-phenyl)-4-(naphthalen-1-yl)-5- |
| | mercapto-triazole |
| 84 | 3-[2-Hydroxy-4-(2-hydroxy-ethoxy)-phenyl]-4-(naphthalen-1-yl)-5- |
| | mercapto-triazole |
| 85 | 3-(2,4-Dihydroxyphenyl)-4-(7-methoxy-naphthalen-1-yl)-5- |
| | mercapto-triazole |
| 86 | 3-(2,4-Dihydroxyphenyl)-4-(5-methoxy-naphthalen-1-yl)-5-mercapto- |
| | triazole |
| 87 | 3-(2,4-Dihydroxyphenyl)-4-(4-hydroxy-naphthalen-1-yl)-5-mercapto- |
| | triazole |
| 88 | 3-(2,4-Dihydroxyphenyl)-4-(1-isopropyl-indol-4-yl)-5-mercapto- |
| | triazole |
| 89 | 3-(2,4-Dihydroxy-5-tert-butyl-phenyl)-4-(naphthalen-1-yl)-5- |
| | mercapto-triazole |
| 90 | 3-(2,4-Dihydroxy-5-propyl-phenyl)-4-(naphthalen-1-yl)-5-mercapto- |
| | triazole |
| 91 | 3-(2,4-Dihydroxy-3-methyl-5-ethyl-phenyl)-4-(naphthalen-1-yl)-5- |
| | mercapto-triazole |
| 92 | 3-(2,4-Dihydroxy-5-isobutyl-phenyl)-4-(naphthalen-1-yl)-5-mercapto- |
| | triazole |
| 93 | 3-(2,4-Dihydroxy-phenyl)-4-(2,3-dimethoxy-phenyl)-5-mercapto- |
| | triazole |
| 94 | 3-(2,4-Dihydroxy-phenyl)-4-(2-methoxy-3-chloro-phenyl)-5- |
| | mercapto-triazole |
| 95 | 3-(2,4-Dihydroxy-phenyl)-4-(indol-4-yl)-5-mercapto-triazole |
| 96 | 3-(2,4-Dihydroxy-phenyl)-4-[1-(2-methoxyethoxy)-indol-4-yl]-5- |
| | mercapto-triazole |
| 97 | 3-(2,4-Dihydroxy-phenyl)-4-(naphthalen-1-yl)-5-hydroxy-triazole |
| 98 | 3-(1-Oxo-3-hydroxy-pyridin-4-yl)-4-(naphthalen-1-yl)-5-mercapto- |
| | triazole |
| 99 | 3-(2,5-Dihydroxy-4-carboxy)-4-(naphthalen-1-yl)-5-mercapto-triazole |
| 100 | 3-(2,4-Dihydroxy-5-ethyl-phenyl)-4-(1-isopropyl-indol-4-yl)-5- |
| | mercapto-triazole |
| 101 | 3-(2,4-Dihydroxy-5-ethyl-phenyl)-4-[1-(dimethyl-carbamoyl)-indol- |
| | 4-yl]-5-mercapto-triazole |
| 102 | 3-(2,4-Dihydroxy-5-ethyl-phenyl)-4-(1-ethyl-benzoimidazol-4-yl)-5- |
| | mercapto-triazole |
| 103 | 3-(2,4-Dihydroxy-5-ethyl-phenyl)-4-(1,2,3-trimethyl-indol-5-yl)-5- |
| | mercapto-triazole |
| 104 | 3-(2,5-Dihydroxy-4-hydroxymethyl-phenyl)-4-(naphthalen-1-yl)-5- |
| | mercapto-triazole |
| 105 | 3-(2-Hydroxy-4-amino-phenyl)-4-(naphthalen-1-yl)-5-mercapto- |
| | triazole |
| 106 | 3-(2-Hydroxy-4-acetylamino-phenyl)-4-(naphthalen-1-yl)-5-mercapto- |
| | triazole |
| 107 | 3-(2,4-Dihydroxy-3-chloro-phenyl)-4-(naphthalen-1-yl)-5-mercapto- |
| | triazole |
| 108 | 3-(2,4-Dihydroxy-phenyl)-4-(naphthalen-1-yl)-5-mercapto-triazole |
| 109 | 3-(2,4-Dihydroxy-phenyl)-4-(2-methyl-phenyl)-5-mercapto-triazole |
| 110 | 3-(2,4-Dihydroxy-phenyl)-4-(2,5-dimethoxy-phenyl)-5-mercapto- |
| | triazole |
| 111 | 3-(2,4-Dihydroxy-phenyl)-4-phenyl-5-mercapto-triazole |
| 112 | 3-(2-Hydroxy-phenyl)-4-(2-methoxy-phenyl)-5-mercapto-triazole |
| 113 | 3-(2-Hydroxy-phenyl)-4-(4-methyl-phenyl)-5-mercapto-triazole |
| 114 | 3-(2-Hydroxy-phenyl)-4-(4-bromo-phenyl)-5-mercapto-triazole |
| 115 | 3-(2,4-Dihydroxy-phenyl)-4-(naphthalen-1-yl)-5-(methyl sulfanyl)- |
| | triazole |
| 116 | 3-(2,4-Dimethoxy-phenyl)-4-(naphthalen-1-yl)-5-mercapto-triazole |
| 117 | 3-[2,4-Di-(dimethyl-carbamoyloxy)-phenyl]-4-(naphthalen-1-yl)-5- |
| | (dimethyl-carbamoylsulfanyl)-triazole |
| 118 | 3-(2,4-Dihydroxy-phenyl)-4-(naphthalen-1-yl)-5- |
| | (dimethylcarbamoylsulfanyl)-triazole |
| 119 | 3-(2,4-Diethoxycarbonyloxy-phenyl)-4-(naphthalen-1-yl)-5- |
| | (ethoxycarbonylsulfanyl)-triazole |
| 120 | 3-(2,4-Di-isobutyryloxy-phenyl)-4-(naphthalen-1-yl)-5- |
| | (isobutyrylsulfanyl)-triazole |
| 121 | 3-[2,4-Di-(dimethyl-carbamoyloxy)-phenyl]-4-(quinolin-5-yl)-5- |
| | (dimethyl-carbamoylsulfanyl)-triazole |
| 122 | 3-(2,4-Diacetoxy-phenyl)-4-(naphthalen-1-yl)-5-(acetylsulfanyl)- |
| | triazole |
| 123 | 3-(2,4-Diacetoxy-phenyl)-4-(naphthalen-1-yl)-5-mercapto-triazole |
| 124 | 3-(2,4-Diethylcarbamoyloxy-phenyl)-4-(naphthalen-1-yl)-5- |
| | (ethylcarbamoylsulfanyl)-triazole |
| 125 | 3-(2,4-Dihydroxy-phenyl)-4-(naphthalen-1-yl)-5-(2- |
| | hydroxyethylsulfanyl)-triazole |
| 126 | 3-(2,4-Dihydroxy-phenyl)-4-ethyl-5-mercapto-triazole |
| 127 | 3-(2,4-Dihydroxy-phenyl)-4-propyl-5-mercapto-triazole |
| 128 | 3-(2,4-Dihydroxy-phenyl)-4-isopropyl-5-mercapto-triazole |
| 129 | 3-(2,4-Dihydroxy-phenyl)-4-butyl-5-mercapto-triazole |
| 130 | 3-(2,4-Dihydroxy-phenyl)-4-cyclopropyl-5-mercapto-triazole |
| 131 | 3-(2,4-Dihydroxy-phenyl)-4-(naphthalen-1-yl)-5- |
| | (carboxyethysulfanyl)-triazole |
| 132 | 3-(2,6-Dimethoxy-5-fluoro-pyridin-3-yl)-4-(naphthalen-1-yl)-5- |
| | mercapto-triazole |
| 133 | 3-(2-Methanesulfonyloxy-4-methanesulfonylamino-phenyl)-4- |
| | (naphthalen-1-yl)-5-mercapto-triazole |
| 134 | 3-(2-Methoxy-phenyl)-4-(4-methoxy-phenyl)-5-mercapto-triazole |
| 135 | 3-(3-Hydroxy-naphthalen-2-yl)-4-phenyl-5-mercapto-triazole |
| 136 | 3-(2-Methoxy-phenyl)-4-(4-methyl-phenyl)-5-mercapto-triazole |
| 137 | 3-(2,4-Dihydroxy-5-ethyl-phenyl)-4-(3-methox-phenyl)-5-hydroxy- |
| | triazole |
| 138 | 3-(2,4-Dihydroxy-5-ethyl-phenyl)-4-(naphthalen-1-yl)-5-hydroxy- |
| | triazole |
| 139 | 3-(2,4-Dihydroxy-5-ethyl-phenyl)-4-(1-isopropyl-indol-3-yl)-5- |
| | hydroxy-triazole |
| 140 | 3-(2,4-Dihydroxy-5-ethyl-phenyl)-4-(1-isopropyl-indol-4-yl)-5- |
| | amino-triazole |
| 141 | 3-(2,4-Dihydroxy-5-ethyl-phenyl)-4-(3-methoxy-phenyl)-5-amino- |
| | triazole |
| 142 | 3-(2,4-Dihydroxy-5-ethyl-phenyl)-4-(naphthalen-1-yl)-5-amino- |
| | triazole |
| 143 | 3-(2-Hydroxy-5-ethyloxy-phenyl)-4-(naphthalen-1-yl)-5-hydroxy- |
| | triazole |
| 144 | 3-(2-Hydroxy-5-isopropyl-phenyl)-4-(naphthalen-1-yl)-5-hydroxy- |
| | triazole |
| 145 | 3-(2-Dihydroxy-phenyl)-4-(7-fluoro-naphthalen-1-yl)-5-hydroxy- |
| | triazole |
| 146 | 3-(2,4-Dihydroxy-phenyl)-4-(2,3-difluorophenyl)-5-hydroxy-triazole |
| 147 | 3-(2,4-Dihydroxy-phenyl)-4-[2-(1H-tetrazol-5-yl)-phenyl]-5- |
| | hydroxy-triazole |
| 148 | 3-(2,4-Dihydroxy-phenyl)-4-(benzothiazol-4-yl)-5-hydroxy-triazole |
| 149 | 3-(2,4-Dihydroxy-phenyl)-4-(9H-purin-6-yl)-5-hydroxy-triazole |
| 150 | 3-(2,4-Dihydroxy-phenyl)-4-{4-[2-(moropholin-1-yl)-ethoxy]- |
| | phenyl}-5-hydroxy-triazole |
| 151 | 3-(2,4-Dihydroxy-phenyl)-4-cyclopentyl-5-hydroxy-triazole |
| 152 | 3-(2,4-Dihydroxy-phenyl)-4-phenyl-5-(sulfamoylamino)-triazole |
| 153 | 3-(2,4-Dihydroxy-5-methoxy-phenyl)-4-(naphthalene-1-yl)-5- |
| | ureido-triazole |
| 154 | 3-(2,4-Dihydroxy-5-methoxy-phenyl)-4-(2,3-difluorophenyl)-5- |
| | ureido-triazole |
| 155 | 3-(2,4-Dihydroxy-5-ethyl-phenyl)-4-(1-isopropyl-indol-4-yl)-5- |
| | ureido-triazole |
| 156 | 3-(2,4-Dihydroxy-5-ethyl-phenyl)-4-(quinolin-5-yl)-5-ureido-triazole |
| 157 | 3-(2,4-Dihydroxy-5-methoxy-phenyl)-4-(naphthalene-1-yl)-5- |
| | carbamoyloxy-triazole |
| 158 | 3-(2,4-Dihydroxy-5-ethyl-phenyl)-4-(3-trifluoromethyl-phenyl)-5- |
| | carbamoyloxy-triazole |
| 159 | 3-(2,4-Dihydroxy-5-ethyl-phenyl)-4-(1-methyl-indol-4-yl)-5- |
| | carbamoyloxy-triazole |
| 160 | 3-(2,4-Dihydroxy-5-methoxy-phenyl)-4-(8-methoxy-quinolin-5-yl)-5- |
| | carbamoyloxy-triazole |
| 161 | 3-(2,4-Dihydroxy-5-isopropyl-phenyl)-4-(3-methyl-quinolin-5-yl)-5- |
| | carboxyamino-triazole |
| 162 | 3-(2,4-Dihydroxy-phenyl)-4-(1-methyl-2-chloro-indol-4-yl)-5- |
| | carbamoyloxy-triazole |
| 163 | 3-(2,4-Dihydroxy-5-methoxy-phenyl)-4-[3,5-di-(trifluoromethyl)- |
| | phenyl]-5-carbamoyloxy-triazole |
| 164 | 3-(2,4-Dihydroxy-5-methoxy-phenyl)-4-(3-trifluoromethyl-phenyl)-5- |
| | (sulfamoylamino)-triazole |
| 165 | 3-(2,4-Dihydroxy-5-methoxy-phenyl)-4-(naphthalene-1-yl)-5- |
| | (sulfamoylamino)-triazole |
| 166 | 3-(2,4-Dihydroxy-5-methoxy-phenyl)-4-(1-isopropyl-benzoimidazol- |
| | 4-yl)-5-(sulfamoylamino)-triazole |
| 167 | 3-(2,4-Dihydroxy-5-methoxy-phenyl)-4-(3-isopropylphenyl)-5- |
| | (thiocarboxyamino)-triazole |
| 168 | 3-(2,4-Dihydroxy-5-methoxy-phenyl)-4-(3-isopropyloxy-phenyl)-5- |
| | (sulfamoyloxy)-triazole |
| 169 | 3-(2,4-Dihydroxy-5-methoxy-phenyl)-4-(naphthalene-1-yl)-5- |
| | (sulfamoyloxy)-triazole |
| 170 | 3-(2,4-Dihydroxy-5-methoxy-phenyl)-4-(1-isopropyl-benzoimidazol- |
| | 4-yl)-5-(sulfamoyloxy)-triazole |
| 171 | 3-(2-Hydroxy-4-ethoxycarbonyoxy-5-methoxy-phenyl)-4-(1- |
| | isopropyl-benzoimidazol-4-yl)-5-hydroxy-triazole |
| 172 | 3-(2-Hydroxy-4-ethoxycarbonyoxy-5-ethyl-phenyl)-4-(naphthalin-2- |
| | yl)-5-hydroxy-triazole |
| 173 | 3-[2-Hydroxy-4-(dimethyl-carbamoyoxy)-5-ethyl-phenyl]-4- |
| | (naphthalin-2-yl)-5-hydroxy-triazole |
| 174 | 3-[2-Hydroxy-4-(dimethyl-carbamoyoxy)-5-chloro-phenyl]-4- |
| | (quinolin-5-yl)-5-mercapto-triazole |
| 175 | 3-[2-Hydroxy-4-(dimethyl-carbamoyoxy)-5-ethyl-phenyl]-4-(2,3- |
| | difluoro-phenyl)-5-mercapto-triazole |
| 176 | 3-[2-Hydroxy-4-isobutyryloxy-5-ethyl-phenyl]-4-(1-methyl-benzo- |
| | imidazol-4-yl)-5-hydroxy-triazole |
| 177 | 3-(2,4-Dihydroxy-5-methoxy-phenyl)-4-(naphthalen-1-yl)-5- |
| | mercapto-triazole |
| 178 | 3-(2,4-Dihydroxy-5-ethyl-phenyl)-4-(5-hydroxy-naphthalen-1-yl)-5- |
| | mercapto-triazole |
| 179 | 3-(2,4-Dihydroxy-phenyl)-4-(naphthalen-1-ylmethyl)-5-mercapto- |
| | triazole |
| 180 | 3-(2-Hydroxy-4-methoxyphenyl)-4-(naphthalen-1-yl)-5-mercapto- |
| | triazole |
| 181 | 3-(2,4-Dihydroxy-phenyl)-4-(biphenyl-3-yl)-5-mercapto-triazole |
| 182 | 3-(2,4-Dihydroxy-phenyl)-4-(2-methyl-5-hydroxymethyl-phenyl)-5- |
| | mercapto-triazole |
| 183 | 3-(2,4-Dihydroxy-phenyl)-4-(1-dimethylcarbamoyl-indol-4-yl)-5- |
| | mercapto-triazole |
| 184 | 3-(2,4,5-Trihydroxy-phenyl)-4-(naphthalene-1-yl)-5-mercapto-triazole |
| 185 | 3-(2,4-Dihydroxy-5-ethyl-phenyl)-4-(2,3-dimethyl-indol-5-yl)-5- |
| | mercapto-triazole |
| 186 | 3-(2,4-Dihydroxy-5-ethyl-phenyl)-4-(3-t-butyl-4-methoxy-phenyl)-5- |
| | mercapto-triazole |
| 187 | 3-(2,4-Dihydroxy-5-ethyl-phenyl)-4-(1-ethyl-1H-benzoimidazol-4-yl)- |
| | 5-mercapto-triazole, HCl salt |
| 188 | 3-(2,4-Dihydroxy-5-ethyl-phenyl)-4-(1-isopropyl-7-methoxy-indol- |
| | 4-yl)-5-mercapto-triazole |
| 189 | 3-(2,4-Dihydroxy-5-cyclopropyl-phenyl)-4-(naphthalene-1-yl)-5- |
| | mercapto-triazole |
| 190 | 3-(2,4-dihydroxy-5-ethyl-phenyl)-4-(1-propyl-indol-4-yl)-5-mercapto- |
| | [1,2,4] triazole |
| 191 | 3-(2,4-dihydroxy-5-ethyl-phenyl)-4-(1-acetyl-2,3-dimethyl-indol-5- |
| | yl)-5-mercapto-[1,2,4] triazole |
| 192 | 3-(2,4-dihydroxy-5-ethyl-phenyl)-4-(2-methyl-3-ethyl- benzimidazol- |
| | 5-yl)-5-mercapto-[1,2,4] triazole |
| 193 | 3-(2,4-dihydroxy-5-ethyl-phenyl)-4-(1-ethyl-2-methyl- benzimidazol- |
| | 5-yl)-5-mercapto-[1,2,4] triazole |
| 194 | 3-(2,4-dihydroxy-5-ethyl-phenyl)-4-(1-propyl-2,3-dimethyl-indol-5- |
| | yl)-5-mercapto-[1,2,4] triazole |
| 195 | 3-(2,4-dihydroxy-5-ethyl-phenyl)-4-(N-methyl-tetrahydrocarbozol-7- |
| | yl)-5-mercapto-[1,2,4] triazole |
| 196 | 3-(2,4-dihydroxy-5-ethyl-phenyl)-4-(N-methyl-cyclononan[a]indol-5- |
| | yl)-5-mercapto-[1,2,4] triazole |
| 197 | 3-(2,4-dihydroxy-5-ethyl-phenyl)-4-(1-n-butyl-indol-4-yl)-5- |
| | mercapto-[1,2,4] triazole |
| 198 | 3-(2,4-dihydroxy-5-ethyl-phenyl)-4-(1-n-pentyl-indol-4-yl)-5- |
| | mercapto-[1,2,4] triazole |
| 199 | 3-(2,4-dihydroxy-5-ethyl-phenyl)-4-(1-n-hexyl-indol-4-yl)-5- |
| | mercapto-[1,2,4] triazole |
| 200 | 3-(2,4-dihydroxy-5-cyclopropyl-phenyl)-4-(1-(1-methylcyclopropyl)- |
| | indol-4-yl)-5-mercapto-[1,2,4] triazole |
| 201 | 3-(2,4-dihydroxy-5-cyclopropyl-phenyl)-4-(1-isopropyl-7-methoxy- |
| | indol-4-yl)-5-mercapto-[1,2,4] triazole |
| 202 | 3-(2,4-dihydroxy-5-cyclopropyl-phenyl)-4-(1,2,3-trimethyl-indol-5- |
| | yl)-5-mercapto-[1,2,4] triazole |
| 203 | 3-(2,4-dihydroxy-5-ethyl-phenyl)-4-(1-isopropyl-7-methoxy-indol-4- |
| | yl)-5-mercapto-[1,2,4] triazole disodium salt |
| 204 | 3-(2,4-dihydroxy-5-tert-butyl-phenyl)-4-(1-isopropyl-7-methoxy- |
| | indol-4-yl)-5-mercapto-[1,2,4] triazole |
| 205 | 3-(2,4-dihydroxy-5-cyclopropyl-phenyl)-4-(1-propyl-7-methoxy- |
| | indol-4-yl)-5-mercapto-[1,2,4] triazole |
| 206 | 3-(2,4-dihydroxy-5-ethyl-phenyl)-4-(1-methyl-3-ethyl-indol-5-yl)-5- |
| | mercapto-[1,2,4] triazole |
| 207 | 3-(2,4-dihydroxy-5-ethyl-phenyl)-4-(1,3-dimethyl-indol-5-yl)-5- |
| | mercapto-[1,2,4] triazole |
| 208 | 3-(2,4-dihydroxy-5-isopropyl-phenyl)-4-(1-isopropyl-7-methoxy- |
| | indol-4-yl)-5-mercapto-[1,2,4] triazole |
| 209 | 3-(2,4-dihydroxy-5-ethyl-phenyl)-4-(1-methyl-3-isopropyl-indol-5- |
| | yl)-5-mercapto-[1,2,4] triazole |
| 210 | 3-(2,4-dihydroxy-5-ethyl-phenyl)-4-(N-ethyl-carbozol-7-yl)-5- |
| | mercapto-[1,2,4] triazole |
| 211 | 3-(2,4-dihydroxy-5-ethyl-phenyl)-4-(1-isopropyl-7-hydroxy-indol-4- |
| | yl)-5-mercapto-[1,2,4] triazole |
| 212 | 3-(2,4-dihydroxy-5-ethyl-phenyl)-4-(1-isopropyl-7-ethoxy-indol-4- |
| | yl)-5-mercapto-[1,2,4] triazole |
| 213 | 3-(2,4-dihydroxy-5-ethyl-phenyl)-4-(1,2-dimethyl-indol-5-yl)-5- |
| | mercapto-[1,2,4] triazole |
| 214 | 3-(2,4-dihydroxy-5-ethyl-phenyl)-4-(N-methyl-indol-5-yl)-5- |
| | mercapto-[1,2,4] triazole |
| 215 | 3-(2,4-dihydroxy-5-ethyl-phenyl)-4-(2-methyl-7-methoxy- |
| | benzofuran-4-yl)-5-mercapto-[1,2,4] triazole |
| 216 | 3-(2,4-dihydroxy-5-ethyl-phenyl)-4-(benzofuran-5-yl)-5-mercapto- |
| | [1,2,4] triazole |
| 217 | 3-(2,4-dihydroxy-5-ethyl-phenyl)-4-(2-methyl-1,3-benzoxaz-5-yl)-5- |
| | mercapto-[1,2,4] triazole |
| 218 | 3-(2,4-dihydroxy-5-isopropyl-phenyl)-4-(1,3-dimethyl-indol-5-yl)-5- |
| | mercapto-[1,2,4] triazole |
| 219 | 3-(2,4-dihydroxy-5-cyclopropyl-phenyl)-4-(1,3-dimethyl-indol-5-yl)- |
| | 5-mercapto-[1,2,4] triazole |
| 220 | 3-(2,4-dihydroxy-5-ethyl-phenyl)-4-(1,3-dimethyl-indol-5-yl)-5- |
| | hydroxy-[1,2,4] triazole |
| 221 | 3-(2,4-dihydroxy-5-isopropyl-phenyl)-4-(N-methyl-indol-5-yl)-5- |
| | mercapto-[1,2,4] triazole |
| 222 | 3-(2,4-dihydroxy-5-isopropyl-phenyl)-4-(1,2-dimethyl-indol-5-yl)-5- |
| | mercapto-[1,2,4] triazole |
| 223 | 3-(2,4-dihydroxy-5-isopropyl-phenyl)-4-(1,3-dimethyl-indol-5-yl)-5- |
| | hydroxy-[1,2,4] triazole |
| 224 | 3-(2,4-dihydroxy-5-cyclopropyl-phenyl)-4-(1-methyl-indol-5-yl)-5- |
| | mercapto-[1,2,4] triazole |
| 225 | 3-(2,4-dihydroxy-5-isopropyl-phenyl)-4-(1H-indol-5-yl)-5-mercapto- |
| | [1,2,4] triazole |
| 226 | 3-(2,4-dihydroxy-5-isopropyl-phenyl)-4-(1-methyl-indol-5-yl)-5- |
| | hydroxy-[1,2,4] triazole |
| 227 | 3-(2,4-dihydroxy-5-isopropyl-phenyl)-4-(1-ethyl-indol-5-yl)-5- |
| | mercapto-[1,2,4] triazole |
| 228 | 3-(2,4-dihydroxy-5-isopropyl-phenyl)-4-(1-propyl-indol-5-yl)-5- |
| | mercapto-[1,2,4] triazole |
| 229 | 3-(2,4-dihydroxy-5-isopropyl-phenyl)-4-(1-methyl-2-trifluoromethyl- |
| | benzimidazol-5-yl)-5-mercapto-[1,2,4] triazole |
| 230 | 3-(2,4-dihydroxy-5-isopropyl-phenyl)-4-(1-methyl-indazol-5-yl)-5- |
| | mercapto-[1,2,4] triazole |
| 231 | 3-(2,4-dihydroxy-5-isopropyl-phenyl)-4-(1-methyl-indazol-6-yl)-5- |
| | mercapto-[1,2,4] triazole |
| 232 | 3-(2,4-dihydroxy-5-isopropyl-phenyl)-4-(1-isopropyl-indol-4-yl)-5- |
| | hydroxy-[1,2,4] triazole |
| 233 | 3-(2,4-dihydroxy-5-isopropyl-phenyl)-4-(1,3-benzodiaxol-5-yl)-5- |
| | mercapto-[1,2,4] triazole |
| 234 | 3-(2,4-dihydroxy-5-isopropyl-phenyl)-4-(indan-5-yl)-5-mercapto- |
| | [1,2,4] triazole |
| 235 | 3-(2,4-dihydroxy-5-isopropyl-phenyl)-4-(2-methyl-indazol-6-yl)-5- |
| | mercapto-[1,2,4] triazole |
| 236 | 3-(2,4-dihydroxy-5-ethyl-phenyl)-4-(3-oxo-benzo[1,4]oxazin-6-yl)- |
| | 5-mercapto-[1,2,4] triazole |
| 237 | 3-(2,4-dihydroxy-5-ethyl-phenyl)-4-(2-oxo-1,3-dihydro- |
| | benzoimidazol-5-yl)-5-mercapto-[1,2,4] triazole |
| 238 | 3-(2,4-dihydroxy-5-isopropyl-phenyl)-4-(2H-benzo[1,4]oxazin-6- |
| | yl)-5-mercapto-[1,2,4] triazole |
| 239 | 4-Ethyl-6-[5-mercapto-4-(1-methyl-2,3-dihydro-1H-indol-5-yl)-4H- |
| | [1,2,4]triazol-3-yl]-benzene-1,3-diol |
| 240 | 5-(3-(5-ethyl-2,4-dihydroxyphenyl)-5-mercapto-4H-1,2,4-triazol-4- |
| | yl)indolin-2-one |
| 241 | 5-(3-(5-ethyl-2,4-dihydroxyphenyl)-5-mercapto-4H-1,2,4-triazol-4- |
| | yl)-1H-benzo[d]imidazol-2(3H)-one |
| 242 | 5-(3-(5-ethyl-2,4-dihydroxyphenyl)-5-mercapto-4H-1,2,4-triazol-4- |
| | yl)-1-methylindolin-2-one |
| 243 | 4-isopropyl-6-(5-mercapto-4-(4-propyl-3,4-dihydro-2H- |
| | benzo[b][1,4]oxazin-6-yl)-4H-1,2,4-triazol-3-yl)benzene-1,3-diol |
| 244 | 6-(3-(5-ethyl-2,4-dihydroxyphenyl)-5-mercapto-4H-1,2,4-triazol-4- |
| | yl)-2H-benzo[b][1,4]oxazin-3(4H)-one |
| 245 | 6-(3-(5-ethyl-2,4-dihydroxyphenyl)-5-mercapto-4H-1,2,4-triazol-4- |
| | yl)-3-methylbenzo[d]thiazol-2(3H)-one |
| 246 | 6-(3-(5-ethyl-2,4-dihydroxyphenyl)-5-mercapto-4H-1,2,4-triazol-4- |
| | yl)benzo[d]thiazol-2(3H)-one |
| 247 | 4-(4-(3-(diethylamino)-4-methoxyphenyl)-5-mercapto-4H-1,2,4- |
| | triazol-3-yl)-6-ethylbenzene-1,3-diol |
| 248 | 4-(4-(3-(N-isopropyl-N-propylamino)-4-methoxyphenyl)-5-mercapto- |
| | 4H-1,2,4-triazol-3-yl)-6-ethylbenzene-1,3-diol |
| 249 | 4-(4-(3-(N-isopropyl-N-methylamino)-4-methoxyphenyl)-5-mercapto- |
| | 4H-1,2,4-triazol-3-yl)-6-ethylbenzene-1,3-diol |
| 250 | 4-(4-(3-(N-ethyl-N-methylamino)-4-methoxyphenyl)-5-mercapto-4H- |
| | 1,2,4-triazol-3-yl)-6-ethylbenzene-1,3-diol |
| 251 | 4-(4-(3-(dimethylamino)-4-methoxyphenyl)-5-mercapto-4H-1,2,4- |
| | triazol-3-yl)-6-ethylbenzene-1,3-diol |
| 252 | 4-(4-(3-(dimethylamino)phenyl)-5-mercapto-4H-1,2,4-triazol-3-yl)- |
| | 6-ethylbenzene-1,3-diol |
| 253 | 4-(4-(3-(N-ethyl-N-isopropylamino)-4-methoxyphenyl)-5-mercapto- |
| | 4H-1,2,4-triazol-3-yl)-6-ethylbenzene-1,3-diol |
| 254 | 4-ethyl-6-(5-mercapto-4-(3-(pyrrolidin-1-yl)phenyl)-4H-1,2,4- |
| | triazol-3-yl)benzene-1,3-diol |
| 255 | 4-ethyl-6-(5-mercapto-4-(4-methoxy-3-morpholinophenyl)-4H-1,2,4- |
| | triazol-3-yl)benzene-1,3-diol |
| 256 | 4-(4-(3-(N-isopropyl-N-propylamino)-4-methoxyphenyl)-5-mercapto- |
| | 4H-1,2,4-triazol-3-yl)-6-isopropylbenzene-1,3-diol |
| 257 | 4-(4-(3-(N-methyl-N-propylamino)-4-methoxyphenyl)-5-mercapto- |
| | 4H-1,2,4-triazol-3-yl)-6-isopropylbenzene-1,3-diol |
| 258 | 4-(4-(3-(N-methyl-N-ethylamino)-4-methoxy-phenyl)-5-mercapto-4H- |
| | 1,2,4-triazol-3-yl)-6-isopropylbenzene-1,3-diol |
| 259 | 4-(4-(4-(dimethylamino)-3-methoxyphenyl)-5-mercapto-4H-1,2,4- |
| | triazol-3-yl)-6-ethylbenzene-1,3-diol |
| 260 | N-ethyl-N-(5-(3-(5-ethyl-2,4-dihydroxyphenyl)-5-mercapto-4H- |
| | 1,2,4-triazol-4-yl)-2-methoxyphenyl)acetamide |
| 261 | 4-(4-(3-aminophenyl)-5-mercapto-4H-1,2,4-triazol-3-yl)-6- |
| | ethylbenzene-1,3-diol |
| 262 | 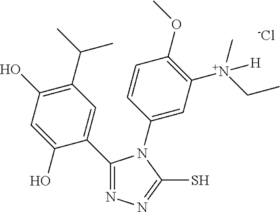 |
| 263 | 4-(4-(3-(N-isopentyl-N-methylamino)-4-methoxyphenyl)-5-mercapto- |
| | 4H-1,2,4-triazol-3-yl)-6-isopropylbenzene-1,3-diol |
| 264 | 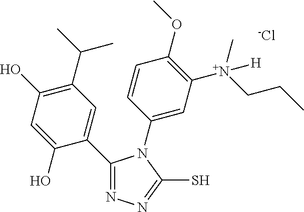 |
| 265 | 4-(4-(3-(N-(2-(dimethylamino)ethyl)-N-methylamino)-4- |
| | methoxyphenyl)-5-mercapto-4H-1,2,4-triazol-3-yl)-6- |
| | isopropylbenzene-1,3-diol |
| 266 | 4-(4-(3-(N-(2-methoxyethyl)-N-methylamino)-4-methoxyphenyl)-5- |
| | mercapto-4H-1,2,4-triazol-3-yl)-6-isopropylbenzene-1,3-diol |
| 267 | 4-(4-(3-(N-(cyclopropylmethyl)-N-methylamino)-4-methoxyphenyl)- |
| | 5-mercapto-4H-1,2,4-triazol-3-yl)-6-isopropylbenzene-1,3-diol |
| 268 | 4-(4-(3-(N-butyl-N-methylamino)-4-methoxyphenyl)-5-mercapto-4H- |
| | 1,2,4-triazol-3-yl)-6-isopropylbenzene-1,3-diol |
| 269 | 4-(4-(3-(N-isobutyl-N-methylamino)-4-methoxyphenyl)-5-mercapto- |
| | 4H-1,2,4-triazol-3-yl)-6-isopropylbenzene-1,3-diol |
| 270 | 4-(4-(3-(N-(2-(1H-imidazol-1-yl)ethyl)-N-methylamino)-4- |
| | methoxyphenyl)-5-mercapto-4H-1,2,4-triazol-3-yl)-6- |
| | isopropylbenzene-1,3-diol |
| 271 | 4-(4-(3-(N-methyl-N-propylamino)-4-methoxyphenyl)-5-mercapto- |
| | 4H-1,2,4-triazol-3-yl)-6-isopropylbenzene-1,3-diol |
| 272 | 4-(4-(3-(dimethylamino)-4-(methylthio)phenyl)-5-mercapto-4H-1,2,4- |
| | triazol-3-yl)-6-isopropylbenzene-1,3-diol |
| 273 | 4-(4-(3-(1H-pyrrol-1-yl)phenyl)-5-hydroxy-4H-1,2,4-triazol-3-yl)-6- |
| | ethylbenzene-1,3-diol |
| 274 | 4-(4-(3-(1H-imidazol-1-yl)phenyl)-5-mercapto-4H-1,2,4-triazol-3-yl)- |
| | 6-isopropylbenzene-1,3-diol |
| 275 | 1-(3-(3-(2,4-dihydroxy-5-isopropylphenyl)-5-mercapto-4H-1,2,4- |
| | triazol-4-yl)phenyl)-3-methyl-1H-pyrazol-5(4H)-one |
| 276 | N-(4-(3-(5-ethyl-2,4-dihydroxyphenyl)-5-mercapto-4H-1,2,4-triazol- |
| | 4-yl)phenyl)-N-methylacetamide |
| 277 | 4-(4-(4-(dimethylamino)phenyl)-5-mercapto-4H-1,2,4-triazol-3-yl)-6- |
| | ethylbenzene-1,3-diol |
| 278 | 4-(4-(4-(diethylamino)phenyl)-5-mercapto-4H-1,2,4-triazol-3-yl)-6- |
| | ethylbenzene-1,3-diol |
| 279 | 4-ethyl-6-(5-mercapto-4-(4-morpholinophenyl)-4H-1,2,4-triazol-3- |
| | yl)benzene-1,3-diol |
| 280 | 4-(4-(4-(1H-imidazol-1-yl)phenyl)-5-mercapto-4H-1,2,4-triazol-3-yl)- |
| | 6-ethylbenzene-1,3-diol |
| 281 | 4-(4-(2,5-diethoxy-4-morpholinophenyl)-5-mercapto-4H-1,2,4-triazol- |
| | 3-yl)-6-ethylbenzene-1,3-diol |
| 282 | 4-(4-(3-(1H-pyrrol-1-yl)phenyl)-5-mercapto-4H-1,2,4-triazol-3-yl)-6- |
| | ethylbenzene-1,3-diol |
| 283 | 4-(4-(4-(1H-pyrazol-1-yl)phenyl)-5-mercapto-4H-1,2,4-triazol-3-yl)-6- |
| | ethylbenzene-1,3-diol |
| 284 | 4-(4-(4-(amino)-3-hydroxyphenyl)-5-mercapto-4H-1,2,4-triazol-3-yl)- |
| | 6-ethylbenzene-1,3-diol |
| 285 | 4-(4-(4-(methylamino)-3-hydroxyphenyl)-mercapto-4H-1,2,4-triazol- |
| | 3-yl)-6-ethylbenzene-1,3-diol |
| 286 | 4-(4-(4-(dimethylamino)-3-methylphenyl)-5-mercapto-4H-1,2,4- |
| | triazol-3-yl)-6-ethylbenzene-1,3-diol |
| 287 | 4-{4-[methyl-(2-dimethylamino-ethyl)-amino]-phenyl}-5-hydroxy- |
| | 4H-1,2,4-triazol-3-yl)-6-isopropyl-benzene-1,3-diol |

Exemplary pyrazole compounds useful in the method of the invention are depicted in Table 6 below. Tautomers, pharmaceutically acceptable salts, solvates, clathrates, hydrates, polymorphs or prodrugs of the compounds in Table 6 are also useful in the method of the invention.

TABLE 6

| No. | Name |
|---|---|
| 288 | 4-[3-(N,N-diethylamino)-4-methoxy-phenyl]-3-(5-ethyl-2,4-dihydroxy-phenyl)-5- |
| | mercapto-2H-pyrazole |
| 289 | 4-[3-(isopropyl-propyl-amino)-4-methoxy-phenyl]-3-(5-ethyl-2,4-dihydroxy-phenyl)- |
| | 5-mercapto-2H-pyrazole |
| 290 | 4-[3-(isopropyl-methyl-amino)-4-methoxy-phenyl]-3-(5-ethyl-2,4-dihydroxy-phenyl)- |
| | 5-mercapto-2H-pyrazole |
| 291 | 4-[3-(ethyl-methyl-amino)-4-methoxy-phenyl]-3-(5-ethyl-2,4-dihydroxy-phenyl)-5- |
| | mercapto-2H-pyrazole |
| 292 | 4-[3-(N,N-methylamino)-4-methoxy-phenyl]-3-(5-ethyl-2,4-dihydroxy-phenyl)-5- |
| | mercapto-2H-pyrazole |
| 293 | 4-[3-(N,N-methylamino)-phenyl]-3-(5-ethyl-2,4-dihydroxy-phenyl)-5-mercapto-2H- |
| | pyrazole |
| 294 | 4-[4-(N,N-methylamino)-3-methoxy-phenyl]-3-(5-ethyl-2,4-dihydroxy-phenyl)-5- |
| | mercapto-2H-pyrazole |
| 295 | 4-[3-(isopropyl-ethyl-amino)-4-methoxy-phenyl]-3-(5-ethyl-2,4-dihydroxy-phenyl)-5- |
| | mercapto-2H-pyrazole |
| 296 | 4-[3-(pyrrolidin-1-yl)-phenyl]-3-(5-ethyl-2,4-dihydroxy-phenyl)-5-mercapto-2H- |
| | pyrazole |
| 297 | 4-[3-(isopropyl-propyl-amino)-4-methoxy-phenyl]-3-(5-isopropyl-2,4-dihydroxy- |
| | phenyl)-5-mercapto-2H-pyrazole |
| 298 | 4-[3-(methyl-propyl-amino)-4-methoxy-phenyl]-3-(5-isopropyl-2,4-dihydroxy- |
| | phenyl)-5-mercapto-2H-pyrazole |
| 299 | 4-[3-(ethyl-methyl-amino)-4-methoxy-phenyl]-3-(5-isopropyl-2,4-dihydroxy-phenyl)- |
| | 5-mercapto-2H-pyrazole |
| 300 | 4-[3-(morpholino-1-yl)-4-methoxy-phenyl]-3-(5-ethyl-2,4-dihydroxy-phenyl)-5- |
| | mercapto-2H-pyrazole |
| 301 | 4-[3-(ethyl-methyl-amino)-4-methoxy-phenyl]-3-(5-isopropyl-2,4-dihydroxy-phenyl)- |
| | 5-hydroxy-2H-pyrazole |
| 302 | 4-[3-(N,N-diethyl-amino)-4-methoxy-phenyl]-3-(5-ethyl-2,4-dihydroxy-phenyl)-5- |
| | hydroxy-2H-pyrazole |
| 303 | 4-[3-(pyrrolidin-1-yl)-4-methoxy-phenyl]-3-(5-ethyl-2,4-dihydroxy-phenyl)-5- |
| | hydroxy-2H-pyrazole |
| 304 | 4-[3-(ethyl-methyl-amino)-4-methoxy-phenyl]-3-(5-cyclopropyl-2,4-dihydroxy- |
| | phenyl)-5-hydroxy-2H-pyrazole |
| 305 | 4-[3-(ethyl-methyl-amino)-4-methoxy-phenyl]-3-(5-cyclopropyl-2,4-dihydroxy- |
| | phenyl)-5-mercapto-2H-pyrazole |
| 306 | Phosphoric acid mono {4-[3-(ethyl-methyl-amino)-4-methoxy-phenyl]-3-(5-isopropyl- |
| | 2,4-dihydroxy-phenyl)-2H-pyrazol-5-yl} ester |
| 307 | Phosphoric acid {4-[3-(ethyl-methyl-amino)-4-methoxy-phenyl]-3-(5-isopropyl-2,4- |
| | dihydroxy-phenyl)-2H-pyrazol-5-yl} ester ethyl ester |
| 308 | 4-[3-(N,N-methylamino)-4-methoxy-phenyl]-3-(5-isopropyl-2-hydroxy-4- |
| | dimethylaminocarbamoyloxy-phenyl)-5-mercapto-2H-pyrazole |
| 309 | 4-[3-(pyrrolidin-1-yl)-4-methoxy-phenyl]-3-(5-isopropyl-2-hydroxy-4- |
| | dimethylaminocarbamoyloxy-phenyl)-5-mercapto-2H-pyrazole |
| 310 | 4-[3-(N,N-methylamino)-4-methoxy-phenyl]-3-(5-isopropyl-2,4-dihydroxy-phenyl)-5- |
| | (2-hydroxy-ethylsulfanyl)-2H-pyrazole |
| 311 | 4-(1-isopropyl-1H-indol-4-yl)-3-(2,4-dihydroxy-phenyl)-5-mercapto-2H-pyrazole |
| 312 | 4-(1H-indol-4-yl)-3-(2,4-dihydroxy-phenyl)-5-mercapto-2H-pyrazole |
| 313 | 4-[1-(2-methoxy-ethyl)-1H-indol-4-yl]-3-(2,4-dihydroxy-phenyl)-5-mercapto-2H- |
| | pyrazole |
| 314 | 4-(1-isopropyl-1H-indol-4-yl)-3-(5-ethyl-2,4-dihydroxy-phenyl)-5-mercapto-2H- |
| | pyrazole |
| 315 | 4-(1-dimethylcarbamoyl-1H-indol-4-yl)-3-(2,4-dihydroxy-phenyl)-5-mercapto-2H- |
| | pyrazole |
| 316 | 4-(1-propyl-1H-indol-4-yl)-3-(5-ethyl-2,4-dihydroxy-phenyl)-5-mercapto-2H-pyrazole |
| 317 | 4-(1-ethyl-1H-indol-4-yl)-3-(5-ethyl-2,4-dihydroxy-phenyl)-5-mercapto-2H-pyrazole |
| 318 | 4-(1,2,3-trimethyl-1H-indol-4-yl)-3-(5-ethyl-2,4-dihydroxy-phenyl)-5-mercapto-2H- |
| | pyrazole |
| 319 | 4-(2,3-dimethyl-1H-indol-4-yl)-3-(5-ethyl-2,4-dihydroxy-phenyl)-5-mercapto-2H- |
| | pyrazole |
| 320 | 4-(1-ethyl-1H-benzoimidazol-4-yl)-3-(5-ethyl-2,4-dihydroxy-phenyl)-5-mercapto-2H- |
| | pyrazole |
| 321 | 4-(1-carboxy-2,3-dimethyl-1H-indol-5-yl)-3-(5-ethyl-2,4-dihydroxy-phenyl)-5- |
| | mercapto-2H-pyrazole |
| 322 | 4-(1-ethyl-2-methyl-1H-benzoimidazol-6-yl)-3-(5-ethyl-2,4-dihydroxy-phenyl)-5- |
| | mercapto-2H-pyrazole |
| 323 | 4-(1-isopropy-7-methoxy-1H-indol-4-yl)-3-(5-ethyl-2,4-dihydroxy-phenyl)-5- |
| | mercapto-2H-pyrazole |
| 324 | 4-(1-propy-2,3-dimethyl-1H-indol-5-yl)-3-(5-ethyl-2,4-dihydroxy-phenyl)-5-mercapto- |
| | 2H-pyrazole |
| 325 | 4-(1-ethyl-1H-indol-4-yl)-3-(5-isopropyl-2,4-dihydroxy-phenyl)-5-hydroxy-2H- |
| | pyrazole |
| 326 | 4-(1-ethyl-1H-indol-4-yl)-3-(5-cyclopropyl-2,4-dihydroxy-phenyl)-5-hydroxy-2H- |
| | pyrazole |
| 327 | 4-(1,2,3-trimethyl-1H-indol-5-yl)-3-(5-ethyl-2,4-dihydroxy-phenyl)-5-amino-2H- |
| | pyrazole |
| 328 | 4-(1-isopropyl-7-methoxy-1H-indol-4-yl)-3-(5-ethyl-2,4-dihydroxy-phenyl)-5-amino- |
| | 2H-pyrazole |
| 329 | 4-(1-isopropyl-7-methoxy-1H-indol-4-yl)-3-(5-isopropyl-2,4-dihydroxy-phenyl)-5- |
| | hydroxy-2H-pyrazole |
| 330 | 4-(1,3-dimethyl-1H-indol-5-yl)-3-(5-isopropyl-2,4-dihydroxy-phenyl)-5-hydroxy-2H- |
| | pyrazole |
| 331 | 4-(1-methyl-1H-indol-5-yl)-3-(5-isopropyl-2,4-dihydroxy-phenyl)-5-hydroxy-2H- |
| | pyrazole |
| 332 | 4-(1-methyl-1H-indol-5-yl)-3-(5-isopropyl-2,4-dihydroxy-phenyl)-5-mercapto-2H- |
| | pyrazole |
| 333 | 4-(1-methyl-1H-indol-5-yl)-3-(5-isopropyl-2,4-dihydroxy-phenyl)-5-amino-2H- |
| | pyrazole |
| 334 | 4-(7-methoxy-benzofuran-4-yl)-3-(5-isopropyl-2,4-dihydroxy-phenyl)-5-hydroxy-2H- |
| | pyrazole |
| 335 | 4-(5-methoxy-naphthalene-1-yl)-3-(5-isopropyl-2,4-dihydroxy-phenyl)-5-mercapto- |
| | 2H-pyrazole |
| 336 | 4-(benzo[1,4]dioxin-5-yl)-3-(5-ethyl-2,4-dihydroxy-phenyl)-5-mercapto-2H-pyrazole |
| 337 | 4-(acenaphthen-5-yl)-3-(5-isopropyl-2,4-dihydroxy-phenyl)-5-hydroxy-2H-pyrazole |
| 338 | 4-(9H-purin-6-yl)-3-(5-isopropyl-2,4-dihydroxy-phenyl)-5-hydroxy-2H-pyrazole |
| 339 | 4-(benzothiazol-4-yl)-3-(5-isopropyl-2,4-dihydroxy-phenyl)-5-mercapto-2H-pyrazole |
| 340 | 4-(7-fluoro-naphthylen-1-yl)-3-(5-cyclopropyl-2,4-dihydroxy-phenyl)-5-mercapto-2H- |
| | pyrazole |
| 341 | 4-(quinolin-4-yl)-3-(5-isopropyl-2,4-dihydroxy-phenyl)-5-mercapto-2H-pyrazole |
| 342 | 4-(1-methyl-1H-indol-5yl)-3-(5-isopropyl-2,4-dihydroxy-phenyl)-5-carbamoyloxy- |
| | 2H-pyrazole |
| 343 | 4-(1-methyl-1H-indol-5-yl)-3-(5-cyclopropyl-2,4-dihydroxy-phenyl)-5-carboxyamino- |
| | 2H-pyrazole |
| 344 | 4-(1-methyl-1H-indol-5-yl)-3-(5-methoxy-2,4-dihydroxy-phenyl)-5-aminosulfamido- |
| | 2H-pyrazole |
| 345 | 4-(4-methoxy-naphthalene-1-yl)-3-(5-isopropyl-2-hydroxy-4-ethoxycarbonyloxy- |
| | phenyl)-5-mercapto-2H-pyrazole |
| 346 | 4-(naphthalene-1-yl)-3-(5-isopropyl-2,4-ethylcarbamoyloxy-phenyl)-5-mercapto-2H- |
| | pyrazole |
| 347 | 4-(1-methyl-1H-indol-4-yl)-3-(5-isopropyl-2,4-ethylcarbamoyloxy-phenyl)-5- |
| | dimethylcarbamoylsulfanyl-2H-pyrazole |
| 348 | 4-(1,2-dimethyl-1H-indol-4-yl)-3-(5-isopropyl-2,4-ethyloxycarbonyloxy-phenyl)-5- |
| | ethoxycarbamoylsulfanyl-2H-pyrazole |
| 349 | 4-(naphthalen-1-yl)-3-(5-ethyl-2,4-dihydroxy-phenyl)-5-hydroxy-2H-pyrazole |
| 350 | 4-(2-methyl-4-fluorophenyl)-3-(5-ethyl-2,4-dihydroxy-phenyl)-5-mercapto-2H- |
| | pyrazole |
| 351 | 4-(3,5-dimethoxyphenyl)-3-(5-ethyl-2,4-dihydroxy-phenyl)-5-amino-2H-pyrazole |
| 352 | 4-[2-(1H-tetrazol-5-yl)-phenyl]-3-(5-ethyl-2,4-dihydroxy-phenyl)-5-hydroxy-2H- |
| | pyrazole |

Exemplary imidazolyl compounds useful in the method of the invention are depicted in Table 7 below. Tautomers, pharmaceutically acceptable salts, solvates, clathrates, hydrates, polymorphs or prodrugs of the compounds in Table 7 are also useful in the method of the invention.

TABLE 7

| No. | Name |
|---|---|
| 353 | 1-(3-diethylamino-4-methoxy-phenyl)-2-mercapto-5-(2,4-dihydroxy-5-ethyl- |
| | phenyl)-1H-imidazole |
| 354 | 1-[4-3-(propyl-isopropylamino)-4-methoxy-phenyl]-2-mercapto-5-(2,4-dihydroxy-5- |
| | ethyl-phenyl)-1H-imidazole |
| 355 | 1-[3-(methyl-isopropylamino)-4-methoxy-phenyl]-2-mercapto-5-(2,4-dihydroxy-5- |
| | ethyl-phenyl)-1H-imidazole |
| 356 | 1-[3-(methyl-ethylamino)-4-methoxy-phenyl]-2-mercapto-5-(2,4-dihydroxy-5-ethyl- |
| | phenyl)-1H-imidazole |
| 357 | 1-(3-dimethylamino-4-methoxy-phenyl)-2-mercapto-5-(2,4-dihydroxy-5-ethyl- |
| | phenyl)-1H-imidazole |
| 358 | 1-(3-dimethylamino-phenyl)-2-mercapto-5-(2,4-dihydroxy-5-ethyl-phenyl)-1H- |
| | imidazole |
| 359 | 1-(3-methoxy-4-dimethylamino-phenyl)-2-mercapto-5-(2,4-dihydroxy-5-ethyl- |
| | phenyl)-1H-imidazole |
| 360 | 1[3-(ethyl-isopropylamino)-4-methoxy-phenyl]-2-mercapto-5-(2,4-dihydroxy-5- |
| | ethyl-phenyl)-1H-imidazole |
| 361 | 1-(3-pyrrolidin-1-yl-phenyl)-2-mercapto-5-(2,4-dihydroxy-5-ethyl-phenyl)-1H- |
| | imidazole |
| 362 | 1-[3-(propyl-isopropylamino)-4-methoxy-phenyl]-2-mercapto-5-(2,4-dihydroxy-5- |
| | isopropyl-phenyl)-1H-imidazole |
| 363 | 1-[3-(methyl-propylamino)-4-methoxy-phenyl]-2-mercapto-5-(2,4-dihydroxy-5- |
| | isopropyl-phenyl)-1H-imidazole |
| 364 | 1[3-(methyl-ethylamino)-4-methoxy-phenyl]-2-mercapto-5-(2,4-dihydroxy-5- |
| | isopropyl-phenyl)-1H-imidazole |
| 365 | 1-[3-(morpholino-1-yl)-4-methoxy-phenyl]-2-mercapto-5-(2,4-dihydroxy-5-ethyl- |
| | phenyl)-1H-imidazole |
| 366 | 1-[3-(methyl-ethylamino)-4-methoxy-phenyl]-2-hydroxy-5-(2,4-dihydroxy-5- |
| | isopropyl-phenyl)-1H-imidazole |
| 367 | 1-(3-diethylamino-4-methoxy-phenyl)-2-hydroxy-5-(2,4-dihydroxy-5-ethyl-phenyl)- |
| | 1H-imidazole |
| 368 | 1-[3-(pyrrolidin-1-yl)-4-methoxy-phenyl]-2-hydroxy-5-(2,4-dihydroxy-5-ethyl- |
| | phenyl)-1H-imidazole |
| 369 | 1-[3-(methyl-ethylamino)-4-methoxy-phenyl]-2-hydroxy-5-(2,4-dihydroxy-5- |
| | cyclopropyl-phenyl)-1H-imidazole |
| 370 | 1-[3-(methyl-ethylamino)-4-methoxy-phenyl]-2-mercapto-5-(2,4-dihydroxy-5- |
| | cyclopropyl-phenyl)-1H-imidazole |
| 371 | 1-[3-(methyl-ethylamino)-4-methoxy-phenyl]-2-phosphonooxy-5-(2,4-dihydroxy-5- |
| | isopropyl-phenyl)-1H-imidazole |
| 372 | 1-[3-(methyl-ethylamino)-4-methoxy-phenyl]-2-(ethoxy-hydroxy-phosphoryloxy)-5- |
| | (2,4-dihydroxy-5-isopropyl-phenyl)-1H-imidazole |
| 373 | 1-(3-dimethylamino-4-methoxy-phenyl)-2-mercapto-5-(2-hydroxy-4- |
| | dimethylcarbamoyloxy-5-isopropyl-phenyl)-1H-imidazole |
| 374 | 1-[3-(pyrrolidin-1-yl)-4-methoxy-phenyl]-2-mercapto-5-(2-hydroxy-4- |
| | isobutyryloxy-5-isopropyl-phenyl)-1H-imidazole |
| 375 | 1-(3-dimethylamino-4-methoxy-phenyl)-2-(2-hydroxy-ethylsulfanyl)-5-(2,4- |
| | dihydroxy-5-isopropyl-phenyl)-1H-imidazole |
| 376 | 1-(1-ethyl-1H-indol-4-yl)-2-mercapto-5-(2,4-dihydroxy-phenyl)-1H-imidazole |
| 377 | 1-(1-isopropyl-1H-indol-4-yl)-2-mercapto-5-(2,4-dihydroxy-phenyl)-1H-imidazole |
| 378 | 1-(1H-indol-4-yl)-2-mercapto-5-(2,4-dihydroxy-phenyl)-1H-imidazole |
| 379 | 1-[1-(2-methoxy-ethyl)-1H-indol-4-yl]-2-mercapto-5-(2,4-dihydroxy-phenyl)-1H- |
| | imidazole |
| 380 | 1-(1-isopropyl-1H-indol-4-yl)-2-mercapto-5-(2,4-dihydroxy-5-ethyl-phenyl)-1H- |
| | imidazole |
| 381 | 1-(1-dimethylcarbamoyl-1H-indol-4-yl)-2-mercapto-5-(2,4-dihydroxy-phenyl)-1H- |
| | imidazole |
| 382 | 1-(1-propyl-1H-indol-4-yl)-2-mercapto-5-(2,4-dihydroxy-5-ethyl-phenyl)-1H- |
| | imidazole |
| 383 | 1-(1-ethyl-1H-indol-4-yl)-2-mercapto-5-(2,4-dihydroxy-5-ethyl-phenyl)-1H- |
| | imidazole |
| 384 | 1-(1,2,3-trimethyl-1H-indol-5-yl)-2-mercapto-5-(2,4-dihydroxy-5-ethyl-phenyl)-1H- |
| | imidazole |
| 385 | 1-(2,3-dimethyl-1H-indol-5-yl)-2-mercapto-5-(2,4-dihydroxy-5-ethyl-phenyl)-1H- |
| | imidazole |
| 386 | 1-(1-ethyl-1H-benzoimidazol-4-yl)-2-mercapto-5-(2,4-dihydroxy-5-ethyl-phenyl)- |
| | 1H-imidazole |
| 387 | 1-(1-carboxy-2,3-dimethyl-1H-indol-5-yl)-2-mercapto-5-(2,4-dihydroxy-5-ethyl- |
| | phenyl)-1H-imidazole |
| 388 | 1-(1-ethyl-2-methyl-1H-benzoimidazol-6-yl)-2-mercapto-5-(2,4-dihydroxy-5-ethyl- |
| | phenyl)-1H-imidazole |
| 389 | 1-(1-isopropyl-7-methoxy-1H-indol-4-yl)-2-mercapto-5-(2,4-dihydroxy-5-ethyl- |
| | phenyl)-1H-imidazole |
| 390 | 1-(1-propyl-2,3-dimethyl-1H-indol-5-yl)-2-mercapto-5-(2,4-dihydroxy-5-ethyl- |
| | phenyl)-1H-imidazole |
| 391 | 1-(1-ethyl-1H-indol-4-yl)-2-hydroxy-5-(2,4-dihydroxy-5-isopropyl-phenyl)-1H- |
| | imidazole |
| 392 | 1-(1-ethyl-1H-indol-4-yl)-2-hydroxy-5-(2,4-dihydroxy-5-cyclopropyl-phenyl)-1H- |
| | imidazole |
| 393 | 1-(1,2,3-trimethyl-1H-indol-5-yl)-2-amino-5-(2,4-dihydroxy-5-ethyl-phenyl)-1H- |
| | imidazole |
| 394 | 1-(1-isopropyl-7-methoxy-1H-indol-4-yl)-2-amino-5-(2,4-dihydroxy-5-ethyl- |
| | phenyl)-1H-imidazole |
| 395 | 1-(1-isopropyl-7-methoxy-1H-indol-4-yl)-2-hydroxy-5-(2,4-dihydroxy-5-isopropyl- |
| | phenyl)-1H-imidazole |
| 396 | 1-(1,3-dimethyl-1H-indol-5-yl)-2-hydroxy-5-(2,4-dihydroxy-5-isopropyl-phenyl)- |
| | 1H-imidazole |
| 397 | 1-(1-methyl-1H-indol-5-yl)-2-hydroxy-5-(2,4-dihydroxy-5-isopropyl-phenyl)-1H- |
| | imidazole |
| 398 | 1-(1-methyl-1H-indol-5-yl)-2-mercapto-5-(2,4-dihydroxy-5-isopropyl-phenyl)-1H- |
| | imidazole |
| 399 | 1-(9-methyl-6,7,8,9-tetrahydro-5H-carbazol-3-yl)-2-mercapto-5-(2,4-dihydroxy-5- |
| | ethyl-phenyl)-1H-imidazole |
| 400 | 1-(1-methyl-1H-indol-5-yl)-2-amino-5-(2,4-dihydroxy-5-isopropyl-phenyl)-1H- |
| | imidazole |
| 401 | 1-(7-methoxy-benzofuran-4-yl)-2-hydroxy-5-(2,4-dihydroxy-5-isopropyl-phenyl)- |
| | 1H-imidazole |
| 402 | 1-(5-methoxy-naphthylen-1-yl)-2-mercapto-5-(2,4-dihydroxy-5-isopropyl-phenyl)- |
| | 1H-imidazole |
| 403 | 1-(2,3-dihydro-benzo[1,4]dioxin-5-yl)-2-mercapto-5-(2,4-dihydroxy-5-ethyl- |
| | phenyl)-1H-imidazole |
| 404 | 1-(3-acenaphthylen-5-yl)-2-hydroxy-5-(2,4-dihydroxy-5-isopropyl-phenyl)-1H- |
| | imidazole |
| 405 | 1-(9H-purin-6-yl)-2-hydroxy-5-(2,4-dihydroxy-5-isopropyl-phenyl)-1H-imidazole |
| 406 | 1-(benzothiazol-4-yl)-2-mercapto-5-(2,4-dihydroxy-5-isopropyl-phenyl)-1H- |
| | imidazole |
| 407 | 1-(7-fluoro-naphthylen-1-yl)-2-mercapto-5-(2,4-dihydroxy-5-cyclopropyl-phenyl)- |
| | 1H-imidazole |
| 408 | 1-(quinolin-4-yl)-2-mercapto-5-(2,4-dihydroxy-5-isopropyl-phenyl)-1H-imidazole |
| 409 | 1-(1-methyl-indol-5-yl)-2-carbamoyloxy-5-(2,4-dihydroxy-5-isopropyl-phenyl)-1H- |
| | imidazole |
| 410 | 1-(1-methyl-indol-5-yl)-2-carboxyamino-5-(2,4-dihydroxy-5-cycolpropyl-phenyl)- |
| | 1H-imidazole |
| 411 | 1-(1-methyl-1H-indol-5-yl)-2-aminosulfamido-5-(5-methoxy-2,4-dihydroxy- |
| | phenyl)-1H-imidazole |
| 412 | 1-(4-methoxy-naphthylen-1-yl)-2-mercapto-5-(2-hydroxy-4-ethoxycarbonyloxy-5- |
| | isopropyl-phenyl)-1H-imidazole |
| 413 | 1-(naphthylen-1-yl)-2-mercapto-5-[2,4-di-(ethoxycarbarnoyloxy)-5-isopropyl- |
| | phenyl]-1H-imidazole |
| 414 | 1-(1-methyl-1H-indol-4-yl)-2-dimethylcarbamoylsulfanyl-5-[2,4-di- |
| | (ethoxycarbamoyloxy)-5-isopropyl-phenyl]-1H-imidazole |
| 415 | 1-(1,2-dimethyl-1H-indol-4-yl)-2-ethoxycarbonylsulfanyl-5-[2,4-di- |
| | (ethoxycarbonyloxy)-5-isopropyl-phenyl]-1H-imidazole |
| 416 | 1-(naphthylen-1-yl)-2-hydroxy-5-(2,4-dihydroxy-5-ethyl-phenyl)-1H-imidazole |
| 417 | 1-(2,5-dimethoxyphenyl)-2-amino-5-(2,4-dihydroxy-5-ethyl-phenyl)-1H-imidazole |
| 418 | 1-(2-methyl-4-fluoro-phenyl)-2-mercapto-5-(2,4-dihydroxy-5-ethyl-phenyl)-1H- |
| | imidazole |
| 419 | 1-[2-(1H-tetrazol-5-yl)-phenyl]-2-hydroxy-5-(2,4-dihydroxy-5-ethyl-phenyl)-1H- |
| | imidazole |

Preferred triazole compounds of the invention are those compounds that can form a tautomeric structure as shown below and as exemplified by the tautomeric structures shown in Table 5:

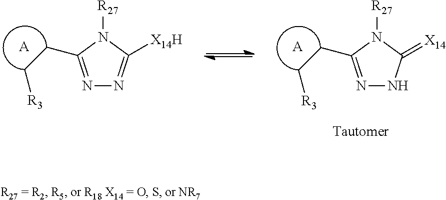

Also preferred are compounds which can be metabolized or hydrolyzed in vivo to a compound which can form the tautomeric structure shown above. For example, the following embodiments of a compound of formula (I) can be produced in vivo in the following reaction:

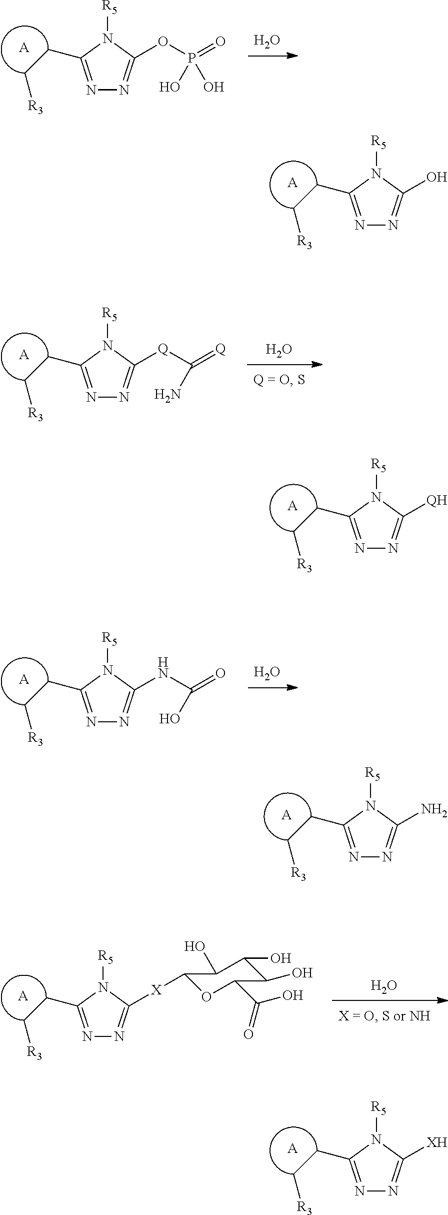

Without wishing to be bound by any theory, it is believed that the compounds of the invention preferentially bind to Hsp90 in the tautomeric form shown above, and thereby inhibit the activity of Hsp90.

It is understood that the pyrazole compounds of the present invention, including compounds of formulas (VI) through (VIII) and Table 6 can be purified, isolated, obtained and used in a form of a tautomer, or a pharmaceutically acceptable salt, solvate, clathrate, or a prodrug.

For example, a compound of formula (VI) can undergo the following tautomerization:

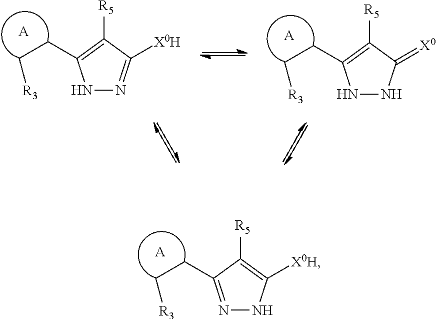

where X⁰ is O, S, or NR₇. It is understood that where a structural formula is depicted, all possible tautomeric forms of the compound are encompassed within that formula.

Similarly, prodrugs, i.e. compounds which can be metabolized or hydrolyzed in vivo to a compound of the present invention are encompassed by the present description. For example, the following embodiments of a compound of formula (VI) can be produced in vivo in the following reaction:

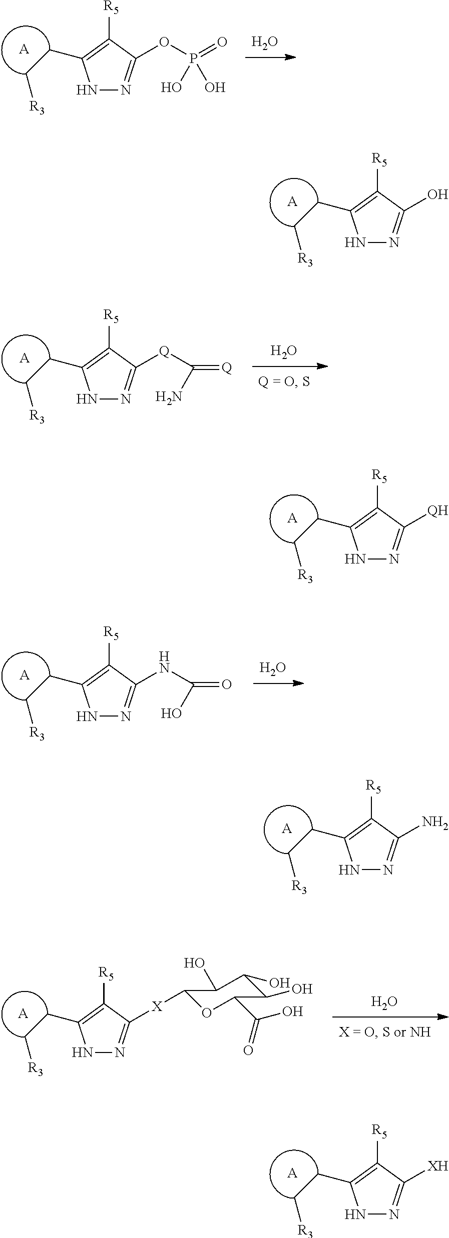

One skilled in the art will understand that other hydrolyzable protecting groups can be employed with the compounds of the present invention to obtain prodrugs encompassed by the present description.

It is understood that the compounds of the present invention, including compounds of formulas (IX) through (XI) and Tables 7 can be purified, isolated, obtained and used in a form of a tautomer or a pharmaceutically acceptable salt, solvate, clathrate, or prodrug.

For example, a compound of formula (IX) can undergo the following tautomerization:

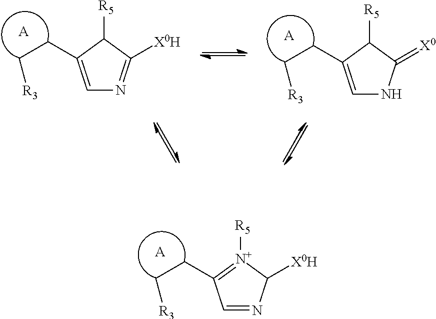

where X⁰ is O, S, or NR₇. It is understood that where a structural formula is depicted, all possible tautomeric forms of the compound are encompassed within that formula.

Similarly, prodrugs, i.e. compounds which can be metabolized or hydrolyzed in vivo to a compound of the present invention are encompassed by the present description. For example, the following embodiments of a compound of formula (IX) can be produced in vivo in the following reaction:

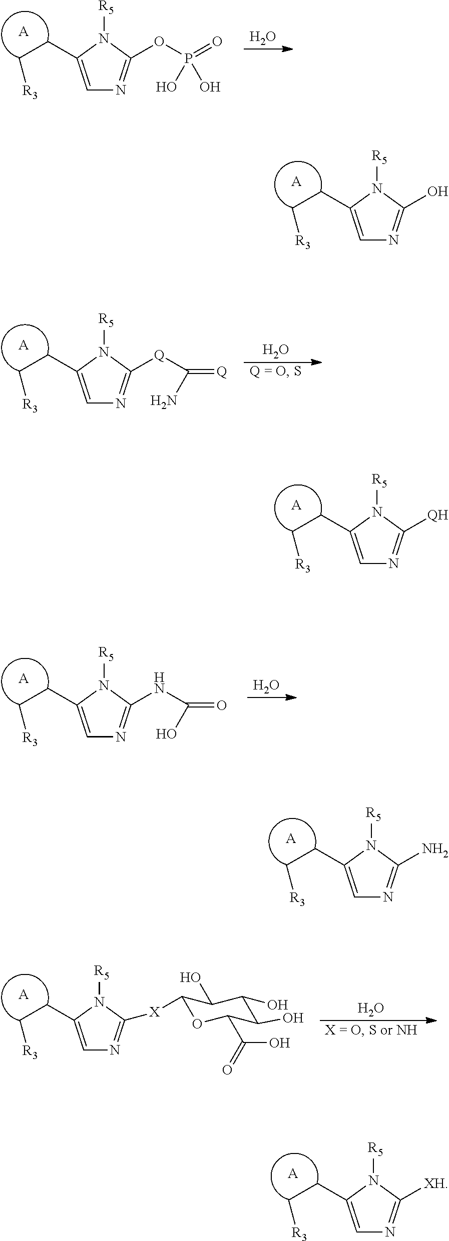

One skilled in the art will understand that other hydrolyzable protecting groups can be employed with the compounds of the present invention to obtain prodrugs encompassed by the present description.

C. Methods for Making Compounds of the Invention

Methods of making the compounds of the invention are disclosed in U.S. patent application Ser. No. 11/282,119, filed on Nov. 17, 2005; U.S. patent application Ser. No. 11/506,185, filed Aug. 17, 2006; U.S. Provisional Patent Application Ser. No. 60/709,358, filed Aug. 18, 2005; U.S. Provisional Patent Application Ser. No. 60/725,044, filed Oct. 6, 2005; U.S. patent application Ser. No. 11/502,346, filed Aug. 10, 2006; U.S. patent application Ser. No. 11/502,347, filed Aug. 10, 2006; the entire teachings of each of these patent applications is incorporated herein by reference.

Additional methods of preparing the compounds of the invention can be found in U.S. Provisional Patent Application Ser. No. 60/808,376, filed on May 25, 2006; U.S. Provisional Patent Application Ser. No. 60/808,342, filed on May 25, 2006; and U.S. Provisional Patent Application Ser. No. 60/808,375, filed on May 25, 2006, the entire teachings of each of these applications are incorporated herein by reference.

D. Uses of Compounds of the Invention

The present invention is directed to therapies which involve administering one or more compounds of the invention, or compositions comprising said compounds to a subject, preferably a human subject, to inhibit the activity of Hsp90 and thereby reduce the expression of glucocorticoid expression in cells, particularly in lymphocytes, or to prevent, treat, manage, or ameliorate an inflammatory or immune disorder, or one or more symptoms thereof. The present invention is also directed to therapies which involve administering one or more compounds of the invention, or compositions comprising said compounds to a subject, preferably a human subject, to suppress the immune system (for example, before or after the subject has received an organ transplant).

In one aspect, the invention provides a method of modulating the activity of glucocorticoid receptors in a cell, comprising administering to the cell an effective amount of an Hsp90 inhibitor represented by any one of formula (I) through (LXXII), or any embodiment thereof, or a compound shown in Table 5, 6, or 7. In one embodiment, the compound is administered to a cell in a subject, preferably a mammal, and more preferably a human.

In another aspect, the invention provides a method of treating or preventing an inflammatory disorder in a subject, comprising administering to the subject an effective amount of a compound represented by any one of formula (I) through (LXXII), or any embodiment thereof, or a compound shown in Table 5, 6, or 7. In one embodiment, the compound is administered to a human to treat or prevent an inflammatory disorder. In another embodiment, the inflammatory disorder is selected from the group consisting of transplant rejection, skin graft rejection, arthritis, rheumatoid arthritis, osteoarthritis and bone diseases associated with increased bone resorption; inflammatory bowel disease, ileitis, ulcerative colitis, Barrett's syndrome, Crohn's disease; asthma, adult respiratory distress syndrome, chronic obstructive airway disease; corneal dystrophy, trachoma, onchocerciasis, uveitis, sympathetic ophthalmitis, endophthalmitis; gingivitis, periodontitis; tuberculosis; leprosy; uremic complications, glomerulonephritis, nephrosis; sclerodermatitis, psoriasis, eczema; chronic demyelinating diseases of the nervous system, multiple sclerosis, AIDS-related neurodegeneration, Alzheimer's disease, infectious meningitis, encephalomyelitis, Parkinson's disease, Huntington's disease, amyotrophic lateral sclerosis viral or autoimmune encephalitis; autoimmune disorders, immune-complex vasculitis, systemic lupus and erythematodes; systemic lupus erythematosus (SLE); cardiomyopathy, ischemic heart disease hypercholesterolemia, atherosclerosis, preeclampsia; chronic liver failure, brain and spinal cord trauma. In another embodiment, a compound represented by any one of formula (I) through (LXXII), or any embodiment thereof, or a compound shown in Table 5, 6, or 7, is administered with an additional therapeutic agent. In another embodiment, the additional therapeutic agent is an anti-inflammatory agent.

In another aspect, the invention provides a method of treating or preventing an immune disorder in a subject, comprising administering to the subject an effective amount of a compound represented by any one of formula (I) through (LXXII), or any embodiment thereof, or a compound shown in Table 5, 6, or 7. In one embodiment, the compound is administered to a human to treat or prevent an immune disorder. In another embodiment, the immune disorder is selected from the group consisting of multiple sclerosis, myasthenia gravis, Guillain-Barré, autoimmune uveitis, autoimmune hemolytic anemia, pernicious anemia, autoimmune thrombocytopenia, temporal arteritis, anti-phospholipid syndrome, vasculitides such as Wegener's granulomatosis, Behcet's disease, psoriasis, dermatitis herpetiformis, pemphigus vulgaris, vitiligo, Crohn's disease, ulcerative colitis, primary biliary cirrhosis, autoimmune hepatitis, Type 1 or immune-mediated diabetes mellitus, Grave's disease. Hashimoto's thyroiditis, autoimmune oophoritis and orchitis, autoimmune disorder of the adrenal gland, rheumatoid arthritis, systemic lupus erythematosus, scleroderma, polymyositis, dermatomyositis, ankylosing spondylitis, and Sjogren's syndrome. In another embodiment, a compound represented by any one of formula (I) through (LXXII), or any embodiment thereof, or a compound shown in Table 5, 6, or 7, is administered with an additional therapeutic agent. In another embodiment, the additional therapeutic agent is an anti-inflammatory agent.

In another aspect, the invention provides a method of suppressing the immune system in a subject in need thereof, comprising administering to the subject an effective amount of a compound represented by any one of formula (I) through (LXXII), or any embodiment thereof, or a compound shown in Table 5, 6, or 7. In one embodiment, the compound is administered to a human in need of immunosuppression. In another embodiment, the subject is in need of immunosuppression because the subject is an organ transplant recipient. The organ transplant may be an allographic, xenographic or syngeneic organ transplant. In one embodiment, the organ transplanted is selected from the group consisting of a skin graft, heart, kidney, lung, liver, pancreas, cornea, bowel, and stomach. In another embodiment, the subject is in need of immunosuppression because the subject is the recipient of stem cell transplantation. In another embodiment, a compound represented by any one of formula (I) through (LXXII), or any embodiment thereof, or a compound shown in Table 5, 6, or 7, is administered with an additional therapeutic agent. In another embodiment, the additional therapeutic agent is an immunosuppressant.

In another aspect, the invention provides a method for monitoring the treatment of a patient who is being treated with an Hsp90 inhibitor, comprising the steps of:
a) obtaining a sample of peripheral blood mononuclear cells (PBMCs) from the patients before administering a dosage of an Hsp90 inhibitor;
b) obtaining a sample of PBMCs from the patients after administering a dosage of an Hsp90 inhibitor;
c) determining the amount of glucocorticoid receptor protein in the PBMCs of the patient before receiving the dosage;
d) determining the amount of glucocorticoid receptor protein in the PBMCs of the patient after receiving the dosage; and
e) comparing the amount of glucocorticoid receptor protein in the PBMCs before receiving the dosage with the amount of glucocorticoid receptor protein after receiving the dosage, wherein the greater a decreased in glucocorticoid receptor protein after receiving the dosage indicates greater activity of the Hsp90 inhibitor.

One method of determining the amount of glucocorticoid receptor protein in the PBMCs of a patient is to isolate the PBMCs from a sample of whole blood and do a Western Blot as described in Examples 114 and 115 herein. In one embodiment, the sample of PBMCs is obtained about 8 hours to about 24 hours after the patient has received the dosage of the Hsp90 inhibitor. The method of monitoring treatment of a patient with an Hsp90 inhibitor can be done one a patient who is receiving their first dose of an Hsp90 inhibitor or a patient who has already received one or more doses of an Hsp90 inhibitor. In one embodiment, the sample of PBMCs from the patient may be obtained before and after a single dose of the Hsp90 inhibitor. In another embodiment, the sample of PBMCs from the patient may be obtained before the first dose of the Hsp90 inhibitor and after every succeeding dose of the Hsp90 inhibitor. In one embodiment, the Hsp90 inhibitor is a compound represented by any one of formula (I) through (LXXII), or any embodiment thereof, or a compound shown in Table 5, 6, or 7. In one embodiment, the subject being treated with the Hsp90 inhibitor is being treated for cancer.

In another aspect, the invention relates to a method for optimizing dosing for a subject undergoing cancer therapy, wherein the dosing includes administration of an Hsp90 inhibitor, comprising the steps of:
a) changing dosing of the Hsp90 inhibitor during therapy;
b) comparing the amount of glucocorticoid receptor protein in PBMCs in a control sample with the amount of glucocorticoid receptor protein in PBMCs in a test sample;
c) comparing side effects from the Hsp90 inhibitor between the test sample and the control sample;
d) optimizing dosing of the Hsp90 inhibitor based on the dosing in step a) in combination with the results of step b) or step c), wherein the test sample is taken from the subject after changing the dosing; and the control sample is taken from the subject before changing the dosing.
In one embodiment, the Hsp90 inhibitor is a compound represented by any one of formula (I) through (LXXII), or any embodiment thereof, or a compound shown in Table 5, 6, or 7.
   1) Agents Useful in Combination with the Compounds of the Invention

The methods for immunosuppression or for treating or preventing inflammatory conditions and immune disorders in a patient in need thereof can further comprise administering to the patient being administered represented by any one of formula (I) through (LXXII), or any embodiment thereof, or a compound shown in Table 5, 6, or 7, and an effective amount of one or more other active agents. Such active agents may include those used conventionally for immunosuppression or for inflammatory conditions or immune disorders. These other active agents may also be those that provide other benefits when administered in combination with a compound represented by any one of formula (I) through (LXXII), or any embodiment thereof, or a compound shown in Table 5, 6, or 7. For example, other therapeutic agents may include, without limitation, steroids, non-steroidal anti-inflammatory agents, antihistamines, analgesics, immunosuppressive agents and suitable mixtures thereof. In such combination therapy treatment, both the compounds of this invention and the other drug agent(s) are administered to a subject (e.g., humans, male or female) by conventional methods. The agents may be administered in a single dosage form or in separate dosage forms. Effective amounts of the other therapeutic agents and dosage forms are well known to those skilled in the art. It is well within the skilled artisan's purview to determine the other therapeutic agent's optimal effective-amount range.

In one embodiment of the invention where another therapeutic agent is administered to a subject, the effective amount of a compound represented by any one of formula (I) through (LXXII), or any embodiment thereof, or a compound shown in Table 5, 6, or 7 is less than its effective amount when the other therapeutic agent is not administered. In another embodiment, the effective amount of the conventional agent is less than its effective amount when a compound represented by any one of formula (I) through (LXXII), or any embodiment thereof, or a compound shown in Table 5, 6, or 7 is not administered. In this way, undesired side effects associated with high doses of either agent may be minimized. Other potential advantages (including without limitation improved dosing regimens and/or reduced drug cost) will be apparent to those of skill in the art.

In one embodiment relating to autoimmune, allergic and inflammatory conditions, the other therapeutic agent may be a steroid or a non-steroidal anti-inflammatory agent. Particularly useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin; sodium salicylate, choline magnesium tri salicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9^{th} ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Of particular relevance to allergic disorders, the other therapeutic agent may be an antihistamine. Useful antihistamines include, but are not limited to, loratadine, cetirizine, fexofenadine, desloratadine, diphenhydramine, chlorpheniramine, chlorcyclizine, pyrilamine, promethazine, terfenadine, doxepin, carbinoxamine, clemastine, tripelennamine, brompheniramine, hydroxyzine, cyclizine, meclizine, cyproheptadine, phenindamine, acrivastine, azelastine, levocabastine, and mixtures thereof. For a more detailed description of antihistamines, see Goodman & Gilman's The Pharmacological Basis of Therapeutics (2001) 651-57, 10^{th} ed).

Immunosuppressive agents include glucocorticoids, corticosteroids (such as Prednisone or Solumedrol), T cell blockers (such as cyclosporin A and FK506), purine analogs (such as azathioprine (Imuran)), pyrimidine analogs (such as cytosine arabinoside), alkylating agents (such as nitrogen mustard, phenylalanine mustard, buslfan, and cyclophosphamide), folic acid antagonists (such as aminopterin and methotrexate), antibiotics (such as rapamycin, actinomycin D, mitomycin C, puramycin, and chloramphenicol), human IgG, antilymphocyte globulin (ALG), and antibodies (such as anti-CD3 (OKT3), anti-CD4 (OKT4), anti-CD5, anti-CD7, anti-IL-2 receptor, anti-alpha/beta TCR, anti-ICAM-1, anti-CD20 (Rituxan), anti-IL-12 and antibodies to immunotoxins).

The foregoing and other useful combination therapies will be understood and appreciated by those of skill in the art. Potential advantages of such combination therapies include a different efficacy profile, the ability to use less of each of the individual active ingredients to minimize toxic side effects, synergistic improvements in efficacy, improved ease of administration or use and/or reduced overall expense of compound preparation or formulation.
2) Compositions and Methods for Administering Therapies

The present invention provides methods for the treatment, prophylaxis, and amelioration of inflammatory and immune disorders and methods for immunosuppression. In a specific embodiment, a composition comprises one or more compounds of the invention, or a tautomer, or a pharmaceutically acceptable salt, solvate, clathrate, hydrate or prodrug thereof. In another embodiment, a composition of the invention comprises one or more prophylactic or therapeutic agents other than a compound of the invention, or a tautomer, or a pharmaceutically acceptable salt, solvate, clathrate, hydrate, or prodrug thereof. In another embodiment, a composition of the invention comprises one or more compounds of the invention, or a tautomer, or a pharmaceutically acceptable salt, solvate, clathrate, hydrate or prodrug thereof, and one or more other prophylactic or therapeutic agents. In another embodiment, the composition comprises a compound of the invention, or a tautomer, or a pharmaceutically acceptable salt, solvate, clathrate, hydrate, or prodrug thereof, and a pharmaceutically acceptable carrier, diluent or excipient.

In a preferred embodiment, a composition of the invention is a pharmaceutical composition or a single unit dosage form. Pharmaceutical compositions and dosage forms of the invention comprise one or more active ingredients in relative amounts and formulated in such a way that a given pharmaceutical composition or dosage form can be used to treat or prevent an inflammatory or immune disorder or for immunosuppression. Preferred pharmaceutical compositions and dosage forms comprise a compound of formula (I) through (LXXII), or any embodiment thereof, or a compound shown in Table 5, 6, or 7, or a tautomer, or a pharmaceutically acceptable prodrug, salt, solvate, clathrate, hydrate, or prodrug thereof, optionally in combination with one or more additional active agents.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Easton Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms.

The suitability of a particular excipient may also depend on the specific active ingredients in the dosage form. For example, the decomposition of some active ingredients can be accelerated by some excipients such as lactose, or when exposed to water. Active ingredients that comprise primary or secondary amines (e.g., N-desmethylvenlafaxine and N,N-didesmethylvenlafaxine) are particularly susceptible to such accelerated decomposition. Consequently, this invention encompasses pharmaceutical compositions and dosage forms that contain little, if any, lactose. As used herein, the term “lactose-free” means that the amount of lactose present, if any, is insufficient to substantially increase the degradation rate of an active ingredient. Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen (1995) Drug Stability: Principles & Practice, 2d. Ed., Marcel Dekker, NY, N.Y., 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.
i) Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.
ii) Controlled Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be-readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance.

Most controlled-release formulations are designed to initially release an amount of drug (active ingredient) that promptly produces the desired therapeutic effect, and gradually and continually release of other amounts of drug to maintain this level of therapeutic,or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

A particular extended release formulation of this invention comprises a therapeutically or prophylactically effective amount of a compound of formula (I) through (LXXII), or any embodiment thereof, or a compound shown in Table 5, 6, or 7, or a tautomer, or a pharmaceutically acceptable salt, solvate, hydrate, clathrate, or prodrug thereof, in spheroids which further comprise microcrystalline cellulose and, optionally, hydroxypropylmethyl-cellulose coated with a mixture of ethyl cellulose and hydroxypropylmethylcellulose. Such extended release formulations can be prepared according to U.S. Pat. No. 6,274,171, the entirely of which is incorporated herein by reference.

A specific controlled-release formulation of this invention comprises from about 6% to about 40% a compound of formula (I) through (LXXII), or any embodiment thereof, or a compound shown in Table 5, 6, or 7, or a tautomer, or a pharmaceutically acceptable salt, solvate, hydrate, clathrate, or prodrug thereof, by weight, about 50% to about 94% microcrystalline cellulose, NF, by weight, and optionally from about 0.25% to about 1% by weight of hydroxypropyl-methylcellulose, USP, wherein the spheroids are coated with a film coating composition comprised of ethyl cellulose and hydroxypropylmethylcellulose.
iii) Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.
iv) Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences (1980 & 1990) 16th and 18th eds., Mack Publishing, Easton Pa. and Introduction to Pharmaceutical Dosage Forms (1985) 4th ed., Lea & Febiger, Philadelphia. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990) 16th and 18th eds., Mack Publishing, Easton Pa.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.
v) Dosage & Frequency of Administration

The amount of the compound or composition of the invention which will be effective for immunosuppression or in the prevention, treatment, management, or amelioration of an inflammatory or immune disorder or one or more symptoms thereof, will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of a compound of the invention for the conditions described herein lie within the range of from about 0.01 mg to about 1000 mg per day, given as a single once-a-day dose preferably as divided doses throughout a day. In one embodiment, the daily dose is administered twice daily in equally divided doses. In another embodiment, the compounds of the invention are administered one to three times a week. Specifically, a dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different proliferative disorders, as will be readily known by those of ordinary skill in the art. Similarly, amounts sufficient to prevent, manage, treat or ameliorate such proliferative disorders, but insufficient to cause, or sufficient to reduce, adverse effects associated with the compounds of the invention are also encompassed by the above described dosage amounts and dose frequency schedules. Further, when a patient is administered multiple dosages of a compound of the invention, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition of the invention or a compound of the invention administered to suppress a patient's immune system or to prevent, treat, manage, or ameliorate a an inflammatory or immune disorder, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition of the invention or a compound of the invention administered to suppress a patient's immune system or to prevent, treat, manage, or ameliorate an inflammatory or immune disorder, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosages of prophylactic or therapeutic agents other than compounds of the invention, which have been or are currently being used to suppress a patient's immune system or to prevent, treat, manage, or ammeliorate an inflammatory or immune disorder, or one or more symptoms thereof can be used in the combination therapies of the invention. Preferably, dosages lower than those which have been or are currently being used to suppress the patient's immune system or to prevent, treat, manage, or ameliorate an inflammatory or immune disorder, or one or more symptoms thereof, are used in the combination therapies of the invention. The recommended dosages of agents currently used to suppress a patient's immune system or for the prevention, treatment, management, or amelioration of an inflammatory or immune disorder, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

In certain embodiments, when the compounds of the invention are administered in combination with another therapy, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies (e.g., prophylactic or therapeutic agents) are administered within the same patent visit.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of suppressing a patient's immune system or preventing, treating, managing, or ameliorating an inflammatory or immune disorder, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, preferably, once every 2 days, once every 3 days, once every 4 days, once every 5 days, once every 6 days, once every 7 days, once every 8 days, once every 10 days, once every two weeks, once every three weeks, or once a month.

D. Other Embodiments

The compounds of the invention may be used as research tools (for example, to evaluate the mechanism of action of new drug agents, to isolate new drug discovery targets using affinity chromatography, as antigens in an ELISA or ELISA-like assay, or as standards in in vitro or in vivo assays). These and other uses and embodiments of the compounds and compositions of this invention will be apparent to those of ordinary skill in the art.

The invention is further defined by reference to the following examples describing in detail the preparation of compounds of the invention. It will be apparent to those skilled in the art that many modifications, both to materials and methods, may be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 1. EXAMPLES

Reagents and solvents used below can be obtained from commercial sources such as Aldrich Chemical Co. (Milwaukee, Wis., USA). ¹H-NMR and ¹³C-NMR spectra were recorded on a Varian 300 MHz NMR spectrometer. Significant peaks are tabulated in the order: δ (ppm): chemical shift, multiplicity (s, singlet; d, doublet; t, triplet; q, quartet; m, multiplet; br s, broad singlet), coupling constant(s) in Hertz (Hz) and number of protons.

### Example 1

### Synthesis of Compound 76

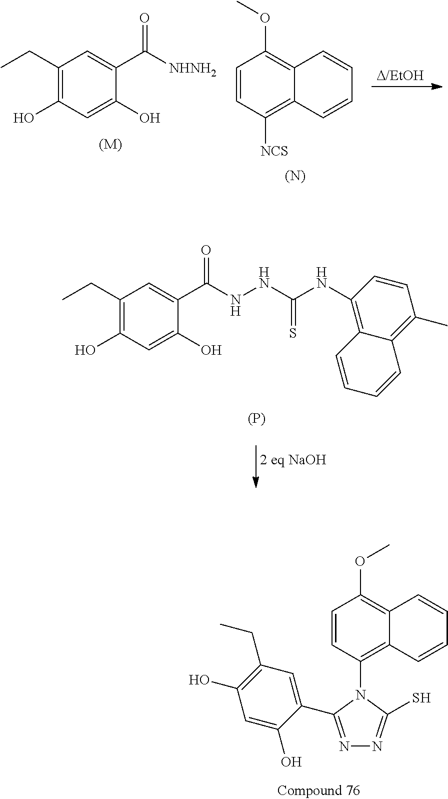

The hydrazide (M) (1.45 g, 7.39 mmol) and the isothiocyanate (N) (1.59 g, 7.39 mmol) were dissolved in ethanol (20 ml) with heating. When the starting materials were dissolved the solution was allowed to cool to room temperature and a precipitate formed. This precipitate was filtered then washed with ether to provide the intermediate (P) as a white solid (2.85 g, 97%). The intermediate (VII) (1.89 g, 4.77 mmol) was heated in a solution of sodium hydroxide (0.38 g, 9.54 mmol) in water (20 mL) at 110° C. for 2 hours. The solution was allowed to cool to room temperature then acidified with conc. HCl. The resulting precipitate was filtered then washed with water (100 mL) and dried. The crude product was recrystallized from ethanol to produce compound 76 as a white solid (1.4 g, 75%).

¹H NMR (DMSO-d₆) δ 9.43-9:53 (bs, 2H),8.11-8.16 (m, 1H), 7.47-7.55 (m, 2H), 7.38 (d, J=8.1 Hz, 1H), 7.31-7.36 (m, 1H), 6.98 (d, J=8.1 Hz, 1H), 6.71 (s, 1H), 6.17 (s, 1H), 3.98 (s, 3H), 2.17 (q, J=7.5 Hz, 2H), 0.73 (t, J=7.5 Hz, 3H);

ESMS calculated for (C₂₁H₁₉N₃O₃S) 393.11; Found 394.1 (M+1)⁺.

### Example 2

### Synthesis of Compound 124

3-(2,4-Dihydroxy-phenyl)-4-(naphthalen-1-yl)-5-mercapto-triazole (505 mg, 1.5 mmol), which is commercially available from Scientific Exchange, Inc., Center Ossipee, NH 03814, and Et₃N (0.84 ml, 6.0 mmol) in 15 ml CH₂Cl₂ were treated dropwise with ethyl isocyanate (360 mg, 5.0 mmol) at 0° C. The mixture was then warmed to room temperature and stirred for 3 h. The reaction mixture was diluted with CH₂Cl₂, washed with H₂O and saturated brine, dried with Na₂SO₄, and concentrated in vacuo. The residue was chromatographed (Hexane/EtOAc 3:1) to give Compound 124 as a white solid (480 mg, 58%).

¹H-NMR (CDCl₃) δ 10.13 (s, 1H), 7.96 (d, J=9.0 Hz, 2H), 7.61-7.57 (m, 3H), 7.49-7.36(m, 2H), 7.01(s, 1H), 6.88 (d, J=8.4 Hz, 1H), 6.70 (d, J=8.4 Hz, 1H), 4.98-4.96(m, 2H), 3.56(q, J=7.2 Hz, J=12.6 Hz, 2H), 3.28-3.10(m, 4H), 1.33(t, J=7.2 Hz, 3H), 1.13 (q, J=15.0 Hz, J=7.2 Hz, 6H);

ESMS calculated for C₂₇H₂₈N₆O₅S: 548.18; Found: 549.1 (M+1)⁻.

### Example 3

### Synthesis of Compound 188

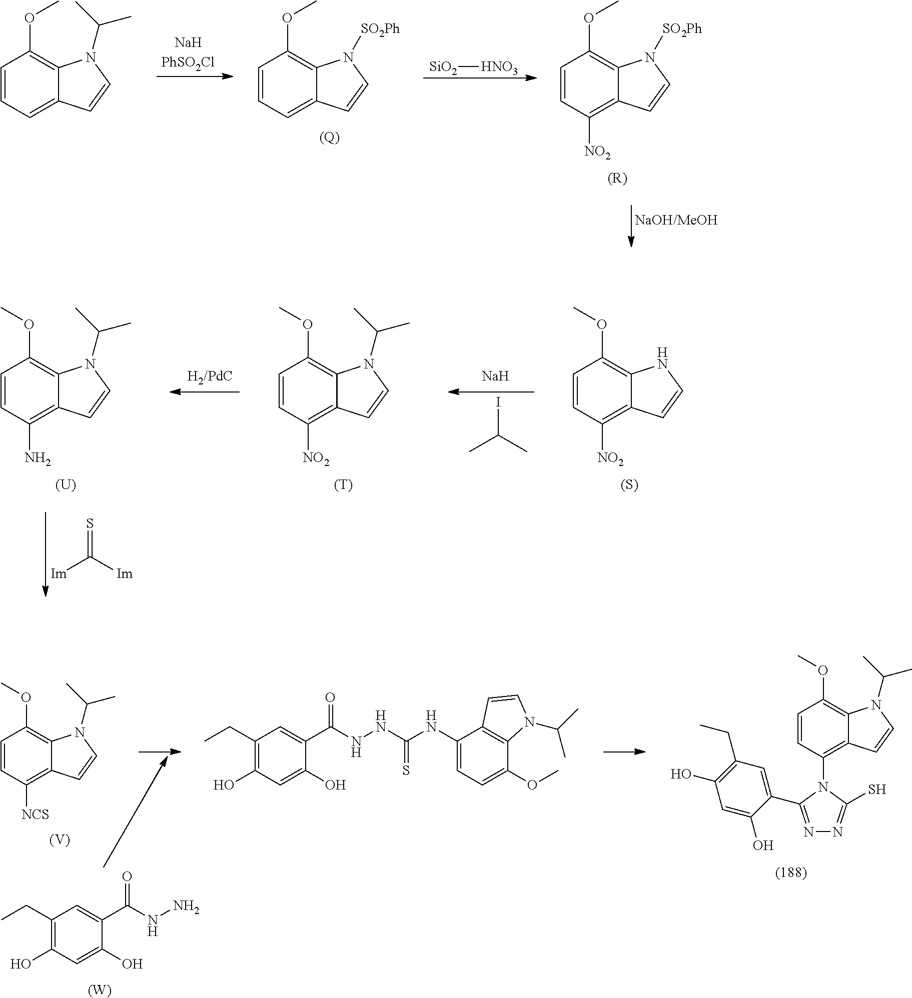

### 1-Benzenesulfonyl-7-methoxy-1H-indole (Q)

To a solution of 7-methoxyindole (1 eq) in DMF cooled in an ice bath was added NaH (60% dispersion in oil, 1.2 eq). The reaction was stirred for 1 hr at room temperature then recooled in an ice bath. Benzenesulfonyl chloride (1.1 eq) was added then the reaction was stirred for 2 hrs at room temperature. Water/ethyl acetate were added and the ethyl acetate layer was washed repeatedly (3×) with water. The ethyl acetate layer was concentrated and evaporated to dryness.

### 1-Benzenesulfonyl-7-methoxy-4-nitro-1H-indole (R)

To a solution of 1-benzenesulfonyl-7-methoxy-1H-indole (Q) (1 eq) in dichloromethane cooled in an ice bath was added SiO₂—HNO₃ (2 wt eq) in small portions. The reaction was stirred for 1 hr at room temperature. Activated carbon (2 wt eq) was added then the entire mixture was stirred for 1 hr. The mixture was then filtered and evaporated to dryness. Separation of the isomers was achieved by column chromatography.

### 7-Methoxy-4-nitro-1H-indole (S)

To a solution of 1-benzenesulfonyl-7-methoxy-4-nitro-1H-indole (R) (1 eq) in methanol was added a solution of sodium hydroxide (5 eq) in water. The solution was heated to reflux for 3 hrs. Methanol was removed under reduced pressure then water and ethyl acetate were added. The ethyl acetate layer separated and washed repeatedly (3×) with water. The ethyl acetate layer was concentrated and evaporated to dryness to produce the desired product.

### 1-Isopropyl-7-methoxy-4-nitro-1H-indole (T)

To a solution of 7-methoxy-4-nitro-1H-indole (S) (1 eq) in DMF cooled in an ice bath was added NaH (60% dispersion in oil, 1.2 eq). The reaction was stirred for 1 hr at room temperature then recooled in an ice bath. 2-Iodopropane (1.1 eq) was added then the reaction was stirred for 2 hrs at room temperature. Water and ethyl acetate were added. The ethyl acetate layer was separated and washed repeatedly (3×) with water. The ethyl acetate layer was concentrated then evaporated to dryness. Further purification by column chromatography produced the pure desired product.

### 1-Isopropyl-7-methoxy-1H-indol-4-ylamine (U)

A solution of 1-isopropyl-7-methoxy-4-nitro-1H-indole (T) (1 eq) and palladium 10% on activated carbon (0.1 wt eq) in methanol/ethyl acetate (1:1) was shaken on a Parr hydrogenation apparatus under hydrogen for 1 hr. The reaction was then filtered through Celite and evaporated to dryness to produce the desired product.

### 1-Isopropyl-4-isothiocyanato-7-methoxy-1H-indole (V)

To a solution of 1-isopropyl-7-methoxy-1H-indol-4-ylamine (U) (1 eq) in dichloromethane was added 1,1′-thiocarbonyldiimidazole (1.2 eq). The reaction was stirred for 2 hrs at room temperature then evaporated to dryness. Further purification by column chromatography produced the pure desired product.

### 3-(2,4-Dihydroxy-5-ethyl-phenyl)-4-(1-isopropyl-7-methoxy-indol-4-yl)-5-mercapto-[1,2,4] triazole (Compound 188)

5-Ethyl-2,4-dihydroxy-benzoic acid hydrazide (W) (1 eq) and 1-isopropyl-4-isothiocyanato-7-methoxy-1H-indole (V) (1.01 eq) were heated in ethanol (0.02 M based on isothiocyanate) at 80° C. for 1 hr. The solution was allowed to cool to room temperature overnight. The resulting precipitate was filtered, washed with ether, dried and used without further purification (yield 80%). The precipitate was suspended in aqueous NaOH solution (2 eq NaOH) and nitrogen was bubbled through this suspension for 10 min. The reaction was then heated to 110° C. for 1 hr under a nitrogen atmosphere then allowed to cool to room temperature. Neutralisation with conc. HCl produced a white precipitate which was filtered and washed with water. Repeated recrystallisation from EtOH/water produced the desired product (purity >95%, yield 50-70%)

¹H-NMR (DMSO-d₆) δ (ppm), 9.52 (s, 1H), 9.42 (s, 1H), 7.40 (d, J=3.3 Hz, 1H); 6.82 (d, J=8.4 Hz, 1H), 6.61 (s, 1H), 6.20 (s, 1H), 6.05 (d, J=3.3 Hz, 1H), 5.30 (qn, J=6.6 Hz, 1H), 3.89 (s, 3H), 2.14 (q, J=7.5 Hz, 2H), 1.41-1.47 (m, 6H), 0.68 (t, J=7.5 Hz, 3H);

### 2. ESMS Calculated. for C₂₂H₂₄N₄O₃S: 424.16; Found: 425.1 (M+1)⁺.

### Example 4

### Synthesis of Compound 223

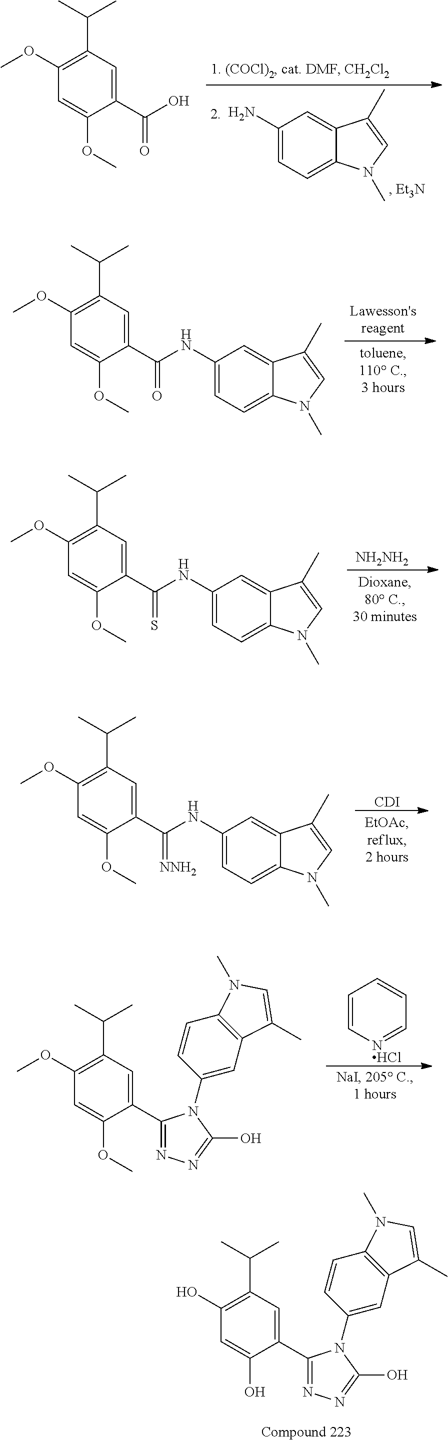

2,4-Dimethoxy-5-isopropylbenzoic acid (2.24 g, 10.0 mmol, 1.00 equiv.) in 50 mL CH₂Cl₂ at room temperature was treated with (COCl)₂ (1.40 g, 11.0 mmol, 1.10 equiv.) and catalytic amount of DMF (0.1 mL) for 1 hour. Solvent and excess (COCl)₂ were removed in vacuo. The residue was dissolved in 100 mL CH₂Cl₂, and treated with 1,3-dimethyl-5-aminoindole (1.60 g, 10.0 mmol, 1.00 equiv.) and triethylamine (1.55 g, 15.0 mmol, 1.50 equiv.) at 0° C. for one hour. Aqueous workup and removal of solvent gave a light brown solid which was washed with ether to yield off-white solid (2.28 g, 6.22 mmol, 62%).

¹H NMR (CDCl₃) δ (ppm) 9.78 (br s, 1H), 8.21 (s, 1H), 8.09 (d, J=2.1 Hz, 1H), 7.31 (dd, J=8.7 Hz, 2.1 Hz, 1H), 7.22 (d, J=8.7 Hz, 1H), 6.82 (s, 1H), 6.50 (s, 1H), 4.09 (s, 3H), 3.92 (s, 3H), 3.73 (s, 3H), 3.26 (hept, J=6.9 Hz, 1H), 2.32 (s, 3H), 1.24 (d, J=6.9 Hz, 6H).

The off-white solid obtained above was treated with Lawesson's reagent (1.51 g, 3.74 mmol, 0.6 equiv.) in 50 mL toluene at 110° C. for three hours. Toluene was removed on rotary evaporator and vacuum pump, and the residue was treated with hydrazine (anhydrous, 3.0 g, 94 mmol, 15.0 equiv.) in 20 mL dioxane at 80° C. for 30 minutes. The reaction mixture was extracted with ethyl acetate and water to remove excess hydrazine. The organic layer was dried over MgSO₄, and filtered to remove drying agent. Carbodiimidazole (CDI) (3.02 g, 18.7 mmol, 3.00 equiv.) was added to the solution, and the solution was refluxed (65° C.) for 2 hours. Solvent was removed, and the residue was treated with 20 mL THF and 10 mL NaOH (2M) to destroy excess CDI. Extraction with ethyl acetate (EtOAc) and water, followed by chromatography purification gave the desired product 3-(2,4-methoxy-5-isopropyl-phenyl)-4-(1,3-dimethyl-indol-5-yl)-5-hydroxy-[1,2,4] triazole as light brown solid (2.20 g, 5.42 mmol, 87%).

¹H NMR (CDCl₃), δ (ppm) 9.63 (br s, 1H), 7.34 (d, J=2.1 Hz, 1H), 7.20 (s, 1H), 7.18 (d, J=8.4 Hz, 1H), 7.00 (dd, J=8.4 Hz, 2.1 Hz, 1H), 6.80 (s, 1H), 6.19 (s, 1H), 3.76 (s, 3H), 3.69 (s, 3H), 3.40 (s, 3H), 3.15 (hept, J=6.9 Hz, 1H), 2.20 (s, 3H), 1.10 (d, J=6.9 Hz, 6H).

3-(2,4-methoxy-5-isopropyl-phenyl)-4-(1,3-dimethyl-indol-5-yl)-5-hydroxy-[1,2,4] triazole obtained above was treated with pyridine hydrochloride (12.53 g, 108.3 mmol, 20.0 equiv.), NaI (0.812 g, 5.42 mmol, 1.0 equiv.) and 0.5 mL water at 205° C. under nitrogen protection for 1 hour. The reaction mixture was treated with 200 mL water. The solid was collected by filtration, washed with 3×20 mL water, and dissolved in 50 mL 2M NaOH solution. The aqueous solution was extracted with 100 mL EtOAc, and the EtOAc layer was extracted with 2×20 mL 0.5M NaOH. EtOAc layer was discarded. The aqueous layer were combined, neutralized with HCl to PH around 5, and extracted with 3×100 mL EtOAc. The combined EtOAc layer was diluted with 50 mL THF, dried over MgSO₄, and filtered through silica gel plug. Most of solvents were removed to form a slurry with around 2 mL of solvent left. Solid was collected by filtration, washed with 2 mL EtOAc, and dried. The desired product 3-(2,4-dihydroxy-5-isopropyl-phenyl)-4-(1,3-dimethyl-indol-5-yl)-5-hydroxy-[1,2,4] triazole (Compound 223) was obtained as an off-white solid (1.75 g, 4.63 mmol, 85%).

¹H NMR (CD₃OD), δ (ppm) 7.46 (d, J=1.8 Hz, 1H), 7.41 (d, J=8.4 Hz, 1H), 7.04 (dd, J=8.4 Hz, 1.8 Hz, 1H), 7.02 (s, 1H), 6.53 (s, 1H), 6.26 (s, 1H), 3.74 (s, 3H), 2.88 (sept, J=6.9 Hz, 1H), 2.24 (s, 3H), 0.62 (d, J=6.9 Hz, 6H);

ESMS calculated. for C₂₁H₂₃N₄O_{3:} 378.1; Found: 379.1 (M+1)⁺.

The following compounds were prepared as described above in the section entitled “Methods of Making the Compounds of the Invention” and as exemplified in Examples 1 through 4.

### Example 5

### Compound 1

ESMS calcd for C₁₈H₁₃N₃OS: 319.1; Found: 320.0 (M+1)⁺.

### Example 6

### Compound 2

ESMS calcd for C₂₁H₁₉N₃O₄S: 409.11; Found: 410.0 (M+H)⁺.

### Example 7

### Compound 5

ESMS calcd for C₁₉H₁₅N₃O₂S: 365.08; Found: 266.0 (M+H)⁺.

### Example 8

### Compound 6

ESMS calcd for C₂₀H₁₇N₃O₂S: 379.10; Found: 380.0 (M+H)⁺.

### Example 9

### Compound 7

ESMS calcd for C₂₁H₁₉N₃O₂S: 393.11; Found: 394.0 (M+H)⁺.

### Example 10

### Compound 8

ESMS calcd for C₂₁H₁₉N₃O₃S: 393.11; Found: 394.0(M+H)⁺.

### Example 11

### Compound 9

ESMS calcd for C₂₁H₁₉N₃O₂S: 393.11; Found: 394.0 (M+H)⁺.

### Example 12

### Compound 13

¹H-NMR (DMSO-d₆) δ 9.65 (s, 1H), 9.57 (s, 1H), 7.50 (d, J=8.1 Hz, 1H), 7.35 (d, J=3.3 Hz, 1H), 7.14 (t, J=7.8 Hz, 1H), 6.96 (d, J=7.5 Hz, 1H), 6.88 (d, J=8.1 Hz, 1H), 6.09-6.11 (m, 2H), 6.01 (dd, J₁=2.1 Hz, J₂=8.1 Hz, 1H), 4.13-4.22 (m, 2H), 1.36 (t, J=7.2 Hz, 3H);

ESMS calcd for C₁₈H₁₆N₄O₂S: 352.10; Found: 353.1 (M+1)⁺.

### Example 13

### Compound 14

¹H NMR (DMSO-d₆) δ 9.72(s, 1H), 9.67(s, 1H), 7.04-7.01(m, 1H), 6.83-6.78(m, 2H), 6.66-6.63(m, 1H), 6.20-6.19(m, 2H), 4.22(s, 4H);

ESMS calcd for C₁₆H₁₃N₃O₄S: 343.06; Found: 344.0 (M+1)⁺.

### Example 14

### Compound 15

ESMS calcd for C₁₅H₁₃N₃O₂S: 299.07; Found: 300.0 (M+H)⁺.

### Example 15

### Compound 16

ESMS calcd for C₁₅H₁₃N₃O₂S: 299.07; Found: 300.0 (M+H)⁺.

### Example 16

### Compound 17

ESMS calcd for C₁₄H₁₀ClN₃O₂S: 319.02; Found: 320.0 (M+H)⁺.

### Example 17

### Compound 18

ESMS calcd for C₁₄H₁₀ClN₃O₂S: 319.02; Found: 320.0 (M+H)⁺.

### Example 18

### Compound 19

ESMS calcd for C₁₄H₁₀ClN₃O₂S: 319.02; Found: 320.1 (M+H)⁺.

### Example 19

### Compound 20

ESMS calcd for C₁₅H₁₃N₃O₃S: 315.07; Found: 316.0 (M+H)⁺.

### Example 20

### Compound 21

ESMS calcd for C₁₅H₁₃N₃O₃S: 315.07; Found: 316.0 (M+H)⁺.

### Example 21

### Compound 22

ESMS calcd for C₁₅H₁₃N₃O₃S: 315.07; Found: 316.0 (M+H)⁺.

### Example 22

### Compound 23

ESMS calcd for C₁₄H₁₀FN₃O₂S: 303.05; Found: 304.0 (M+H)⁺.

### Example 23

### Compound 23

¹H NMR (DMSO-d₆) δ 9.69 (s, 1H), 9.65 (s, 1H), 7.16 (d, J=7.2 Hz, 1H), 7.05 (t, J=7.2 Hz, 1H), 6.93 (d, J=8.1 Hz, 2H), 6.11-6.16 (m, 2H), 2.21 (s, 3H), 1.89 (s, 3H);

ESMS Calcd C₁₆H₁₅N₃O₂S: 313.09, Found 314.1(M+1)⁺.

### Example 24

### Compound 24

ESMS calcd for C₁₆H₁₅N₃O₂S: 313.09; Found: 314.0 (M+H)⁺.

### Example 25

### Compound 25

¹H NMR (DMSO-d₆) δ 10.44 (m, 1H), 8.00-7.95 (m, 2H), 7.55-7.37 (m, 5H), 6.61 (d, J=7.8 and 1.8 Hz, 1H), 6.51 (t, J=8.6 Hz, 1H), 6.41(d, J=10.8 Hz, 1H);

ESMS calcd for C₁₈H₁₂FN₃OS: 337.07; Found: 338.0 (M+1)⁺.

### Example 26

### Compound 26

¹H NMR (DMSO-d₆) δ 9.57 (s, 1H), 7.99 (d, J=8.4 Hz, 1H), 7.96 (d, J=6.9 Hz, 1H), 7.55-7.37 (m, 5H), 6.61 (d, J=8.1 Hz, 1H), 5.83 (d, J=2.1 Hz, 1H), 5.73(dd, J=8.1 and 1.8 Hz, 1H), 5.24 (s, 2H);

ESMS calcd for C₁₈H₁₄N₄OS: 334.09; Found: 335.0 (M+1)⁺.

### Example 27

### Compound 27

ESMS calcd for C₁₈H₁₉N₃O₂S: 341.12; Found: 342.0 (M+H)⁺.

### Example 28

### Compound 28

ESMS calcd for C₁₆H₁₅N₃O₂S: 313.09; Found: 314.0 (M+H)⁺.

### Example 29

### Compound 29

ESMS calcd for C₁₆H₁₅N₃O₂S: 313.09; Found: 314.0 (M+H)⁺.

### Example 30

### Compound 30

ESMS calcd for C₁₆H₁₅N₃O₂S: 313.09; Found: 314.0 (M+H)⁺.

### Example 31

### Compound 31

ESMS calcd for C₁₄H₁₀FN₃O₂S: 303.05; Found: 304.0 (M+H)⁺.

### Example 32

### Compound 32

ESMS calcd for C₁₅H₁₃N₃O₂S: 331.04; Found: 332.0 (M+H)⁺.

### Example 33

### Compound 33

ESMS calcd for C₁₈H₁₃N₃O₂S: 335.07; Found: 336.0 (M+H)⁺.

### Example 34

### Compound 34

ESMS calcd for C₁₆H₁₅N₃O₂S: 313.09; Found: 314.0 (M+H)⁺.

### Example 35

### Compound 35

ESMS calcd for C₁₅H₁₂FN₃O₂S: 317.06; Found: 317.0 (M+H)⁺.

### Example 36

### Compound 36

ESMS calcd for C20H15N3O2S: 361.1; Found: 362.0 (M+1)⁺.

### Example 37

### Compound 37

¹H NMR (DMSO-d₆) δ 10.03 (s, 1H), 8.00-7.96 (m, 2H), 7.55-7.37 (m, 5H), 7.00 (d, J=8.1 Hz, 1H), 6.20 (m, 2H), 3.57 (s, 3H);

ESMS calcd for C₁₉H₁₅N₃O₂S: 349.09; Found: 350.0 (M+1)⁺.

### Example 38

### Compound 38

ESMS calcd for C₁₄H₉Cl₂N₃O₂S: 352.98; Found: 353.9 (M+H)⁺.

### Example 39

### Compound 39

¹H NMR (DMSO-d₆) δ 9.74 (s, 1H), 9.63 (s, 1H), 8.14 (m, 1H), 7.52-7.48 (m, 2H), 7.37 (d, J=8.4 Hz, 1H), 7.32 (m, 1H), 6.96 (d, =8.1 Hz, 1H), 6.90 (d, =8.4 Hz, 1H), 6.08 (d, =1.9 Hz, 1H), 6.01 (d, =8.4 Hz, 1H), 3.98 (s, 3H);

ESMS calcd for C₁₉H₁₅N₃O₃S: 365.08; Found: 366.0 (M+1)⁺.

### Example 40

### Compound 40

ESMS calcd for C₂₅H₁₆N₃O₂S: 409.09; Found: 410.0 (M+1)⁺.

### Example 41

### Compound 42

¹H NMR (DMSO-d₆) δ 9.75(s, 1H), 9.67(s, 1H), 7.08(s, 2H), 6.96-6.94(m, 2H), 6.18-6.13(m, 2H), 2.72-2.50(m, 3H), 2.35-2.28(m, 1H), 1.64-1.60(m, 4H);

ESMS calcd for C₁₈ H₁₇N₃O₂S: 339.10; Found: 340.0 (M+1)⁺.

### Example 42

### Compound 43

ESMS calcd for C₂₂H₁₅N₃O₂S: 385.09; Found: 386.0 (M+1)⁺.

### Example 43

### Compound 44

ESMS calcd for C₂₀H₁₅N₃O₂S: 361.09; Found: 362.0 (M+1)⁺.

### Example 44

### Compound 45

ESMS calcd for C₁₉H₁₅N₃O₂S: 349.09; Found: 350.0 (M+1)⁺.

### Example 45

### Compound 46

ESMS calcd for C₁₉H₂₁N₃O₃S: 371.13; Found: 372.0 (M+1)⁺.

### Example 46

### Compound 47

ESMS calcd for C₂₂H₂₇N₃O₃S: 413.18; Found: 414.1 (M+1)⁺.

### Example 47

### Compound 48

ESMS calcd for C₁₈H₁₂ClN₃O₂S: 369.03; Found: 370.0 (M+H)⁺.

### Example 48

### Compound 49

¹H NMR (DMSO-d₆) δ 9.49 (s, 1H), 9.40 (s, 1H), 7.94-7.99 (m, 2H), 7.38-7.56 (m, 5H), 6.70 (s, 1H), 6.13 (s, 1H), 2.12 (q, J=7.2 Hz, 2H), 0.71 (t, J=7.2 Hz, 3H);

ESMS Calcd for C₂₀H₁₇N₃O₂S: 363.10, Found 364.1(M+1)⁺.

### Example 49

### Compound 50

ESMS calcd for C₂₀H₁₅N₃O₅S: 409.07; Found: 410.0 (M+H)⁺.

### Example 50

### Compound 51

ESMS calcd for C₁₈H₁₄N₄O₂S: 350.08; Found: 351.0 (M+H)⁺.

### Example 51

### Compound 52

ESMS calcd for C₁₇H₁₂N₄OS: 320.07; Found: 320.9 (M+H)⁺.

### Example 52

### Compound 53

¹H NMR (CDCl₃) δ 12.0 (br s, 1H), 9.87 (br s, 1H), 9.83 (br s, 1H), 7.97 (d, J=8.1 Hz, 2H), 7.41-7.56 (m, 5H), 7.13 (d, J=1.5 Hz, 1H), 7.07 (d, J=8.7 Hz, 1H), 6.71 (dd, J =1.8 Hz, 8.1 Hz, 1H), 1.93 (s, 3H);

ESMS calcd for C₂₀H₁₇N₄O₂S: 376.1; Found: 377.0(M+1)⁺.

### Example 53

### Compound 56

ESMS calcd for C₁₆H₁₅N₃O₄S: 345.08; Found: 346.0 (M+1)⁺.

### Example 54

### Compound 57

ESMS calcd for C₁₈H₁₆N₄O₂S: 352.10; Found: 353.0 (M+1)⁺.

### Example 55

### Compound 61

¹H NMR (DMSO-d₆) δ 9.66(s, 1H), 9.60(s, 1H), 7.29-7.27(m, 1H), 7.12-7-10(m, 2H), 7.03-7.00(m, 1H), 6.19-6.17(m, 2H), 1.18(s, 18H);

ESMS calcd for C₂₂H₂₇N₃O₂S: 397.18; Found: 398.1 (M+1)⁺.

### Example 56

### Compound 64

ESMS calcd for C₂₁H₁₅N₃O₃S: 389.08; Found: 390.0 (M+H)⁺.

### Example 57

### Compound 65

ESMS calcd for C₁₉H₁₃N₃O₄S: 379.06; Found: 380.0 (M+1)⁺.

### Example 58

### Compound 66

ESMS calcd for C₂₁H₁₈N₄O₃S: 406.11; Found: 407.0 (M+1)⁺.

### Example 59

### Compound 67

ESMS calcd for C₂₁H₁₉N₃O₃S: 393.11; Found: 394.0 (M+1)⁺.

### Example 60

### Compound 68

ESMS calcd for C₂₁H₁₉N₃O₃S: 393.1 1; Found: 394.0 (M+1)⁺.

### Example 61

### Compound 69

ESMS calcd for C₂₁H₁₉N₃O₃S: 393.11; Found: 394.0 (M+1)⁺.

### Example 62

### Compound 70

ESMS calcd for C₁₇H₁₂N₄O₂S: 336.07; Found: 337.0 (M+H)⁺.

### Example 63

### Compound 71

ESMS calcd for C₂₁H₁₉N₃O₃S: 393.11; Found: 394.0 (M+1)⁺.

### Example 64

### Compound 72

¹H NMR (DMSO-d₆) δ 10.3 (br s, 1H), 7.95-8.19 (m, 2H), 7.48-7.72 (m, 5H), 7.17 (d, J=8.4 Hz, 1H), 6.44 (d, J=8.4 Hz, 1H), 5.95 (d, J=2.1 Hz, 1H), 5.73 (dd, J=2.1 Hz, 8.4 Hz, 1H), 5.47 (br s, 1H), 3.62 (s, 3H);

ESMS calcd for C₁₉H₁₇N₄O₂S₂: 412.1; Found: 413.0(M+1)⁺.

### Example 65

### Compound 73

¹H NMR (DMSO-d₆) δ 9.37 (s, 1H), 8.94 (s, 1H), 7.94-7.98 (m, 2H), 7.43-7.60 (m, 5H), 5.97 (s, 1H), 1.85 (s, 3H), 1.81 (s, 3H);

ESMS calcd for C₂₀H₁₈N₃O₂S: 363.1; Found: 364.0(M+1)⁺.

### Example 66

### Compound 74

ESMS calcd for C₂₁H₁₉N₃O₄S: 409.11; Found: 410.0 (M+H)⁺.

### Example 67

### Compound 75

¹H NMR (DMSO-d₆) δ 9.46 (s, 1H), 9.45 (s, 1H), 7.95-8.00 (m, 2H), 7.38-7.56 (m, 5H), 6.65 (s, 1H), 6.15 (s, 1H), 2.07-2.14 (m, 2H), 081-1.18 (m, 11H);

ESMS calcd for C₂₄H₂₆N₃O₂S: 419.1; Found: 420.1(M+1)⁺.

### Example 68

### Compound 76

ESMS calcd for C₂₁H₁₉N₃O₃S: 393.11; Found: 394.0 (M+H)⁺.

### Example 69

### Compound 77

ESMS calcd for C₂₁H₁₉N₃O₃S: 393.11; Found: 394.0 (M+H)⁺.

### Example 70

### Compound 78

¹H NMR (DMSO-d₆) δ 9.71 (s, 1H), 9.35 (s, 1H), 7.98-8.04 (m, 2H), 7.50-7.62 (m, 5H), 6.58 (s, 1H), 2.15 (q, J=7.5 Hz, 2H), 0.58 (t, J=7.5 Hz, 3H);

ESMS calcd for C₂₀H₁₇ClN₃O₂S: 397.0; Found: 398.0(M+1)⁺.

### Example 71

### Compound 79

ESMS calcd for C₁₉H₂₁N₃O₃S: 371.13; Found: 372.0 (M+H)⁺.

### Example 72

### Compound 80

ESMS calcd for C₂₁H₁₉N₃O₂S: 393.11; Found: 394.0 (M+H)⁺.

### Example 73

### Compound 81

ESMS calcd for C₂₀H₁₇N₃O₂S: 379.10; Found: 380.0 (M+H)⁺.

### Example 74

### Compound 82

ESMS calcd for C₂₁H₁₉N₃O₂S: 393.11; Found: 394.0 (M+H)⁺.

### Example 75

### Compound 83

ESMS calcd for C₂₀H₁₇N₃O₃S: 379.10; Found: 380.0 (M+H)⁺.

### Example 76

### Compound 84

ESMS calcd for C₂₀H₁₇N₃O₃S: 379.10; Found: 380.0 (M+H)⁺.

### Example 77

### Compound 85

ESMS calcd for C₁₉H₁₅N₃O₂S: 365.08; Found: 266.0 (M+H)⁺.

### Example 78

### Compound 86

¹H NMR (DMSO-d₆) δ 9.68 (s, 1H), 9.58 (s, 1H), 8.2 (dd, J=7.0 and 2.4 Hz, 1H), 7.50 (m, 2H), 7.40 (tr, J=8.1 Hz, 1H), 7.32 (m, 1H), 6.97 (d, J=7.5 Hz, 1H), 6.95 (m, 1H), 6.89 (d, =8.4 Hz, 1H), 6.08 (d, =2.1 Hz, 1H), 6.0 (dd, =7.4 and 2.1 Hz, 1H), 3.96 (s, 3H);

ESMS calcd for C₁₉H₁₅N₃O₃S: 365.08; Found: 366.0 (M+1)⁺.

### Example 79

### Compound 87

¹H NMR (MeOH-d₄) δ 8.25 (m, 1H), 7.96 (s, 1H), 7.46-7.44 (m, 2H), 7.26 (d, J =8.4 Hz, 1H), 6.83 (d, J=8.1 Hz, 1H), 6.70 (d, J=8.7 Hz, 1H), 6.17 (d, J=2.1 Hz, 1H), 5.98 (dd, J=8.4 and 2.4 Hz, 1H);

ESMS calcd for C₁₈H₁₃N₃O₃S: 351.07; Found: 352.0 (M+1)⁺.

### Example 80

### Compound 88

¹H-NMR (DMSO-d₆) δ 9.69 (s, 1H), 9.59 (s, 1H), 7.54 (d, J=8.1 Hz, 1H), 7.46 (d, J=3 Hz, 1H), 7.14 (t, J=7.8 Hz, 1H), 6.97 (d, J=7.2 Hz, 1H), 6.89 (d, J=8.7 Hz, 1H), 6.12-6.13 (m, 2H), 6.02 (dd, J₁=2.4 Hz, J₂=8.4 Hz, 1H), 4.74 (qn, J=6.6 Hz, 1H), 1.40-1.46 (m, 6H);

ESMS calcd for C₁₉H₁₈N₄O₂S: 366.12; Found: 367.1 (M+1)⁺.

### Example 81

### Compound 89

ESMS calcd for C₂₂H₂₁N₃O₂S: 391.14; Found: 392.0 (M+H)⁺.

### Example 82

### Compound 90

¹H NMR (DMSO-d₆) δ 9.47 (s, 1H), 9.43 (s, 1H), 7.94-8.00 (m, 2H), 7.39-7.57 (m, 5H), 6.68 (s, 1H), 6.15 (s, 1H), 2.05-2.15 (m, 2H), 1.05-1.17 (m, 2H), 0.50 (t, J=7.5 Hz, 3H); ESMS calcd for C₂₁H₂₀N₃O₂S: 377.1; Found: 378.0(M+1)⁺.

### Example 83

### Compound 91

¹H NMR (DMSO-d₆) δ 9.15 (s, 1H), 8.50 (s, 1H), 8.00 -8.07 (m, 2H), 7.47-7.63 (m, 5H), 6.27 (s, 1H), 2.06 (q, J=7.5 Hz, 2H), 1.93 (s, 3H), 0.45 (t, J=7.5 Hz, 3H);

ESMS calcd for C₂₁H₂₀N₃O₂S: 377.1; Found: 378.0(M+1)⁺.

### Example 84

### Compound 93

ESMS calcd for C₁₆H₁₅N₃O₄S: 345.08; Found: 346.0 (M+H)⁺.

### Example 85

### Compound 95

ESMS calcd for C₁₆H₁₂N₄O₂S: 324.07; Found: 325.0 (M+H)⁺.

### Example 86

### Compound 96

ESMS calcd for C₁₉H₁₈N₄O₃S: 382.11; Found: 383.0 (M+H)⁺.

### Example 87

### Compound 98

ESMS calcd for C₁₇H₁₂N₄O₂S: 336.07; Found: 337.0 (M+H)⁺.

### Example 88

### Compound 99

ESMS calcd for C₁₉H₁₃N₃O₄S: 379.06; Found: 379.9 (M+H)⁺.

### Example 89

### Compound 100

¹H-NMR (DMSO-d₆) δ 9.52 (s, 1H), 9.42 (s, 1H), 7.56 (d, J=8.7 Hz, 1H), 7.49 (d, J=3.3 Hz, 1H), 7.14 (t, J=7.5 Hz, 1H), 6.95 (d, J=8.4 Hz, 1H), 6.61 (s, 1H), 6.21 (s, 1H), 6.14 (dd, J=3.3 Hz, 1H), 4.76 (qn, J=6.6 Hz, 1H), 2.14 (q, J=7.5 Hz, 2H), 1.41-1.47 (m, 6H), 0.66 (t, J=7.5 Hz, 3H);

ESMS calcd for C₂₁H₂₂N₄O₂S: 394.15; Found: 395.1 (M+1)⁺.

### Example 90

### Compound 101

ESMS calcd for C₁₉H₁₇N₅O₃S: 395.11; Found: 396.0 (M+H)⁺.

### Example 91

### Compound 102

ESMS calcd. for C₁₉H₂₀N₅O₂S: 381.1; Found: 382.0 (M+1)⁺.

### Example 92

### Compound 103

¹H NMR (DMSO-d₆) δ 9.48 (s, 1H), 9.38 (s, 1H), 7.29(d, J=8.4 Hz, 1H), 7.25(d, J=1.8 Hz, 1H), 6.85-6.89 (m, 2H), 6.18 (s, 1H), 3.61 (s, 3H), 2.30 (s, 3H), 2.29 (q, J=7.5 Hz, 2H), 2.09 (s, 3H), 0.94 (t, J=7.5 Hz, 3H);

ESMS calcd for C₂₁H₂₃N₄O₂S: 394.1; Found: 395.0(M+1)⁺.

### Example 93

### Compound 104

ESMS calcd for C₁₉H₁₅N₃O₃S: 365.08; Found: 366.0 (M+H)⁺.

### Example 94

### Compound 106

ESMS calcd for C₂₀H₁₇N₄O₂S: 377.1; Found: 378.0(M+H)⁺.

### Example 95

### Compound 107

ESMS calcd for C₁₈H₁₃ClN₃O₂S: 369.0; Found: 370.0(M+H)⁺.

### Example 96

### Compound 116

¹H NMR (DMSO-d₆) δ 7.98-7.56 (m, 2H), 7.55-7.30 (m, 6H), 6.43 (dd, J=8.1 and 1.8 Hz, 1H), 6.29 (m, 1H), 3.65 (s, 3H), 3.16 (s, 3H);

ESMS calcd for C₂₀H₁₇N₃O₂S: 363.10; Found: 364.0 (M+I)⁺.

### Example 97

### Compound 117

¹H-NMR (CDCl₃) δ 7.83(d, J=8.1 Hz, 2H), 7.48-7.34(m, 4H), 7.28-7.20(m, 1H), 6.99 (d, J=1.8 Hz, 1H), 6.80(d, J=8.7 Hz, 1H), 6.62-6.58(m, 1H), 2.94(s, 3H), 2.89(s, 3H), 2.84(s, 3H), 2.81(s, 3H), 2.75-2.69(m, 6H);

ESMS calcd for C₂₇H₂₈N₆O₅S: 548.18; Found: 549.2 (M+1)⁺.

### Example 98

### Compound 122

¹H-NMR (CDCl₃) δ 7.98(m, 2H), 7.60-7.55(m, 3H), 7.51-7.45(m, 1H), 7.36-7.33(m, 1H), 6.98-6.97(m, 1H), 6.86(d, J=9.9 Hz, 1H), 6.70-6.67(m, 1H), 2.86(s, 3H), 2.26(s, 3H), 2.21(s, 3H);

ESMS calcd for C₂₄H₁₉N₃O₅S: 461.10; Found: 462.0 (M+1)⁺.

### Example 99

### Compound 125

ESMS calcd for C₂₀H₁₇N₃O₃S: 379.10; Found: 380.0 (M+H)⁺.

### Example 100

### Compound 126

ESMS calcd for C₁₀H₁₁N₃O₂S: 237.06; Found: 238.0 (M+H)⁺.

### Example 101

### Compound 127

ESMS calcd for C₁₁H₁₃N₃O₂S: 251.07; Found: 252.0 (M+H)⁺.

### Example 102

### Compound 128

ESMS calcd for C₁₁H₁₃N₃O₂S: 251.07; Found: 252.0 (M+H)⁺.

### Example 103

### Compound 129

ESMS calcd for C₁₁H₁₁N₃O₂S: 249.06; Found: 250.0 (M+H)⁺.

### Example 104

### Compound 130

ESMS calcd for C₁₂H₁₅N₃O₂S: 265.09; Found: 266.0 (M+H)⁺.

### Example 105

### Compound 131

ESMS calcd for C₂₀H₁₅N₃O₄S: 393.08; Found: 394.1 (M+H)⁺.

### Example 106

### Compound 177

¹H NMR (DMSO-d₆) δ 9.34(s, 1H), 9.22 (s, 1H), 8.01-7.96 (m, 2H), 7.58-7.44 (m, 5H), 6.56 (s, 1H), 6.14 (s, 1H), 3.29 (s, 3H);

ESMS calcd for C₁₉H₁₅N₃O₃S: 365.08; Found: 366.0(M+1)⁺.

### Example 107

### Compound 178

¹H NMR (DMSO-d₆) δ 10.29 (s, 1H), 9.49 (s, 1H), 9.42 (s, 1H), 8.16 (t, J=5.1 Hz, 1H), 7.45-7.43 (m, 2H), 7.26 (t, J=8.0 Hz, 1H), 6.84 (d, J=7.8 Hz, 1H), 6.75 (d, J=8.7 Hz, 1H), 6.66 (s, 1H), 6.14 (s, 1H), 2.12 (q, J=7.5 Hz, 2H), 0.70 (t, J=7.2 Hz, 3H);

ESMS calcd for C₂₀H₁₇N₃O₃S: 379.10; Found: 379.9 (M+1)⁺.

### Example 108

### Compound 179

ESMS calcd for C₁₉H₁₅N₃O₂S: 349.09; Found: 350.0 (M+1)⁺.

### Example 109

### Compound 180

ESMS calcd for C₁₉H₁₅N₃O₂S: 349.09; Found: 350.0 (M+H)⁺.

### Example 110

### Compound 181

ESMS calcd for C20H15N3O2S: 361.09; Found: 362.0 (M+H)⁺.

### Example 111

### Compound 182

ESMS calcd for C₁₆H₁₅N₃O₃S: 329.08; Found: 330.0 (M+H)⁺.

### Example 112

### Compound 183

ESMS calcd for C₂₀H₁₇N₃O₂S: 363.10; Found: 364.0 (M+H)⁺.

### Example 113

### Compound 184

ESMS calcd for C₁₈H₁₃N₃O₃S: 350.38; Found: 351.9(M+H)⁺.

### Example 114

### Compound 185

ESMS calcd. for C₂₀H₂₁N₄O₂S: 380.1; Found: 381.0 (M+1)⁺.

### Example 115

### Compound 187

ESMS calcd. for C₁₉H₂₀N₅O₂S: 381.1; Found: 382.0 (M+1)⁺.

### Example 116

### Compound 190

ESMS calcd. for C₂₁H₂₂N₄O₂S: 394.15; Found: 395.0 (M+1)⁺.

### Example 117

### Compound 191

ESMS calcd. for C₂₂H₂₃N₄O₄S: 438.1; Found: 439.0 (M+1)⁺.

### Example 118

### Compound 192

ESMS calcd. for C₂₀H₂₂N₅O₂S: 395.1; Found: 396.0 (M+1)⁺.

### Example 119

### Compound 193

ESMS calcd. for C₂₀H₂₂N₅O₂S: 395.1; Found: 396.0 (M+1)⁺.

### Example 120

### Compound 194

ESMS calcd. for C₂₃H₂₇N₄O₂S: 422.1; Found: 423.0 (M+1)⁺.

### Example 121

### Compound 195

ESMS calcd. for C₂₃H₂₅N₄O₂S: 420.1; Found: 421.0 (M+1)⁺.

### Example 122

### Compound 196

ESMS calcd. for C₂₅H₂₉N₄O₂S: 448.1; Found: 449.3 (M+1)⁺.

### Example 123

### Compound 197

ESMS calcd. for C₂₂H₂₄N₄O₂S: 408.16; Found: 409.2 (M+1)⁺.

### Example 124

### Compound 198

ESMS calcd. for C₂₃H₂₆N₄O₂S: 422.18; Found: 423.3 (M+1)⁺.

### Example 125

### Compound 199

ESMS calcd. for C₂₄H₂₈N₄O₂S: 436.19; Found: 437.3 (M+1)⁺.

### Example 126

### Compound 200

ESMS calcd. for C₂₂H₂₂N₄O₂S: 406.15; Found: 407.2 (M+1)⁺.

### Example 127

### Compound 201

ESMS calcd. for C₂₃H₂₄N₄O₃S: 436.16; Found: 437.3 (M+1)⁺.

### Example 128

### Compound 202

ESMS calcd. for C₂₂H₂₃N₄O₂S: 406.1; Found: 407.0 (M+H)⁺.

### Example 129

### Compound 204

ESMS calcd. for C₂₄H₂₈N₄O₃S: 452.19; Found: 453.2 (M+1)⁺.

### Example 130

### Compound 205

ESMS calcd. for C₂₃H₂₄N₄O₃S: 436.16; Found: 437.1 (M+1)⁺.

### Example 131

### Compound 206

ESMS calcd. for C₂₁H₂₃N₄O₂S: 394.1; Found: 395.1 (M+1)⁺.

### Example 132

### Compound 207

ESMS calcd. for C₂₀H₂₁N₄O₂S: 380.1; Found: 381.1 (M+1)⁺.

### Example 133

### Compound 208

ESMS calcd. for C₂₃H₂₆N₄O₃S: 438.17; Found: 439.1 (M+1)⁺.

### Example 134

### Compound 209

ESMS calcd. for C₂₂H₂₄N₄O₂S: 408.1; Found: 409.1 (M+1)⁺.

### Example 135

### Compound 210

ESMS calcd. for C₂₄H₂₃N₄O₂S: 430.1; Found: 431.1 (M+1)⁺.

### Example 136

### Compound 211

ESMS calcd. for C₂₁H₂₂N₄O₃S: 410.14; Found: 411.1 (M+1)⁺.

### Example 137

### Compound 212

ESMS calcd. for C₂₃H₂₆N₄O₃S: 438.17; Found: 439.1 (M+1)⁺.

### Example 138

### Compound 213

ESMS calcd. for C₂₀H₂₁N₄O₂S: 380.1; Found: 381.1 (M+1)⁺.

### Example 139

### Compound 214

ESMS calcd. for C₁₉H₁₉N₄O₂S: 366.1; Found: 367.1 (M+1)⁺.

### Example 140

### Compound 215

ESMS calcd. for C₂₀H₁₉N₃O₄S: 397.1; Found: 398.1 (M+1)⁺.

### Example 141

### Compound 216

¹H NMR (DMSO-d₆): δ (ppm) 9.56 (s, 1H), 9.40 (s, 1H), 8.03 (d, J=2.4 Hz, 1H), 7.58 (d, J=8.4 Hz, 1H), 7.54 (d, J=2.1 Hz, 1H), 7.11 (dd, J=8.4, 2.1 Hz, 1H), 6.97 (d, J=2.4 Hz, 1H), 6.89 (s, 1H), 6.17 (s, 1H), 2.23 (q, J=7.2 Hz, 2H), 0.93 (t, J=7.2 Hz, 3H);

ESMS calcd. for C₁₈H₁₅N₃O₃S: 353.08; Found: 354.0 (M+1)⁺.

### Example 142

### Compound 217

¹H NMR (DMSO-d₆): δ (ppm) 9.59 (s, 1H), 9.43 (s, 1H), 7.67 (d, J=8.7 Hz, 1H), 7.54 (d, J=2.1 Hz, 1H), 7.20 (dd, J=8.4, 2.1 Hz, 1H), 6.96 (s, 1H), 6.18 (s, 1H), 2.60 (s, 3H), 2.34 (q, J=7.2 Hz, 2H), 0.98 (t, J=7.2 Hz, 3H);

ESMS calcd. for C₁₈H₁₆N₄O₃S: 368.09; Found: 369.0 (M+1)⁺.

### Example 143

### Compound 218

ESMS calcd. for C₂₁H₂₃N₄O₂S: 394.1; Found: 395.1 (M+1)⁺.

### Example 144

### Compound 219

ESMS calcd. for C₂₁H₂₁N₄O₂S: 392.1; Found: 393.1 (M+1)⁺.

### Example 145

### Compound 220

ESMS calcd. for C₂₀H₂₁N₄O₃: 364.1; Found: 365.1 (M+1)⁺.

### Example 146

### Compound 221

ESMS calcd. for C₂₀H₂₁N₄O₂S: 379.1; Found: 381.1 (M+1)⁺.

### Example 147

### Compound 222

ESMS calcd. for C₂₁H₂₃N₄O₂S: 394.1; Found: 395.1(M+1)⁺.

### Example 148

### Compound 224

ESMS calcd. for C₁₉H₂₁N₄O₂S: 368.1; Found: 369.1 (M+1)⁺.

### Example 149

### Compound 225

ESMS calcd. for C₁₉H₁₉N₄O₂S: 366.1; Found: 367.1(M+1)⁺.

### Example 150

### Compound 226

ESMS calcd. for C₂₀H₂₁N₄O₃: 364.1; Found: 365.1 (M+1)⁺.

### Example 151

### Compound 227

ESMS calcd. for C₂₁H₂₂N₄O₂S: 394.15; Found: 395.1 (M+1)⁺.

### Example 152

### Compound 228

ESMS calcd. for C₂₂H₂₄N₄O₂S: 408.16; Found: 409.1 (M+1)⁺.

### Example 153

### Compound 229

ESMS calcd. for C₂₀H₁₈F₃N₅O₂S: 449.11; Found: 450.1 (M+1)⁺.

### Example 154

### Compound 230

ESMS calcd. for C₁₉H₁₉N₅O₂S: 381.13; Found: 382.1 (M+1)⁺.

### Example 155

### Compound 231

ESMS calcd. for C₁₉H₁₉N₅O₂S: 381.13; Found: 382.1 (M+1)⁺.

### Example 156

### Compound 232

ESMS calcd. for C₂₂H₂₄N₄O₃S: 392.18; Found: 393.1 (M+1)⁺.

### Example 157

### Compound 233

ESMS calcd. for C18H17N3O4S: 371.09; Found: 372.1 (M+1)+.

### Example 158

### Compound 234

ESMS calcd. for C20H21N3O2S: 367.14; Found: 368.1 (M+1)+.

### Example 159

### Compound 235

ESMS calcd. for C₁₉H₁₉N₅O₂S: 381.13; Found: 382.1 (M+1)⁺.

### Example 160

### Compound 239

ESMS clcd for C₁₉H₂₁N₄O₂S: 368.1; Found: 369.1 (M+H)⁺.

### Example 161

### Compound 240

ESMS clcd for C₁₈H₁₆N₄O₃S: 368.09.10; Found: 369.1 (M+H)⁺.

### Example 162

### Compound 241

ESMS clcd for C₁₇H₁₅N₅O₃S: 369.09; Found: 370.1 (M+H)⁺.

### Example 163

### Compound 242

ESMS clcd for C₁₉H₁₈N₄O₃S: 382.11; Found: 383.1 (M+H)⁺.

### Example 164

### Compound 243

ESMS clcd for C₂₂H₂₆N₄O₃S: 426.17; Found: 427.1 (M+H)⁺.

### Example 165

### Compound 244

ESMS clcd for C₁₈H₁₆N₄O₄S: 384.09; Found: 385.1 (M+H)⁺

### Example 166

### Compound 245

ESMS clcd for C₁₈H₁₆N₄O₃S₂: 400.07; Found: 401.1 (M+H)⁺

### Example 167

### Compound 245

ESMS clcd for C₁₇H₁₄N₄O₃S₂: 386.05; Found: 387.0 (M+H)⁺.

### Example 168

### 4-{5-Hydroxy-4-[4-methoxy-3-(methylpropylamino)phenyl]-4H-[1,2,4]triazol-3-yl}-6-isopropyl-benzene-1,3-diol

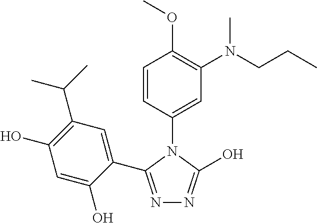 [SMILES: CCCN(C)c1cc(-n2c(O)nnc2-c2cc(C(C)C)c(O)cc2O)ccc1OC]

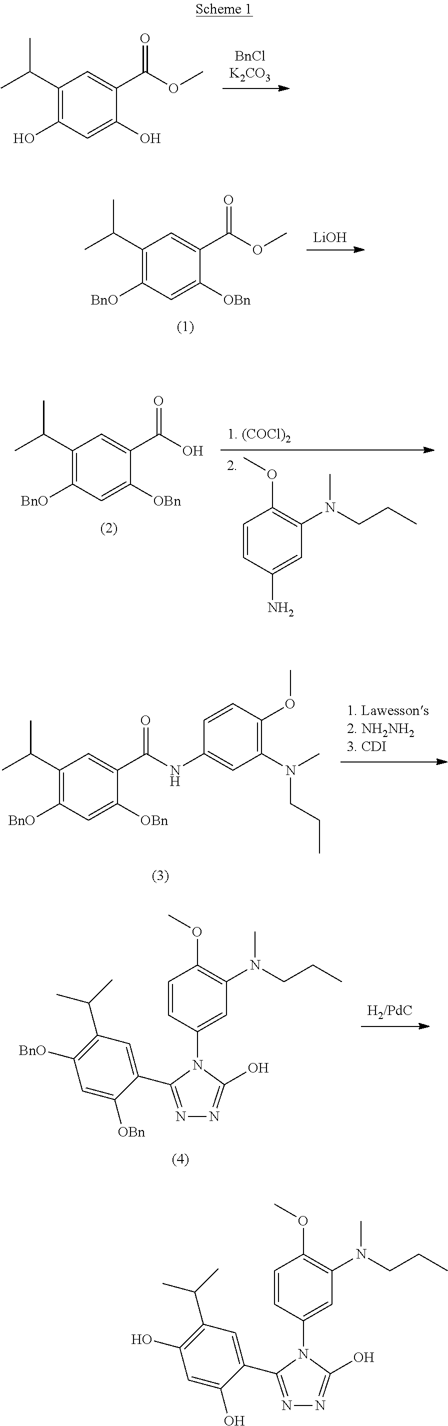

To a solution of 2,4-dihydroxy-5-isopropylbenzoic acid methyl ester (1.63 g, 7.75 mmol) in dimethylformamide (DMF) (100 mL) was added potassium carbonate (3.21 g, 23 mmol) then benzyl chloride (1.95 ml, 17 mmol). The suspension was heated to 80° C. for 16 hrs under a nitrogen atmosphere. Ethyl acetate (100 ml) and water (100 ml) were added, and then the ethyl acetate layer was washed with water (3×50 mL), and then dried over magnesium sulfate, filtered and evaporated-to dryness to produce the desired compound as brown oil (2.9 g, 97%).

2,4-Bis-benzyloxy-5-isopropylbenzoic acid methyl ester (3.23 g, 8.27 mmol) and LiOH (1.0 g, 24.8 mmol) were heated in a mixture of tetrahydrofuranyl (THF)/methanol/water (100 mL, 3:1:1) for 16 hrs. Ethyl acetate (100 mL) and water (100 ml) were added, then the ethyl acetate layer was washed with water (3×50 mL), dried over magnesium sulfate, filtered and evaporated to dryness to produce the desired compound as a yellow solid (2.6 g, 83%).

2,4-Bis-benzyloxy-5-isopropylbenzoic acid (1.25 g, 3.32 mmol) was dissolved in dichloromethane (50 mL) and cooled in an ice bath. Oxalyl chloride (0.32 mL, 3.65 mmol) was added followed by the dropwise addition of DMF (0.1 mL). The reaction was stirred at room temperature for 1 hr then evaporated to dryness under reduced pressure to produce a brown solid. This solid was dissolved in THF (50 mL) and cooled in an ice bath. A solution of 4-Methoxy-N³-methyl-N³-propyl-benzene-1,3-diamine (0.71 g, 3.65 mmol) in THF (20 mL) was added dropwisely followed by the triethylamine (1.6 mL) and the reaction was stirred at room temperature for 16 hrs. Ethyl acetate (50 mL) and water (100 mL) were added. The ethyl acetate layer was washed with water (3×50 mL), dried over magnesium sulfate, filtered and evaporated to dryness to produce the crude product as a brown solid. Purification by silicagel chromatography (elution with 25% ethyl acetate/hexane) provided the desired compound as a white solid (1.8 g, 93%).

2,4-Bis-benzyloxy-5-isopropyl-N-[4-methoxy-3-(methylpropylamino)phenyl]benzamide (700 mg, 1.27 mmol) and Lawesson's reagent (0.31 g, 0.76 mmol) were dissolved in toluene (20 mL) and heated to 110° C. for 3 hrs then evaporated to dryness under reduced pressure to produce a yellow oil. This crude product was dissolved in dioxane (10 mL), anhydrous hydrazine (0.6 mL) was added and the reaction was heated to 80° C. for 30 min. After cooling, ethyl acetate (50 mL) and water (50 mL) were added. The ethyl acetate layer was washed with water (3×50 mL), dried over magnesium sulfate, filtered and evaporated to dryness to produce the crude product as a brown solid. This solid was dissolved in ethyl acetate (50 mL), CDI (0.66 g, 4.08 mmol) was added then the reaction was heated to reflux for 3 hrs. Removal of the solvent under reduced pressure followed by purification by silicagel chromatography (elution with 50% ethyl acetate/hexane) provided the desired compound as a white solid (250 mg, 33% over 3 steps).

5-(2,4-Bis-benzyloxy-5-isopropyl-phenyl)-4-[4-methoxy-3-(methylpropylamino)phenyl]-4H-[1,2,4]triazol-3-ol (240 mg, 0.4 mmol) was dissolved in methanol (10 mL) then 10% palladium on charcoal (200 mg) was added and the reaction was stirred under an atmosphere of hydrogen for 16 hrs. Filtration was carried out through a silca gel plug and removal of the solvent under reduced pressure produced the desired compound as a white solid (150 mg, 94%).

¹H NMR (300 MHz, DMSO-d₆), δ (ppm):11.8 (s, 1H), 9.55 (s, 1H), 9.39 (s, 1H), 6.88 (d, J=8.7 Hz, 1H), 6.77-6.79 (m, 2H), 6.5 (s, 1H), 6.24 (s, 1H), 3.73 (s, 3H), 2.97 (qn, J=6.9 Hz, 1H), 2.79 (t, J=7.5 Hz, 2H), 2.48 (s, 3H), 1.30 (m, 2H), 0.97 (d, J=6.9 Hz, 6H), 0.73 (t, J=7.5 Hz, 3H).

ESMS clcd for C₂₂H₂₈N₄O₄: 412.21; Found: 413.2 (M+H)⁺.

### Example 169

### 4-Isopropyl-6-{5-mercapto4-[4-methoxy-3-(methyl-propyl-amino)-phenyl]-4H-[1,2,4]triazol-3-yl}-benzene-1,3-diol

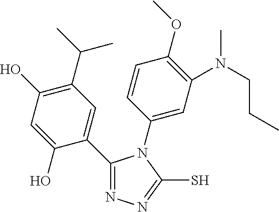 [SMILES: CCCN(C)c1cc(-n2c(S)nnc2-c2cc(C(C)C)c(O)cc2O)ccc1OC]

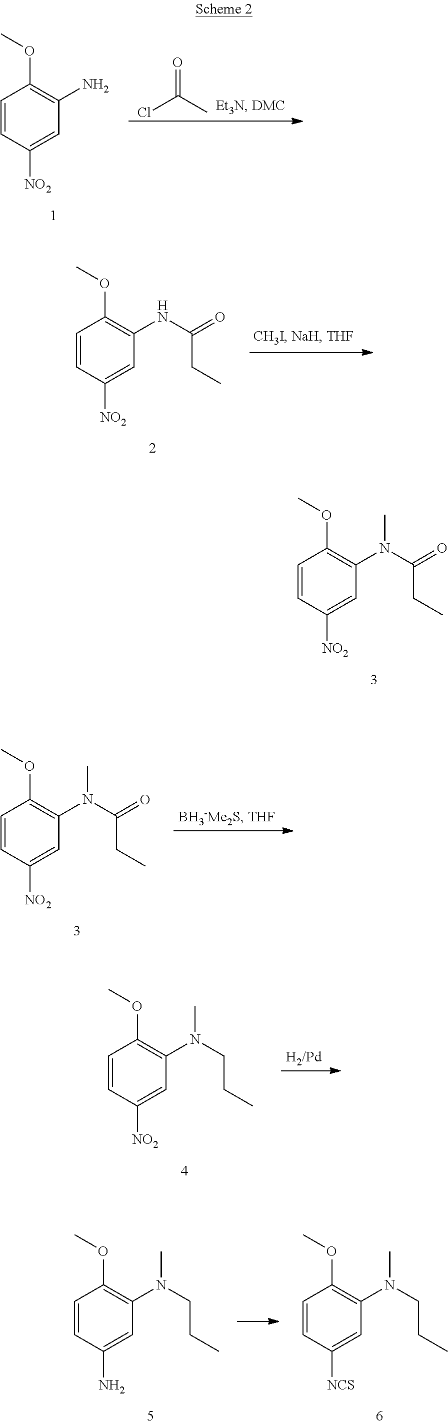

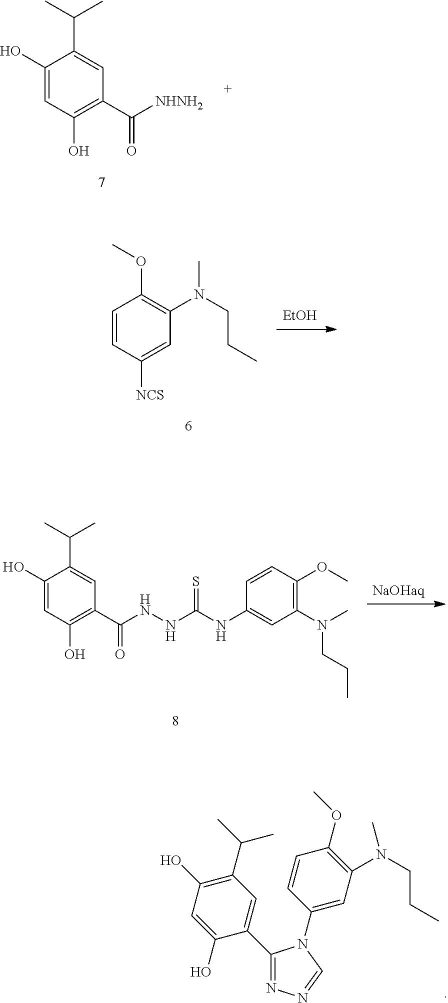

2-methoxy-5-nitroaniline (1) (10.1 g, 60.0 mmol) in 250 mL dichloromethane at 0°-5° C. was treated with triethylamine (10.0 g, 100.0 mmol) and propionyl chloride (6.7 g, 6.3 mL, 72.0 mmol) for 1 hour and 0.5 h at room temperature (RT). Normal aqueous workup and removal of solvent gave a light yellow solid which was washed with hexane/EtOAc (9:1) to yield solid N-(2-Methoxy-5-nitro-phenyl)-propionamide (2) (13.2 g, 98%).

To a stirred solution of 11.2 g (50.0 mmol) of (2) in 150 mL of anhydrous THF at 0° C. under the nitrogen, was added 3.0 g (75 mmol) of NaH (60% in oil). The suspension was stirred for 0.5 h at 0° C. and 10 mL (150 mmol) of iodomethane was added at 0° C. After the mixture warmed to room temperature and stirred for 3 h, the reaction was quenched by ice brine and extracted with EtOAc (200 mL). The organic phase was washed with brine, dried (Na₂SO₄), filtered, evaporated in vacuo and the solid was washed with hexane/EtOAc (9:1) to give pure product N-(2-Methoxy-5-nitro-phenyl)-N-methyl-propionamide (3) as a light yellow solid (11.3 g, 95% yield).

N-(2-Methoxy-5-nitro-phenyl)-N-methyl-propionamide (3) (10.0 g 42 mmol) and borane-methyl sulfide complex (21 mL of 2.0M solution in tetrahydrofurane) in 50 mL THF were heated under reflux for 30 min, cooled and quenched by ice-water (slowly). Extraction with EtOAc and the organic layer washed with brine dried (Na₂SO₄), filtered and evaporated in vacuo to give (9.1 g, 96%) (2-Methoxy-5-nitro-phenyl)-methyl-propyl-amine (4) as a yellow oil.

A solution of 9.0 g (40.1 mmol mmol) of (2-Methoxy-5-nitro-phenyl)-methyl-propyl-amine (4) in 200 mL of MeOH/EtOAc (1:1) containing 5% w/w of Pd—C (10%) was subjected to hydrogenation (1 atm, balloon) overnight. The contents of the flask were passed through a short pad of celite and washed with EtOAc. The filtrate was evaporated under reduced pressure to give 7.7 g (92%) of crude amine 4-Methoxy-N3-methyl-N3-propyl-benzene-1,3-diamine (5) of an oil.

To a stirred solution of 6.8 g (35.0 mmol) of (5) in 150 mL of CH₂Cl₂ at RT was added 6.4 g (35 mmol) of 1,1′-thiocarbonyldiimidazole. The mixture was stirred at room temperature for 15 minutes and then evaporated under reduced pressure and the residue was passed through a short pad of silica gel, eluting with a gradient of hexane/EtOAc, which gave (5-Isothiocyanato-2-methoxy-phenyl)-methyl-propyl-amine (6) (7.85 g, 95%) as a colorless oil.

To a stirred solution of 4.5 g (19.0 mmol) of the isothiocyanate (6) in 60 mL of ethanol was added 4.0 g (19.0 mmol) of the hydrazide (7) portion wise. The resultant mixture was then heated at 70° C. for 1 h, then cooled. Solvent was removed on rotary evaporator and the residue was treated with hexane/EtoAc (9:1). The white precipitate thus obtained was filtered, washed with ether (2×50 mL) and vacuum dried to 7.6 g (90%) of (8) as white solid.

To a solution of 1.36 g (34 mmol) of NaOH in 80 mL of water was added 7.5 g (16.8 mmol) of the intermediate (8) portion-wise. After the dissolution of the solid (1-2 min), the flask was flushed with nitrogen and heated to 110° C. for 3 h. The reaction mixture was cooled, an additional 100 mL of water was added and the whole mixture was acidified with conc. HCl pH 7. The white precipitate thus obtained was filtered, washed with water (3×75 mL) and dried. The crude product was then re-dissolved in a mixture of 200 mL of ethyl acetate, dried over anhydrous Na₂SO₄ and passed through a short pad of silica gel with an additional 150 mL of ethyl acetate as eluent. The filtrates were concentrated and crude product was re-precipitated in 3:1 hexane/ethyl acetate to give 6.83 g (95%) of 4-isopropyl-6-{5-mercapto-4-[4-methoxy-3-(methyl-propyl-amino)-phenyl]-4H-[1,2,4]triazol-3-yl}-benzene-1,3-diol as white solid.

¹H NMR (300 MHz, DMSO-d₆), (ppm): 9.58 (s, 1H); 9.39 (s, 1H); 6.92-6.83 (m, 3H); 6.56(d, J=1.8 Hz, 1H); 6.23 (s, 1H); 3.74 (s, 3H); 3.0-2.93(m, 1H); 2.81(t, J=6.9 Hz, 2H); 2.48(s, 3H); 1.31-1.24 (m, 2H); 0.96 (d, J=6.9 Hz, 6H); 0.72 (t, J=7.2 Hz, 3H);

ESMS clcd for C₂₂H₂₈N₄O₃S: 428.19; Found: 429.2 (M+H)⁺.

### Example 170

### 4-(4-{3-[(2-Dimethylamino-ethyl)-methyl-amino]-4-methoxy-phenyl}-5-mercapto-4H-[1,2,4]triazol-3-yl)-6-isopropyl-benzene-1,3-diol

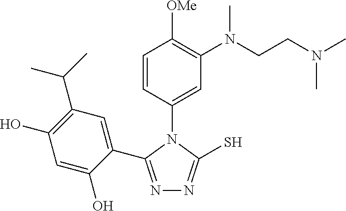 [SMILES: COc1ccc(-n2c(S)nnc2-c2cc(C(C)C)c(O)cc2O)cc1N(C)CCN(C)C]

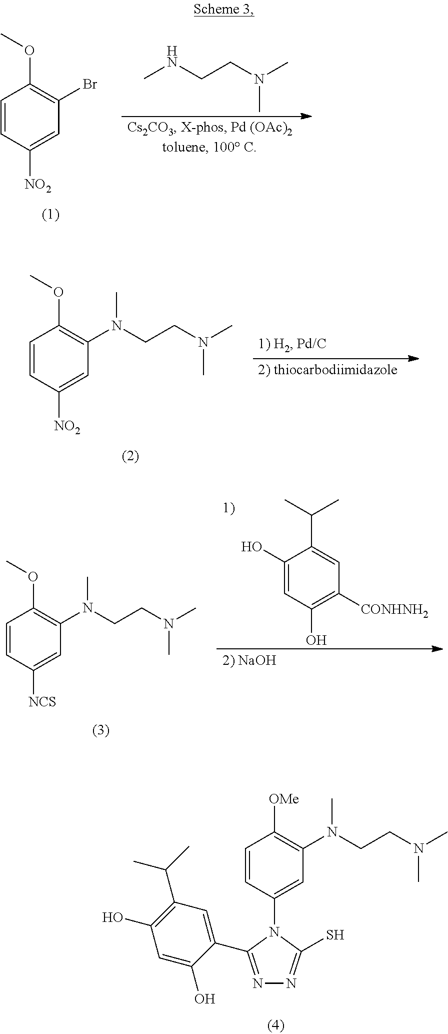

An oven-dried flask was charged with cesium carbonate (2.28 g, 7 mmol, 1.4 eq), Pd(OAc)₂ (79 mg, 0.35 mmol, 0.07 eq), and X-phos (238 mg, 0.5 mmol, 0.1 eq) under nitrogen. 2-bromo-1-methoxy-4-nitrobenzene (1.16 g, 5 mmol, 1 eq), N¹,N²,N²-trimethylethane-1,2-diamine (613 mg, 6 mmol, 1.2 eq) and toluene (20 mL, 0.25 M) were added, and the mixture was heated to 100° C. with stirring overnight. The reaction mixture was cooled to room temperature and concentrated. The crude product was then purified by flash chromatography on silica gel to give N¹-(2-methoxy-5-nitrophenyl)-N¹,N²,N²-trimethylethane-1,2-diamine (2) (340 mg, 1.34 mmol, 27%).

A solution of 340 mg of N¹-(2-methoxy-5-nitrophenyl)-N¹,N²,N²-trimethylethane-1,2-diamine (2) in 20 mL of ethanol containing 5% w/w of Pd—C (10%) was subjected to hydrogenation (1 atm, balloon) for 1.5 h. The contents of the flask were passed through a short pad of celite and washed with MeOH. The filtrate was evaporated under reduced pressure and crude amine obtained was carried over to the next reaction without further purification. Thiocarbodiimidazole (260 mg, 1.46 mmol) was added to the crude amine in dichloromethane (10 mL) at room temperature. The reaction mixture was stirred at room temperature for 1 h, and concentrated. The crude product was then purified by flash chromatography on silica gel to give N¹-(5-isothiocyanato-2-methoxyphenyl)-)-N¹,N²,N²-trimethylethane-1,2-diamine (3) (110 mg, 0.42 mmol, 31%).

To a stirred solution of 110 mg (0.54 mmol) of the isothiocyanate (3) in 5 mL of ethanol was added 105 mg (0.54 mmol) of 2,4-dihydroxy-5-isopropyl-benzoic acid hydrazide portion wise. The resultant mixture was then heated at 80° C. for 1 h, and then cooled. Solvent was removed on rotary evaporator and the residue was treated with hexane/EtOAc (9:1). The white precipitate thus obtained was filtered, washed with ether (2×20 mL) and vacuum dried to crude product as white solid. This solid was added to a solution of 44 mg (1.08 mmol) of NaOH in 5 mL of water portion-wise. After the dissolution of the solid (1-2 min), the flask was flushed with nitrogen and heated to 110° C. for 1.5 h. The reaction mixture was cooled, an additional 20 mL of water was added and the whole mixture was acidified with conc. HCl to pH 7. The white precipitate thus obtained was filtered, washed with water (3×20 mL) and dried. The crude product was then re-dissolved in a mixture of 20 mL of ethyl acetate, dried over anhydrous Na₂SO₄ and passed through a short pad of silica gel with an additional 15 mL of ethyl acetate as eluent. The filtrates were concentrated and crude product was re-precipitated in 3:1 hexane/ethyl acetate to give 97 mg of 4-(4-(3-((2-(dimethylamino)ethyl)(methyl)amino)-4-methoxyphenyl)-5-mercapto-4H-1,2,4-triazol-3-yl)-6-isopropylbenzene-1,3-diol (4) as white solid.

¹H-NMR 300 MHz, DMSO-d₆) δ (ppm): 9.80 (s, 1H), 9.62 (br s, 1H), 6.85 (m. 3H), 6.63 (m, 1H), 6.41 (s, 1H), 3.78 (s, 3H), 3.06 (m, 2H), 2.97 (q, J=6.9 Hz, 1H), 2.55 (s, 3H), 2.47 (m, 2H), 2.24 (s, 6H), 0.99 (s, 3H), 0.97 (s, 3H).

ESMS clcd for C₂₃H₃₁N₅O₃S: 457.21; Found: 458.2 (M+H)⁺.

### Example 171

### 4-Isopropyl-6-(5-mercapto-4-{4-methoxy-3-[(2-methoxy-ethyl)methylamino]phenyl}-4H-[1,2,4]triazol-3-yl)-benzene-1,3-diol

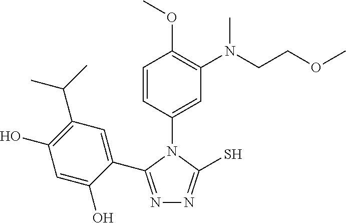 [SMILES: COCCN(C)c1cc(-n2c(S)nnc2-c2cc(C(C)C)c(O)cc2O)ccc1OC]

¹H NMR (300 MHz, DMSO-d₆) δ (ppm): 9.57 (s, 1H), 9.39 (s, 1H), 6.83-6.90 (m, 3H), 6.59 (d, J=2.1 Hz, 1H), 6.23 (s, 1H), 3.74 (s, 3H), 3.39 (t, J=6 Hz, 2H), 3.14 (s, 3H), 3.07 (t, J=6 Hz, 2H), 2.96 (qn, J=6.9 Hz, 1H), 2.54 (s, 3H), 0.97 (d, J=6.9 Hz, 6H). ESMS clcd for C₂₂H₂₈N₄O₄S: 444.18; Found: 445.2 (M+H)⁺.

### Example 172

### 4-{4-[3-(Cyclopropylmethylmethylamino)-4-methoxy-phenyl]-5-mercapto-4H-[1,2,4]triazol-3-yl}-6-isopropylbenzene-1,3-diol

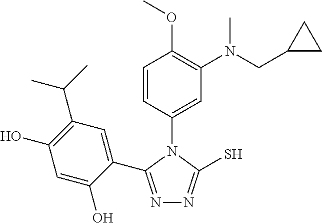 [SMILES: COc1ccc(-n2c(S)nnc2-c2cc(C(C)C)c(O)cc2O)cc1N(C)CC1CC1]

¹H NMR (300 MHz, DMSO-d₆) δ (ppm): 9.56 (s, 1H), 9.39 (s, 1H), 6.85-6.90 (m, 3H), 6.58 (d, J=2.1 Hz, 1H), 6.23 (s, 1H), 3.76 (s, 3H), 2.96 (qn, J=6.9 Hz, 1H), 2.76 (d, J=6.3 Hz, 2H), 2.57 (s, 3H), 0.99 (d, J=6.9 Hz, 6H), 0.58-0.64 (m, 1H), 0.32-0.34 (m, 2H), −0.03-0.01 (m, 2H).

ESMS clcd for C₂₃H₂₈N₄O₃S: 440.19; Found: 441.1 (M+H)⁺.

### Example 173

### N-{4-[3-(5-Ethyl-2,4-dihydroxy-phenyl)-5-mercapto-[1,2,4]triazol-4-yl]-phenyl}-N-methyl-acetamide,

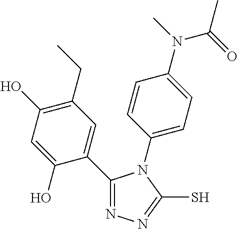 [SMILES: CCc1cc(-c2nnc(S)n2-c2ccc(N(C)C(C)=O)cc2)c(O)cc1O]

ESMS clcd for C₁₉H₂₀N₄O₃S: 384.13; Found: 385.1 (M+H)⁺.

### Example 174

### N-Ethyl-N-{5-[3-(5-ethyl-2,4-dihydroxy-phenyl)-5-mercapto-[1,2,4]triazol-4-yl]-2-methoxy-phenyl}-acetamide,

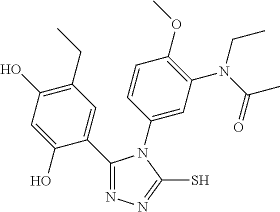 [SMILES: CCc1cc(-c2nnc(S)n2-c2ccc(OC)c(N(CC)C(C)=O)c2)c(O)cc1O]

ESMS clcd for C₂₁H₂₄N₄O₄S: 428.15; Found: 429.2 (M+H)⁺.

### Example 175

### 4-[4-(3-Diethylamino-4-methoxy-phenyl)-5-mercapto-4H-[1,2,4]triazol-3-yl]-6-ethyl-benzene-1,3-diol

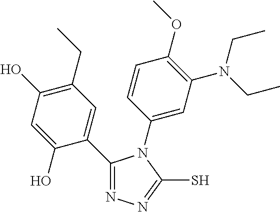 [SMILES: CCc1cc(-c2nnc(S)n2-c2ccc(OC)c(N(CC)CC)c2)c(O)cc1O]

ESMS clcd for C₂₁H₂₆N₄O₃S: 414.17; Found: 415.2 (M+H)⁺.

### Example 176

### 4-[4-(4-Dimethylamino-phenyl)-5-mercapto-4H-[1,2,4]triazol-3-yl]-6-ethyl-benzene-1,3-diol

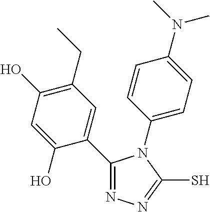 [SMILES: CCc1cc(-c2nnc(S)n2-c2ccc(N(C)C)cc2)c(O)cc1O]

ESMS clcd for C₁₈H₂₀N₄O₂S: 356.13; Found: 357.2 (M+H)⁺.

### Example 177

### 4-[4-(4-Diethylamino-phenyl)-5-mercapto-4H-[1,2,4]triazol-3-yl]-6-ethyl-benzene-1,3-diol

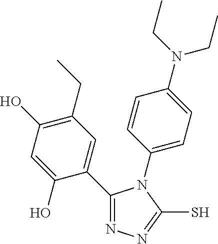 [SMILES: CCc1cc(-c2nnc(S)n2-c2ccc(N(CC)CC)cc2)c(O)cc1O]

ESMS clcd for C₂₀H₂₄N₄O₂S: 384.16; Found: 385.2 (M+H)⁺.

### Example 178

### 4-Ethyl-6-[5-mercapto-4-(4-morpholin-4-yl-phenyl)-4H-[1,2,4]triazol-3-yl]-benzene-1,3-diol

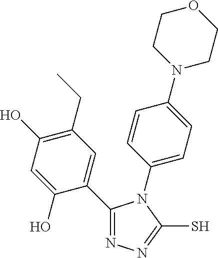 [SMILES: CCc1cc(-c2nnc(S)n2-c2ccc(N3CCOCC3)cc2)c(O)cc1O]

ESMS clcd for C₂₀H₂₂N₄O₃S: 398.14; Found: 399.2 (M+H)⁺.

### Example 179

### 4-Ethyl-6-[4-(4-imidazol-1-yl-phenyl)-5-mercapto-4H-[1,2,4]triazol-3-yl]-benzene-1,3-diol

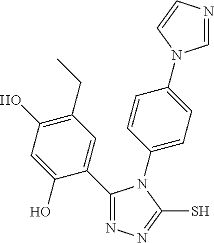 [SMILES: CCc1cc(-c2nnc(S)n2-c2ccc(-n3ccnc3)cc2)c(O)cc1O]

ESMS clcd for C₁₉H₁₇N₅O₂S: 379.11; Found: 380.2 (M+H)⁺.

### Example 180

### 4-[4-(2,5-Diethoxy-4-morpholin-4-yl-phenyl)-5-mercapto-4H-[1,2,4]triazol-3-yl]-6-ethyl-benzene-1,3-diol

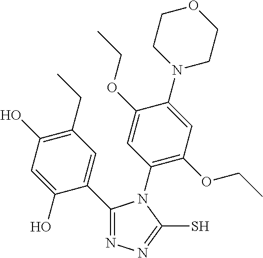 [SMILES: CCOc1cc(-n2c(S)nnc2-c2cc(CC)c(O)cc2O)c(OCC)cc1N1CCOCC1]

ESMS clcd for C₂₄H₃₀N₄O₅S: 486.19; Found: 487.3 (M+H)⁺.

### Example 181

### 4-Ethyl-6-{4-[3-(isopropyl-propyl-amino)-4-methoxy-phenyl]-5-mercapto-4H-[1,2,4]triazol-3-yl}-benzene-1,3-diol

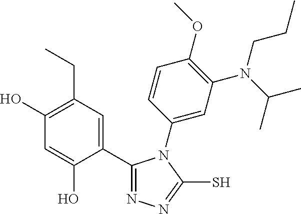 [SMILES: CCCN(c1cc(-n2c(S)nnc2-c2cc(CC)c(O)cc2O)ccc1OC)C(C)C]

ESMS clcd for C₂₃H₃₀N₄O₃S: 442.20; Found: 443.3 (M+H)⁺.

### Example 182

### 4-[4-(4-Dimethylamino-3-methoxy-phenyl)-5-mercapto-4H-[1,2,4]triazol-3-yl]-6-ethyl-benzene-1,3-diol

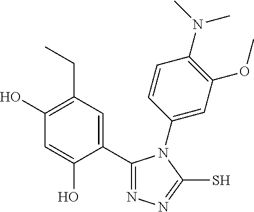 [SMILES: CCc1cc(-c2nnc(S)n2-c2ccc(N(C)C)c(OC)c2)c(O)cc1O]

ESMS clcd for C₁₉H₂₂N₄O₃S: 386.14; Found: 387.2 (M+H)⁺.

### Example 183

### 4-Ethyl-6-[5-mercapto-4-(3-pyrrolidin-1-yl-phenyl)-4H-[1,2,4]triazol-3-yl]-benzene-1,3-diol

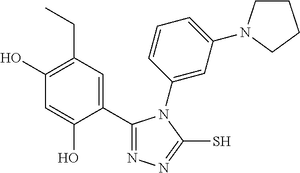 [SMILES: CCc1cc(-c2nnc(S)n2-c2cccc(N3CCCC3)c2)c(O)cc1O]

ESMS clcd for C₂₀H₂₂N₄O₂S: 382.15; Found: 383.2 (M+H)⁺.

### Example 184

### 4-[4-(3-Dimethylamino-phenyl)-5-mercapto-4H-[1,2,4]triazol-3-yl]-6-ethyl-benzene-1,3-diol

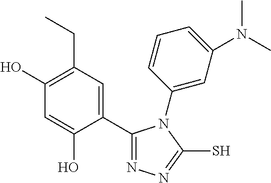 [SMILES: CCc1cc(-c2nnc(S)n2-c2cccc(N(C)C)c2)c(O)cc1O]

ESMS clcd for C₁₈H₂₀N₄O₂S: 356.13; Found: 357.2 (M+H)⁺.

### Example 185

### 4-Ethyl-6-{4-[3-(isopropyl-methyl-amino)-4-methoxy-phenyl]-5-mercapto-4H-[1,2,4]triazol-3-yl}-benzene-1,3-diol

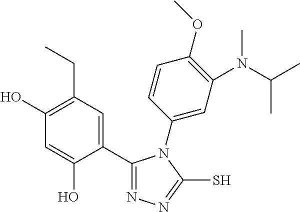 [SMILES: CCc1cc(-c2nnc(S)n2-c2ccc(OC)c(N(C)C(C)C)c2)c(O)cc1O]

ESMS clcd for C₂₁H₂₆N₄O₃S: 414.17; Found: 415.2 (M+H)⁺.

### Example 186

### 4-[4-(3-Dimethylamino-4-methoxy-phenyl)-5-mercapto-4H-[1,2,4]triazol-3-yl]-6-ethyl-benzene-1,3-diol

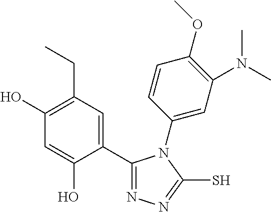 [SMILES: CCc1cc(-c2nnc(S)n2-c2ccc(OC)c(N(C)C)c2)c(O)cc1O]

ESMS clcd for C₁₉H₂₂N₄O₃S: 386.14; Found: 387.2 (M+H)⁺.

### Example 187

### 4-Ethyl-6-{4-[3-(ethyl-methyl-amino)-4-methoxy-phenyl]-5-mercapto-4H-[1,2,4]triazol-3-yl}-benzene-1,3-diol

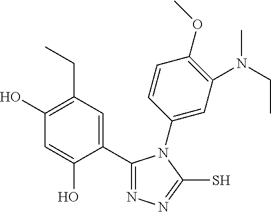 [SMILES: CCc1cc(-c2nnc(S)n2-c2ccc(OC)c(N(C)CC)c2)c(O)cc1O]

ESMS clcd for C₂₀H₂₄N₄O₃S: 400.16; Found: 401.2 (M+H)⁺.

### Example 188

### 4-Isopropyl-6-{4-[3-(isopropyl-propyl-amino)-4-methoxy-phenyl]-5-mercapto-4H-[1,2,4]triazol-3-yl}-benzene-1,3-diol

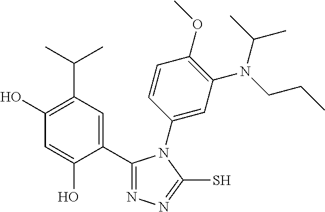 [SMILES: CCCN(c1cc(-n2c(S)nnc2-c2cc(C(C)C)c(O)cc2O)ccc1OC)C(C)C]

ESMS clcd for C₂₄H₃₂N₄O₃S: 456.22; Found: 457.3 (M+H)⁺.

### Example 189

### 4-Ethyl-6-{4-[3-(ethyl-isopropyl-amino)-4-methoxy-phenyl]-5-mercapto-4H-[1,2,4]triazol-3-yl}-benzene-1,3-diol

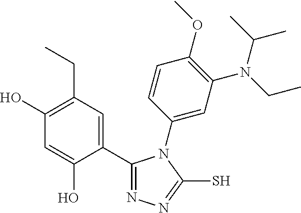 [SMILES: CCc1cc(-c2nnc(S)n2-c2ccc(OC)c(N(CC)C(C)C)c2)c(O)cc1O]

ESMS clcd for C₂₂H₂₈N₄O₃S: 428.19; Found: 429.3 (M+H)⁺.

### Example 190

### 4-Ethyl-6-[5-mercapto-4-(4-methoxy-3-morpholin-4-yl-phenyl)-4H-[1,2,4]triazol-3-yl]-benzene-1,3-diol

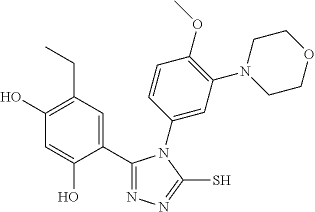 [SMILES: CCc1cc(-c2nnc(S)n2-c2ccc(OC)c(N3CCOCC3)c2)c(O)cc1O]

ESMS clcd for C₂₁H₂₄N₄O₄S: 428.15; Found: 429.2 (M+H)⁺.

### Example 191

### 4-Isopropyl-6-{5-mercapto-4-[4-methoxy-3-(methyl-propyl-amino)-phenyl]-4H-[1,2,4]triazol-3-yl}-benzene-1,3-diol

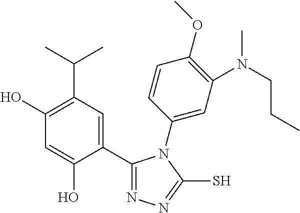 [SMILES: CCCN(C)c1cc(-n2c(S)nnc2-c2cc(C(C)C)c(O)cc2O)ccc1OC]

¹H NMR (300 MHz, DMSO-d₆) δ (ppm): 9.58 (s, 1H); 9.39 (s, 1H); 6.92-6.83 (m, 3H); 6.56(d, J=1.8 Hz, 1H); 6.23 (s, 1H); 3.74 (s, 3H); 3.0-2.93(m, 1H); 2.81(t, J=6.9 Hz, 2H); 2.48(s, 3H); 1.31-1.24 (m, 2H); 0.96 (d, J=6.9 Hz, 6H); 0.72 (t, J=7.2 Hz, 3H);

ESMS clcd for C₂₂H₂₈N₄O₃S: 428.19; Found: 429.2 (M+H)⁺.

### Example 192

### 4-{4-[3-(Ethyl-methyl-amino)-4-methoxy-phenyl]-5-mercapto-4H-[1,2,4]triazol-3-yl}-6-isopropyl-benzene-1,3-diol

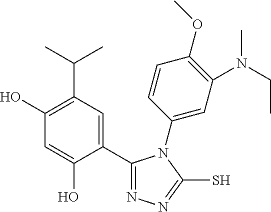 [SMILES: CCN(C)c1cc(-n2c(S)nnc2-c2cc(C(C)C)c(O)cc2O)ccc1OC]

¹H NMR (300 MHz, DMSO-d₆) δ (ppm): 9.58 (s, 1H); 9.40 (s, 1H); 6.92-6.85 (m, 3H); 6.58 (d, J=1.8 Hz, 1H); 6.24 (s, 1H); 3.76 (s, 3H); 3.02-2.90 (m, 3H); 2.49(s, 3H) 0.99 (d, J=6.9 Hz, 6H); 0.86 (t, J=7.2 Hz, 3H).

ESMS clcd for C₂₁H₂₆N₄O₃S: 414.17; Found: 415.1 (M+H)⁺.

### Example 193

### 4-Isopropyl-6-(5-mercapto-4-{4-methoxy-3-[methyl-(3-methyl-butyl)-amino]-phenyl}-4H-[1,2,4]triazol-3-yl)-benzene-1,3-diol

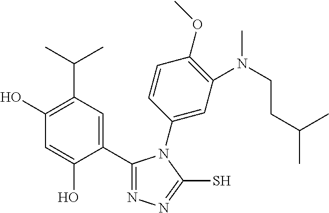 [SMILES: COc1ccc(-n2c(S)nnc2-c2cc(C(C)C)c(O)cc2O)cc1N(C)CCC(C)C]

ESMS clcd for C₂₄H₃₂N₄O₃S: 456.22; Found: 457.2 (M+H)⁺.

### Example 194

### 4-Isopropyl-6-{5-mercapto-4-[4-methoxy-3-(methyl-propyl-amino)-phenyl]-4H-[1,2,4]triazol-3-yl}-benzene-1,3-diol; Compound with Hydrogen Chloride

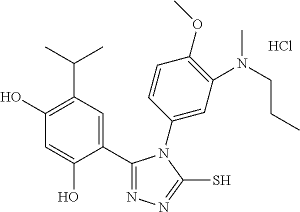 [SMILES: CCCN(C)c1cc(-n2c(S)nnc2-c2cc(C(C)C)c(O)cc2O)ccc1OC ; Cl]

ESMS clcd for C₂₂H₂₉ClN₄O₃S: 464.16; Found: 429.3 (M+H)⁺.

### Example 195

### 4-{4-[3-(Butyl-methyl-amino)-4-methoxy-phenyl]-5-mercapto-4H-[1,2,4]triazol-3-yl}-6-isopropyl-benzene-1,3-diol

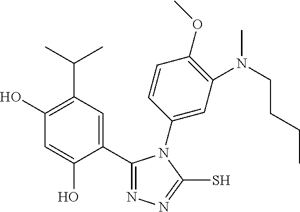 [SMILES: CCCCN(C)c1cc(-n2c(S)nnc2-c2cc(C(C)C)c(O)cc2O)ccc1OC]

ESMS clcd for C₂₃H₃₀N₄O₃S: 442.20; Found: 443.3 (M+H)⁺.

### Example 196

### 4-{4-[3-(Isobutyl-methyl-amino)-4-methoxy-phenyl]-5-mercapto-4H-[1,2,4]triazol-3-yl}-6-isopropyl-benzene-1,3-diol

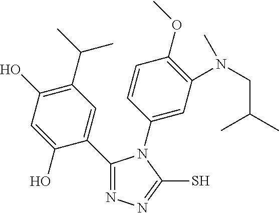 [SMILES: COc1ccc(-n2c(S)nnc2-c2cc(C(C)C)c(O)cc2O)cc1N(C)CC(C)C]

ESMS clcd for C₂₃H₃₀N₄O₃S: 442.20; Found: 443.1 (M+H)⁺.

### Example 197

### 4-(4-{3-[(2-Imidazol-1-yl-ethyl)-methyl-amino]-4-methoxy-phenyl}-5-mercapto-4H-[1,2,4]triazol-3-yl)-6-isopropyl-benzene-1,3-diol

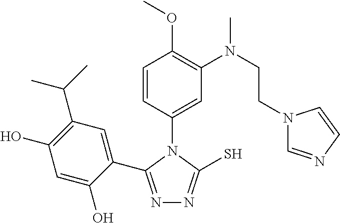 [SMILES: COc1ccc(-n2c(S)nnc2-c2cc(C(C)C)c(O)cc2O)cc1N(C)CCn1ccnc1]

ESMS clcd for C₂₄H₂₈N₆O₃S: 480.19; Found: 481.1 (M+H)⁺.

### Example 198

### 4-(4-(3-(1H-pyrrol-1-yl)phenyl)-5-mercapto-4H-1,2,4-triazol-3-yl)-6-ethylbenzene-1,3-diol

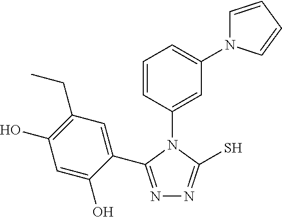 [SMILES: CCc1cc(-c2nnc(S)n2-c2cccc(-n3cccc3)c2)c(O)cc1O]

ESMS clcd for C₂₀H₁₈N₄O₂S: 378.12; Found: 379.1 (M+H)⁺.

### Example 199

### 4-(4-(4-(1H-pyrazol-1-yl)phenyl)-5-mercapto-4H-1,2,4-triazol-3-yl)-6-ethylbenzene-1,3-diol

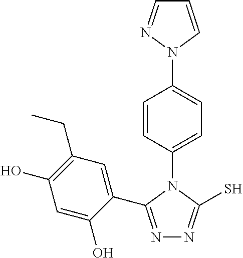 [SMILES: CCc1cc(-c2nnc(S)n2-c2ccc(-n3cccn3)cc2)c(O)cc1O]

ESMS clcd for C₁₉H₁₇N₅O₂S: 379.11; Found: 380.1 (M+H)⁺.

### Example 200

### 4-(4-(3-(dimethylamino)-4-(methylthio)phenyl)-5-mercapto-4H-1,2,4-triazol-3-yl)-6-isopropylbenzene-1,3-diol

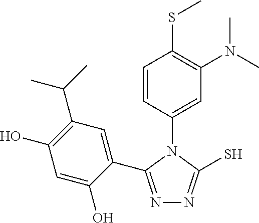 [SMILES: CSc1ccc(-n2c(S)nnc2-c2cc(C(C)C)c(O)cc2O)cc1N(C)C]

ESMS clcd for C₂₀H₂₄N₄O₂S₂: 416.13; Found: 417.1 (M+H)⁺.

### Example 201

### 4-isopropyl-6-(5-mercapto-4-(4-methoxy-3-(propylamino)phenyl)-4H-1,2,4-triazol-3-yl)benzene-1,3-diol

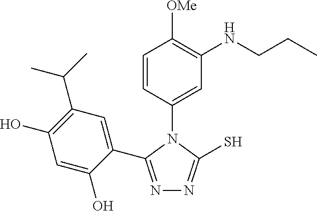 [SMILES: CCCNc1cc(-n2c(S)nnc2-c2cc(C(C)C)c(O)cc2O)ccc1OC]

ESMS clcd for C₂₁H₂₆N₄O₃S: 414.17; Found: 415.1 (M+H)⁺.

### Example 202

### 4-[4-(4-Amino-3-hydroxy-phenyl)-5-mercapto-4H-[1,2,4]triazol-3-yl]-6-ethyl-benzene-1,3-diol

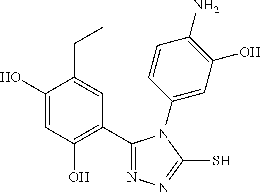 [SMILES: CCc1cc(-c2nnc(S)n2-c2ccc(N)c(O)c2)c(O)cc1O]

ESMS clcd for C₁₆H₁₆N₄O₃S: 344.09; Found: 345.1 (M+H)⁺.

### Example 203

### 4-ethyl-6-(4-(3-hydroxy-4-(methylamino)phenyl)-5-mercapto-4H-1,2,4-triazol-3-yl)benzene-1,3-diol

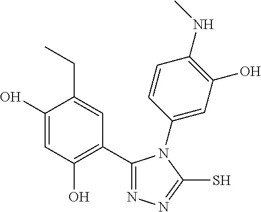 [SMILES: CCc1cc(-c2nnc(S)n2-c2ccc(NC)c(O)c2)c(O)cc1O]

ESMS clcd for C₁₇H₁₈N₄O₃S: 358.11; Found: 359.1 (M+H)⁺

### Example 204

### 4-(4-(3-aminophenyl)-5-mercapto-4H-1,2,4-triazol-3-yl)-6-ethylbenzene-1,3-diol

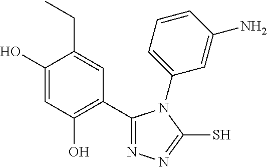 [SMILES: CCc1cc(-c2nnc(S)n2-c2cccc(N)c2)c(O)cc1O]

ESMS clcd for C₁₆H₁₆N₄O₂S: 328.10; Found: 329.1 (M+H)⁺.

### Example 205

### 4-[4-(4-Dimethylamino-3-methyl-phenyl)-5-mercapto-4H-[1,2,4]triazol-3-yl]-6-ethyl-benzene-1,3-diol

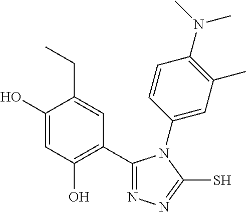 [SMILES: CCc1cc(-c2nnc(S)n2-c2ccc(N(C)C)c(C)c2)c(O)cc1O]

ESMS clcd for C₁₉H₂₃N₄O₂S: 371.1; Found: 371.1 (M+H)⁺.

### Example 206

### 4-[4-(3-Imidazol-1-yl-phenyl)-5-mercapto-4H-[1,2,4]triazol-3-yl]-6-isopropyl-benzene-1,3-diol

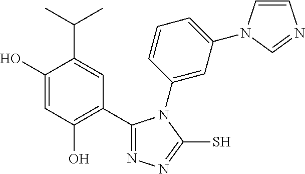 [SMILES: CC(C)c1cc(-c2nnc(S)n2-c2cccc(-n3ccnc3)c2)c(O)cc1O]

ESMS clcd. for C₂₀H₂₀N₅O₂S: 394.1; Found: 394.1 (M+H)⁺.

### Example 207

### 4-[4-(3-Imidazol-1-yl-phenyl)-5-mercapto-4H-[1,2,4]triazol-3-yl]-6-isopropyl-benzene-1,3-diol

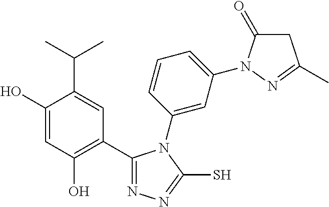 [SMILES: CC1=NN(c2cccc(-n3c(S)nnc3-c3cc(C(C)C)c(O)cc3O)c2)C(=O)C1]

### 2-{3-[3-(2,4-Dihydroxy-5-isopropyl-phenyl)-5-mercapto-[1,2,4]triazol-4-yl]-phenyl}-5-methyl-2,4-dihydro-pyrazol-3-one

¹H NMR (300 MHz, DMSO-d₆) δ (ppm): 9.63 (br s, 1H); 7.70-7.80 (m, 2H); 7.37-7.43 (m, 1H); 6.99-7.02 (m, 1H); 6.91 (s, 1H); 6.25 (s, 1H); 5.35 (s, 1H); 3.70 (s, 2H); 2.96 (hept, J=6.9 Hz, 1H); 2.09 (s, 3H); 0.99 (d, J=6.9 Hz, 6H);

ESMS clcd. for C₂₁H₂₂N₅O₃S: 424.1; Found: 424.1 (M+H)⁺.

### Example 208

### 3-(2,4-dihydroxy-5-isopropyl-phenyl)-4-(1-methyl-indol-5-yl)-5-hydroxy-[1,2,4]triazole (Compound 226)

### Step 1: Synthesis of phenyl 1-methyl-1H-indol-5-ylcarbamate c:

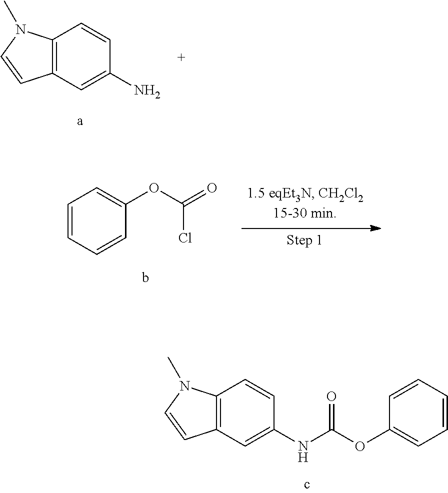 [SMILES: Cn1ccc2cc(N)ccc21 ; O=C(Cl)Oc1ccccc1 ; Cn1ccc2cc(NC(=O)Oc3ccccc3)ccc21]

To a solution of 5.62 g (35.91 mmols) of phenylchloroformate b in 25 mL of dichloromethane at 0° C. was added, a solution of 5.0 g (34.20 mmols) of indoleamine a in 25 mL of dichloromethane drop wise (20 min) at 0° C. The resultant mixture was then stirred for 10 min at 0° C. and a solution of 6 mL (42.75 mmols) of triethylamine in 10 mL of dichloromethane was added drop wise (15 min) at 0° C. and stirred for 5 min. To the mixture was then added 50 mL of water and organic layer separated. The aqueous layer was then extracted with 20 mL of dichloromethane and organic layers combined and dried over Na₂SO₄. The solution was then passed through a pad of silica gel, eluted with additional 50 mL of 3:1 hexane:ethylacetate and concentrated. The crude product was then crystallized with 4:1 hexane:ethyl acetate to obtain 7.8 g (85.7%, 99.5% pure, I crop) and 0.78 g (8.5%, 98% pure, II crop) with a combined yield of 94% product.

### Step 2: Synthesis of N-(1-methyl-1H-indol-5-yl)hydrazinecarboxamide e

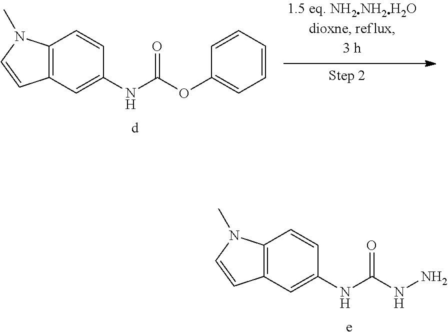 [SMILES: Cn1ccc2cc(NC(=O)Oc3ccccc3)ccc21 ; Cn1ccc2cc(NC(=O)NN)ccc21]

To a stirred suspension of 35.0 g (0.131 mols) of the carbamate d in 120 mL of 1,4-dioxane was added 32 mL (0.657 mols) of hydrazine hydrate and the resultant mixture was refluxed for 3 h and concentrated. To the crude mixture was added approx. 250 mL of cold water and the resultant light brown precipitate was filtered and vacuum dried. The crude solid was again treated with 150 mL of ether and stirred for 1 h and filtered. Drying in vacuum afforded 21.6 g (80%) of e as grey solid.

### Step 3: Synthesis of 3-(2,4-Bis-benzyloxy-5-isopropyl)benzylideneamino-1-(1-Methyl-1H-indol-′-yl)-urea g

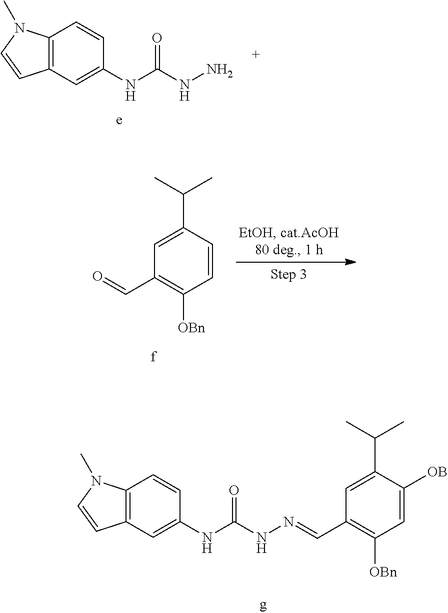

To a suspension of 23.0 g (63.8 mmols) of the aldehyde f in 150 mL of ethanol was added 2 mL of acetic acid (AcOH) and stirred. To the resultant mixture was added 13.0 g (63.8 mmols) of e portion wise (solid, 10 min) at room temperature and the resultant mixture was heated at 80° C. for 1 h. During this time, stirring was difficult due to precipitate formation, therefore an additional 50 mL of ethanol was added. The mixture was cooled to room temperature and filtered the precipitate, washed with 50 mL of cold ethanol and 100 mL of ether and dried. Vacuum drying afforded 33.7 g (97%) of the product g as off-white solid.

ESMS calcd. for C34H34N4O3 (M+H)⁺: 546.26; Found: 547.3

### Step 4: Synthesis of 5-(2,4-Bis-benzyloxy-5-isopropylphenyl)-4-(1-methyl-1H-indol-5-yl)-4H-[1,2,4]triazol-3-ol h

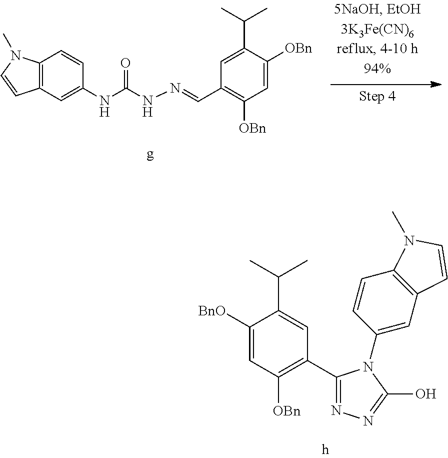 [SMILES: CC(C)c1cc(/C=N/NC(=O)Nc2ccc3c(ccn3C)c2)c(OCc2ccccc2)cc1OCc1ccccc1 ; CC(C)c1cc(-c2nnc(O)n2-c2ccc3c(ccn3C)c2)c(OCc2ccccc2)cc1OCc1ccccc1]

To a stirred suspension of 32.5 g (59.49 mmols) of g in 200 mL of ethanol was added 7.14 g (0.178 mmols) of NaOH and stirred. To the resultant mixture, was added 39.17 g (0.118 mmols) of K₃Fe(CN)₆ at once and the resultant mixture was stirred at reflux temperature (100° C. oil bath external temperature) for 8 h (till the reaction is complete, checked by TLC). The mixture was cooled and the inorganics were filtered off. The residues were thoroughly washed with ethanol (EtOH) (50 mL) and a 1:1 mixture of ethyl acetate:methanol (150 mL) and filtrates were collected. The combined filtrates were concentrated and crude mixture was dissolved in approx 200 mL of water (still a suspension). The mixture was then acidified with concentrated HCl until pH 2-3 was reached. The resultant precipitate was filtered, washed thoroughly with water and dried. The crude product was then taken up in 90 mL of methanol (MeOH) and stirred at 50° C. for 30 min and the solid obtained was filtered washed with cold MeOH and dried to obtain 27 g of the off white solid. From the mother liquor another 3.8 g of the grey solid h was isolated. Total yield=30.8 g (95%).

ESMS calcd. for C34H32N4O3 (M+H)⁺: 544.25; Found: 545.3.

### Step 5: Synthesis of 3-(2,4-dihydroxy-5-isopropyl-phenyl)-4-(1-methyl-indol-5-yl)-5-hydroxy-[1,2,4]triazole (Compound 226)

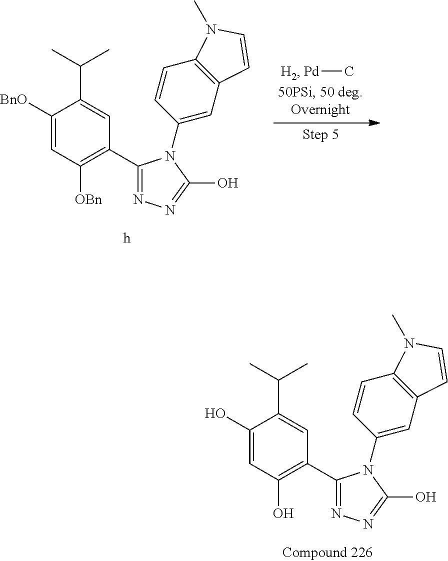

Compound h (1 g, 1.84 mmol, 1.0 eq) was hydrogenated by balloon pressure of hydrogen at room temperature in 8 mL of THF and 4 mL of methanol for 6 h. The reaction mixture was filtered through Celite, and washed with tetrahydrofuran (THF) and EtOAc. After removal solvents, the reaction mixture was dissolved in 20 mL of 1 N NaOH solution, and acidified with 1N HCl until pH 3˜4 was reached. The white precipitate thus obtained was filtered, washed with water and dried using the vacuum oven to produce off-white solid of 3-(2,4-dihydroxy-5-isopropyl-phenyl)-4-(1-methyl-indol-5-yl)-5-hydroxy-[1,2,4]triazole (Compound 226) (0.638 g, 1.75 mmol, 95%).

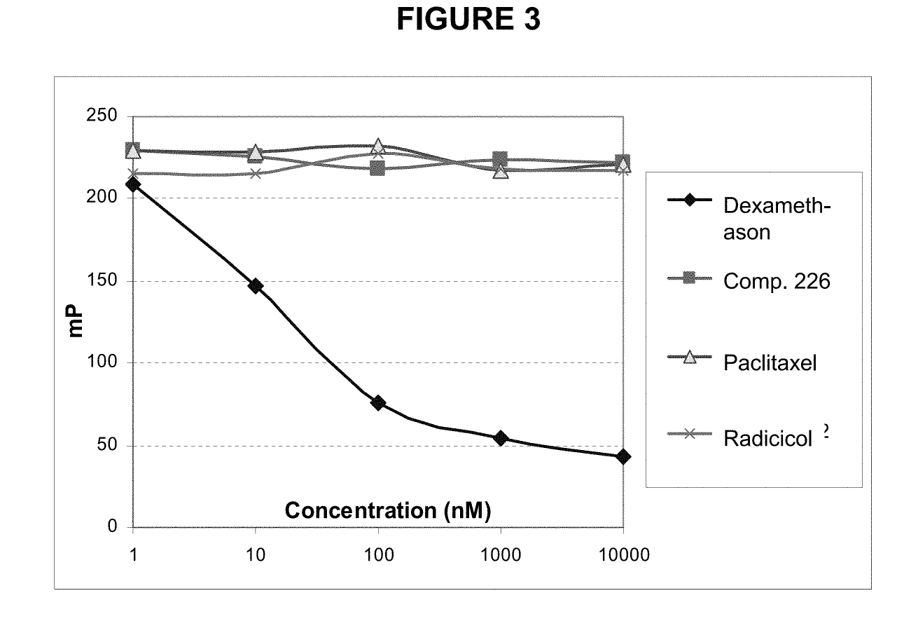

1H-NMR (DMSO, 300 MHz) of Compound 226 is shown in FIG. 3. δ 11.86 (s, 1H), 9.53 (s, 1H), 9.41 (s, 1H), 9.40-9.36 (m, 3H), 6.91 (dd, J=2.1, 9 Hz, 1H), 6.77 (s, 1H), 6.40 (d, J=3 Hz, 1H), 6.20 (s, 1H), 3.77 (s, 3H), 2.90 (hept., J=6.9 Hz, 1H), 0.87 (d, J=6.9 Hz, 6H).

ESMS calcd. for C20H20N4O3 (M+H)⁺: 364.15; Found: 365.2

### Example 209

### Synthesis of 2,4-Dihydroxy-5-Isopropyl-Benzaldehyde j and 2,4-Bis-Benzyloxy-5-Isopropyl-Benzaldehyde f

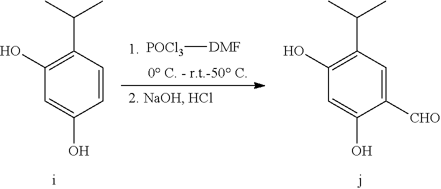

To 70 mL of cold and stirred DMF (ice-bath) was added 31 mL (0.328 mols, 2.5 eq. of reagent) of POCl₃ drop wise over 15 min. The resultant mixture was stirred at ice-bath temperature (0-5° C.) for 30 min. To the mixture was then added 20 g (0.13 mols) of i in 40 mL of anhydrous DMF drop wise at ice-bath temperature (0-5° C.) over 25 min. The resultant viscous mixture was stirred at room temperature for 1 h and at 50° C. for 1 h.

The mixture was then poured cautiously to a cold solution of 63 g (12 eq.) of NaOH in 400 mL of water (over 10 min) with vigorous stirring. A red colored solution was then obtained. The mixture was then heated at 70° C. for 15 min and then cooled. It was then acidified with ice-bath cooling with concentrated HCl until pH 2-3 was reached. The solution turned yellow-orange with same colored precipitate formed. The mixture was stirred further (over weekend; alternatively, anywhere between 15 min. to 1 h stirring should be fine) and filtered. The orange colored precipitate was washed successively with water and vacuum dried at 50° C. to obtain 17.25 g (73%) of orange-light brown powder.

The hydroxyl groups of Compound j were protected with benzyl groups by heating Compound j with benzyl chloride in a solution of K₂CO₃ in acetonitrile as shown in the following scheme:

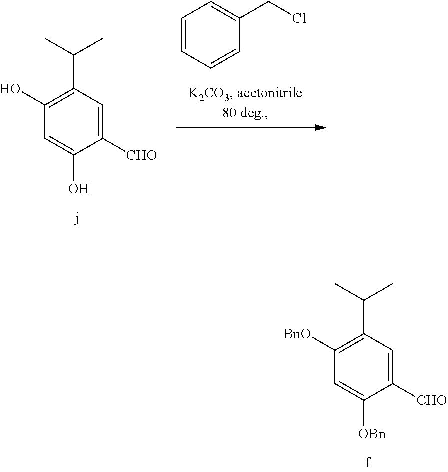 [SMILES: CC(C)c1cc(C=O)c(O)cc1O ; ClCc1ccccc1 ; CC(C)c1cc(C=O)c(OCc2ccccc2)cc1OCc1ccccc1]

### Example 210

### Inhibition of Hsp90

Hsp90 protein was obtained from Stressgen (Cat #SPP-770). Assay buffer: 100 mM Tris-HCl, Ph 7.4, 20 mM KCl, 6 mM MgCl₂. Malachite green (0.0812% w/v) (M9636) and polyvinyl alcohol USP (2.32% w/v) (P1097) were obtained from Sigma. A Malachite Green Assay (see Methods Mol Med, 2003, 85:149 for method details) was used for examination of ATPase activity of Hsp90 protein. Briefly, Hsp90 protein in assay buffer (100 mM Tris-HCl, Ph 7.4, 20 mM KCl, 6 mM MgCl₂) was mixed with ATP alone (negative control) or in the presence of Geldanamycin (a positive control) or Compound 108 in a 96-well plate. Malachite green reagent was added to the reaction. The mixtures were incubated at 37° C. for 4 hours and sodium citrate buffer (34% w/v sodium citrate) was added to the reaction. The plate was read by an ELISA reader with an absorbance at 620 nm.

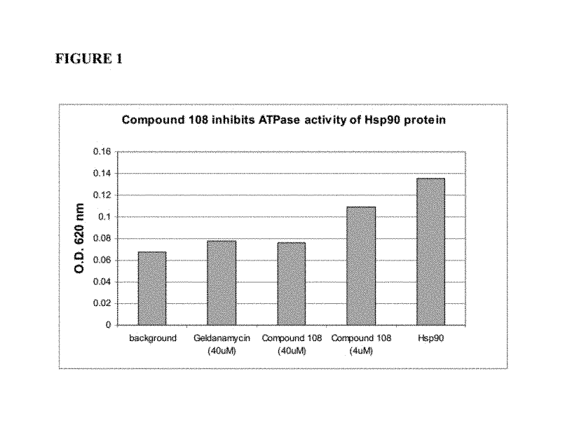

As can be seen in FIG. 1, 40 μM of geldanamycin, a natural product known to inhibit Hsp90 activity, the ATPase activity of Hsp90 was only slightly higher than background. 40 μM Compound 108 showed an even greater inhibition of ATPase activity of Hsp90 than geldanamycin, and even at 4 μM Compound 108 showed significant inhibition of ATPase activity of Hsp90 protein.

### Example 211

### Degradation of Her2 via Inhibition of Hsp90 Activity

A. Cells and Cell Culture

Human high-Her2 breast carcinoma BT474 (HTB-20), SK-BR-3 (HTB-30) and MCF-7 breast carcinoma (HTB-22) from American Type Culture Collection, VA, USA were grown in Dulbecco's modified Eagle's medium with 4 mM L-glutamine and antibiotics (100 IU/ml penicillin and 100 ug/ml streptomycine; GibcoBRL). To obtain exponential cell growth, cells were trypsinized, counted and seeded at a cell density of 0.5×10⁶ cells/ml regularly, every 3 days. All experiments were performed on day 1 after cell passage.
B. Degradation of Her2 in Cells After Treatment with a Compound of the Invention
   1. Method 1

BT-474 cells were treated with 0.5 μM, 2 μM, or 5 μM of 17AAG (a positive control) or 0.5 μM, 2 μM, or 5 μM of Compound 108 or Compound 49 overnight in DMEM medium. After treatment, each cytoplasmic sample was prepared from 1×10⁶ cells by incubation of cell lysis buffer (#9803, cell Signaling Technology) on ice for 10 minutes. The resulting supernatant used as the cytosol fractions were dissolved with sample buffer for SDS-PAGE and run on a SDS-PAGE gel, blotted onto a nitrocellulose membrane by using semi-dry transfer. Non-specific binding to nitrocellulose was blocked with 5% skim milk in TBS with 0.5% Tween at room temperature for 1 hour, then probed with anti-Her2/ErB2 mAb (rabbit IgG, #2242, Cell Signaling) and anti-Tubulin (T9026, Sigma) as housekeeping control protein. HRP-conjugated goat anti-rabbit IgG (H+L) and HRP-conjugated horse anti-mouse IgG (H+L) were used as secondary Ab (#7074, #7076, Cell Signaling) and LumiGLO reagent, 20× Peroxide (#7003, Cell Signaling) was used for visualization.

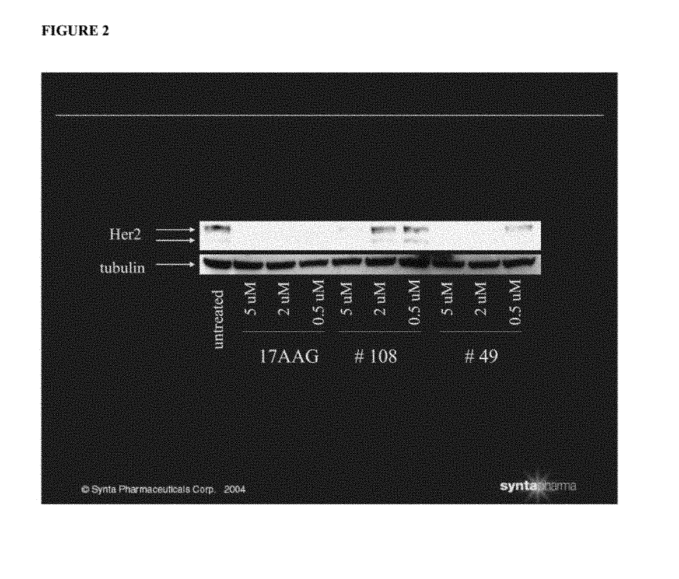

As can be seen from FIG. 2, Her2, an Hsp90 client protein, is almost completely degraded when cells are treated with 5 μM of Compound 108 and partially degradated when cells are treated with 2 μM and 0.5 μM of Compound 108. Compound 49 which is even more active than Compound 108 causes complete degradation of Her2 when cells are treated with 2 μM and 5 μM and causes partial degradated when cells are treated with 0.5 μM 17AAG is a known Hsp90 inhibitor and is used as a positive control.
2. Method 2

MV-4-11 cells (20,000 cells/well) are cultured in 96-well plates and maintained at 37° C. for several hours. The cells are treated with a compound of the invention or 17AAG (a positive control) at various concentrations and incubated at 37° C. for 72 hours. Cell survival is measured with Cell Counting Kit-8 (Dojindo Laboratories, Cat. #CK04).

TABLE 8
IC₅₀ range of compounds of the invention for
inhibition of Her2 degradation

| IC₅₀ Range | Compound Number |
|---|---|
| <3 μM | 8, 13, 39, 49, 63, 76, 77, 79, 87, 88, 95, 96, 100, 103, |
| | 177, 178, 185, 188, 189, 247, 248, 249, 250, 251, 252, |
| | 259 |
| 3 μM to 10 μM | 2, 5, 6, 7, 9, 14, 27, 28, 34, 36, 38, 70, 93, 97, |
| | 108, 122, 183, 184 |
| 10 μM to 100 μM | 21, 22, 30, 51, 59, 60, 61, 62, 94, 98, 99, 102, 104, |
| | 123, 181, 182, 186, 187, 348 |

C. Fluorescent Staining of Her2 on the Surface of Cells Treated with a Compound of the Invention

After treatment with a compound of the invention, cells were washed twice with 1× PBS/1% FBS, and then stained with anti-Her2-FITC (#340553, BD) for 30 min at 4° C. Cells were then washed three times in FACS buffer before the fixation in 0.5 ml 1% paraformadehydrede. Data was acquired on a FACSCalibur system. Isotype-matched controls were used to establish the non-specific staining of samples and to set the fluorescent markers. A total 10,000 events were recorded from each sample. Data were analysed by using CellQuest software (BD Biosciences). The IC₅₀ range for Hsp90 inhibition by compounds of the invention are listed below in Table 8.
D. Apoptosis Analysis

After treatment with the compounds of the invention, cells were washed once with 1× PBS/1% FBS, and then stained in binding buffer with FITC-conjugated Annexin V and Propidium iodide (PI) (all obtained from BD Biosciences) for 30 min at 4° C. Flow cytometric analysis was performed with FACSCalibur (BD Biosciences) and a total 10,000 events were recorded from each sample. Data were analyzed by using CellQuest software (BD Biosciences). The relative fluorescence was calculated after subtraction of the fluorescence of control.

### Example 112

The compounds of the invention do not bind directly to the glucocorticoid receptor.

To determine whether the compounds of the invention bind directly to the glucocorticoid receptor, GR competitive assay was performed using a kit from Invitrogen (Cat #P2816). GR (human recombinant) was added to a fluorescent glucocorticoid (Fluormone™ GS1) ligand with a test compound at various concentrations (a serial dilution of the test compound was done on a 96-well plate) and incubated for 2-4 hours in the dark. The plates were then read at 485 nm excitation and 535 nm emission interference filters on PE Victor3 to determine the fluorescence polarization value (mP). Test compounds that were effective competitors prevented the binding of Fluormone™ GS1 to the glucocorticoid receptor and resulted in a decrease of the polarization value. The shift in polarization value in the presence of a test compound was used to determine relative affinity of test compounds for glucocorticoid receptors. The buffer plus Fluormone™ GS1 and glucocorticoid receptor in the absent of s test compound was used as negative control presenting 0% competition. Dexamethasone (1 mM) in the buffer plus Fluormone™ GS1 and glucocorticoid receptor was used as positive control representing 100% competition. The IC₅₀ was calculated based on the inhibition rate of binding activity of the testing compound.

We tested Dexamethasome (positive control), Compound 226, Compound 264, Radicical, 17-DMAG, and Paclitaxel (negative control) in the above described competitive binding assay using recommended protocol. The IC₅₀ of Dexamethasone was 2 nM to 10 nM calculated with the XLfit, which was within the range as reported. There was no binding activity observed on the other compounds tested up to 10 uM concentration (see FIG. 3).

### Example 113

The compounds of the invention inhibit the production of inflammatory cytokines in human PBMCs.

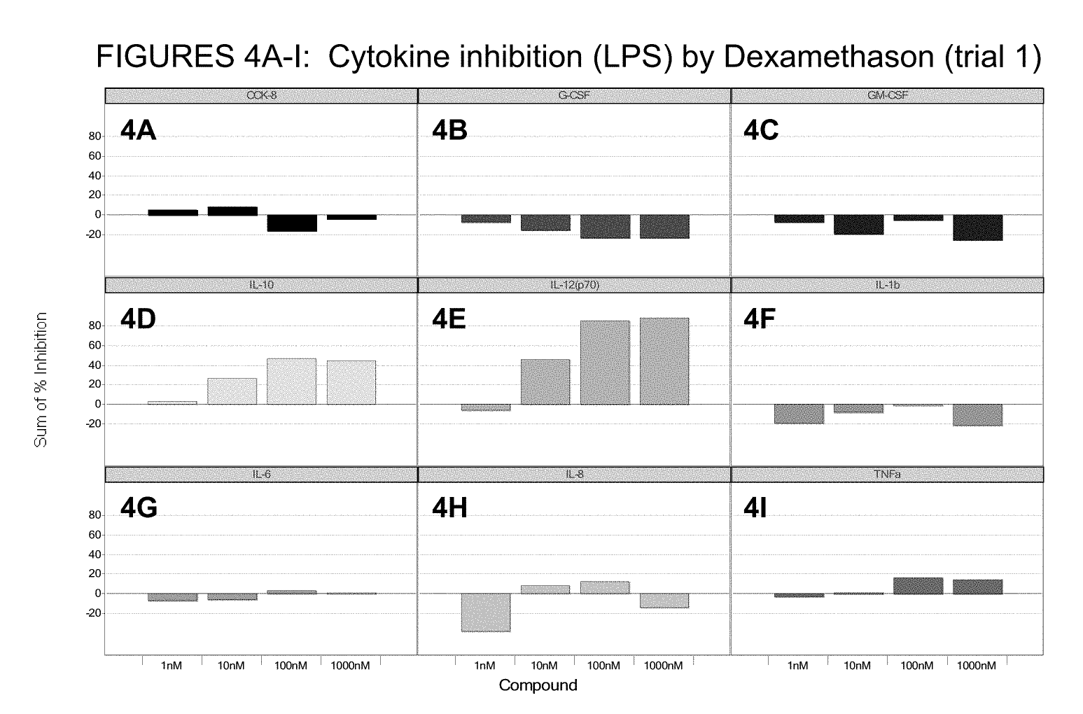

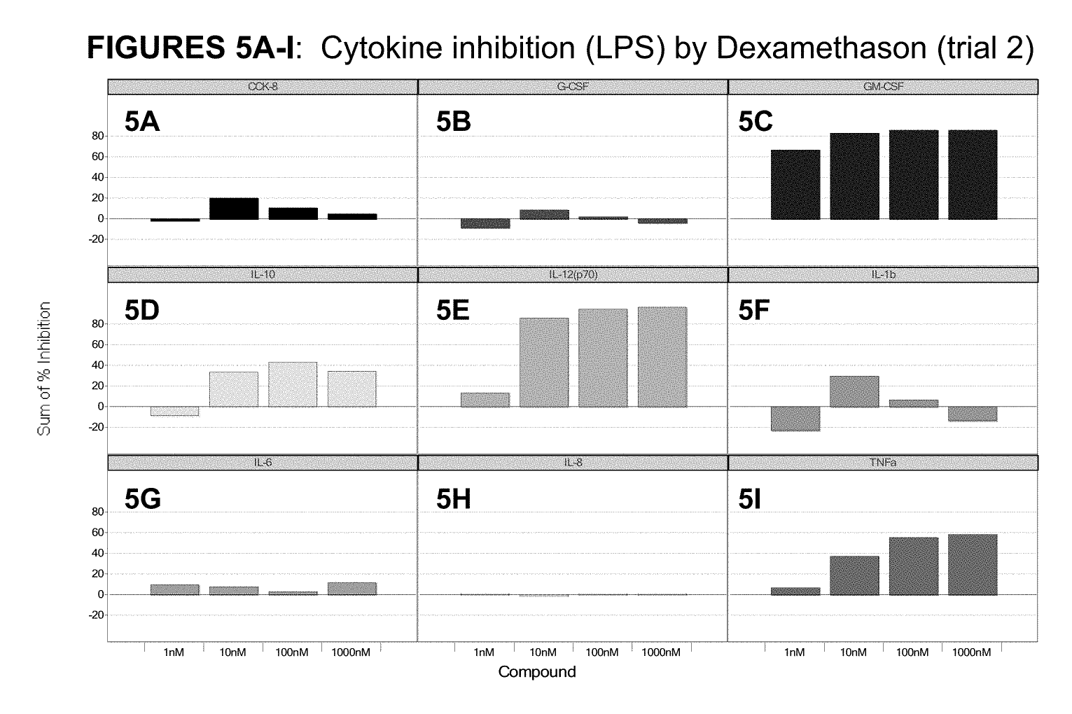

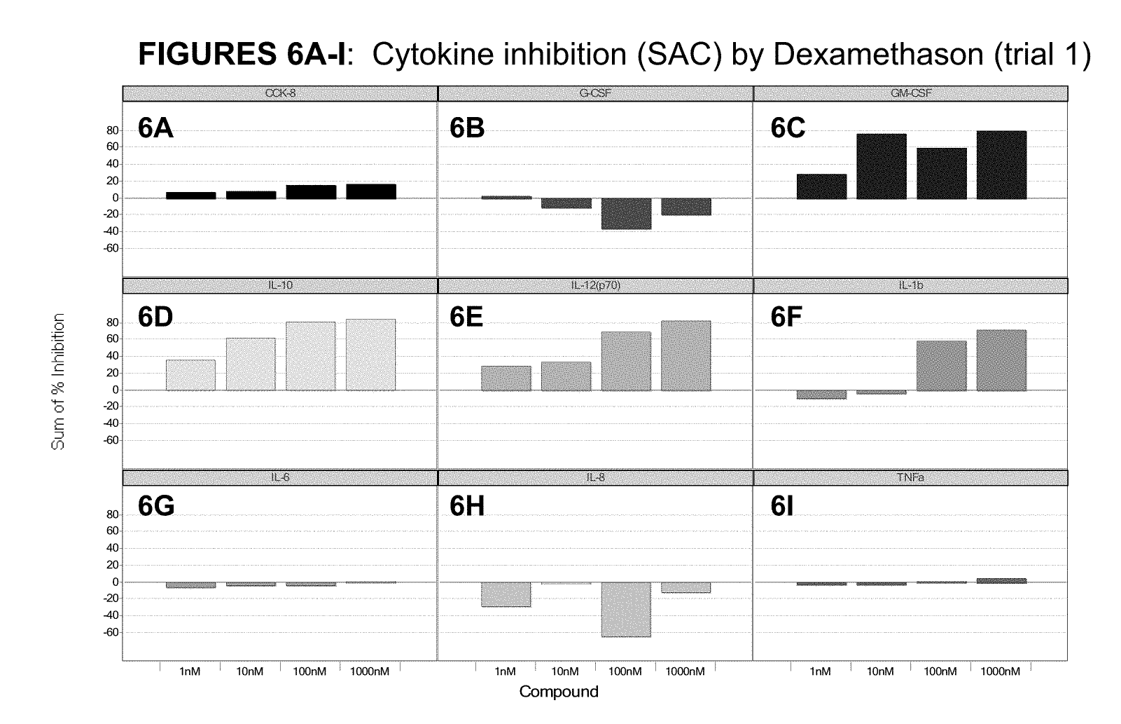

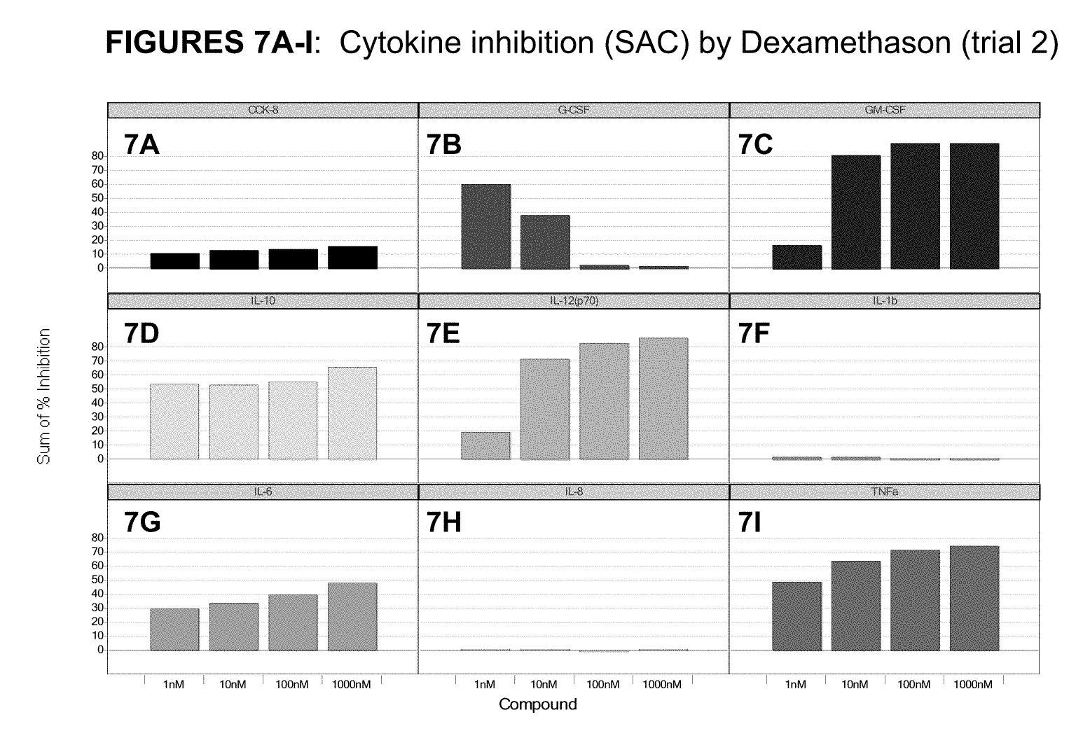

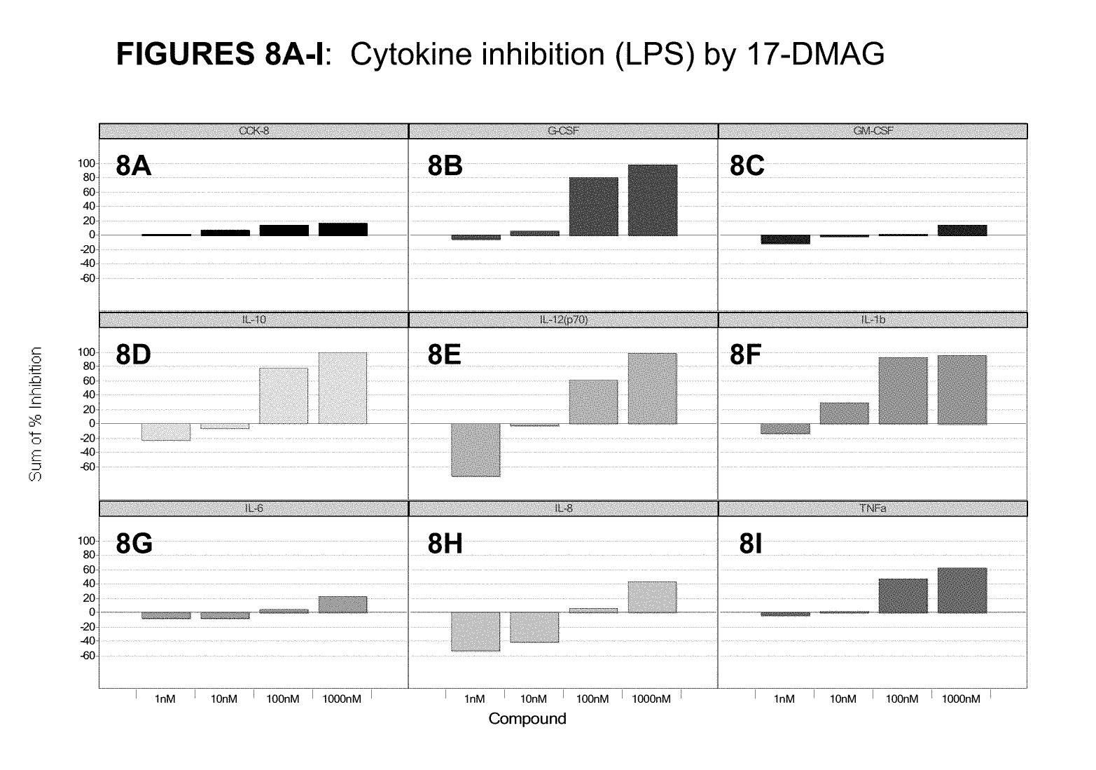

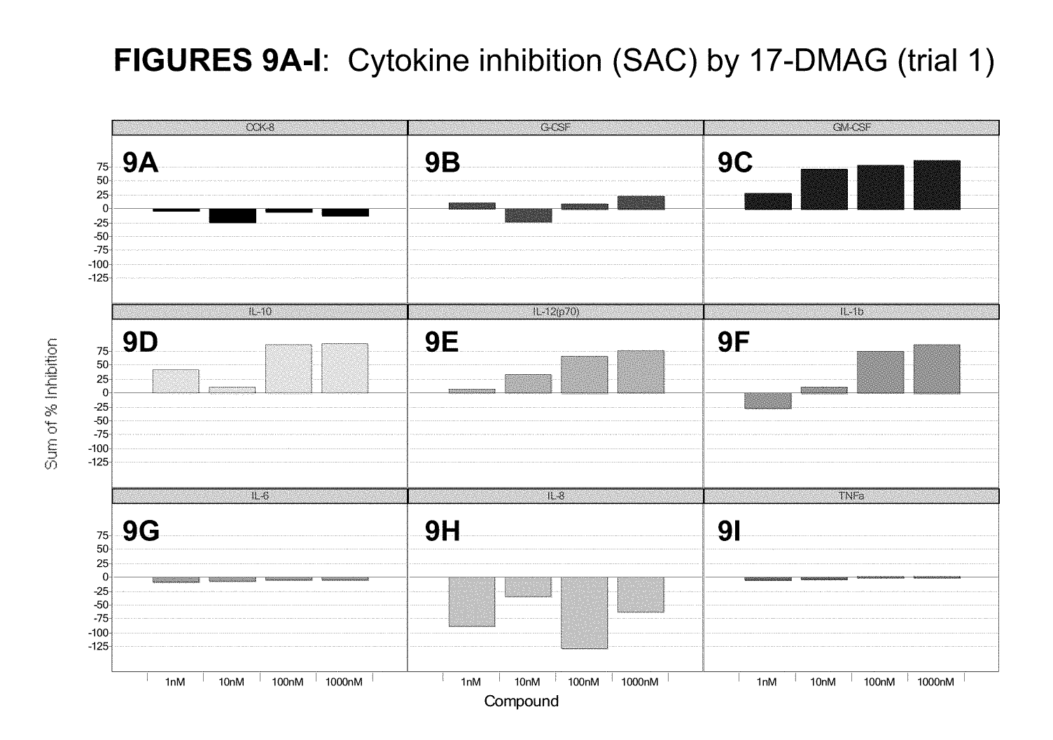

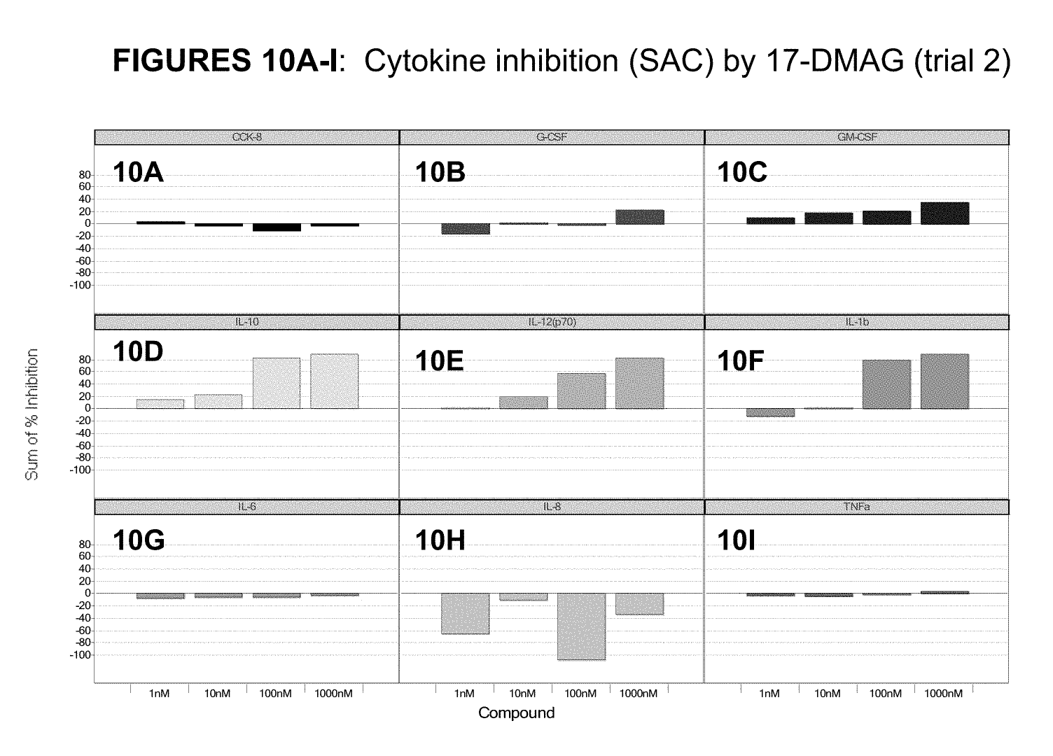

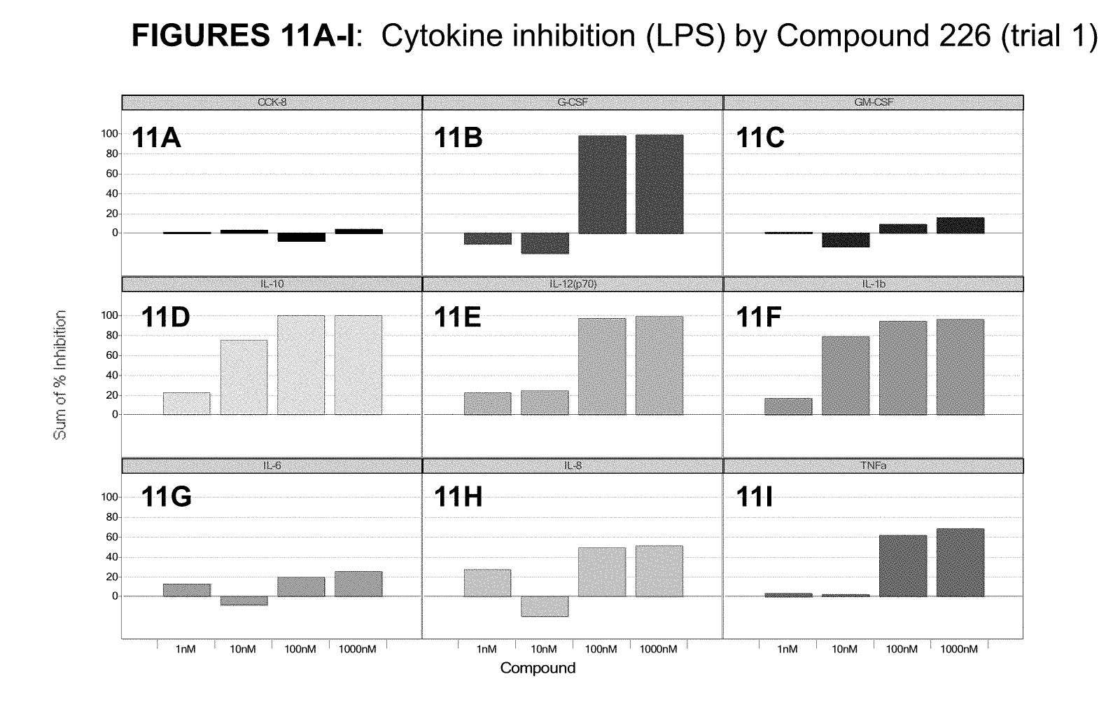

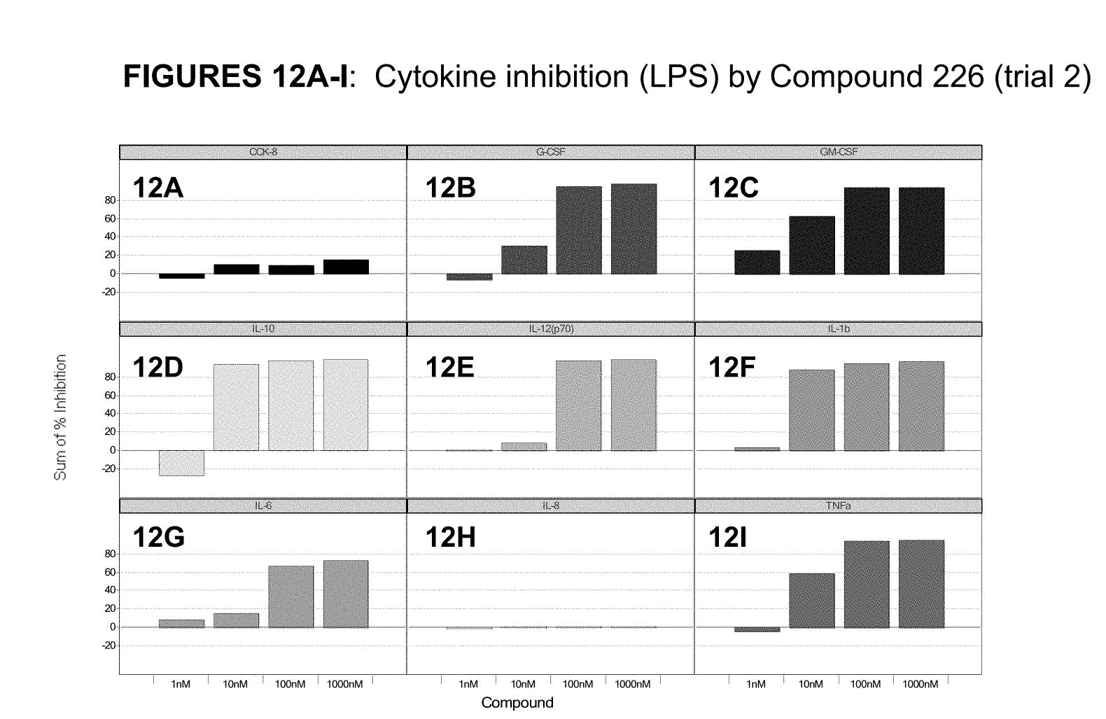

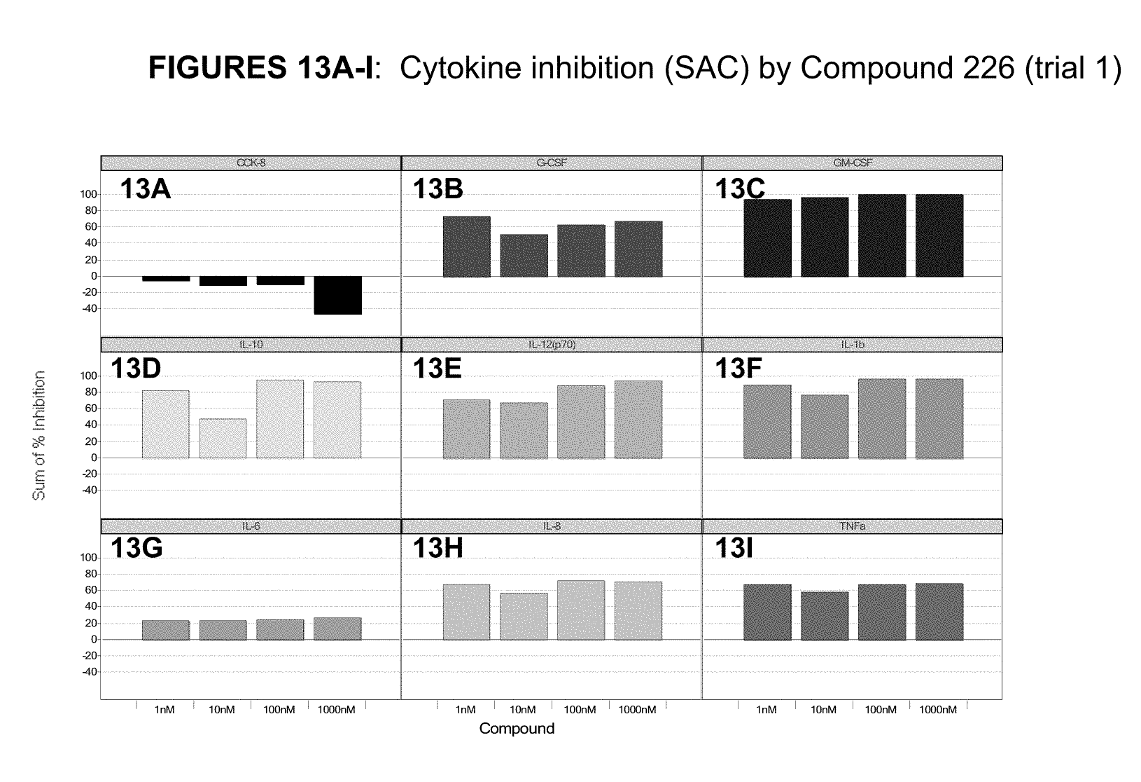

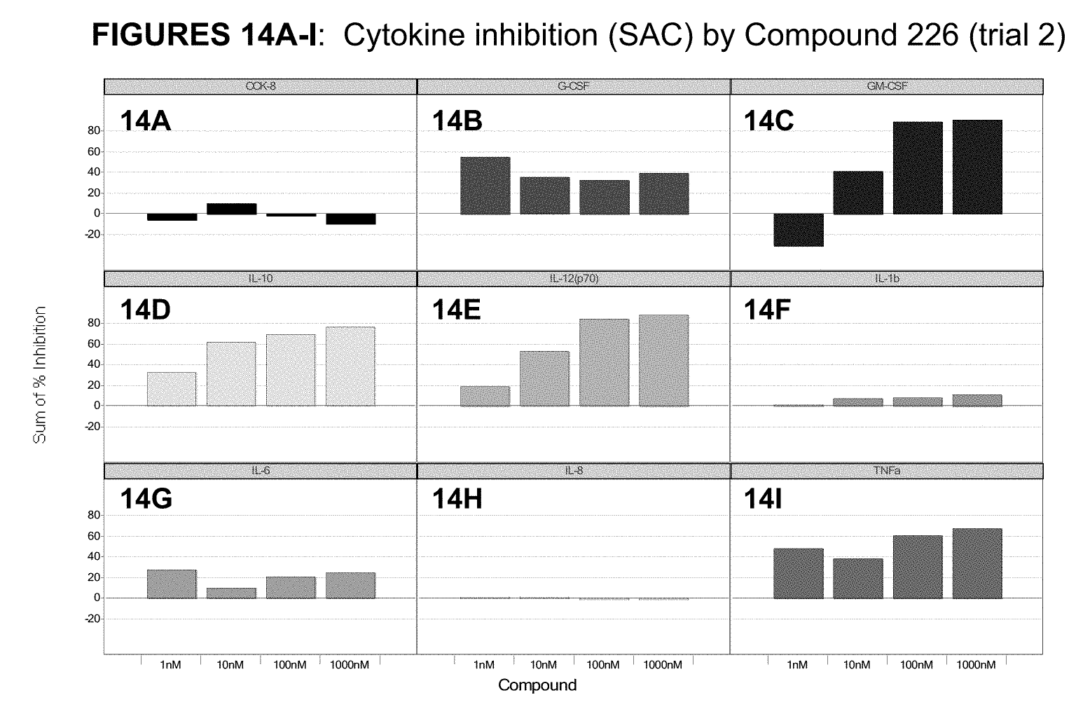

Human PBMC were isolated using Ficoll 400 and diatrizoate sodium (density 1.077 g/ml) solution and purified with RosetteSep (StemCell Technologies). The PBMCs were primed with human IFN-γ (800 U/ml, Pierce Biotechnology #R-IFNG-50), seeded at 0.5×10⁶/100 μL/well in 96-well U-bottom plate with culture medium (RPMI 1640, 10% FBS, 1% Pen/Strep), and incubated in 37° C. for overnight. The cells were then stimulated with 1 μg/ml of LPS (Lipopolysaccharide, Sigma #L2654-1MG) or 0.025% of SAC (Staphylococcus Aureus Cowan, Calbiochem-Novabiochem Corp. #507858), and treated with a test compound at different concentrations with final DMSO concentration less than 0.5% for 16-18 hrs. About 180 ul/well of supernatant was collected and measured using ELISA kit or Bio-plex (Bio-Rad) to determine the levels of cytokine production. The cell survival was determined using Cell Counting Kit-8 (Dojindo Molecular Technologies, Inc.). The compounds tested in this assay were dexamethasone (positive control; see FIGS. 4A-7I), 17-DMAG (a known Hsp90 inhibitor; see FIGS. 8A-10I), and Compound 226 (see FIGS. 11A-14I). Cholecystokinin-8 (CCK-8) is a neurotransmitter and is included here as a negative control. As can be seen from FIGS. 4A-14I, Compound 226 more broadly inhibits the production of proinflammatory cytokines than dexamethasone or 17-DMAG.

### Example 114

Compounds of the invention suppress glucocorticoid receptor levels in rat and human PBMCs.

### Cell Preparation:

The whole blood samples from healthy human volunteer and male SD rat were collected and the PBMCs were isolated immediately as follows. 5 ml of whole blood was diluted with an equal volume of sterile 1× PBS. The diluted blood was overlayed carefully into a sterile centrifuge tube without disturbing the bottom layer that containing 5 ml of Ficoll-paque plus density gradient solution. The layered blood was centrifuged at 1500×g for 30 minutes at room temperature. The middle thin layer containing PBMCs was carefully removed, transferred to another sterile centrifuge tube, and washed twice with PBS to remove Percoll. Isolated rat and human PBMCs were cultured in 10% fetal bovine serum/DMEM.

### Treatment:

The rat and human PBMCs were treated with DMSO (control), Compound 226, Compound 287, or 17-DMAG at concentrations of 0, 1, 5, 25, or 100 nM (in DMSO) for 16 hours. The cells were then collected and rinsed in ice-cold PBS and stored in liquid nitrogen until further analysis.

### Immunoblot

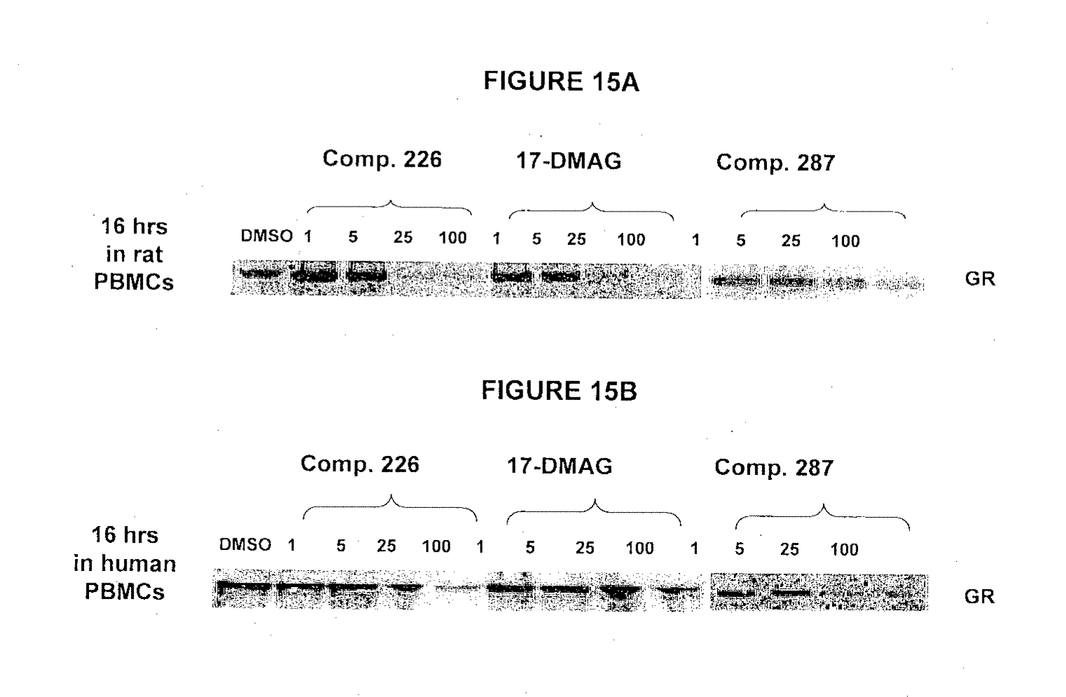

PBMC were prepared in Western lysis buffer (10 mmol/L HEPES, 42 mmol/L KCl, 5 mmol/L MgCl₂, 0.1 mmol/L EDTA, 0.1 mmol/L EGTA, 1 mmol/L DTT, 1% Triton X-100, freshly supplemented with 1× protease inhibitor cocktail from Pierce, Rockford, Ill.). Lysate protein concentrations were quantified by bicinchoninic acid assay (Pierce) and normalized. Equal amounts of protein were loaded onto 10% NuPAGE Bis-Tris Gels (Invitrogen) and subsequently transferred onto polyvinylidene difluoride membranes. The membranes were blocked in 5% milk in TBST. Primary antibody of glucocorticod receptor from Santa Cruz Biotechnology, Inc. was added and incubated at room temperature for 1 hour with shaking. The blots were washed extensively in TBST before secondary antibodies were added for overnight incubation at 4° C. with gentle shaking. The blots were again washed extensively and developed with SuperSignal West Femto substrate (Pierce). The immunoblot analysis was performed to measure the level of total GRs by Quantity One software from Bio-Rad. 17-DMAG, Compound 226 and Compound 287 decreased the expression of glucocorticoid receptor in a dose dependent manner in rat and human PBMCs (see FIGS. 15A and 15B).

### Example 115

Compounds of the invention suppress glucocorticoid receptor levels in human PBMCs and renal cells, as well as in several human cancer cell lines.

### Cell Preparation:

Normal human renal proximal tubule epithelial cells and tumor cell lines of MV-4-11, Kasumi-1, and Hela were obtained from Cambrex Bioproducts and American Type Culture Collection, respectively. Cells were cultured with 10% fetal bovine serum/DMEM.

The whole blood samples from healthy human volunteer were collected and the PBMCs were isolated immediately as described in Example 116. Isolated human PBMCs were cultured in 10% fetal bovine serum/DMEM.

### Treatment:

Human PBMCs, kasumi-1, My-4-11, Hela, and human renal proximal tubule epithelial cells were treated with DMSO (control), Compound 226, Compound 287, 17-DMAG at concentrations of 0, 5, 25, or 100 nM (in DMSO) for 16 hours. The cells were then collected and rinsed in ice-cold PBS and stored in liquid nitrogen until further analysis.

### Immunoblot

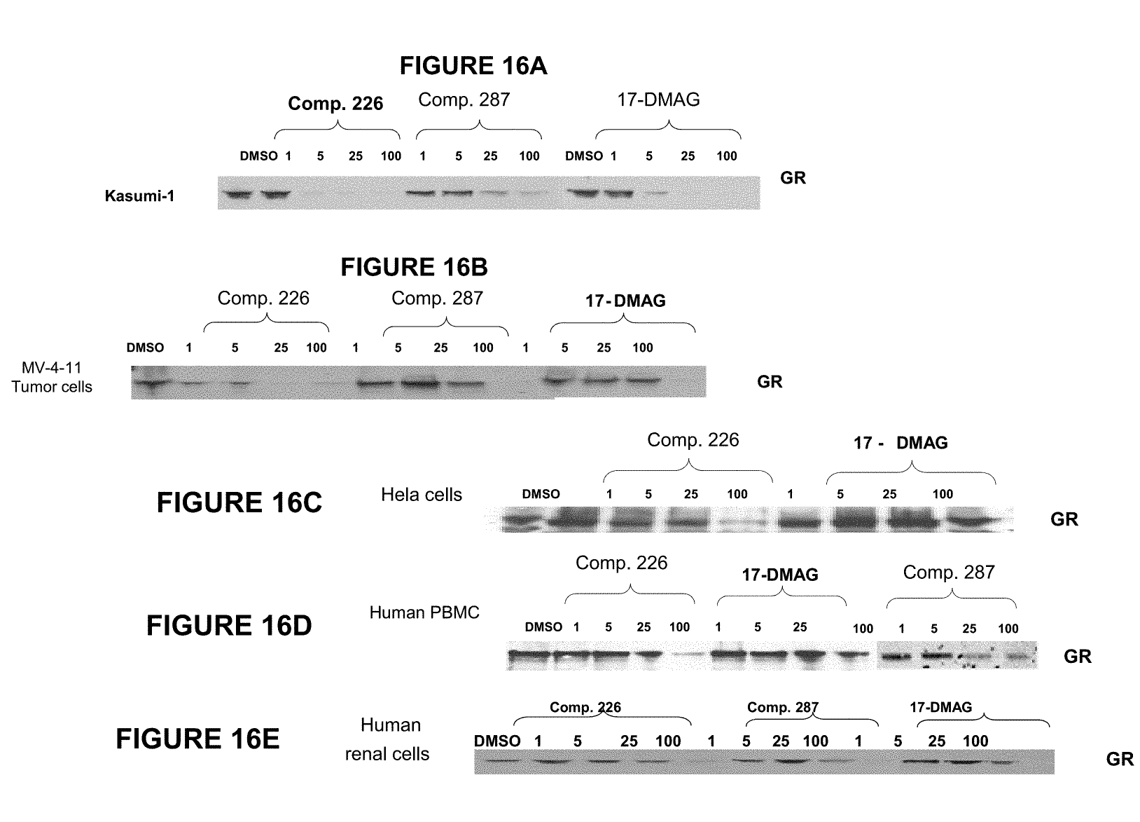

PBMC, renal and tumor cell pellets were prepared in Western lysis buffer (10 mmol/L HEPES, 42 mmol/L KCl, 5 mmol/L MgCl₂, 0.1 mmol/L EDTA, 0.1. mmol/LEGTA, 1 mmol/L DTT, 1% Triton X-100, freshly supplemented with 1× protease inhibitor cocktail from Pierce, Rockford, Ill.). Lysate protein concentrations were quantified by bicinchoninic acid assay (Pierce) and normalized. Equal amounts of protein were loaded onto 10% NuPAGE Bis-Tris Gels (Invitrogen) and subsequently transferred onto polyvinylidene difluoride membranes. The membranes were blocked in 5% milk in TBST. Primary antibody of glucocorticoid receptor from Santa Cruz Biotechnology, Inc. was added and incubated at room temperature for 1 hour with shaking. The blots were washed extensively in TBST before secondary antibodies were added for overnight incubation at 4° C. with gentle shaking. The blots were again washed extensively and developed with SuperSignal West Femto substrate (Pierce). As can be seen from FIGS. 16A-16E, Compound 226, Compound 287, and 17-DMAG suppress the expression of glucocorticoid receptors in cancer cells as well as in normal PBMCs and renal cells. The suppression of glucocorticoid receptors is more pronounced in cancer cell than in normal cells.

### Example 116

Compounds of the invention suppress glucocorticoid receptor levels in vivo.

Male adult Sprague-Dawley (SD) rats, five per group (except for group 4 which had 3 animals), were randomly assigned into five testing groups which received treatments as shown in Table 9:

TABLE 9

| Treatment | |
|---|---|
| group | Treatment received |
| G1 | 5 mL/kg of vehicle (5% DMSO/13.5% Cr-RH40/D5W) |
| G2 | 6 mg/kg of 17-DMAG |
| G3 | 5 mg/kg of Paclitaxel |
| G4 | 80 mg/kg of Compound 264 (n=3) |
| G5 | 50 mg/kg of Compound 226 |

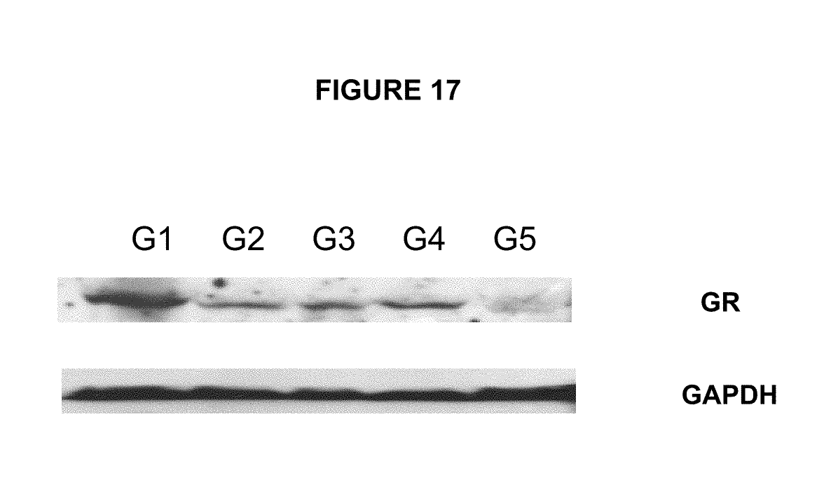

The test compounds were administered daily intravenously via tail vein for four days. There was no animal death in, the study. All rats were sacrificed at the study day 5. About 1-2 mL of blood samples were collected per animal. The blood samples were then pulled together as a group for PBMC isolation. PBMCs were isolated and an immunoblot (see FIG. 17) using an antibody that recognizes the glucocorticoid receptor was prepared, as described in Examples 114 and 115.

The immunoblot shown in FIG. 17 shows that when compared with vehicle treat animals, the expressions on glucocorticoid receptor (GR) in isolated PBMC was decreased in animals treated with 17-DMAG, Paclitaxel, Compound 264, and Compound 226. However, the decreases were most significant in the animals treated with Compound 226.

All publications, patent applications, patents, and other documents cited herein are incorporated by reference in their entirety. In case of conflict, the present specification, including definitions, will control. In addition, the materials, methods, and examples are illustrative only and not intended to be limiting.

1. A method of modulating the activity of glucocorticoid receptors in a cell, comprising administering to the cell an effective amount of an Hsp90 inhibitor represented by formula (I): 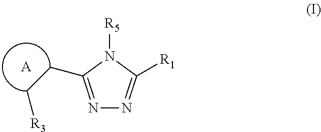
or a tautomer, a pharmaceutically acceptable salt, solvate, clathrate, or prodrug thereof, wherein
ring A is an aryl or a heteroaryl, wherein the aryl or the heteroaryl are optionally further substituted with one or more substituents in addition to R₃;
R₁ is —OH, —SH, —NR₇H, —OR₂₆, —SR₂₆, —NHR₂₆, —O(CH₂)ₘOH, —O(CH₂)ₘSH, —(CH₂)ₘNR₇H, —S(CH₂)ₘOH, —S(CH₂)ₘSH, —S(CH₂)ₘNR₇H, —OC(O)NR₁₀R₁₁, —C(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;
R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;
R₅ is an optionally substituted heteroaryl or an optionally substituted 8 to 14 membered aryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₂₆ is a lower alkyl;
p, for each occurrence, is, independently, 0, 1 or 2; and
m, for each occurrence, is independently, 1, 2, 3, or 4.

2.-9. (canceled)

10. The method of claim 1, wherein the cell is in a subject in need of treatment for an inflammatory disorder.

11. The method of claim 1, wherein the cell is in a subject in need of treatment for an immune disorder.

12. The method of claim 1, wherein the cell is in a subject in need of immunosuppression.

13. A method of treating an inflammatory disorder or an immune disorder in a subject, comprising administering to the subject an effective amount of an Hsp90 inhibitor represented by formula (I): 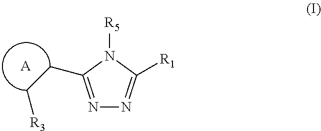
or a tautomer, a pharmaceutically acceptable salt, solvate, clathrate, or prodrug thereof, wherein:
ring A is an aryl or a heteroaryl, wherein the aryl or the heteroaryl are optionally further substituted with one or more substituents in addition to R₃;
R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —OS(O)ₚR₇, —SCH₂C(O)NR₁₀R₁₁, —NR₇CH₂C(O)NR₁₀R₁₁, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;
R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;
R₂₆ is a lower alkyl;
p, for each occurrence, is, independently, 0, 1 or 2; and
m, for each occurrence, is independently, 1, 2, 3, or 4.

14.-21. (canceled)

22. The method of claim 13, wherein the inflammatory disorder is selected from the group consisting of transplant rejection, skin graft rejection, arthritis, rheumatoid arthritis, osteoarthritis and bone diseases associated with increased bone resorption; inflammatory bowel disease, ileitis, ulcerative colitis, Barrett's syndrome, Crohn's disease; asthma, adult respiratory distress syndrome, chronic obstructive airway disease; corneal dystrophy, trachoma, onchocerciasis, uveitis, sympathetic ophthalmitis, endophthalmitis; gingivitis, periodontitis; tuberculosis; leprosy; uremic complications, glomerulonephritis, nephrosis; sclerodermatitis, psoriasis, eczema; chronic demyelinating diseases of the nervous system, multiple sclerosis, AIDS-related neurodegeneration, Alzheimer's disease, infectious meningitis, encephalomyelitis, Parkinson's disease, Huntington's disease, amyotrophic lateral sclerosis viral or autoimmune encephalitis; autoimmune disorders, immune-complex vasculitis, systemic lupus and erythematodes; systemic lupus erythematosus (SLE); cardiomyopathy, ischemic heart disease hypercholesterolemia, atherosclerosis, preeclampsia; chronic liver failure, brain and spinal cord trauma.

23. The method of claim 22, wherein the compound or pharmaceutically acceptable salt is administered with an additional therapeutic agent.

24. The method of claim 23, wherein the additional therapeutic agent is an anti-inflammatory agent.

25.-33. (canceled)

34. The method of claim 13, wherein the immune disorder is selected from the group consisting of multiple sclerosis, myasthenia gravis, Guillain-Barré, autoimmune uveitis, autoimmune hemolytic anemia, pernicious anemia, autoimmune thrombocytopenia, autoimmune lymphoproliferative syndrome (ALPS), temporal arteritis, anti-phospholipid syndrome, vasculitides such as Wegener's granulomatosis, Behcet's disease, psoriasis, dermatitis herpetiformis, pemphigus vulgaris, vitiligo, Crohn's disease, ulcerative colitis, primary biliary cirrhosis, autoimmune hepatitis, Type 1 or immune-mediated diabetes mellitus, Grave's disease. Hashimoto's thyroiditis, autoimmune oophoritis and orchitis, autoimmune disorder of the adrenal gland, rheumatoid arthritis, systemic lupus erythematosus, scleroderma, polymyositis, dermatomyositis, ankylosing spondylitis, and Sjogren's syndrome.

35. The method of claim 34, wherein the compound or pharmaceutically acceptable salt is administered with an additional therapeutic agent.

36. A method of suppressing the immune system in a subject in need thereof, comprising administering to the subject an effective amount of an Hsp90 inhibitor represented by formula (I): 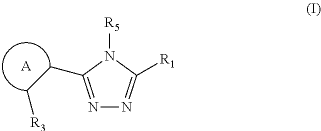
or a tautomer, a pharmaceutically acceptable salt, solvate, clathrate, or prodrug thereof, wherein:
ring A is an aryl or a heteroaryl, wherein the aryl or the heteroaryl are optionally further substituted with one or more substituents in addition to R₃;
R₁ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —OS(O)ₚR₇, —SCH₂C(O)NR₁₀R₁₁, —NR₇CH₂C(O)NR₁₀R₁₁, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;
R₃ is —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —C(O)OH, —C(O)NHR₈, —C(O)SH, —S(O)OH, —S(O)₂OH, —S(O)NHR₈, —S(O)₂NHR₈, —OP(O)(OR₇)₂, or —SP(O)(OR₇)₂;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₈ is an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;
R₂₆ is a lower alkyl;
p, for each occurrence, is, independently, 0, 1 or 2; and
m, for each occurrence, is independently, 1, 2, 3, or 4.

37.-44. (canceled)

45. The method of claim 36, wherein the subject is a transplant recipient.

46. The method of claim 45, wherein a transplant for the transplant recipient is an allograft transplant, a xenograft transplant or a syngeneic transplant.

47.-48. (canceled)

49. The method of claim 45, wherein the transplant is a skin graft, heart, kidney, lung, liver, pancreas, cornea, bowel, or stomach.

50. The method of claim 36, wherein the subject is a stem cell recipient.

51. The method of claim 36, wherein the compound or pharmaceutically acceptable salt is administered with an additional therapeutic agent.

52.-62. (canceled)

63. The method of claim 1, wherein the compound of formula I is 3-(2,4-dihydroxy-5-isopropyl-phenyl)-4-(1-methyl-indol-5-yl)-5-hydroxy-[1,2,4]triazole, or a pharmaceutically acceptable salt thereof.

64. The method of claim 13, wherein the compound of formula I is 3-(2,4-dihydroxy-5-isopropyl-phenyl)-4-(1-methyl-indol-5-yl)-5-hydroxy-[1,2,4]triazole, or a pharmaceutically acceptable salt thereof.

65. The method of claim 36, wherein the compound of formula I is 3-(2,4-dihydroxy-5-isopropyl-phenyl)-4-(1-methyl-indol-5-yl)-5-hydroxy-[1,2,4]triazole, or a pharmaceutically acceptable salt thereof.
